(19) United States
(12) Patent Application Publication
(10) Pub. No.: US 2020/0121681 A1
(43) Pub. Date: Apr. 23, 2020

(54) METHODS FOR HAIR FOLLICLE STEM CELL PROLIFERATION

(71) Applicant: Frequency Therapeutics, Inc., Woburn, MA (US)

(72) Inventors: Christopher LOOSE, Winchester, MA (US); Bradley TAIT, North Andover, MA (US); Rajesh MANCHANDA, Acton, MA (US); Will MCLEAN, North Haven, CT (US); Megan HARRISON, Middletown, CT (US); Sara STRECKER, Wethersfield, CT (US)

(21) Appl. No.: 16/656,007

(22) Filed: Oct. 17, 2019

Related U.S. Application Data
(60) Provisional application No. 62/746,806, filed on Oct. 17, 2018.

(51) Int. Cl. A61K 31/506; A61K 31/5377; A61K 31/4436; A61K 31/575; A61P 17/14

(52) U.S. Cl. CPC A61K 31/506; A61K 31/5377; A61K 45/06; A61K 31/575; A61P 17/14; A61K 31/4436

(57) ABSTRACT

The present invention relates to compositions of minoxidil and Sonic Hedgehog (Shh) pathway activators and optionally, Wnt agonists and methods of using them to induce self-renewal of hair follicle stem cells, including inducing the hair follicle stem cells to proliferate while maintaining, in the daughter cells, the capacity to differentiate into hair follicle epithelial cells.

### CROSS-REFERENCE TO RELATED APPLICATIONS

This application claims priority to, and benefit of, U.S. Provisional Application No. 62/746,806 filed on Oct. 17, 2018. The contents of this application is incorporated by reference herein, in its entirety and for all purposes.

### FIELD OF THE INVENTION

The present invention relates to methods of using minoxidil and one or more sonic hedgehog (Shh) pathway activator(s) and optionally one or more Wnt pathway activator(s) for inducing, promoting, or enhancing the growth, proliferation, and/or regeneration of stem cells in hair follicles.

### BACKGROUND OF THE INVENTION

Stem cells exhibit an extraordinary ability to generate multiple cell types in the body. Besides embryonic stem cells, tissue specific stem cells serve a critical role during development as well as in homeostasis and injury repair in the adult. Stem cells renew themselves through proliferation as well as generate tissue specific cell types through differentiation. The characteristics of different stem cells vary from tissue to tissue, and are determined by their intrinsic genetic and epigenetic status. However, the balance between self-renewal and differentiation of different stem cells are all stringently controlled. Uncontrolled self-renewal may lead to overgrowth of stem cells and possibly tumor formation, while uncontrolled differentiation may exhaust the stem cell pool, leading to an impaired ability to sustain tissue homeostasis. Thus, stem cells continuously sense their environment and appropriately respond with proliferation, differentiation or apoptosis. It would be desirable to drive regeneration by controlling the timing and extent of stem cell proliferation and differentiation. Controlling the proliferation with small molecules that are cleared over time would allow for control of the timing and extent of stem cell proliferation and differentiation. Remarkably, tissue stem cells from different tissues share a limited number of signaling pathways for the regulation of their self-renewal and differentiation, albeit in a very context dependent manner. Some of these pathways are the Wnt and GSK3 pathways.

Hair loss, e.g. alopecia, is a disorder caused by an interruption in the body's cycle of hair production. Hair loss can occur anywhere on the body, but most commonly effects the scalp. On average, the scalp has 100,000 hairs that cycle through periods of growing, resting, falling out, and regenerating. Although not a life-threatening condition, and primarily a ‘cosmetic’ issue, hair loss affects quality of life. In an image-oriented society, hair loss has a significant impact on an individual's emotional state. Hair loss may be linked to a person's genetics, although many medical and behavioral conditions may interrupt the growth cycle and cause hair loss.

There is no curative therapy for hair loss. Current treatments involve topical medications such as the antihypertensive vasodilator minoxidil or immunosuppressive steroid cream. Corticosteroid therapy can also be administered by injections. Oral prescription medications such as finasteride, antiandrogens and antifungal medications are also used. However, the treatments can be accompanied by adverse effects of the medications and patients can relapse if treatment is discontinued. Hair loss can also be treated cosmetically by dermatography and hairpieces, but these cosmetic solutions do not offer a cure either.

Thus, there remains a long felt need for new compounds that can preserve/promote the function of cells in the hair follicle to combat hair loss.

### SUMMARY OF THE INVENTION

The present disclosure provides methods of using minoxidil and one or more Sonic Hedgehog (Shh) pathway activators and optionally one or more Wnt agonists(s) to induce the self-renewal of stem cells in hair follicles. The present disclosure also provides pharmaceutical compositions of minoxidil and one or more Shh pathway activator(s) and optionally one or more Wnt agonists(s). These compositions are useful, for example, in treating diseases associated with hair loss, such as alopecia.

In one aspect, the present disclosure provides a method of expanding a population of stem cells of hair follicles, said method comprising contacting the stem cells with minoxidil and one or more Shh pathway activator(s) and optionally one or more Wnt agonists(s)

In another aspect, the present disclosure provides a method of facilitating the generation of hair follicle epithelial cells, the method comprising treating stem cells of hair follicles with minoxidil and one or more Shh pathway activator(s) and optionally one or more Wnt agonists(s).

In one embodiment, the stem cells are dermal papilla stem cells. In another embodiment, the stem cells are hair follicle stem cells. In some embodiments, the stem cells comprise keratinocytes, melanocytes, dermal papilla cells, bulge cells, or a combination thereof. In some embodiments, the stem cells are in a subject. In some embodiments of the methods described herein, the expression of Gli1, Krt15, CD34, Lgr5, Lgr6, Lrig1, Sox2, CD133, Vimentin, Versican and/or alkaline phosphatase is increased in hair follicles.

In another aspect, the present disclosure provides a method of treating a subject who has, or is at risk of developing, a disease associated with absence or lack of hair follicle epithelial cells, the method comprising administering to said subject minoxidil and one or more Shh pathway activator(s) and optionally, one or more Wnt agonists(s). In some embodiments, the disease is selected from telogen effluvium, anagen effluvium, androgenetic alopecia, alopecia areata, tinea capitis, lichen planopilaris, cicatricial alopecia, discoid lupus erythematosus, folliculitis decalvans, dissecting cellulitis of the scalp, frontal fibrosing alopecia, central centrifugal cicatricial alopecia, trichotillomania, traction alopecia, and hypotrichosis.

In another aspect, the present disclosure provides a method of treating a subject who has, or is at risk of developing, alopecia, the method comprising administering to said subject minoxidl and one or more Shh pathway activator(s) and optionally, one or more Wnt agonists(s).

In some embodiments, the subject administered the minoxidil and one or more Shh pathway activator(s) has improved hair growth, improved hair density and/or improved regenerative cycling of hair follicles compared to a subject not administered minoxidil and the one or more Shh pathway activator(s).

In yet another aspect, the present disclosure provides a pharmaceutical composition comprising: a pharmaceutically-acceptable carrier and (i) minoxidil or a pharmaceutically-acceptable salt thereof, and (ii) a Sonic Hedgehog (Shh) pathway activator, or a pharmaceutically-acceptable salt thereof. Optionally, the pharmaceutical composition further includes a Wnt agonist, or a pharmaceutically-acceptable salt thereof.

In some embodiments of the methods and compositions disclosed herein, minoxidil is at a concentration of about 0.001× to about 10× of an FDA approved minoxidil concentration. In other embodiments of the methods and compositions disclosed herein, minoxidil is at a concentration of about 0.01× to about 5× of an FDA approved minoxidil concentration. In some embodiments of the methods and compositions disclosed herein, minoxidil is at a concentration of about 0.1× to about 1× of an FDA approved minoxidil concentration.

In some embodiments of the methods and compositions disclosed herein, the one or more Shh pathway activator(s) is at a concentration of about 5× to about 1000× of an effective in vitro Shh pathway activation concentration. In certain embodiments, the one or more Shh pathway activator(s) is at a concentration of about 10× to about 100× of an effective in vitro Shh pathway activation concentration. In some embodiments, the one or more Shh pathway activator(s) is at a concentration of about 20× to about 50× of an effective in vitro Shh pathway activation concentration. In certain embodiments, the one or more Wnt agonists(s) is at a concentration of about 5× to about 1000× of an effective in vitro Wnt agonist concentration. In some embodiments, the one or more Wnt agonists(s) is at a concentration of about 10× to about 100× of an effective in vitro Wnt agonist concentration. In some embodiments, the one or more Wnt agonists(s) is at a concentration of about 20× to about 50× of an effective in vitro Wnt agonist concentration.

In some embodiments of the methods and compositions disclosed herein, the Shh pathway activator comprises a Smoothened agonist. In other embodiments, the Shh pathway activator comprises Smoothened ciliary accumulation enhancers. In certain embodiments of the methods and compositions disclosed herein, the one or more Shh pathway activator(s) is selected from Table 1 or Table 2. In further embodiments of the methods and compositions disclosed herein, the one or more Shh pathway activator(s) is selected from Purmorphamine, SAG, 20-alpha hydroxy cholesterol, and SAG HCl.

In some embodiments of the methods and compositions disclosed herein, the one or more Wnt agonists(s) is selected from Table 3. In certain embodiments of the methods and compositions disclosed herein, the one or more Wnt agonists(s) is a GSK3-alpha inhibitor or a GSK3-beta inhibitor. In further embodiments of the methods and compositions disclosed herein, the GSK3-alpha inhibitor is selected from Table 5. In yet further embodiments of the methods and compositions disclosed herein, the GSK3-beta inhibitor is selected from Table 4.

In some embodiments of the methods and compositions disclosed herein, the one or more Wnt agonists(s) is a compound of Formula (I),

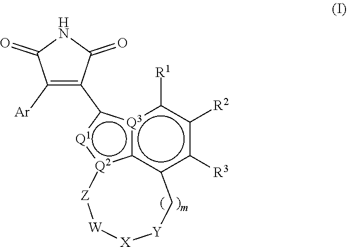

and pharmaceutically acceptable salts and tautomers thereof, wherein:

Q¹ is CH or N;

Q² is C or N;

Q³ is C or N;
wherein at least one of Q¹, Q², and Q³ is N;

R¹ is selected from the group consisting of hydrogen, halo, C₁-C₄alkyl, C₁-C₄alkenyl, C₁-C₄alkynyl, —CN, —OH, —O—C₁-C₄alkyl, —NH₂, —NHC(O)R^{1a}, and —S(O)₂NH₂; wherein the alkyl is optionally substituted with one to 3 substituents independently selected from the group consisting of halo and —OH; and wherein R^{1a} is C₁-C₄alkyl;

R² is selected from the group consisting of halo, C₁-C₄alkyl, C₁-C₄alkenyl, C₁-C₄alkynyl, —CN, —OH, —O—C₁-C₄alkyl, —NH₂, —NH(C₁-C₄alkyl), —N(C₁-C₄alkyl)₂, —NHC(O)R^{2a}, and —S(O)₂NH₂; wherein the alkyl is optionally substituted with one to 3 substituents independently selected from the group consisting of halo and —OH; and wherein R^{2a} is C₁-C₄alkyl;

R³ is selected from the group consisting of hydrogen, halo, C₁-C₄alkyl, C₁-C₄alkenyl, C₁-C₄alkynyl, —CN, —OH, —O—C₁-C₄alkyl, —NH₂, —NHC(O)R^{3a}, and —S(O)₂NH₂; wherein the alkyl is optionally substituted with one to 3 substituents independently selected from the group consisting of halo and —OH; and wherein R^{3a} is C₁-C₄alkyl;

Ar is selected from the group consisting of

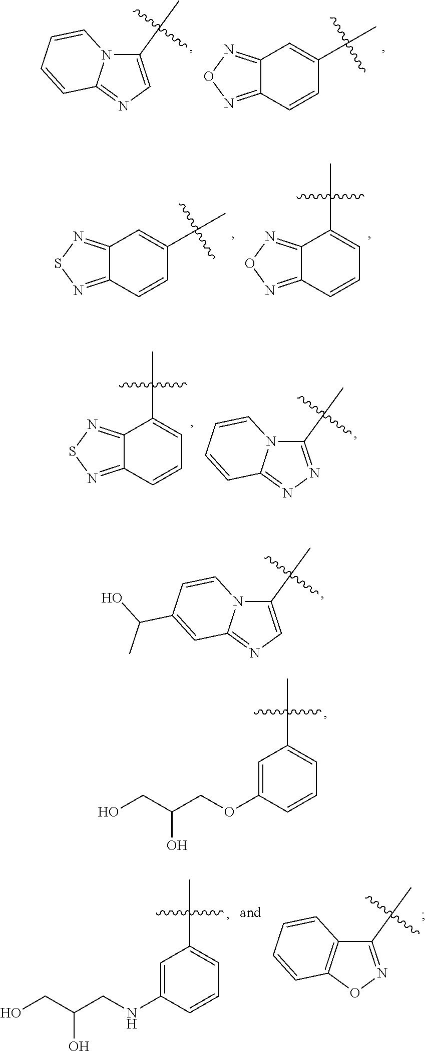

—Z—W—X—Y— is —C(R^{Z})₂—C(R^{W})₂—N(R^{X})—C(R^{Y})₂—, —C(R^{Z})₂—C(R^{W})₂—CH(R^{X})—C(R^{Y})₂—, or —C(R^{W})₂—CH(R^{X})—C(R^{Y})₂—;

each R^{Z} is independently selected from the group consisting of hydrogen, deuterium, halo, and C₁-C₄alkyl, or both R^{Z} groups together form C₃-C₆cycloalkyl or oxo;

each R^{W} is independently selected from the group consisting of hydrogen, deuterium, halo, and C₁-C₄alkyl, or both R^{W} groups together form C₃-C₆cycloalkyl or oxo;

or R^{Z} and R^{W} together with the carbons to which they are attached form a C₃-C₆cycloalkyl;

R^{X} is selected from the group consisting of —COR^{X1}, —SO₂R^{X1}, heteroaryl, and —(C₁-C₄alkylene)-(C₃-C₈cycloalkyl), and wherein the —(C₁-C₄alkylene)-(C₃-C₈cycloalkyl) is optionally substituted with one to four halo on the C₁-C₄alkylene;

wherein R^{X1} is heterocyclic, wherein the heterocyclic is optionally substituted with one to twelve substituents independently selected from the group consisting of deuterium, halo, —[C(R^{X1a})₂]ₚ—CN, —CF₃, C₁-C₄alkyl, —(CH₂)ₚ—OH, —[C(R^{X1a})₂]ₚ—OH, —[C(R^{X1a})₂]ₚ—O—C₁-C₄alkyl, —NHCOC₁-C₄alkyl, —CONHC₁-C₄alkyl, —COH, —CO₂H, —[C(R^{X1a})₂]ₚ—COO—C₁-C₄alkyl, —(CH₂)ₚ—NH₂, —[C(R^{X1a})₂]ₚ—NH₂, —[C(R^{X1a})₂]ₚ—NH—C₁-C₄alkyl, —[C(R^{X1a})₂]ₚ—N—(C₁-C₄alkyl)₂; wherein p is 0, 1, 2, or 3; wherein each R^{X1a} is independently selected from the group consisting of hydrogen, deuterium, halo, and C₁-C₄alkyl, or both R^{X1a} groups together form C₃-C₆cycloalkyl;

each R^{Y} is independently selected from the group consisting of hydrogen, deuterium, halo, and C₁-C₄alkyl, or both R^{Y} groups together form C₃-C₆cycloalkyl or oxo; and

m is 0, 1, or 2.

In some embodiments of the methods and compositions disclosed herein, the compounds of Formula I have one or more of the following features:

a) provided that the compound is not

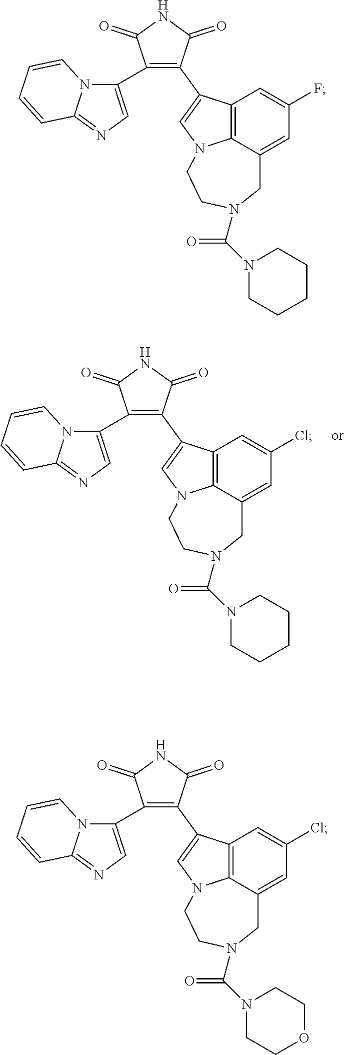 [SMILES: O=C1NC(=O)C(c2cnc3ccccn23)=C1c1cn2c3c(cc(F)cc13)CN(C(=O)N1CCCCC1)CC2 ; O=C1NC(=O)C(c2cnc3ccccn23)=C1c1cn2c3c(cc(Cl)cc13)CN(C(=O)N1CCCCC1)CC2 ; O=C1NC(=O)C(c2cnc3ccccn23)=C1c1cn2c3c(cc(Cl)cc13)CN(C(=O)N1CCOCC1)CC2]

b) provided that when Ar is

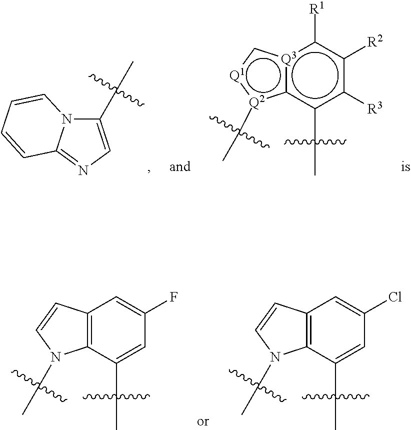

then R^{X1} is not

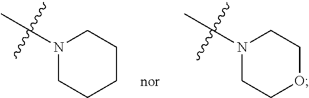 [SMILES: *N1CCCCC1 ; *N1CCOCC1]

In some embodiments of the methods and compositions disclosed herein, the one or more Wnt agonists(s) is a compound of Formula Ia:

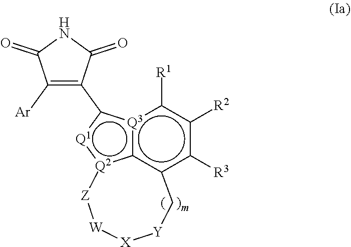

and pharmaceutically acceptable salts and tautomers thereof, wherein:

Q¹ is CH or N;

Q² is C or N;

Q³ is C or N;
wherein at least one of Q¹, Q², and Q³ is N;

R¹ is selected from the group consisting of hydrogen, halo, C₁-C₄alkyl, C₁-C₄alkenyl, C₁-C₄alkynyl, —CN, —OH, —O—C₁-C₄alkyl, —NH₂, —NHC(O)R^{3a}, and —S(O)₂NH₂; wherein the alkyl is optionally substituted with one to 3 substituents independently selected from the group consisting of halo and —OH; and wherein R^{1a} is C₁-C₄alkyl;

R² is selected from the group consisting of hydrogen, halo, C₁-C₄alkyl, C₁-C₄alkenyl, C₁-C₄alkynyl, —CN, —OH, —O—C₁-C₄alkyl, —NH₂, —NH(C₁-C₄alkyl), —N(C₁-C₄alkyl)₂, —NHC(O)R^{2a}, and —S(O)₂NH₂; wherein the alkyl is optionally substituted with one to 3 substituents independently selected from the group consisting of halo and —OH; and wherein R^{2a} is C₁-C₄alkyl;

R³ is selected from the group consisting of hydrogen, halo, C₁-C₄alkyl, C₁-C₄alkenyl, C₁-C₄alkynyl, —CN, —OH, —O—C₁-C₄alkyl, —NH₂, —NHC(O)R^{3a}, and —S(O)₂NH₂; wherein the alkyl is optionally substituted with one to 3 substituents independently selected from the group consisting of halo and —OH; and wherein R^{3a} is C₁-C₄alkyl;

Ar is selected from the group consisting of

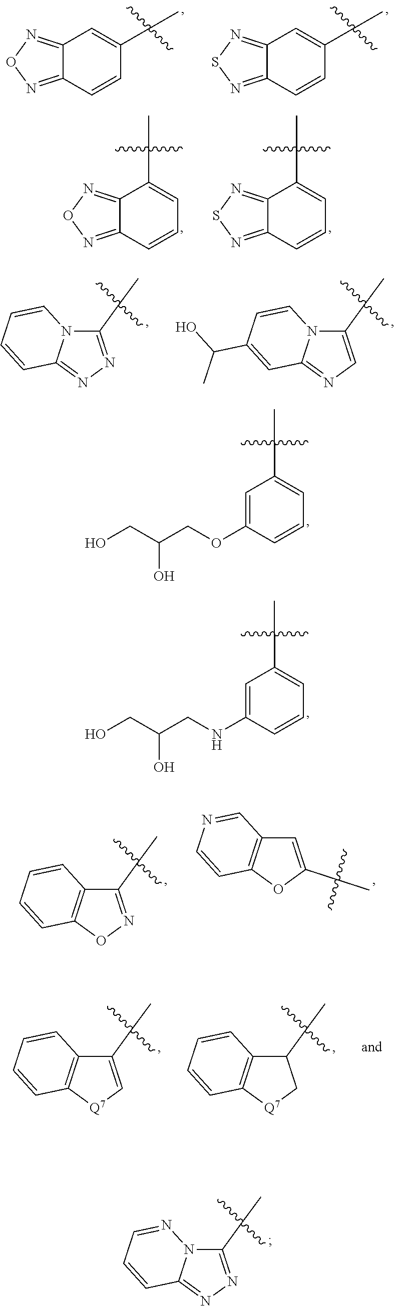

wherein Ar is optionally substituted with deuterium, halo, alkyl, alkoxy, and CN;

Q⁷ is selected from S, O, CH₂, and NR^{Q7}; wherein R^{Q7} is hydrogen or optionally substituted C₁-C₄alkyl;

—Z—W—X—Y— is —C(R^{Z})₂—C(R^{W})₂—N(R^{X})—C(R^{Y})₂—, —C(R^{Z})₂—C(R^{W})₂—CH(R^{X})—C(R^{Y})₂—, or —C(R^{W})₂—CH(R^{X})—C(R^{Y})₂—;

each R^{Z} is independently selected from the group consisting of hydrogen, deuterium, halo, and C₁-C₄alkyl, or both R^{Z} groups together form C₃-C₆cycloalkyl or oxo;

each R^{W} is independently selected from the group consisting of hydrogen, deuterium, halo, and C₁-C₄alkyl, or both R^{W} groups together form C₃-C₆cycloalkyl or oxo;

or R^{Z} and R^{W} together with the carbons to which they are attached form a C₃-C₆cycloalkyl;

R^{X} is selected from the group consisting of hydrogen, R^{X1}, —COR^{X1}, —SO₂R^{X1}, —(C₁-C₄alkylene)-R^{X1}, and wherein the —(C₁-C₄alkylene)-R^{X1} is optionally substituted with one to four halo on the C₁-C₄alkylene;

wherein R^{X1} is C₃-C₈cycloalkyl, heteroaryl, or heterocyclic, wherein the heterocyclic is optionally substituted with one to twelve substituents independently selected from the group consisting of deuterium, halo, —[C(R^{X1a})₂]ₚ—CN, —CF₃, —C₁-C₄alkyl, —(CH₂)ₚ—OH, —[C(R^{X1a})₂]ₚ—OH, —[C(R^{X1a})₂]ₚ—O—C₁-C₄alkyl, NHCOC₁-C₄alkyl, CONHC₁-C₄alkyl, COH, —CO₂H, —[C(R^{X1a})₂]ₚ—COO—C₁-C₄alkyl, —(CH₂)ₚ—NH₂, —[C(R^{X1a})₂]ₚ—NH₂, —[C(R^{X1a})₂]ₚ—NH—C₁-C₄alkyl, —[C(R^{X1a})₂]ₚ—N—(C₁-C₄alkyl)₂; wherein p is 0, 1, 2, or 3; wherein each R^{X1}a is independently selected from the group consisting of hydrogen, deuterium, halo, and C₁-C₄alkyl, or both R^{X1a} groups together form C₃-C₆cycloalkyl;

each R^{Y} is independently selected from the group consisting of hydrogen, deuterium, halo, and C₁-C₄alkyl, or both R^{Y} groups together form C₃-C₆cycloalkyl or oxo; and

m is 0, 1, or 2.

In some embodiments of the methods and compositions disclosed herein, the one or more Wnt agonists(s) is a compound of Formula Ib:

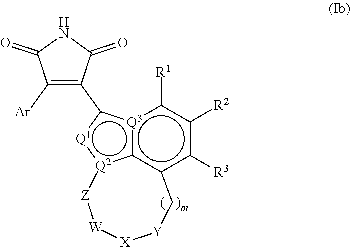

and pharmaceutically acceptable salts and tautomers thereof, wherein:

Q¹ is CH or N;

Q² is C or N;

Q³ is C or N;

wherein at least one of Q¹, Q², and Q³ is N; and provided that when Q¹ is CH and Q³ is C, Q² is not N;

R¹ is selected from the group consisting of hydrogen, halo, C₁-C₄alkyl, C₁-C₄alkenyl, C₁-C₄alkynyl, —CN, —OH, —O—C₁-C₄alkyl, —NH₂, —NHC(O)R^{3a}, and —S(O)₂NH₂; wherein the alkyl is optionally substituted with one to 3 substituents independently selected from the group consisting of halo and —OH; and wherein R^{1a} is C₁-C₄alkyl;

R² is selected from the group consisting of hydrogen, halo, C₁-C₄alkyl, C₁-C₄alkenyl, C₁-C₄alkynyl, —CN, —OH, —O—C₁-C₄alkyl, —NH₂, —NH(C₁-C₄alkyl), —N(C₁-C₄alkyl)₂, —NHC(O)R^{2a}, and —S(O)₂NH₂; wherein the alkyl is optionally substituted with one to 3 substituents independently selected from the group consisting of halo and —OH; and wherein R^{2a} is C₁-C₄alkyl;

R³ is selected from the group consisting of hydrogen, halo, C₁-C₄alkyl, C₁-C₄alkenyl, C₁-C₄alkynyl, —CN, —OH, —O—C₁-C₄alkyl, —NH₂, —NHC(O)R^{3a}, and —S(O)₂NH₂; wherein the alkyl is optionally substituted with one to 3 substituents independently selected from the group consisting of halo and —OH; and wherein R^{3a} is C₁-C₄alkyl;

Ar is selected from the group consisting of

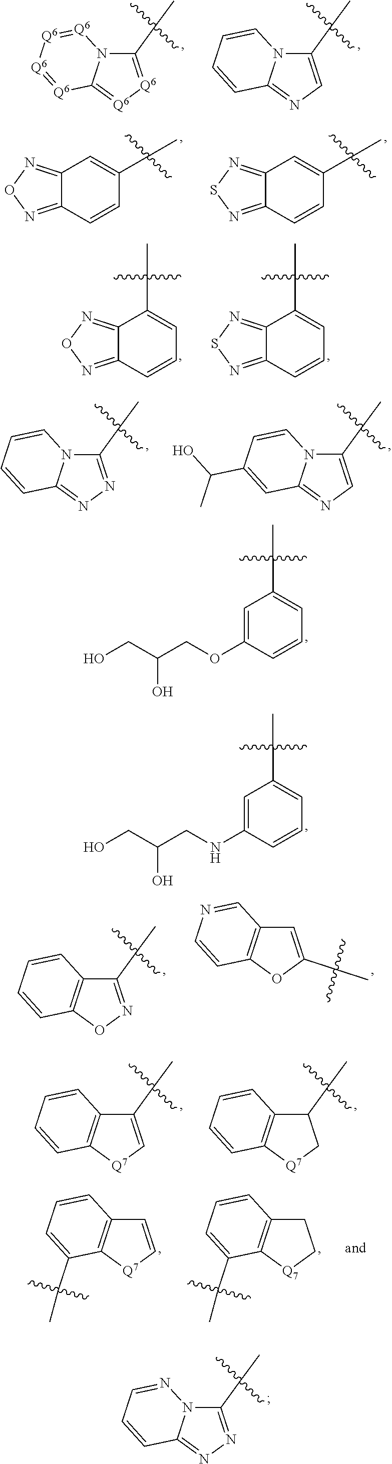

wherein Ar is optionally substituted with deuterium, halo, alkyl, alkoxy, and CN;

each Q⁶ is independently selected from CR^{Q6} and N; wherein R^{Q6} is hydrogen, halo, —CN, lower alkyl, or substituted alkyl;

Q⁷ is selected from S, O, CH₂, and NR^{Q7}; wherein R^{Q7} is hydrogen or optionally substituted C₁-C₄alkyl;

—Z—W—X—Y— is —C(R^{Z})₂—C(R^{W})₂—N(R^{X})—C(R^{Y})₂—, —C(R^{Z})₂—C(R^{W})₂—CH(R^{X})—C(R^{Y})₂—, or —C(R^{W})₂—CH(R^{X})—C(R^{Y})₂—;

each R^{Z} is independently selected from the group consisting of hydrogen, deuterium, halo, and C₁-C₄alkyl, or both R^{Z} groups together form C₃-C₆cycloalkyl or oxo;

each R^{W} is independently selected from the group consisting of hydrogen, deuterium, halo, and C₁-C₄alkyl, or both R^{W} groups together form C₃-C₆cycloalkyl or oxo;

or R^{Z} and R^{W} together with the carbons to which they are attached form a C₃-C₆cycloalkyl;

R^{X} is selected from the group consisting of hydrogen, R^{X1}, —COR^{X1}, —SO₂R^{X1}, —(C₁-C₄alkylene)-R^{X1}, and wherein the —(C₁-C₄alkylene)-R^{X1} is optionally substituted with one to four halo on the C₁-C₄alkylene;

wherein R^{X1} is C₃-C₈cycloalkyl, heteroaryl, or heterocyclic, wherein the heterocyclic is optionally substituted with one to twelve substituents independently selected from the group consisting of deuterium, halo, —[C(R^{X1a})₂]ₚ—CN, —CF₃, C₁-C₄alkyl, —(CH₂)ₚ—OH, —[C(R^{X1a})₂]ₚ—OH, —[C(R^{X1a})₂]ₚ—O—C₁-C₄alkyl, —NHCOC₁-C₄alkyl, CONHC₁-C₄alkyl, COH, —CO₂H, —[C(R^{X1a})₂]ₚ—COO—C₁-C₄alkyl, —(CH₂)ₚ—NH₂, —[C(R^{X1a})₂]ₚ—NH₂, —[C(R^{X1a})₂]ₚ—NH—C₁-C₄alkyl, —[C(R^{1a})₂]ₚ—N—(C₁-C₄alkyl)₂; wherein p is 0, 1, 2, or 3; wherein each R^{X1a} is independently selected from the group consisting of hydrogen, deuterium, halo, and C₁-C₄alkyl, or both R^{X1a} groups together form C₃-C₆cycloalkyl;

each R^{Y} is independently selected from the group consisting of hydrogen, deuterium, halo, and C₁-C₄alkyl, or both R^{Y} groups together form C₃-C₆cycloalkyl or oxo; and

m is 0, 1, or 2.

In some embodiments of the methods and compositions disclosed herein, the one or more Wnt agonists(s) is selected from Table 6. In some embodiments of the methods and compositions disclosed herein, the one or more Wnt agonists(s) is selected from CHIR99021, LY2090314, AZD1080, GSK3 inhibitor XXII, Compound I-6, Compound I-7, and Compound I-12.

In some embodiments of the methods and compositions disclosed herein, the one or more Wnt agonists(s) is selected from CHIR99021, LY2090314, AZD1080, GSK3 inhibitor XXII, Compound I-6, Compound I-7, and Compound I-12 and the one or more Shh pathway activator(s) is selected from Purmorphamine, SAG, 20-alpha hydroxy cholesterol, and SAG HCl.

In some embodiments of the methods and compositions disclosed herein, the one or more Shh pathway activator(s) is Purmorphamine. In some embodiments, minoxidil is at a concentration of about 10 mM to about 250 mM and Purmorphamine is at a concentration of about 100 nM to about 10 μM. In certain embodiments, minoxidil is at a concentration of about 10 mM to about 250 mM and Purmorphamine is at a concentration of about 100 μM to about 10 mM.

In some embodiments of the methods and compositions disclosed herein, the one or more Shh pathway activator(s) is SAG. In some embodiments, minoxidil is at a concentration of about 10 mM to about 250 mM and SAG is at a concentration of about 1 nM to about 100 nM. In certain embodiments, minoxidil is at a concentration of about 10 mM to about 250 mM and SAG is at a concentration of about 1 μM to about 100 μM.

In some embodiments of the methods and compositions disclosed herein, the one or more Shh pathway activator(s) is 20-alpha hydroxy cholesterol. In some embodiments, minoxidil is at a concentration of about 10 mM to about 250 mM and 20-alpha hydroxy cholesterol is at a concentration of about 1 μM to about 100 μM. In certain embodiments, minoxidil is at a concentration of about 10 mM to about 250 mM and 20-alpha hydroxy cholesterol is at a concentration of about 1 mM to about 100 mM.

In some embodiments of the methods and compositions disclosed herein, the one or more Shh pathway activator(s) is SAG HCl. In some embodiments minoxidilis at a concentration of about 10 mM to about 250 mM and SAG HCl is at a concentration of about 10 nM to about 1 μM. In certain embodiments, minoxidil is at a concentration of about 10 mM to about 250 mM and SAG HCl is at a concentration of about 10 μM to about 1 mM.

In some embodiments of the methods and compositions disclosed herein, the one or more Wnt agonists(s) is CHIR99021 and the one or more Shh pathway activator(s) is Purmorphamine. In some embodiments, minoxidil is at a concentration of about 10 mM to about 250 mM, CHIR99021 is at a concentration of about 100 nM to about 10 μM and Purmorphamine is at a concentration of about 100 nM to about 10 μM. In certain embodiments, minoxidil is at a concentration of about 10 mM to about 250 mM, CHIR99021 is at a concentration of about 100 μM to about 10 mM and Purmorphamine is at a concentration of about 100 μM to about 10 mM.

In some embodiments of the methods and compositions disclosed herein, the one or more Wnt agonists(s) is CHIR99021 and the one or more Shh pathway activator(s) is SAG. In some embodiments, minoxidil is at a concentration of about 10 mM to about 250 mM, CHIR99021 is at a concentration of about 100 nM to about 10 μM and SAG is at a concentration of about 1 nM to about 100 nM. In certain embodiments, minoxidil is at a concentration of about 10 mM to about 250 mM, CHIR99021 is at a concentration of about 100 μM to about 10 mM and SAG is at a concentration of about 1 μM to about 100 μM.

In some embodiments of the methods and compositions disclosed herein, the one or more Wnt agonists(s) is CHIR99021 and the one or more Shh pathway activator(s) is 20-alpha hydroxy cholesterol. In some embodiments, minoxidil is at a concentration of about 10 mM to about 250 mM, CHIR99021 is at a concentration of about 100 nM to about 10 μM and 20-alpha hydroxy cholesterol is at a concentration of about 1 μM to about 100 μM. In certain embodiments, minoxidil is at a concentration of about 10 mM to about 250 mM, CHIR99021 is at a concentration of about 100 μM to about 10 mM and 20-alpha hydroxy cholesterol is at a concentration of about 1 mM to about 100 mM.

In some embodiments of the methods and compositions disclosed herein, the one or more Wnt agonists(s) is CHIR99021 and the one or more Shh pathway activator(s) is SAG HCl. In some embodiments, minoxidil is at a concentration of about 10 mM to about 250 mM, CHIR99021 is at a concentration of about 100 nM to about 10 μM and SAG HCl is at a concentration of about 10 nM to about 1 μM. In certain embodiments, minoxidil is at a concentration of about 10 mM to about 250 mM, CHIR99021 is at a concentration of about 100 μM to about 10 mM and SAG HCl is at a concentration of about 10 μM to about 1 mM.

In some embodiments of the methods and compositions disclosed herein, the one or more Wnt agonists(s) is LY2090314 and the one or more Shh pathway activator(s) is Purmorphamine. In some embodiments, minoxidil is at a concentration of about 10 mM to about 250 mM, LY2090314 is at a concentration of about 1 nM to about 100 nM and Purmorphamine is at a concentration of about 100 nM to about 10 μM. In certain embodiments, minoxidil is at a concentration of about 10 mM to about 250 mM, LY2090314 is at a concentration of about 1 μM to about 100 μM and Purmorphamine is at a concentration of about 100 μM to about 10 mM.

In some embodiments of the methods and compositions disclosed herein, the one or more Wnt agonists(s) is LY2090314 and the one or more Shh pathway activator(s) is SAG. In some embodiments, minoxidil is at a concentration of about 10 mM to about 250 mM, LY2090314 is at a concentration of about 1 nM to about 100 nM and SAG is at a concentration of about 1 nM to about 100 nM. In certain embodiments, minoxidil is at a concentration of about 10 mM to about 250 mM, LY2090314 is at a concentration of about 1 μM to about 100 μM and SAG is at a concentration of about 1 μM to about 100 μM.

In some embodiments of the methods and compositions disclosed herein, the one or more Wnt agonists(s) is LY2090314 and the one or more Shh pathway activator(s) is 20-alpha hydroxy cholesterol. In some embodiments, minoxidil is at a concentration of about 10 mM to about 250 mM, LY2090314 is at a concentration of about 1 nM to about 100 nM and 20-alpha hydroxy cholesterol is at a concentration of about 1 μM to about 100 μM. In certain embodiments, minoxidil is at a concentration of about 10 mM to about 250 mM, LY2090314 is at a concentration of about 1 μM to about 100 μM and 20-alpha hydroxy cholesterol is at a concentration of about 1 mM to about 100 mM.

In some embodiments of the methods and compositions disclosed herein, the one or more Wnt agonists(s) is LY2090314 and the one or more Shh pathway activator(s) is SAG HCl. In some embodiments, minoxidil is at a concentration of about 10 mM to about 250 mM, LY2090314 is at a concentration of about 1 nM to about 100 nM and SAG HCl is at a concentration of about 10 nM to about 1 μM. In certain embodiments, minoxidil is at a concentration of about 10 mM to about 250 mM, LY2090314 is at a concentration of about 1 μM to about 100 μM and SAG HCl is at a concentration of about 10 μM to about 1 mM.

In some embodiments of the methods and compositions disclosed herein, the one or more Wnt agonists(s) is AZD1080 and the one or more Shh pathway activator(s) is Purmorphamine. In some embodiments, minoxidil is at a concentration of about 10 mM to about 250 mM, AZD1080 is at a concentration of about 1 μM to about 100 μM and Purmorphamine is at a concentration of about 100 nM to about 10 μM. In certain embodiments, minoxidil is at a concentration of about 10 mM to about 250 mM, AZD1080 is at a concentration of about 1 mM to about 100 mM and Purmorphamine is at a concentration of about 100 μM to about 10 mM.

In some embodiments of the methods and compositions disclosed herein, the one or more Wnt agonists(s) is AZD1080 and the one or more Shh pathway activator(s) is SAG. In some embodiments, minoxidil is at a concentration of about 10 mM to about 250 mM, AZD1080 is at a concentration of about 1 μM to about 100 μM and SAG is at a concentration of about 1 nM to about 100 nM. In certain embodiments, minoxidil is at a concentration of about 10 mM to about 250 mM, AZD1080 is at a concentration of about 1 mM to about 100 mM and SAG is at a concentration of about 1 μM to about 100 μM.

In some embodiments of the methods and compositions disclosed herein, the one or more Wnt agonists(s) is AZD1080 and the one or more Shh pathway activator(s) is 20-alpha hydroxy cholesterol. In some embodiments, minoxidil is at a concentration of about 10 mM to about 250 mM, AZD1080 is at a concentration of about 1 μM to about 100 μM and 20-alpha hydroxy cholesterol is at a concentration of about 1 μM to about 100 μM. In certain embodiments, minoxidil is at a concentration of about 10 mM to about 250 mM, AZD1080 is at a concentration of about 1 mM to about 100 mM and 20-alpha hydroxy cholesterol is at a concentration of about 1 mM to about 100 mM.

In some embodiments of the methods and compositions disclosed herein, the one or more Wnt agonists(s) is AZD1080 and the one or more Shh pathway activator(s) is SAG HCl. In some embodiments, minoxidil is at a concentration of about 10 mM to about 250 mM, AZD1080 is at a concentration of about 1 μM to about 100 μM and SAG HCl is at a concentration of about 10 nM to about 1 μM. In certain embodiments, minoxidil is at a concentration of about 10 mM to about 250 mM, AZD1080 is at a concentration of about 1 mM to about 100 mM and SAG HCl is at a concentration of about 10 μM to about 1 mM.

In some embodiments of the methods and compositions disclosed herein, the one or more Wnt agonists(s) is GSK3 inhibitor XXII and the one or more Shh pathway activator(s) is Purmorphamine. In some embodiments, minoxidil is at a concentration of about 10 mM to about 250 mM, GSK3 inhibitor XXII is at a concentration of about 100 nM to about M and Purmorphamine is at a concentration of about 100 nM to about 10 μM. In certain embodiments, minoxidil is at a concentration of about 10 mM to about 250 mM, GSK3 inhibitor XXII is at a concentration of about 100 μM to about 10 mM and Purmorphamine is at a concentration of about 100 μM to about 10 mM.

In some embodiments of the methods and compositions disclosed herein, the one or more Wnt agonists(s) is GSK3 inhibitor XXII and the one or more Shh pathway activator(s) is SAG. In some embodiments, minoxidil is at a concentration of about 10 mM to about 250 mM, GSK3 inhibitor XXII is at a concentration of about 100 nM to about 10 μM and SAG is at a concentration of about 1 nM to about 100 nM. In certain embodiments, minoxidil is at a concentration of about 10 mM to about 250 mM, GSK3 inhibitor XXII is at a concentration of about 100 μM to about 10 mM and SAG is at a concentration of about 1 μM to about 100 μM.

In some embodiments of the methods and compositions disclosed herein, the one or more Wnt agonists(s) is GSK3 inhibitor XXII and the one or more Shh pathway activator(s) is 20-alpha hydroxy cholesterol. In some embodiments, minoxidil is at a concentration of about 10 mM to about 250 mM, GSK3 inhibitor XXII is at a concentration of about 100 nM to about 10 μM and 20-alpha hydroxy cholesterol is at a concentration of about 1 μM to about 100 μM. In certain embodiments, minoxidil is at a concentration of about 10 mM to about 250 mM, GSK3 inhibitor XXII is at a concentration of about 100 μM to about 10 mM and 20-alpha hydroxy cholesterol is at a concentration of about 1 mM to about 100 mM.

In some embodiments of the methods and compositions disclosed herein, the one or more Wnt agonists(s) is GSK3 inhibitor XXII and the one or more Shh pathway activator(s) is SAG HCl. In some embodiments, minoxidil is at a concentration of about 10 mM to about 250 mM, GSK3 inhibitor XXII is at a concentration of about 100 nM to about 10 μM and SAG HCl is at a concentration of about 10 nM to about 1 μM. In certain embodiments, minoxidil is at a concentration of about 10 mM to about 250 mM, GSK3 inhibitor XXII is at a concentration of about 100 μM to about 10 mM and SAG HCl is at a concentration of about M to about 1 mM.

In some embodiments of the methods and compositions disclosed herein, the one or more Wnt agonists(s) is Compound I-6 and the one or more Shh pathway activator(s) is Purmorphamine. In some embodiments, minoxidil is at a concentration of about 10 mM to about 250 mM, Compound I-6 is at a concentration of about 1 nM to about 100 nM and Purmorphamine is at a concentration of about 100 nM to about 10 μM. In certain embodiments, minoxidil is at a concentration of about 10 mM to about 250 mM, Compound I-6 is at a concentration of about 1 μM to about 100 μM and Purmorphamine is at a concentration of about 100 μM to about 10 mM.

In some embodiments of the methods and compositions disclosed herein, the one or more Wnt agonists(s) is Compound I-6 and the one or more Shh pathway activator(s) is SAG. In some embodiments, minoxidil is at a concentration of about 10 mM to about 250 mM, Compound I-6 is at a concentration of about 1 nM to about 100 nM and SAG is at a concentration of about 1 nM to about 100 nM. In certain embodiments, minoxidil is at a concentration of about 10 mM to about 250 mM, Compound I-6 is at a concentration of about 1 μM to about 100 μM and SAG is at a concentration of about 1 μM to about 100 μM.

In some embodiments of the methods and compositions disclosed herein, the one or more Wnt agonists(s) is Compound I-6 and the one or more Shh pathway activator(s) is 20-alpha hydroxy cholesterol. In some embodiments, minoxidil is at a concentration of about 10 mM to about 250 mM, Compound I-6 is at a concentration of about 1 nM to about 100 nM and 20-alpha hydroxy cholesterol is at a concentration of about 1 μM to about 100 μM. In certain embodiments, minoxidil is at a concentration of about 10 mM to about 250 mM, Compound I-6 is at a concentration of about 1 μM to about 100 μM and 20-alpha hydroxy cholesterol is at a concentration of about 1 mM to about 100 mM.

In some embodiments of the methods and compositions disclosed herein, the one or more Wnt agonists(s) is Compound I-6 and the one or more Shh pathway activator(s) is SAG HCl. In some embodiments, minoxidil is at a concentration of about 10 mM to about 250 mM, Compound I-6 is at a concentration of about 1 nM to about 100 nM and SAG HCl is at a concentration of about 10 nM to about 1 μM. In certain embodiments, minoxidil is at a concentration of about 10 mM to about 250 mM, Compound I-6 is at a concentration of about 1 μM to about 100 μM and SAG HCl is at a concentration of about 10 μM to about 1 mM.

In some embodiments of the methods and compositions disclosed herein, the one or more Wnt agonists(s) is Compound I-7 and the one or more Shh pathway activator(s) is Purmorphamine. In some embodiments, minoxidil is at a concentration of about 10 mM to about 250 mM, Compound I-7 is at a concentration of about 1 nM to about 100 nM and Purmorphamine is at a concentration of about 100 nM to about 10 μM. In certain embodiments, minoxidil is at a concentration of about 10 mM to about 250 mM, Compound I-7 is at a concentration of about 1 μM to about 100 μM and Purmorphamine is at a concentration of about 100 μM to about 10 mM.

In some embodiments of the methods and compositions disclosed herein, Compound I-7 and the one or more Shh pathway activator(s) is SAG. In some embodiments, minoxidil s at a concentration of about 10 mM to about 250 mM, Compound I-7 is at a concentration of about 1 nM to about 100 nM and SAG is at a concentration of about 1 nM to about 100 nM. In certain embodiments, minoxidil is at a concentration of about 10 mM to about 250 mM, Compound I-7 is at a concentration of about 1 μM to about 100 μM and SAG is at a concentration of about 1 μM to about 100 μM.

In some embodiments of the methods and compositions disclosed herein, the one or more Wnt agonists(s) is Compound I-7 and the one or more Shh pathway activator(s) is 20-alpha hydroxy cholesterol. In some embodiments, minoxidil is at a concentration of about 10 mM to about 250 mM, Compound I-7 is at a concentration of about 1 nM to about 100 nM and 20-alpha hydroxy cholesterol is at a concentration of about 1 μM to about 100 μM. In certain embodiments, minoxidil is at a concentration of about 10 mM to about 250 mM, Compound I-7 is at a concentration of about 1 μM to about 100 μM and 20-alpha hydroxy cholesterol is at a concentration of about 1 mM to about 100 mM.

In some embodiments of the methods and compositions disclosed herein, the one or more Wnt agonists(s) is Compound I-7 and the one or more Shh pathway activator(s) is SAG HCl. In some embodiments, minoxidil is at a concentration of about 10 mM to about 250 mM, Compound I-7 is at a concentration of about 1 nM to about 100 nM and SAG HCl is at a concentration of about 10 nM to about 1 μM. In certain embodiments, minoxidil is at a concentration of about 10 mM to about 250 mM, Compound I-7 is at a concentration of about 1 μM to about 100 μM and SAG HCl is at a concentration of about 10 μM to about 1 mM.

In some embodiments of the methods and compositions disclosed herein, the one or more Wnt agonists(s) is Compound I-12 and the one or more Shh pathway activator(s) is Purmorphamine. In some embodiments, minoxidil is at a concentration of about 10 mM to about 250 mM, Compound I-12 is at a concentration of about 10 nM to about 1000 nM and Purmorphamine is at a concentration of about 100 nM to about 10 μM. In certain embodiments, minoxidil is at a concentration of about 10 mM to about 250 mM, Compound I-12 is at a concentration of about 10 μM to about 1000 μM and Purmorphamine is at a concentration of about 100 μM to about 10 mM.

In some embodiments of the methods and compositions disclosed herein, the one or more Wnt agonists(s) is Compound I-12 and the one or more Shh pathway activator(s) is SAG. In some embodiments, minoxidil is at a concentration of about 10 mM to about 250 mM, Compound I-12 is at a concentration of about 10 nM to about 1000 nM and SAG is at a concentration of about 1 nM to about 100 nM. In certain embodiments, minoxidil is at a concentration of about 10 mM to about 250 mM, Compound I-12 is at a concentration of about 10 μM to about 1000 μM and SAG is at a concentration of about 1 μM to about 100 μM.

In some embodiments of the methods and compositions disclosed herein, the one or more Wnt agonists(s) is Compound I-12 and the one or more Shh pathway activator(s) is 20-alpha hydroxy cholesterol. In some embodiments, minoxidil is at a concentration of about 10 mM to about 250 mM, Compound I-12 is at a concentration of about 10 nM to about 1000 nM and 20-alpha hydroxy cholesterol is at a concentration of about 1 μM to about 100 μM. In certain embodiments, minoxidil is at a concentration of about 10 mM to about 250 mM, Compound I-12 is at a concentration of about 10 μM to about 1000 μM and 20-alpha hydroxy cholesterol is at a concentration of about 1 mM to about 100 mM.

In some embodiments of the methods and compositions disclosed herein, the one or more Wnt agonists(s) is Compound I-12 and the one or more Shh pathway activator(s) is SAG HCl. In some embodiments, minoxidil is at a concentration of about 10 mM to about 250 mM, Compound I-12 is at a concentration of about 10 nM to about 1000 nM and SAG HCl is at a concentration of about 10 nM to about 1 μM. In certain embodiments, minoxidil is at a concentration of about 10 mM to about 250 mM, Compound I-12 is at a concentration of about 10 μM to about 1000 μM and SAG HCl is at a concentration of about 10 μM to about 1 mM.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present invention, suitable methods and materials are described below. In the case of conflict, the present specification, including definitions, will control. In addition, the materials, methods and examples are illustrative only and are not intended to be limiting.

Other features and advantages of the invention will be apparent from the following detailed description and claims.

### BRIEF DESCRIPTION OF THE DRAWINGS

FIG. 1A is a graph depicting changes in hair score over time in a stem cell proliferation assay in response to treatment with i) vehicle, ii) minoxidil, iii) FHZ-66+minoxidil, iv) FHZ-01+FHZ-66+minoxidil, and v) FHZ-01+minoxidil. Hair score was evaluated 5, 7, 9, and 11 days post-treatment.

FIG. 1B is a table depicting 2-way Anova adjusted P values for each combination therapy compared with minoxidil-only treatment.

FIG. 2A is a graph depicting changes in hair score over time in a stem cell proliferation assay in response to treatment with i) vehicle, ii) minoxidil, iii) FHZ-66+minoxidil, iv) FHZ-01+FHZ-66+minoxidil, and v) FHZ-01+minoxidil. Hair score was evaluated 5, 7, 9, 11, and 13 days post-treatment.

FIG. 2B is a table depicting 2-way Anova adjusted P values for each combination therapy compared with minoxidil-only treatment.

FIG. 3A is a graph depicting changes in hair score over time in a stem cell proliferation assay in response to treatment with i) vehicle, ii) minoxidil, iii) FHZ-66+minoxidil, iv) FHZ-07+FHZ-66+minoxidil, and v) FHZ-07+minoxidil. Hair score was evaluated 5, 7, 9, 11 days post-treatment.

FIG. 3B is a table depicting 2-way Anova adjusted P values for each combination therapy compared with minoxidil-only treatment.

FIG. 4A is a graph depicting changes in hair score over time in a stem cell proliferation assay in response to treatment with i) vehicle, ii) minoxidil, iii) FHZ-66+minoxidil, iv) FHZ-07+FHZ-66+minoxidil, and v) FHZ-07+minoxidil. Hair score was evaluated 5, 7, 9, 11, and 13 days post-treatment.

FIG. 4B is a table depicting 2-way Anova adjusted P values for each combination therapy compared with minoxidil-only treatment.

### DETAILED DESCRIPTION OF THE INVENTION

The present disclosure relates to a method of expanding a population of stem cells of hair follicles, by contacting the stem cells with minoxidil (or anologues thereof) and one or more Sonic Hedgehog pathway (Shh) activator(s) and optionally, one or more Wnt agonists(s).

In another aspect, the present disclosure relates to a method of facilitating the generation of hair follicle epithelial cells, by treating stem cells of hair follicles with with minoxidil (or anologues thereof) and one or more Sonic Hedgehog pathway (Shh) activator(s) and optionally, one or more Wnt agonists(s).

In one embodiment, the stem cells are dermal papilla stem cells. In another embodiment, the stem cells are hair follicle stem cells. In some embodiments, the stem cells comprise keratinocytes, melanocytes, dermal papilla cells, bulge cells, or a combination thereof. In some embodiments, the stem cells are in a subject.

In certain embodiments, therefore, the present disclosure provides methods to induce self-renewal of a population of stem cells in hair follicles by using minoxidil and activating the Shh pathway and optionally, the Wnt pathway. Preferably, the Shh and/or Wnt pathways are activated with small molecules or proteins. For example, a compound(s) when applied in vitro to dermal papilla (DP) stem cells in hair follicles induces the DP stem cells to proliferate to a high degree and in high purity in a Stem Cell Proliferation Assay using dermal papilla cells, and also allows an increase in the number of DP cells and/or DP area within a hair follicle in a DP hair follicle culture assay. In one such embodiment, minoxidil (or anologues thereof), the one or more Shh pathway activator(s) and optionally one or more Wnt agonists(s) induces and maintains stem cell properties by producing stem cells that can divide and maintain the ability to have a high proportion of the resulting cells differentiate into cells of the hair follicle. Further, the proliferating stem cells express stem cell markers which may include one or more of Gli1, Krt15, CD34, Lgr5, Lgr6, Lrig1, Sox2, CD133, Vimentin, Versican and/or alkaline phosphatase.

In another aspect, the present disclosure provides a method of treating a subject who has, or is at risk of developing, a disease associated with absence or lack of hair follicle epithelial cells, the method comprising administering to said subject minoxidil (or anologues thereof); one or more Sonic Hedgehog pathway (Shh) activator(s) and optionally one or more Wnt agonists(s). In some embodiments, the disease is selected from telogen effluvium, anagen effluvium, androgenetic alopecia, alopecia areata, tinea capitis, lichen planopilaris, cicatricial alopecia, discoid lupus erythematosus, folliculitis decalvans, dissecting cellulitis of the scalp, frontal fibrosing alopecia, central centrifugal cicatricial alopecia, trichotillomania, traction alopecia, and hypotrichosis.

In another aspect, the present disclosure provides a method of treating a subject who has, or is at risk of developing, alopecia, the method comprising administering to a subject minoxidil (or anologues thereof); one or more Sonic Hedgehog pathway (Shh) activator(s) and optionally one or more Wnt agonists(s).

In yet another aspect, the present disclosure provides a pharmaceutical composition comprising: a pharmaceutically-acceptable carrier and (i) minoxidil, an analogue thereof or a pharmaceutically-acceptable salt thereof; and (ii) a Sonic Hedgehog (Shh) pathway activator, or a pharmaceutically-acceptable salt thereof. Optionally, the pharmaceutical composition further includes a Wnt agonist or a pharmaceutically-acceptable salt thereof.

In some embodiments of the methods and compositions disclosed herein, minoxidil (or analogue thereof) is at a concentration of about of about 0.001× to about 10× of an FDA approved minoxidil concentration. In certain embodiments, minoxidil (or analogue thereof) is at a concentration of about 0.01× to about 5× of an FDA approved minoxidil concentration. In some embodiments, the minoxidil (or analogue thereof) is at a concentration of about 0.001× to about 1× of an FDA approved minoxidil amount, of about 0.01× to about 1× of an FDA minoxidil approved amount, or of 0.1× to about 1× of an FDA approved minoxidil concentration.

In some embodiments of the methods and compositions disclosed herein, minoxidil (or analogue thereof) is at a concentration of about 0.1%, 1%, 2%, 3%, 4%, 5%, 6%, 7%, 8%, 9% or 10% (w/v)

In some embodiments of the methods and compositions disclosed herein, the one or more Shh pathway activator(s) is at a concentration of about 5× to about 1000× of an effective in vitro Shh pathway activation concentration. In certain embodiments, the one or more Shh pathway activator(s) is at a concentration of about 10× to about 100× of an effective in vitro Shh pathway activation concentration. In some embodiments, the one or more Shh pathway activator(s) is at a concentration of about 20× to about 50× of an effective in vitro Shh pathway activation concentration. In certain embodiments, the one or more Wnt agonists(s) is at a concentration of about 5× to about 1000× of an effective in vitro Wnt agonist concentration. In some embodiments, the one or more Wnt agonists(s) is at a concentration of about 10× to about 100× of an effective in vitro Wnt agonist concentration. In some embodiments, the one or more Wnt agonists(s) is at a concentration of about 20× to about 50× of an effective in vitro Wnt agonist concentration.

In some embodiments of the methods and compositions disclosed herein, the Shh pathway activator comprises a Smoothened agonist. In other embodiments, the Shh pathway activator comprises Smoothened ciliary accumulation enhancers. In some embodiments of the methods and compositions disclosed herein, the one or more Shh pathway activator(s)(s) is selected from the group consisting of a SAG, an oxysterol, a Purmorphamine analogue, a GSA-10 analogue, polydatin, glucocorticoids, hedgehog polypeptides, inositol derivatives, sterols, peroxiredoxin 2, RACK1, Dhh, and Ihh. In some embodiments, the one or more Shh pathway activator(s) is selected from Table 1. In some embodiments, the one or more Shh pathway activator(s) is selected from Table 2. In certain embodiments, the one or more Shh pathway activator(s) is Purmorphamine (CAS 483367-10-8). In further embodiments of the methods and compositions disclosed herein, the one or more Shh pathway activator(s) is selected from Purmorphamine, SAG, 20-alpha hydroxy cholesterol, and SAG HCl.

TABLE 1
Exemplary Shh pathway activators for use in compositions
and methods of the present disclosure

| Compound | Compound Name or | Compound | |
|---|---|---|---|
| Family | Company | CAS number | Reference(s) |
| | SAG (HH-Ag) Family | | |
| SAG (HH-Ag) | HH-Ag 1.1 | 364590-52-3 | Journal of biology (2002) |
| | | | 1 (2): 10 |
| SAG (HH-Ag) | HH-Ag 1.2 | 364590-54-5 | Cell Chemical Biology |
| | | | (2017) 24(3): 252-280 |
| SAG (HH-Ag) | HH-Ag 1.2 (Trans) | 612542-13-9 | Journal of biology (2002) |
| | | | 1 (2): 10 |
| SAG (HH-Ag) | HH-Ag 1.3 (SAG-1.3) | 364590-63-6 | |
| SAG (HH-Ag) | HH-Ag 1.3 (Trans) - | 912545-86-9 | Stem Cells Int. (2012) |
| | SAG (3 nM) | | 140427; |
| | | | Nat. Chem. Biol. (2009) |
| | | | 5(3): 154-156; |
| | | | PNAS (2002) 99(22): |
| | | | 14071-14076 |
| SAG (HH-Ag) | HH-Ag 1.3 (Cis) | 1401532-80-6 | |
| SAG (HH-Ag) | HH-Ag 1.4 | ? | |
| SAG (HH-Ag) | HH-Ag 1.5 | 612542-14-0 | |
| SAG (HH-Ag) | HH-Ag 1.6 | | Journal of |
| | | | Neurochemistry (2009) |
| | | | 108(6): 1539-1549 |
| SAG (HH-Ag) | HH-Ag 1.8 | 869497-97-2 | Journal of Investigative |
| | | | Dermatology (2005) |
| | | | 125(4): 638-646 |
| SAG (HH-Ag) | CUR-0236715 | 946048-09-5 | |
| SAG (HH-Ag) | Curis (trans) | 612542-12-8 | U.S. Pat. No. 6,683,108 B1 |
| SAG (HH-Ag) | Curis/Wyeth | 946047-96-7 | WO 2008057468 A1 |
| SAG (HH-Ag) | Curis/Wyeth | 946047-93-4 | WO 2008057468 A1 |
| SAG (HH-Ag) | Compounds 35 | 566172-68-7 | PLoS Biol (2016) 14(2): |
| | (Cis/trans) | | e1002375 |
| SAG (HH-Ag) | Compounds 35 | 1186398-30-0 | PLoS Biol (2016) 14(2): |
| | (Trans) | | e1002375 |
| SAG (HH-Ag) | Compounds 36 | 946002-48-8 | PLoS Biol (2016) 14(2): |
| | | | e1002375 |
| | STEROLS | | US 2008/013319 |
| STEROLS | Compound 35 | 27241-03-8 | ACS Med. Chem. Lett. |
| | (0.54-0.65 μM) | | (2012) 3(10): 828-833 |
| STEROLS | Oxy34 | 24339-14-8 | Journal of Cellular |
| | | | Biochemistry (2011) |
| | | | 112(6): 1673-1684 |
| STEROLS | Oxy49 (3 μM) | 1059591-82-0 | Bioorganic & Medicinal |
| | | | Chemistry Letters (2012) |
| | | | 22(18): 5893-5897; |
| | | | ACS Med. Chem. Lett. |
| | | | 2012, 3, 828-833 |
| STEROLS | 20(S)- | 516-72-3 | Biomaterials (2014) |
| | hydroxy cholesterol | | 35(26): 7336-7345 |
| STEROLS | Halcinonide | 3093-35-4 | PLoS One (2015) 10(12): |
| | | | e0144550; |
| | | | PNAS (2010) 107(20): |
| | | | 9323-9328 |
| STEROLS | Clobetasol | 25122-41-2 | PLoS One (2015) 10(12): |
| | | | e0144550; |
| | | | PNAS (2010) 107(20): |
| | | | 9323-9328 |
| STEROLS | Fluticasone | 90566-53-3 | PLoS One (2015) 10(12): |
| | | | e0144550; |
| | | | PNAS (2010) 107(20): |
| | | | 9323-9328 |
| STEROLS | Fluocinonide | 356-12-7 | PNAS (2010) 107(20): |
| | | | 9323-9328 |
| STEROLS | Triamcinolone | 76-25-5 | ChemMedChem (2014) |
| | acetonide | | 9(1): 27-37 |
| | Purmorphamine | | |
| | Analogues | | |
| Purmorphamine | Purmorphamine | 483367-10-8 | Biomaterials (2014) |
| | | | 35(26): 7336-7345; |
| | | | Chem. Biol. (2004) 11(9): |
| | | | 1229-1238; |
| | | | JACS (2002) 124(49): |
| | | | 14520-14521 |
| Purmorphamine | Compound 40 | 841221-62-3 | ChemMedChem (2014) |
| | | | 9(1): 27-37; |
| | | | Cells Tissues Organs |
| | | | (2012) 197(2): 89-102 |
| Purmorphamine | Compound 38 | 1394159-22-8 | ChemMedChem (2014) |
| | | | 9(1): 27-37; |
| | | | Cells Tissues Organs |
| | | | (2012) 197(2): 89-102 |
| Purmorphamine | 96547 (#70) | 1160569-67-4 | Molecules and Cells |
| | | | (2008), 26(4): 380-386 |
| | GSA-10 Analogues | | |
| GSA-10 | GSA-10 | 300833-95-8 | European Journal of |
| | | | Medicinal |
| | | | Chemistry (2016) 121: |
| | | | 747-757; |
| | | | Molecular Pharmacology |
| | | | (2013) 84(2): 303 |
| GSA-10 | Compound 11 | 353777-56-7 | European Journal of |
| | | | Medicinal |
| | | | Chemistry (2016) 121: |
| | | | 747-757 |
| GSA-10 | Compound 20 | 1990524-02-1 | European Journal of |
| | | | Medicinal |
| | | | Chemistry (2016) 121: |
| | | | 747-757 |
| GSA-10 | Compound 35 | 331963-09-8 | European Journal of |
| | | | Medicinal |
| | | | Chemistry (2016) 121: |
| | | | 747-757 |
| GSA-10 | Compound 12 | 300837-51-8 | European Journal of |
| | | | Medicinal |
| | | | Chemistry (2016) 121: |
| | | | 747-757 |
| | Polydatin | | |
| Polydatin | Polydatin, a glucoside | 65914-17-2 | Food and Chemical |
| | of resveratrol | | Toxicology (2016), 96: |
| | | | 215-225 |
| | PI4P | | |
| PI4P | phosphatidylinositol 4- | | Cell Chemical Biology |
| | phosphate (PI4P) | | (2017) 24(3): 252-280; |
| | | | PLoS Biol (2016) 14(2): |
| | | | e1002375 |
| | | CAS | |
| | | number or | |
| | Patents | Shh pathway | |
| | Assignee | activator | |
| SAG (HH-Ag) | CUR-0201365 | 946047-59-2 | US2010/038833 |
| SAG (HH-Ag) | CUR-0236715 | 946048-09-5 | US2010/038833 |
| SAG (HH-Ag) | CUR-0201784 | 946047-85-4 | US2010/038833 |
| SAG (HH-Ag) | Curis | HH-Ag | WO 2001/074344 A2 |
| SAG (HH-Ag) | Curis | HH-Ag | WO 2008/057468 A1 |
| SAG (HH-Ag) | Curis | HH-Ag | WO 2008/057469 A1 |
| SAG (HH-Ag) | Curis | HH-Ag | WO 2008/057497 A2 |
| SAG (HH-Ag) | Curis | HH-Ag | US 2002/0198236 A1 |
| SAG (HH-Ag) | Curis | HH-Ag | US 2003/0139457 A1 |
| SAG (HH-Ag) | Aderans Research | HH-Ag | WO 2010/148094 A1 |
| | Institute | | |
| SAG (HH-Ag) | Aderans Research | HH-Ag | US 2012/0095445 |
| | Institute | | |
| GSA-10 | Centre National de la | GSA-10 | U.S. Pat. No. 8,957,091 B2 |
| | Recherche Scientifique | analogues | |
| GSA-10 | Centre National de la | GSA-10 | US 2012/0121693 A1 |
| | Recherche Scientifique | analogues | |
| GSA-10 | Centre National de la | GSA-10 | US 2013/0236912 A1 |
| | Recherche Scientifique | analogues | |
| GSA-10 | Centre National de la | GSA-10 | WO 2012/066479 A1 |
| | Recherche Scientifique | analogues | |
| Sterols | THE REGENTS OF | Oxysterols | U.S. Pat. No. 9,526,737 B2 |
| | THE UNIVERSITY | | |
| | OF CALIFORNIA | | |
| Sterols | THE REGENTS OF | Oxysterols | WO 2008/115469 A2 |
| | THE UNIVERSITY | | |
| | OF CALIFORNIA | | |
| Sterols | THE REGENTS OF | Oxysterols | WO 2009/073186 A1 |
| | THE UNIVERSITY | | |
| | OF CALIFORNIA | | |
| Sterols | THE REGENTS OF | Oxysterols | WO 2014/179756 A1 |
| | THE UNIVERSITY | | |
| | OF CALIFORNIA | | |
| Sterols | Fate Therapeutics | Oxy sterols | WO 2012/024581 A2 |
| Sterols | Fate Therapeutics | Oxysterols | WO 2012/024583 A2 |
| Sterols | Fate Therapeutics | Oxysterols | WO 2012/024584 A2 |
| Sterols | Duke | Glucocorticoids | WO 2011/109711 A1 |
| Peptides | Elizabeth Wang | Hedge Hog | US 2002/0151460 A1 |
| | | Polypeptides | |
| Peptides | Ontogeny | Hedge Hog | WO 99/20298 A1 |
| | | Polypeptides | |
| Peptides | Strasspharma | Hedge Hog | WO 2014/085523 A1 |
| | | Polypeptides | |
| Purmorphamine | Scripps and IRM | Purmorphamine | US 2004/0157864 A1 |
| Purmorphamine | Biomatcell | Purmorphamine | WO 2012/115575 A1 |
| HH-Ag | | HH-Ag | US 2005/0070578 A1 |
| PI4P | Barbara Brooke | Inositol | US 2009/0214474 A1 |
| | Jennings | Derivatives | |
| Sterols | | Sterols | WO 00/41545 A2 |
| | Proteins | | |
| Proteins | Peroxiredoxin 2 | | Oncotarget (2016) 7(52): |
| | | | 86816-86828 |
| Proteins | RACK1 | | Journal of Biological |
| | | | Chemistry (2012) |
| | | | 287(11): 7845-7858 |
| Proteins | Desert Hedgehog | | Journal of Biology |
| | (Dhh) | | (2002). Agonizing |
| | | | Hedgehog. 1(2): 7 |
| Proteins | Indian Hedgehog (Ihh) | | Journal of Biology |
| | | | (2002). Agonizing |
| | | | Hedgehog. 1(2): 7 |

In some embodiments of the methods and compositions disclosed herein, the one or more SAG compounds is a compound of Formula II:

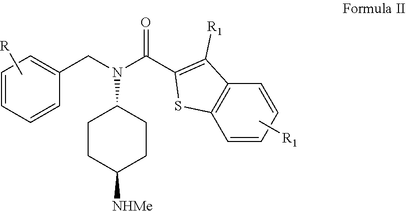

wherein R is independently one or more —H, halogen, —C₁-C₁₀alkyl, —C₃-C₁₀cycloalkyl, —OC₁-C₁₀alkyl, —CN, Aryl, Substituted Aryl, Heteroaryl, Substituted Heteroaryl;

wherein the substitution can one or more —H, halogen, —OH, —OC₁-C₆alkyl, —C₁-C₆alkyl, —CN, NH₂, —NHSO₂—C₁-C₆alkyl, —NHCO—C₁-C₆alkyl, —SO₂—C₁-C₆alkyl, —CONH₂, —CONH—C₁-C₆alkyl, COOH, —COO—C₁-C₆alkyl;

wherein R¹ is independently one or more —H, halogen, —C₁-C₃alkyl, —OH, —O—C₁-C₃ alkyl.

TABLE 2
Exemplary Shh pathway activators for use in compositions
and methods of the present disclosure

| Shh pathway activator | CAS number |
|---|---|
| Purmorphamine | 483367-10-8 |
| Compound 35 | 27241-03-8 |
| HH-Ag 1.1 | 364590-52-3 |
| HH-Ag 1.2 | 364590-54-5 |
| HH-Ag 1.2 (Trans) | 612542-13-9 |
| HH-Ag 1.3 (SAG-1.3) | 364590-63-6 |
| HH-Ag 1.3 (Trans) - SAG | 912545-86-9 |
| HH-Ag 1.3 (Cis) | 1401532-80-6 |
| HH-Ag 1.5 | 612542-14-0 |
| CUR-0236715 | 946048-09-5 |
| Halcinonide | 3093-35-4 |
| Clobetasol | 25122-41-2 |
| Fluticasone | 90566-53-3 |
| Compounds 36 | 946002-48-8 |
| Compound 35 | 331963-09-8 |
| Compound 12 | 300837-51-8 |
| Compound 20 | 1990524-02-1 |

Classes of Wnt agonist (Wnt activator) for use in various embodiments of the compositions and methods disclosed herein include but are not limited to those listed in Column A of Table 3. Specific Wnt agonists for use in various embodiments of the compositions and methods disclosed herein include but are not limited to those listed in Column B of Table 3. All agents listed in Table 3 column B are understood to include derivatives or pharmaceutically-acceptable salts thereof. All classes listed in Table 3 column A are understood to include both agents comprising that class and derivatives or pharmaceutically-acceptable salts thereof.

TABLE 3
Exemplary Wnt agonists (Wnt activators) for use in compositions
and methods of the present disclosure
Wnt agonists

| Column A | Column B | CAS Number |
|---|---|---|
| Wnt Ligand | Wnt-1 | Protein |
| | Wnt-2/Irp (Int-I-related protein) | Protein |
| | Wnt-2b/13 | Protein |
| | Wnt-3/Int-4 | Protein |
| | Wnt-3a | Protein |
| | Wnt-4 | Protein |
| | Wnt-5a | Protein |
| | Wnt-5b | Protein |
| | Wnt-6 | Protein |
| | Wnt-7a | Protein |
| | Wnt-7b | Protein |
| | Wnt-8a/8d | Protein |
| | Wnt-8b | Protein |
| | Wnt-9a/14 | Protein |
| | Wnt-9b/14b/15 | Protein |
| | Wnt-10a | Protein |
| | Wnt-10b/12 | Protein |
| | Wnt-11 | Protein |
| | Wnt-16 | Protein |
| Wnt Related Protein | R-Spondin 1/2/3/4 | Protein |
| | Norrin | Protein |
| Target | Agent | CAS Number |
| Wnt-3a/Dkk-1 | Compound 1 | 1084833-94-2 |
| Wnt-3a/Dkk-1 | Compound 25 | 1084834-05-8 |
| | BML-284 | 853220-52-7 |
| PP2A | IQ 1 | 331001-62-8 |
| beta-catenin | DCA | 56-47-3 |
| ARFGAP1 | QS 11 | 944328-88-5 |
| | WASP-1, ZINC00087877 | 352328-82-6 |
| sFRP-1 inhibitor | WAY 316606 | 915759-45-4 |
| | (Dimethylamino)propyl)-2-ethyl-5- | 915754-88-0 |
| | (phenylsulfonyl)benzenesulfonamide | |
| | Cyclosporine A (CsA) | 59865-13-3 |
| | PSC833 (Valspodar) | 121584-18-7 |
| | Cyclosporine analogs | |
| DKK1 inhibitor | WAY-262611 | 1123231-07-1 |
| Axin | HLY78 | 854847-61-3 |
| Axin | SKL2001 | 909089-13-0 |
| | Cpd1 | 1357473-75-6 |
| | Cpd2 | 1228659-47-9 |
| van-Gogh-like receptor | Compound 109 | 1314885-81-8 |
| proteins (Vangl) | | |
| Disrupts the Axin Complex | ISX 9 | 832115-62-5 |
| | Compound 71 | 1622429-71-3 |
| | Compound 2 | 1360540-82-4 |
| MEK | Selumetinib (AZD6244) | 606143-52-6 |
| | Radicicol | 12772-57-5 |
| Target Undetermined | Diketones | WO 2016029021 A1; |
| | | WO 2012024404 A1 |
| | Diketones | 1622429-56-4 |
| | Diketones | 1360540-88-0 |
| | Diketones | 1360540-89-1 |
| | Diketones | 1622429-79-1 |
| | Diketones | 1622429-75-7 |
| | Diketones | 1622429-74-6 |
| | Diketones | 1622430-76-5 |
| | Diketones | 1622430-31-2 |
| | Diketones | 1622430-52-7 |
| | Diketones | 1622429-67-7 |
| | Diketones | 1622429-65-5 |
| | Diketones | 1622429-69-9 |

In some embodiments of the methods and compositions disclosed herein, the one or more Wnt activator or agonist is one or more GSK inhibitor. In certain embodiments, the GSK inhibitor is a GSK3-beta inhibitor. Classes of GSK3-beta inhibitor for use in various embodiments of the compositions and methods disclosed herein include, but are not limited to, those listed in Column A of Table 4. Specific GSK3-beta inhibitors for use in various embodiments of the compositions and methods disclosed herein include but are not limited to those listed in Column B of Table 4. All agents listed in Table 4 column B are understood to include derivatives or pharmaceutically-acceptable salts thereof. All classes listed in Table 4 column A are understood to include both agents comprising that class and derivatives or pharmaceutically-acceptable salts thereof.

TABLE 4
Exemplary GSK-beta inhibitors for use in compositions
and methods of the present disclosure
Table 4. GSK3β Inhibitors

| Column A | Column B | |
|---|---|---|
| Class | Agent | CAS Number |
| Acid | Valproic Acid, Sodium | 99-66-1 |
| | Salt | |
| Acid | Bikinin | 188011-69-0 |
| Pyrroloazepine | Hymenialdisine | 82005-12-7 |
| Aloisines | Aloisine A | 496864-16-5 |
| Aloisines | Aloisine B | 496864-14-3 |
| Aloisines | TWS119 | 1507095-58-0 |
| Aminopyrimidine | CT20026 | 403808-63-9 |
| Aminopyrimidine | CHIR99021 (CT99021) | 252917-06-9 |
| Aminopyrimidine | CHIR98014 (CT98014) | 252935-94-7 |
| Aminopyrimidine | CHIR98023 (CT98023) | 252904-84-0 |
| Aminopyrimidine | CHIR98024 (CT98024) | 556813-39-9 |
| Aminopyrimidinyl | GSK-3β Inhibitor XVIII | 1139875-74-3 |
| Aminopyrimidinyl | CGP60474 | 164658-13-3 |
| Miscellaneous | AZD2858 (AR28) | 486424-20-8 |
| Miscellaneous | CID 755673 | 521937-07-5 |
| Miscellaneous | TCS 2002 | 1005201-24-0 |
| Miscellaneous | Dibromocantharelline | 101481-34-9 |
| Dihydropyridine | ML320 | 1597438-84-0 |
| Flavone | Flavopiridol | 146426-40-6 |
| Furopyrimidine | Compound 100 | 744255-19-4 |
| Hymenidin | Hymenidin | 107019-95-4 |
| Indirubins | 6-Bromoindirubin-3- | 667463-85-6 |
| | acetoxime | |
| Indirubins | GSK-3 Inhibitor IX | 667463-62-9 |
| Indirubins | Indirubin-3′- | 160807-49-8 |
| | monoxime | |
| Indirubins | 5-Iodo-indirubin-3′- | 331467-03-9 |
| | monoxime | |
| Indirubins | Indirubin-5-sulfonic | 331467-05-1 |
| | acid sodium salt | |
| Indirubins | Indirubin | 479-41-4 |
| Indirubins | GSK-3 Inhibitor X | 740841-15-0 |
| Inorganic atom | Lithium Chloride | |
| Inorganic atom | Beryllium | |
| Inorganic atom | Zinc | |
| Inorganic atom | Tungstate | |
| Isonicotinamides | Compound 39 | 1772824-10-8 |
| Isonicotinamides | Compound 29 | 1772823-37-6 |
| Isonicotinamides | Compound 33 | 1772823-64-9 |
| Azaindolylmaleimide | Compound 29 | 436866-61-4 |
| Azaindolylmaleimide | Compound 46 | 682807-74-5 |
| Bisindolylmaleimide | Compound 5a | 436866-54-5 |
| Bisindolylmaleimide | GF109203X | 176504-36-2 |
| Bisindolylmaleimide | Ro318220 | 125314-64-9 |
| Bisindolylmaleimide | Bisindolylmaleimide X | 131848-97-0 |
| | HCl | |
| Bisindolylmaleimide | Enzastaurin | 170364-57-5 |
| | (LY317615) | |
| Maleimide | I5 | 264217-24-5 |
| Maleimide | SB-216763 | 280744-09-4 |
| Maleimide | SB-415286 (SB-41528) | 264218-23-7 |
| Maleimide | 3F8 | 159109-11-2 |
| Maleimide | TCS 21311 | 1260181-14-3 |
| Maleimide | GSK-3β inhibitor 1 | 603272-51-1 |
| Maleimide | LY2090314 | 603288-22-8 |
| Maleimide | 603281-31-8 | 603281-31-8 |
| Maleimide | IM-12 | 1129669-05-1 |
| Maleimide | Compound 34 | 396091-16-0 |
| Maleimide | KT 5720 | 108068-98-0 |
| Maleimide | Isogranulatimide | 244148-46-7 |
| Maleimide | GSK-3p Inhibitor XI | 626604-39-5 |
| Maleimide | BIP-135 | 941575-71-9 |
| Maleimide | CP21R7 | 125314-13-8 |
| Maleimide | Tivantinib | 905854-02-6 |
| Organometallic | Compound lambda- | 1291104-51-2, |
| | OS1 | 1292843-11-8 |
| Organometallic | HB12 | 800384-87-6 |
| Organometallic | DW12 | 861251-33-4 |
| Organometallic | NP309 | 937810-13-4 |
| Organometallic | (RRu)-HB1229 | |
| Organometallic | (RRu)-NP549 | |
| Organometallic | Compound 3 | 1498285-39-4, |
| | | 1498285-48-5 |
| Organometallic | Compound (R)-DW12 | 1047684-07-0 |
| Isoindolone | Staurosporine | 62996-74-1 |
| Pyrazolone | GSK-3beta Inhibitor | 871843-09-3 |
| | XXVI | |
| Manzamines | Manzamine A | 104196-68-1 |
| Oxadiazol | TC-G 24 | 1257256-44-2 |
| Oxadiazol | Compound 14d | 1374671-64-3 |
| Oxadiazol | Compound 15b | 1374671-66-5 |
| Oxadiazol | Compound 20x | 1005201-80-8 |
| Oxadiazol | GSK-3 Inhibitor II | 478482-75-6 |
| Oxadiazol | GSK3 Inhibitor, 2 | 1377154-01-2 |
| Oxindole | SU9516 | 77090-84-1 |
| Oxindole | AZD1080 | 612487-72-6 |
| Paullone | Kenpaullone | 142273-20-9 |
| Paullone | Cmpd 17b | 408532-42-3 |
| Paullones | Azakenpaullone | 676596-65-9 |
| Paullones | Alsterpaullone | 237430-03-4 |
| Paullones | Alsterpaullone CN | 852529-97-0 |
| | Ethyl | |
| Paullones | Cazpaullone | 914088-64-5 |
| Peptide | FRATtide | |
| Peptide | L803 | |
| Peptides | L803-mts | |
| Pyrazole | GSK-3 Inhibitor XXII | 1195901-31-5 |
| Pyrazole | Compound 4a | 1627557-91-8 |
| Pyrazole | Compound 4t | 1627558-10-4 |
| Pyrazole | Compound 4z | 1627558-16-0 |
| Pyrazole | AT 7519 | 844442-38-2 |
| Pyrazolopyridine | Pyrazolopyridine 9 | 923029-74-7 |
| Pyrazolopyridine | Pyrazolopyridine 18 | 405221-39-8 |
| Pyrazolopyridine | Pyrazolopyridine 34 | 583039-27-4 |
| Pyrazolopyridines | Compound 14 | 583038-63-5 |
| Pyrazolopyridines | Compound 23 | 583038-76-0 |
| Pyrazolopyridines | Compound 14 | 583038-63-5 |
| Pyrazolopyridazines | Compound 18 | 405223-20-3 |
| Pyrazolopyridazines | Compound 19 | 405223-71-4 |
| Pyrazoloquinoxaline | NSC 693868 | 40254-90-8 |
| | (Compound 1) | |
| Pyridinone | Compound 150 | 1282042-18-5 |
| Quinazolin | GSK-3 Inhibitor XIII | 404828-08-6 |
| Quinolinecarb | VP0.7 | 331963-23-6 |
| Quinolinecarboxamide | | 1132813-46-7 |
| Quinolinecarboxamide | | 1132812-98-6 |
| Quinolinecarboxamide | | 950727-66-9 |
| Pyrazoloquinoxaline | NSC 693868 | 40254-90-8 |
| | (Compound 1) | |
| Halomethylketones | Compound 17 | 62673-69-2 |
| Halomethylketones | GSK-3β Inhibitor VII | 99-73-0 |
| Halomethylketones | GSK-3β Inhibitor VI | 62673-69-2 |
| Furanosesquiterpenes | Palinurin | 254901-27-4 |
| Furanosesquiterpenes | Tricantin | 853885-55-9 |
| Thiadiazolidindiones | GSK-3β Inhibitor I | 327036-89-5 |
| Thiadiazolidindiones | NP031115 | 1400575-57-6 |
| Thiadiazolidindiones | NP031112 (Tideglusib) | 865854-05-3 |
| Triazolpyrimidine | Compound 90 | 91322-11-1 |
| Triazolpyrimidine | Compound 92 | 1043429-30-6 |
| Urea | GSK-3P Inh. VIII AR- | 487021-52-3 |
| | A014418 | |
| Urea | A-1070722 | 1384424-80-9 |
| Pyrrolopyridinyl | Compound 27 | 2025388-25-2 |
| Pyrrolopyridinyl | Compound 12 | 2025388-10-5 |
| Publication | NP-103 | No Structure |
| Publication | CG-301338 | No Structure |
| Publication | SAR 502250 | No Structure |
| Publication | XD-4241 | No Structure |
| Publication | CEP-16805 | No Structure |
| Publication | AZ13282107 | No Structure |
| Publication | SAR 502250 (Sanofi) | 1073653-58-3 |
| Publication | AR79 | |
| Publication | AZ13282107 | |
| Patent | GI179186X | |
| Patent | CT118637 | |
| Patent | CP-70949 | |
| Patent | GW784752X | |
| Patent | GW784775X | |
| Publication | CT73911 | |
| Publication | LY2064827 | |
| Publication | 705701 | |
| Publication | 708244 | |
| Publication | 709125 | |
| Patent | WO 2008077138 A1 | |
| Patent | WO 2003037891 A1 | |
| Patent | U.S. Pat. No. 8,207,216 B2 | |
| Patent | U.S. Pat. No. 8,071,591 B2 | |
| Patent | CN 1319968 C | |
| Patent | U.S. Pat. No. 7,514,445 B2 | |
| Patent | CN 101341138 B | |
| Patent | EP 1961748 A2 | |
| Patent | WO 2010104205 A1 | |
| Patent | US 20100292205 A1 | |
| Patent | WO 2014003098 A1 | |
| Patent | WO 2011089416 A1 | |
| Patent | EP 1739087 A1 | |
| Patent | WO 2001085685 A1 | |
| Patent | US 20070088080 A1 | |
| Patent | WO 2006018633 A1 | |
| Patent | WO 2009017453 A1 | |
| Patent | WO 2014050779 A1 | |
| Patent | WO 2006100490A1/EP | |
| | 1863904 A1 | |
| Patent | WO 2014013255 A1 | |
| Patent | WO 2009017455 A1 | |
| Patent | EP 2765188 A1 | |
| Patent | WO 2014083132 A1 | |
| Patent | U.S. Pat. No. 8,771,754 B2 | |
| Patent | WO 2013124413 A1 | |
| Patent | WO 2014059383 A1 | |
| Patent | WO 2010075551 A1 | |
| Patent | U.S. Pat. No. 8,686,042 B2 | |
| Patent | WO 2007102770 A1 | |

J. Med. Chem. 2016, 59, 9018-9034

In some embodiments of the methods and compositions disclosed herein, the one or more Wnt activator or agonist is one or more GSK inhibitor. In certain embodiments, the GSK inhibitor is a GSK3-alpha inhibitor. Classes of GSK3-alpha inhibitor for use in various embodiments of the compositions and methods disclosed herein include, but are not limited to, those listed in Column A of Table 5. Specific GSK3-alpha inhibitors for use in various embodiments of the compositions and methods disclosed herein include but are not limited to those listed in Column B of Table 5. All agents listed in Table 5 column B are understood to include derivatives or pharmaceutically-acceptable salts thereof. All classes listed in Table 5 column A are understood to include both agents comprising that class and derivatives or pharmaceutically-acceptable salts thereof.

TABLE 5
Exemplary GSK3-alpha inhibitors for use in compositions and methods of the present disclosure
GSK3-alpha Inhibitors

| | | | Potency | Potency | |
|---|---|---|---|---|---|
| | | | in nM | in nM | Ratio of |
| Column A | Column B | CAS | GSK3- | GSK3- | Alpha to |
| Class | Agent | Number | alpha | beta | Beta |
| Pyrazole | GSK-3b XXII | 1195901-31-5 | 2.3 | 2.0 | 0.87 |
| Pyrazole | AT 7519 | 844442-38-2 | | 89 | |
| Pyrazole | Compound 4a | 1627557-91-8 | | 8 | |
| Pyrazole | Compound 4t | 1627558-10-4 | | <5 | |
| Pyrazole | Compound 4z | 1627558-16-0 | | 5 | |
| Pyrazolopyridines | Compound 14 | 583038-63-5 | 1 | | |
| Pyrazolopyridines | Compound 23 | 583038-76-0 | 1 | | |
| Pyrazolopyridines | Pyrazolopyridine 34 | 583039-27-4 | 7 | | |
| Pyrazolopyridazines | Compound 18 | 405223-20-3 | 0.95 | | |
| Pyrazolopyridazines | Compound 19 | 405223-71-4 | 0.19 | | |
| Oxadiazoles | Compound 15b | 1374671-66-5 | 2 (230) | 185 (>1K) | 92 (>4.3) |
| Oxadiazoles | Compound 14d | 1374671-64-3 | 6 | 316 | 52 |
| Oxadiazoles | Compound 27 | 1820758-44-8 | 42 | 140 | 3.3 |
| Oxindole | AZD1080 | 612487-72-6 | 6.9 | 31 | 4.5 |
| Isonicotinamides | Compound 39 | 1772824-10-8 | 0.34 | 1.9 | 5.6 |
| Isonicotinamides | Compound 29 | 1772823-37-6 | 1.7 | 5.2 | 3.0 |
| Isonicotinamides | Compound 33 | 1772823-64-9 | 2 | 5.9 | 2.9 |
| Maleimide | Tivantinib | 905854-02-6 | 659 | 1865 | 2.8 |
| Maleimide | I5 | 264217-24-5 | 76 | 160 | 2.1 |
| Triazolpyrimidine | Compound 90 | 91322-11-1 | 330 | 628 | 1.9 |
| Triazolpyrimidine | Compound 92 | 1043429-30-6 | 9 | 13 | 1.4 |
| Organometallic | Compound lambda- | 1291104-51-2 | 0.9 | 6 | 6.8 |
| | OS1 | 1292843-11-8 | | | |
| Organometallic | Compound 3 | 1498285-39-4 | 3 | 10 | 3.3 |
| | | 1498285-48-5 | | | |
| Organometallic | Compound (R)- | 1047684-07-0 | 0.5 | 1 | 2 |
| | DW12 | | | | |
| Pyrazolo- | BRD4003 chiral | 1597439-60-5 | 4800 | 10,200 | 2.1 |
| tetrahydroquinolinone | | | | | |
| Pyrazolo- | BRD4003 chiral | 1597439-59-2 | 161 | 232 | 1.4 |
| tetrahydroquinolinone | | | | | |
| Pyrazolo- | Compound 8 | 1597439-01-4 | 18 | 87 | 4.8 |
| tetrahydroquinolinone | | | | | |
| Pyrazolo- | Compound 9 | 1597439-02-5 | 62 | 156 | 2.5 |
| tetrahydroquinolinone | | 2056261-29-9 | | | |
| Pyrazolo- | Compound 11 | 1597439-12-7 | 32 | 102 | 3.2 |
| tetrahydroquinolinone | | | | | |
| Pyrazolo- | BRD1172 | 1597438-86-2 | 3 | 10 | 3.3 |
| tetrahydroquinolinone | | | | | |
| Pyrazolo- | Compound 16 | 1597440-17-9 | 8 | 26 | 3.2 |
| tetrahydroquinolinone | | | | | |
| Pyrazolo- | BRD1652 | 1597438-93-1 | 0.4 | 4 | 10 |
| tetrahydroquinolinone | | | | | |
| Urea | AR-A014418 | 487021-52-3 | 28 | 116 | 4.1 |
| CREB knockdown | | | | | |

ACS Chem. Biol. (2016) 11: 1952-1963
PLoS One (2016) 11(4): e0153075

In certain embodiments of the methods and compositions described herein, the GSK3 inhibitor is selected from the group consisting of Valproic Acid Sodium Salt, CT20026, CHIR99021 (CT99021), CHIR98014 (CT98014), CHIR98023 (CT98023), CHIR98024 (CT98024), TCS 2002, Compound 39, Compound 29, Compound 33, TCS 21311, LY2090314, 603281-31-8, Compound 34, Compound 14d, Compound 15b, Compound 20x, AZD-1080, Kenpaullone, Cazpaullone, GSK-3 Inhibitor XXII, Compound 4a, Compound 4t, Compound 4z, Pyrazolopyridine 9, Compound 14, Compound 23, Compound 14, Compound 18, and Compound 19.

In certain embodiments of the methods and compositions described herein, the GSK3 inhibitor is selected from the group consisting of Valproic Acid Sodium Salt, CHIR99021 (CT99021), CHIR98014 (CT98014), CHIR98023 (CT98023), CHIR98024 (CT98024), Compound 39, Compound 29, LY2090314, 603281-31-8, Compound 34, Compound 14d, Compound 15b, Compound 20x, AZD-1080, Cazpaullone, GSK-3 Inhibitor XXII, Compound 4t, Compound 4z, Pyrazolopyridine 9, Compound 14, Compound 23, Compound 14, Compound 18, and Compound 19.

In some embodiments of the methods and compositions described herein, the one or more GSK inhibitor is a compound of Formula (I),

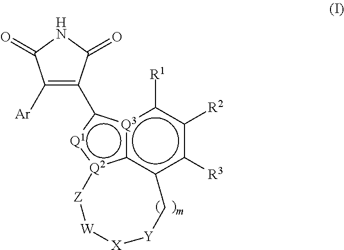

and pharmaceutically acceptable salts and tautomers thereof, wherein Q¹, Q², Q³, R¹, R², R³, Ar, —Z—W—X—Y— and m are as defined above for Formula I.

In some embodiments of the methods and compositions disclosed herein, the compounds of Formula I have one or more of the following features:

a) provided that the compound is not

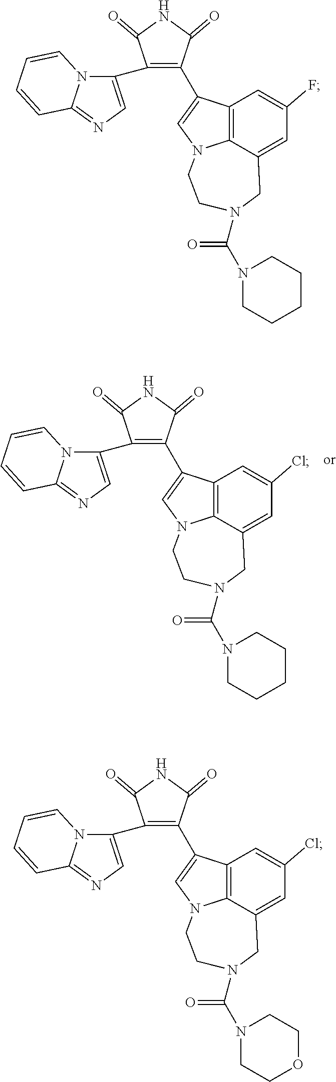 [SMILES: O=C1NC(=O)C(c2cnc3ccccn23)=C1c1cn2c3c(cc(F)cc13)CN(C(=O)N1CCCCC1)CC2 ; O=C1NC(=O)C(c2cnc3ccccn23)=C1c1cn2c3c(cc(Cl)cc13)CN(C(=O)N1CCCCC1)CC2 ; "or" ; O=C1NC(=O)C(c2cnc3ccccn23)=C1c1cn2c3c(cc(Cl)cc13)CN(C(=O)N1CCOCC1)CC2]

b) provided that when Ar is

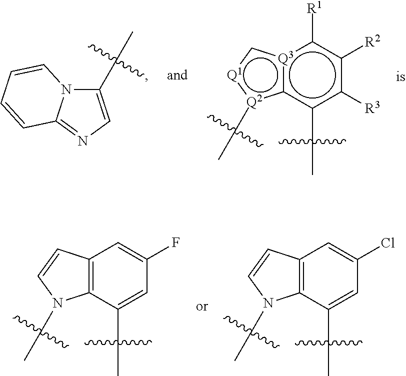

then R^{X1} is not

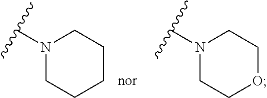 [SMILES: *N1CCCCC1 ; *N1CCOCC1]

In certain embodiments, the present disclosure provides a compound of formula (I) for use in the methods disclosed herein and that is not disclosed in WO 2003/076442 (PCT/US03/05050), which is incorporated herein by reference.

In certain embodiments of formula (I), R^{X} is —COR^{X1} or —SO₂R^{X1}.

In certain embodiments of formula (I), R^{X} is selected

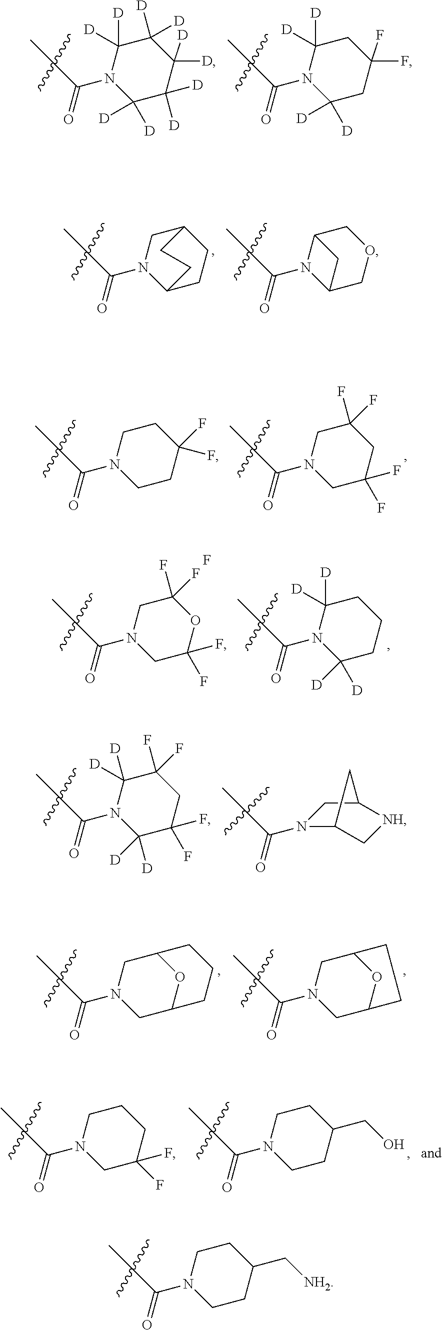

In certain embodiments, R^{X1} is heterocyclic, wherein the heterocyclic is optionally substituted with one to twelve substituents that is halo. In certain embodiments, R^{X1} is heterocyclic which is deuterated. In certain embodiments, the heterocyclic is monocyclic or bicyclic. In certain embodiments, the heterocyclic contains one to three nitrogens (i.e., 1, 2, or 3 nitrogens) and/or one to three oxygens (i.e., 1, 2, or 3 oxygens). In certain embodiments, the heterocyclic contains one nitrogen and/or one oxygen. In certain embodiments, the heterocyclic contains one nitrogen. In certain embodiments, the heterocyclic contains two nitrogens. In certain embodiments, the heterocyclic contains one nitrogen and one oxygen.

In certain embodiments, R^{X1} is piperidine or 8-oxa-3-azabicyclo[3.2.1]octane, both optionally substituted with one to twelve substituents independently selected from the group consisting of deuterium, halo, C₁-C₄alkyl, —(CH₂)ₚ—OH, —(CH₂)ₚ—NH₂; wherein p is 1, 2, or 3. In certain embodiments, R^{X1} is piperidine, optionally substituted with one to two halo substituents. In certain embodiments, R^{X1} is piperidine, optionally substituted with —(CH₂)ₚ—OH.

In certain embodiments of formula (I), the heterocyclic is optionally substituted with C₁-C₄alkyl, —(CH₂)ₚ—OH, or —(CH₂)ₚ—NH₂; wherein p is 1, 2, or 3. In certain embodiments, R^{X1} is heterocyclic substituted with C₁-C₄alkyl. In certain embodiments, R^{X1} is heterocyclic substituted with —(CH₂)ₚ—OH; wherein p is 1, 2, or 3. In certain embodiments, R^{X1} is heterocyclic substituted with —CH₂—OH. In certain embodiments, R^{X1} is heterocyclic substituted with —(CH₂)ₚ—NH₂; wherein p is 1, 2, or 3. In certain embodiments, R^{X1} is heterocyclic substituted with —CH₂—NH₂.

In certain embodiments of formula (I), R^{X1} is heterocyclic, wherein the heterocyclic is optionally substituted with —[C(R^{X1a})₂]ₚ—CN. In certain embodiments, R^{X1} is heterocyclic substituted with —[C(R^{X1a})₂]ₚ—OH, —[C(R^{X1a})₂]ₚ—O—C₁-C₄alkyl, —NHCOC₁-C₄alkyl, —CONHC₁-C₄alkyl, COH, —CO₂H, —[C(R^{X1a})₂]ₚ—COO—C₁-C₄alkyl, —[C(R^{X1a})₂]ₚ—NH₂, —[C(R^{X1a})₂]ₚ—NH—C₁-C₄alkyl, or —[C(R^{X1a})₂]ₚ—N—(C₁-C₄alkyl)₂. In certain embodiments, R^{X1} is heterocyclic, wherein the heterocyclic is optionally substituted with —CONHC₁-C₄alkyl, —COH, —CO₂H, or —[C(R^{X1a})₂]ₚ—COO—C₁-C₄alkyl.

In certain embodiments of formula (I), each R^{X1a} is independently selected from the group consisting of hydrogen and halo. In certain embodiments, both R^{X1a} groups together form C₃-C₆cycloalkyl, such as cyclopropyl, cyclobutyl, cyclopentyl, or cyclohexyl.

In certain embodiments of formula (I), R^{X} is heteroaryl. In certain embodiments, the heteroaryl is monocyclic or bicyclic. In certain embodiments, the heteroaryl contains one to three nitrogens (i.e., 1, 2, or 3 nitrogens) and/or one to three oxygens (i.e., 1, 2, or 3 oxygens). In certain embodiments, the heteroaryl contains one nitrogen and/or one oxygen. In certain embodiments, the heteroaryl contains one nitrogen. In certain embodiments, the heteroaryl contains two nitrogens. In certain embodiments, the heteroaryl contains one nitrogen and one oxygen. In certain embodiments, R^{X} is

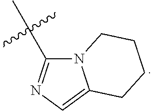 [SMILES: *c1ncc2n1CCCC2]

In certain embodiments of formula (I), R^{X} is —(C₁-C₄alkylene)-(C₃-C₈cycloalkyl). In certain embodiments, the —(C₁-C₄alkylene)-(C₃-C₈cycloalkyl) is substituted with one to two halo on the C₁-C₄alkylene. In certain embodiments, the C₃-C₈cycloalkyl is cyclopropyl, cyclobutyl, cyclopentyl, or cyclohexyl. In certain embodiments, R^{X} is —(C₁-C₄alkylene)-(C₃-C₈cycloalkyl), wherein the —(C₁-C₄alkylene)-(C₃-C₈cycloalkyl) is optionally substituted with one or two halo on the C₁-C₄alkylene and wherein C₃-C₈cycloalkyl is cyclopropyl, cyclobutyl, cyclopentyl, or cyclohexyl. In certain embodiments, R^{X} is

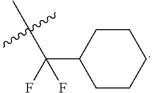 [SMILES: *C(F)(F)C1CCCCC1]

In some embodiments of the methods and compositions disclosed herein, the one or more GSK inhibitor is a compound having the Formula (Ia),

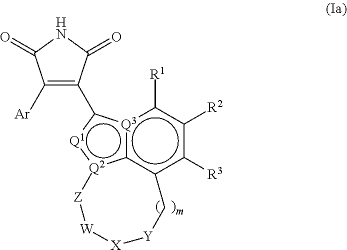

and pharmaceutically acceptable salts and tautomers thereof, wherein Q¹, Q², Q³, R¹, R², R³, Ar, —Z—W—X—Y— and m are as defined above for Formula (Ia).

In some embodiments of the methods and compositions disclosed herein, the one or more GSK inhibitor is a compound having the Formula (Ib),

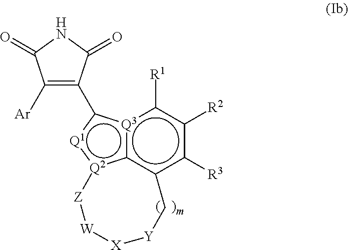

and pharmaceutically acceptable salts and tautomers thereof, wherein Q¹, Q², Q³, R¹, R², R³, Ar, —Z—W—X—Y— and m are as defined above for Formula (Ib).

In certain embodiments, Q¹ is CH; Q² is N; and Q³ is C. In certain embodiments, Q¹ is N; Q² is C; and Q³ is N. In certain embodiments, Q¹ is CH; Q² is C; and Q³ is N. In certain embodiments, ¹ is N; Q² is N; and Q³ is C.

In certain embodiments, the

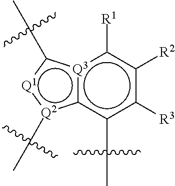

is selected from the group consisting of

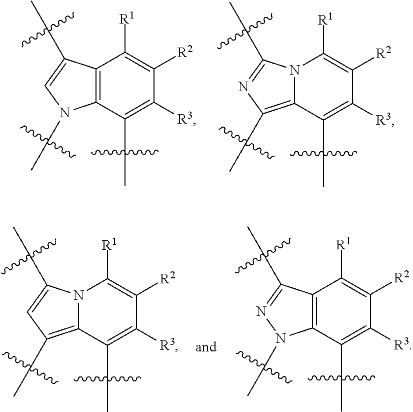

In certain embodiments, R¹ is hydrogen or halo. In certain embodiments, R¹ is C₁-C₄alkyl, wherein the alkyl is optionally substituted with one to 3 substituents independently selected from the group consisting of halo and —OH. In certain embodiments, R¹ is C₁-C₄alkynyl, —CN, —OH, or —S(O)₂NH₂. In certain embodiments, R¹ is —NH₂ or —NHC(O)R^{1a}, wherein R^{1a} is C₁-C₄alkyl. In certain embodiments, R¹ is C₁-C₄alkenyl. In certain embodiments, R¹ is —O—C₁-C₄alkyl.

In certain embodiments, R² is hydrogen or halo. In certain embodiments, R² is C₁-C₄alkyl, wherein the alkyl is optionally substituted with one to 3 substituents independently selected from the group consisting of halo and —OH. In certain embodiments, R² is C₁-C₄alkynyl, —CN, —OH, or —S(O)₂NH₂. In certain embodiments, R² is —NH₂ or —NHC(O)R^{2a}, wherein R^{2a} is C₁-C₄alkyl. In certain embodiments, R² is —S(O)₂NH₂.

In certain embodiments, R² is C₁-C₄alkenyl. In certain embodiments, R² is —O—C₁-C₄alkyl. In certain embodiments, R² is —NH₂, —NH(C₁-C₄alkyl), or —N(C₁-C₄alkyl)₂.

In certain embodiments, R² is selected from the group consisting of halo, C₁-C₄alkyl, C₁-C₄alkenyl, C₁-C₄alkynyl, —CN, —OH, —O—C₁-C₄alkyl, —NH₂, —NH(C₁-C₄alkyl), —N(C₁-C₄alkyl)₂, —NHC(O)R^{2a}, and —S(O)₂NH₂; wherein the alkyl is optionally substituted with one to 3 substituents independently selected from the group consisting of halo and —OH; and wherein R^{2a} is C₁-C₄alkyl. In certain embodiments, R² is selected from the group consisting of halo, C₁-C₄alkyl, C₁-C₄alkynyl, —CN, —OH, —NH₂, —NHC(O)R^{2a}, and —S(O)₂NH₂; wherein the alkyl is optionally substituted with one to 3 substituents independently selected from the group consisting of halo and —OH; and wherein R^{2a} is C₁-C₄alkyl. In certain embodiments, R² is not hydrogen.

In certain embodiments, R³ is hydrogen or halo. In certain embodiments, R³ is C₁-C₄alkyl, wherein the alkyl is optionally substituted with one to 3 substituents independently selected from the group consisting of halo and —OH. In certain embodiments, R³ is C₁-C₄alkynyl, —CN, —OH, or —S(O)₂NH₂. In certain embodiments, R³ is —NH₂ or —NHC(O)R^{3a}, wherein R^{3a} is C₁-C₄alkyl. In certain embodiments, R³ is C₁-C₄alkenyl. In certain embodiments, R³ is —O—C₁-C₄alkyl.

In certain embodiments, Ar is

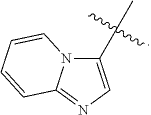 [SMILES: *c1cnc2ccccn12]

In certain embodiments, Ar is

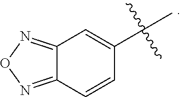 [SMILES: *c1ccc2nonc2c1]

In certain embodiments, Ar is

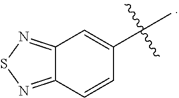 [SMILES: *c1ccc2nsnc2c1]

In certain embodiments, Ar is

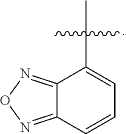 [SMILES: *c1cccc2nonc12]

In certain embodiments, Ar is

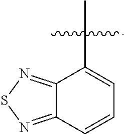 [SMILES: *c1cccc2nsnc12]

In certain embodiments, Ar is

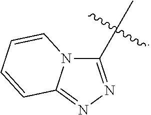 [SMILES: *c1nnc2ccccn12]

In certain embodiments, Ar is

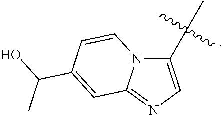 [SMILES: *c1cnc2cc(C(C)O)ccn12]

In certain embodiments, Ar is

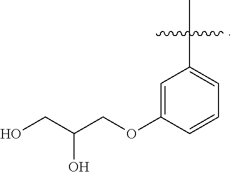 [SMILES: *c1cccc(OCC(O)CO)c1]

In certain embodiments, Ar is

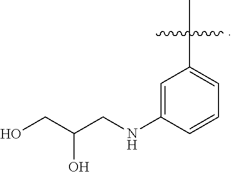 [SMILES: *c1cccc(NCC(O)CO)c1]

In certain embodiments, Ar is

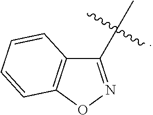 [SMILES: *c1noc2ccccc12]

In certain embodiments, Ar is

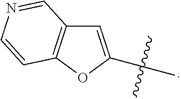 [SMILES: *c1cc2cnccc2o1]

In certain embodiments, Ar is

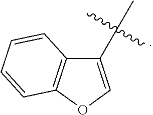 [SMILES: *c1coc2ccccc12]

In certain embodiments, Ar is

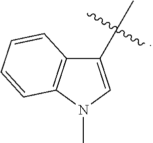 [SMILES: *c1cn(C)c2ccccc12]

In certain embodiments, Ar is

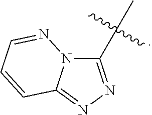 [SMILES: *c1nnc2cccnn12]

In certain embodiments of Formula (Ia), Ar is

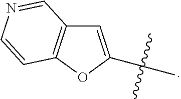 [SMILES: *c1cc2cnccc2o1]

In certain embodiments of Formula (Ia), Ar is

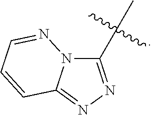 [SMILES: *c1nnc2cccnn12]

In certain embodiments of Formula (Ia), Ar is

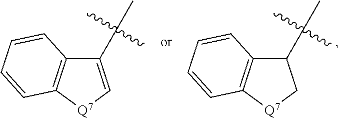

wherein Q⁷ is selected from S, O, CH₂, and NR^{Q7}; wherein R^{Q7} is hydrogen or optionally substituted C₁-C₄alkyl.

In certain embodiments of Formula (Ib), Ar is

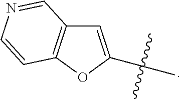 [SMILES: *c1cc2cnccc2o1]

In certain embodiments of Formula (Ib), Ar is

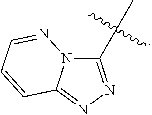 [SMILES: *c1nnc2cccnn12]

In certain embodiments of Formula (Ib), Ar is

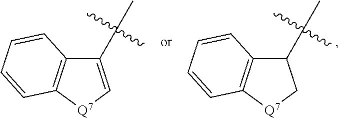

wherein Q⁷ is selected from S, O, CH₂, and NR^{Q7}; wherein R^{Q7} is hydrogen or optionally substituted C₁-C₄alkyl. In certain embodiments of Formula (Ib), Ar is

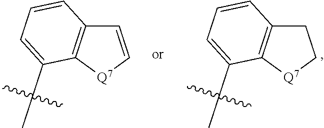

wherein Q⁷ is selected from S, O, CH₂, and NR^{Q7}; wherein R^{Q7} is hydrogen or optionally substituted C₁-C₄alkyl. In certain embodiments of Formula (Ib), Ar is

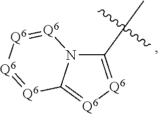

wherein each Q⁶ is independently selected from CR^{Q6} and N; wherein R^{Q6} is hydrogen, halo, —CN, lower alkyl, or substituted alkyl.

In certain embodiments, —Z—W—X—Y— is —C(R^{Z})₂—C(R^{W})₂—N(R^{X})—(R^{Y})₂—. In certain embodiments, —Z—W—X—Y— is —C(R^{Z})₂—C(R^{W})₂—CH(R^{X})—(R^{Y})₂—. In certain embodiments, —Z—W—X—Y— is —C(R^{W})₂—CH(R^{X})—C(R^{Y})₂—.

In certain embodiments, each R^{Z} is independently selected from the group consisting of hydrogen and halo. In certain embodiments, both R^{Z} groups together form C₃-C₆cycloalkyl, such as cyclopropyl, cyclobutyl, cyclopentyl, or cyclohexyl. In certain embodiments, both R^{Z} groups together form oxo. In certain embodiments, R^{Z} and R^{W} together with the carbons to which they are attached form a C₃-C₆cycloalkyl, such as cyclopropyl, cyclobutyl, cyclopentyl, or cyclohexyl.

In certain embodiments, each R^{W} is independently selected from the group consisting of hydrogen and halo. In certain embodiments, both R^{W} groups together form C₃-C₆cycloalkyl, such as cyclopropyl, cyclobutyl, cyclopentyl, or cyclohexyl. In certain embodiments, both R^{W} groups together form oxo. In certain embodiments, R^{Z} and R^{W} together with the carbons to which they are attached form a C₃-C₆cycloalkyl, such as cyclopropyl, cyclobutyl, cyclopentyl, or cyclohexyl.

In certain embodiments, each R^{Y} is independently selected from the group consisting of hydrogen and halo. In certain embodiments, both R^{W} groups together form C₃-C₆cycloalkyl, such as cyclopropyl, cyclobutyl, cyclopentyl, or cyclohexyl. In certain embodiments, both R^{Y} groups together form oxo.

In certain embodiments of Formula (Ia) and (Ib), R^{X} is H. In certain embodiments of Formula (Ia) and (Ib), R^{X} is R^{X1}, which is C₃-C₈cycloalkyl, heteroaryl, or heterocyclic, wherein the heterocyclic is optionally substituted with one to twelve substituents independently selected from the group consisting of deuterium, halo, C₁-C₄alkyl, —(CH₂)ₚ—OH, —[C(R^{X1a})₂]ₚ—OH, —[C(R^{X1a})₂]ₚ—O—C₁-C₄alkyl, —NHCOC₁-C₄alkyl, —CONHC₁-C₄alkyl, —(CH₂)ₚ—NH₂, —[C(R^{X1a})₂]ₚ—NH₂, —[C(R^{X1a})₂]ₚ—NH—C₁-C₄alkyl, —[C(R^{X1a})₂]ₚ—N—(C₁-C₄alkyl)₂; wherein p is 0, 1, 2, or 3; wherein each R^{X1a} is independently selected from the group consisting of hydrogen, deuterium, halo, and C₁-C₄alkyl, or both R^{X1a} groups together form C₃-C₆cycloalkyl.

In certain embodiments of Formula (Ia) and (Ib), R^{X} is —COR^{X1} or —SO₂R^{X1}.

In certain embodiments of Formula (Ia) and (Ib), R^{X} is selected from

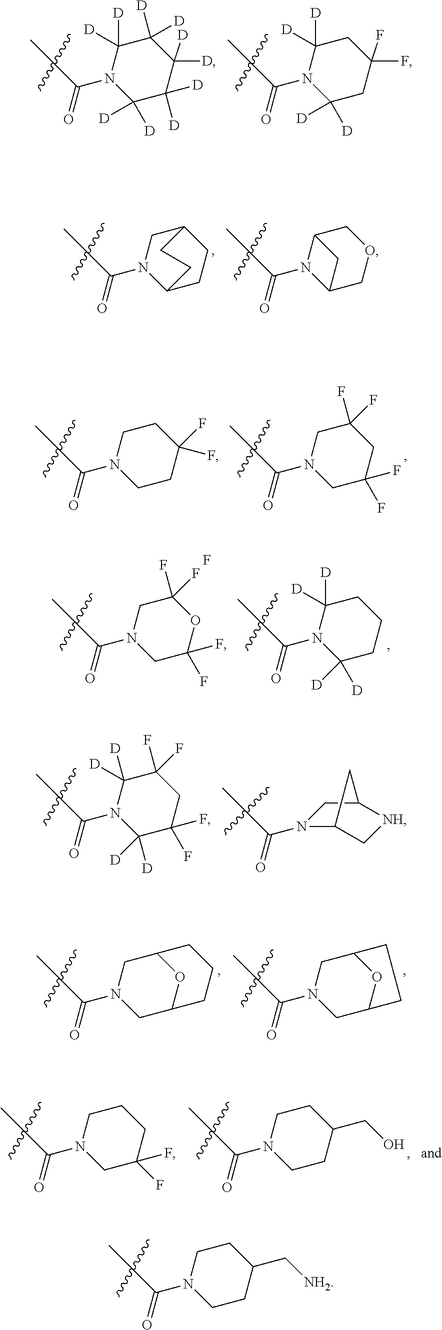

In certain embodiments of Formula (Ia) and (Ib), R^{X} is —(C₁-C₄alkylene)-R^{X1}, wherein the —(C₁-C₄alkylene)-R^{X1} is optionally substituted with one to four halo on the C₁-C₄alkylene. In certain embodiments, the —(C₁-C₄alkylene)-R^{X1} is substituted with one to four halo on the C₁-C₄alkylene. In certain embodiments, the —(C₁-C₄alkylene)-R^{X1} is substituted with one or two halo on the C₁-C₄alkylene. In certain embodiments, R^{X} is —(C₁-C₄alkylene)-R^{X1}, wherein the —(C₁-C₄alkylene)-R^{X1} is optionally substituted with one or two halo on the C₁-C₄alkylene and wherein R^{X1} is cyclopropyl, cyclobutyl, cyclopentyl, or cyclohexyl. In certain embodiments, R^{X} is

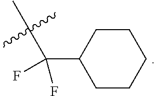 [SMILES: *C(F)(F)C1CCCCC1]

In certain embodiments of Formula (Ia) and (Ib), R^{X1} is C₃-C₈cycloalkyl. In certain embodiments, R^{X1} is cyclopropyl, cyclobutyl, cyclopentyl, or cyclohexyl.

In certain embodiments of Formula (Ia) and (Ib), R^{X1} is heterocyclic, wherein the heterocyclic is optionally substituted with one to twelve substituents that is halo. In certain embodiments, R^{X1} is heterocyclic which is deuterated. In certain embodiments, the heterocyclic is monocyclic or bicyclic. In certain embodiments, the heterocyclic contains one to three nitrogens (i.e., 1, 2, or 3 nitrogens) and/or one to three oxygens (i.e., 1, 2, or 3 oxygens). In certain embodiments, the heterocyclic contains one nitrogen and/or one oxygen. In certain embodiments, the heterocyclic contains one nitrogen. In certain embodiments, the heterocyclic contains two nitrogens. In certain embodiments, the heterocyclic contains one nitrogen and one oxygen.

In certain embodiments of Formula (Ia) and (Ib), R^{X1} is heterocyclic, wherein the heterocyclic is the heterocyclic is optionally substituted with C₁-C₄alkyl, —(CH₂)ₚ—OH, or —(CH₂)ₚ—NH₂; wherein p is 1, 2, or 3. In certain embodiments, R^{X1} is heterocyclic substituted with C₁-C₄alkyl. In certain embodiments, R^{X1} is heterocyclic substituted with —(CH₂)ₚ—OH; wherein p is 1, 2, or 3. In certain embodiments, R^{X1} is heterocyclic substituted with —(CH₂)—OH. In certain embodiments, R^{X1} is heterocyclic substituted with —(CH₂)ₚ—NH₂; wherein p is 1, 2, or 3. In certain embodiments, R^{X1} is heterocyclic substituted with —(CH₂)—NH₂.

In certain embodiments of Formula (Ia) and (Ib), R^{X1} is heterocyclic, wherein the heterocyclic is optionally substituted with —[C(R^{X1a})₂]ₚ—CN. In certain embodiments, R^{X1} is heterocyclic substituted with —[C(R^{X1a})₂]ₚ—OH, —[C(R^{X1a})₂]ₚ—O—C₁-C₄alkyl, —NHCOC₁-C₄alkyl, —[C(R^{X1a})₂]ₚ—NH₂, —[C(R^{X1a})₂]ₚ—NH—C₁-C₄alkyl, or —[C(R^{X1a})₂]ₚ—N—(C₁-C₄alkyl)₂. In certain embodiments, R^{X1} is heterocyclic, wherein the heterocyclic is optionally substituted with —CONHC₁-C₄alkyl, —COH, —CO₂H, or —[C(R^{X1a})₂]ₚ—COO—C₁-C₄alkyl.

In certain embodiments of Formula (Ia) and (Ib), each R^{X1a} is independently selected from the group consisting of hydrogen and halo. In certain embodiments, both R^{X1a} groups together form C₃-C₆cycloalkyl, such as cyclopropyl, cyclobutyl, cyclopentyl, or cyclohexyl.

In certain embodiments of Formula (Ia) and (Ib), R^{X1} is heteroaryl. In certain embodiments, the heteroaryl is monocyclic or bicyclic. In certain embodiments, the heteroaryl contains one to three nitrogens (i.e., 1, 2, or 3 nitrogens) and/or one to three oxygens (i.e., 1, 2, or 3 oxygens). In certain embodiments, the heteroaryl contains one nitrogen and/or one oxygen. In certain embodiments, the heteroaryl contains one nitrogen. In certain embodiments, the heteroaryl contains two nitrogens. In certain embodiments, the heteroaryl contains one nitrogen and one oxygen. In certain embodiments, R^{X1} is

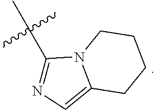 [SMILES: *c1ncc2n1CCCC2]

In certain embodiments of Formula (Ia) and (Ib), R^{X} is —CON(R^{X2})₂. In certain embodiments, R^{X} is —CON(R^{X2})₂, wherein R^{X2} is hydrogen or methyl. In certain embodiments, R^{X} is —CONH₂. In certain embodiments, R^{X} is —CON(R^{X2})₂, wherein R^{X2} is C₁-C₄alkyl. In certain embodiments, R^{X} is —CON(R^{X2})₂, wherein R^{X2} is methyl.

In certain embodiments, m is 0. In certain embodiments, m is 1. In certain embodiments, m is 2.

In one variation of the compounds disclosed herein, Ar is

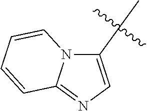 [SMILES: *c1cnc2ccccn12]

and Q¹ is CH; Q² is N; Q³ is C; Q⁴ is C; and Q⁵ is C.

In one variation of the compounds disclosed herein, Ar is

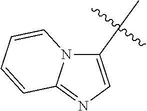 [SMILES: *c1cnc2ccccn12]

and Q¹ is CH; Q² is N; Q³ is C; Q⁴ is C; and Q⁵ is C.

In some embodiments of the methods and compositions disclosed herein, the one or more GSK inhibitor is of formula:

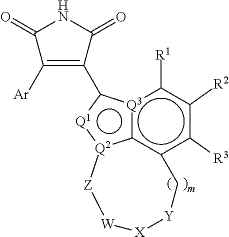

having one, two, three, or more of the following features:

a) Ar is

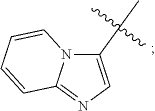 [SMILES: *c1cnc2ccccn12]

b) Q¹ is CH; Q² is N; and Q³ is C;

c) R² is hydrogen or halo;

d) —Z—W—X—Y— is —C(R^{Z})₂—C(R^{W})₂—N(R^{X})—C(R^{Y})₂—;

e) R^{X} is —COR^{X1}.

In some embodiments of the methods and compositions disclosed herein, the one or more GSK inhibitor is of formula:

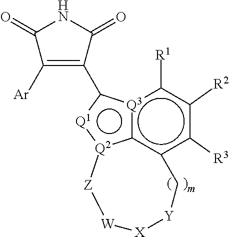

having one, two, three, or more of the following features:

a) Ar is

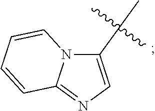 [SMILES: *c1cnc2ccccn12]

b) Q¹ is CH; Q² is N; and Q³ is C;

c) R² is C₁-C₄alkyl, wherein the alkyl is optionally substituted with one to 3 substituents independently selected from the group consisting of halo and —OH;

d) —Z—W—X—Y— is —C(R^{Z})₂—C(R^{W})₂—N(R^{X})—C(R^{X})₂—;

e) R^{X} is —COR^{X1}.

In some embodiments of the methods and compositions disclosed herein, the one or more GSK inhibitor is of formula:

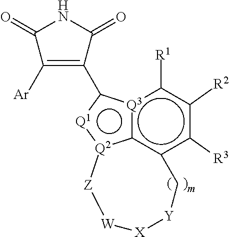

having one, two, three, or more of the following features:

a) Ar is

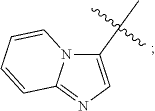 [SMILES: *c1cnc2ccccn12]

b) Q¹ is CH; Q² is N; and Q³ is C;

c) R² is C₁-C₄alkynyl, —CN, —OH, —S(O)₂NH₂, —NH₂ or —NHC(O)R^{2a};

d) —Z—W—X—Y— is —C(R^{Z})₂—C(R^{W})₂—N(R^{X})—C(R^{X})₂—;

e) R^{X} is —COR^{X1}.

Nonlimiting examples of GSK inhibitors for use in the methods of the present disclosure are presented in Table 6.

TABLE 6
Exemplary compounds for use in compositions and methods of the present disclosure

| | | |
|---|---|---|
| Compound I-1 | 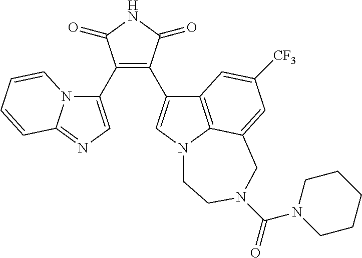 [SMILES: O=C1NC(=O)C(c2cnc3ccccn23)=C1c1cn2c3c(cc(C(F)(F)F)cc13)CN(C(=O)N1CCCCC1)CC2] | 3-(imidazo[1,2-a]pyridin-3-yl)-4-(2- (piperidine-1-carbonyl)-9- (trifluoromethyl)-1,2,3,4-tetrahydro- [1,4]diazepino[6,7,1-hi]indol-7-yl)-1H- pyrrole-2,5-dione |
| Compound I-2 | 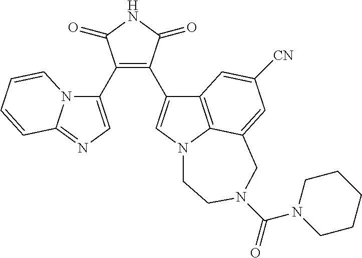 [SMILES: N#Cc1cc2c3c(c1)c(C1=C(c4cnc5ccccn45)C(=O)NC1=O)cn3CCN(C(=O)N1CCCCC1)C2] | 7-(4-(imidazo(1,2-a]pyridin-3-yl)-2,5- dioxo-2,5-dihydro-1H-pyrrol-3-yl)-2- (piperidine-1-carbonyl)-1,2,3,4- tetrahydro-[1,4]diazepino[6,7,1- hi]indole-9-carbonitrile |
| Compound I-3 | 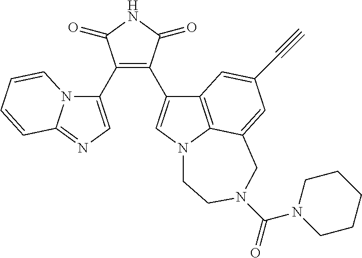 [SMILES: C#Cc1cc2c3c(c1)c(C1=C(c4cnc5ccccn45)C(=O)NC1=O)cn3CCN(C(=O)N1CCCCC1)C2] | 3-(9-ethynyl-2-(piperidine-1-carbonyl)- 1,2,3,4-tetrahydro-[1,4]diazepino[6,7,1- hi]indol-7-yl)-4-(imidazo[l,2-a]pyridin-3- yl)-1H-pyrrole-2,5-dione |
| Compound I-4 | 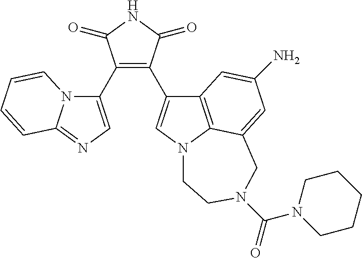 [SMILES: Nc1cc2c3c(c1)c(C1=C(c4cnc5ccccn45)C(=O)NC1=O)cn3CCN(C(=O)N1CCCCC1)C2] | 3-(9-amino-2-(piperidine-1-carbonyl)- 1,2,3,4-tetrahydro-[1,4]diazepino[6,7,1- hi]indol-7-yl)-4-(imidazo[l,2-a]pyridin-3- yl)-1H-pyrrole-2,5-dione |
| Compound I-5 | 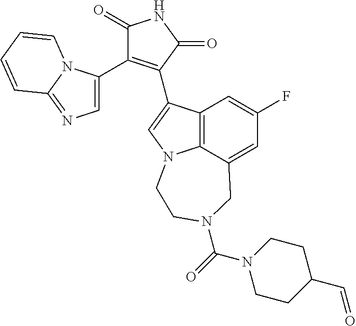 [SMILES: O=CC1CCN(C(=O)N2CCn3cc(C4=C(c5cnc6ccccn56)C(=O)NC4=O)c4cc(F)cc(c43)C2)CC1] | 1-(9-fluoro-7-(4-(imidazo[1,2-a] pyridin-3-yl)-2,5-dioxo-2,5- dihydro-1H-pyrrol-3-yl)- 1,2,3,4-tetrahydro-[1,4]diazepino [6,7,1-hi]indole-2-carbonyl) piperidine-4-carbaldehyde |
| Compound I-6 | 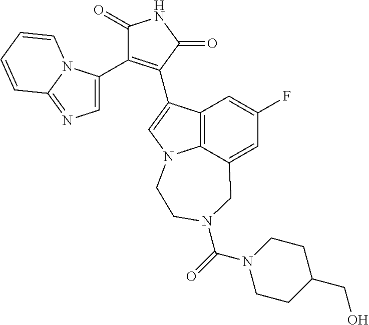 [SMILES: O=C1NC(=O)C(c2cnc3ccccn23)=C1c1cn2c3c(cc(F)cc13)CN(C(=O)N1CCC(CO)CC1)CC2] | 3-(9-fluoro-2-(4- (hydroxymethyl)piperidine-1-carbonyl)- 1,2,3,4-tetrahydro-[1,4]diazepino[6,7,1- hi]indol-7-yl)-4-(imidazo[1,2-a]pyridin-3- yl)-1H-pyrrole-2,5-dione |
| Compound I-7 | 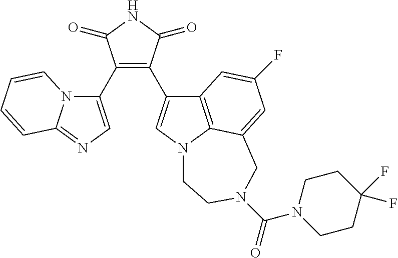 [SMILES: O=C1NC(=O)C(c2cnc3ccccn23)=C1c1cn2c3c(cc(F)cc13)CN(C(=O)N1CCC(F)(F)CC1)CC2] | 3-(2-(4,4-difluoropiperidine-1-carbonyl)- 9-fluoro-1,2,3,4-tetrahydro- [1,4]diazepino[6,7,1-hi]indol-7-yl)-4- (imidazo[1,2-a]pyridin-3-yl)-1H-pyrrole- 2,5-dione |
| Compound I-8 | 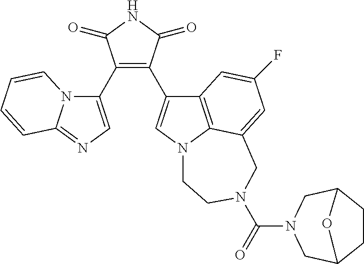 [SMILES: O=C1NC(=O)C(c2cnc3ccccn23)=C1c1cn2c3c(cc(F)cc13)CN(C(=O)N1CC3CCC(C1)O3)CC2] | 3-(2-(8-oxa-3-azabicyclo [3.2.1]octane-3-carbonyl)-9-fluoro- 1,2,3,4-tetrahydro-[1,4]diazepino [6,7,1-hi]indol-7-yl)-4- (imidazo[1,2-a]pyridin-3-yl)- 1H-pyrrole-2,5-dione |
| Compound I-9 | 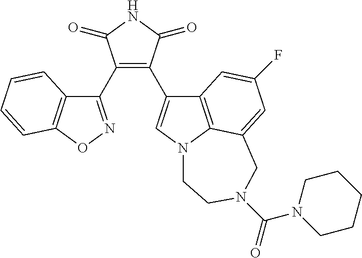 [SMILES: O=C1NC(=O)C(c2cn3c4c(cc(F)cc24)CN(C(=O)N2CCCCC2)CC3)=C1c1noc2ccccc12] | 3-(benzo[d]isoxazol-3-yl)-4-(9-fluoro-2- (piperidine-1-carbonyl)-1,2,3,4- tetrahydro-[1,4]diazepino[6,7,1-hi] indol-7-yl)-1H-pyrrole-2,5-dione |
| Compound I-10 | 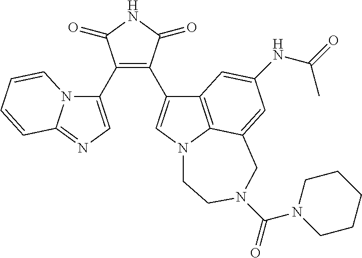 [SMILES: CC(=O)Nc1cc2c3c(c1)c(C1=C(c4cnc5ccccn45)C(=O)NC1=O)cn3CCN(C(=O)N1CCCCC1)C2] | N-(7-(4-(imidazo[1,2-a]pyridin-3-yl)- 2,5-dioxo-2,5-dihydro-1H-pyrrol- 3-yl)-2-(piperidine-1-carbonyl)- 1,2,3,4-tetrahydro-[1,4]diazepino [6,7,1-hi]indol-9-yl)acetamide |
| Compound I-11 | 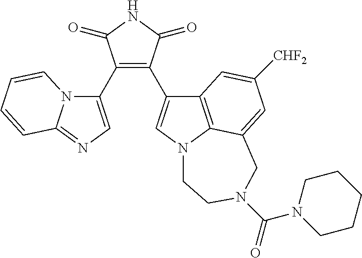 [SMILES: O=C1NC(=O)C(c2cnc3ccccn23)=C1c1cn2c3c(cc(C(F)F)cc13)CN(C(=O)N1CCCCC1)CC2] | 3-(9-(difluoromethyl)-2-(piperidine-1- carbonyl)-1,2,3,4-tetrahydro- [1,4]diazepino[6,7,1-hi]indol-7-yl)-4- (imidazo[1,2-a]pyridin-3-yl)-1H-pyrrole- 2,5-dione |
| Compound I-12 | 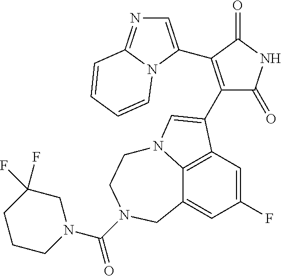 [SMILES: O=C1NC(=O)C(c2cnc3ccccn23)=C1c1cn2c3c(cc(F)cc13)CN(C(=O)N1CCCC(F)(F)C1)CC2] | 3-(2-(3,3-difluoropiperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H- pyrrole-2,5-dione |
| Compound I-13 | 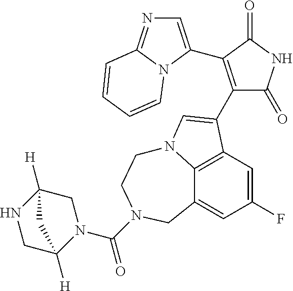 [SMILES: O=C1NC(=O)C(c2cnc3ccccn23)=C1c1cn2c3c(cc(F)cc13)CN(C(=O)N1C[C@H]3C[C@@H]1CN3)CC2] | 3-(2-((1R,4R)-2,5- diazabicyclo[2.2.1]heptane-2-carbonyl)- 9-fluoro-1,2,3,4-tetrahydro- (1,4]diazepino[6,7,1-hi]indol-7-yl)-4- (imidazo[1,2-a]pyridin-3-yl)-1H- pyrrole-2,5-dione |
| Compound I-14 | 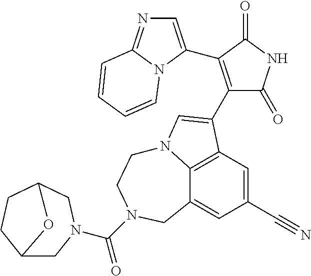 [SMILES: N#Cc1cc2c3c(c1)c(C1=C(c4cnc5ccccn45)C(=O)NC1=O)cn3CCN(C(=O)N1CC3CCC(C1)O3)C2] | 2-(8-oxa-3-azabicyclo[3.2.1]octane-3- carbonyl)-7-(4-(imidazo[1,2-a]pyridin- 3-yl)-2,5-dioxo-2,5-dihydro-1H-pyrrol- 3-yl)-1,2,3,4-tetrahydro- [1,4]diazepino[6,7,1-hi]indole- 9-carbonitrile |
| Compound I-15 | 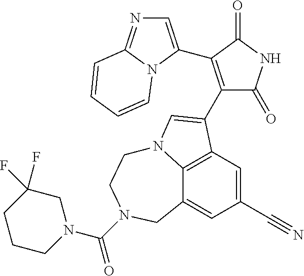 [SMILES: N#Cc1cc2c3c(c1)c(C1=C(c4cnc5ccccn45)C(=O)NC1=O)cn3CCN(C(=O)N1CCCC(F)(F)C1)C2] | 2-(3,3-difluoropiperidine-1-carbonyl)-7- (4-(imidazo(1,2-a]pyridin-3-yl)-2,5- dioxo-2,5-dihydro-1H-pyrrol-3- yl)-1,2,3,4- tetrahydro-[1,4]diazepino[6,7,1- hi]indole-9-carbonitrile |
| Compound I-16 | 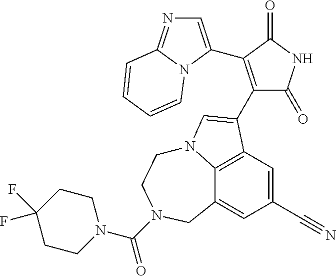 [SMILES: N#Cc1cc2c3c(c1)c(C1=C(c4cnc5ccccn45)C(=O)NC1=O)cn3CCN(C(=O)N1CCC(F)(F)CC1)C2] | 2-(4,4-difluoropiperidine-1-carbonyl)- 7-(4-(imidazo[1,2-a]pyridin-3-yl)- 2,5-dioxo- 2,5-dihydro-1H-pyrrol-3-yl)-1,2,3,4- tetrahydro-[1,4]diazepino[6,7,1- hi]indole-9-carbonitrile |
| Compound I-17 | 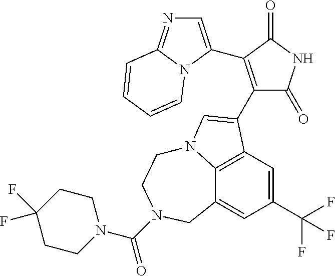 [SMILES: O=C1NC(=O)C(c2cnc3ccccn23)=C1c1cn2c3c(cc(C(F)(F)F)cc13)CN(C(=O)N1CCC(F)(F)CC1)CC2] | 3-(2-(4,4-difluoropiperidine-1-carbonyl)- 9-(trifluoromethyl)-1,2,3,4-tetrahydro- [1,4]diazepino[6,7,1-hi]indol-7-yl)-4- (imidazo[1,2-a]pyridin-3-yl)-1H-pyrrole- 2,5-dione |
| Compound I-18 | 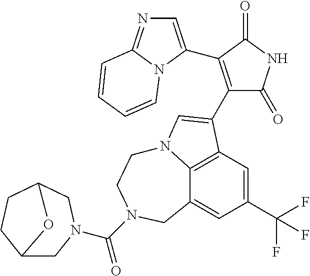 [SMILES: O=C1NC(=O)C(c2cnc3ccccn23)=C1c1cn2c3c(cc(C(F)(F)F)cc13)CN(C(=O)N1CC3CCC(C1)O3)CC2] | 3-(2-(8-oxa-3-azabicyclo[3.2.1]octane- 3-carbonyl)-9-(trifluoromethyl)-1,2,3,4- tetrahydro-[1,4]diazepino[6,7,1-hi]indol- 7-yl)-4-(imidazo[1,2-a]pyridin-3-yl)-1H- pyrrole-2,5-dione |
| Compound I-19 | 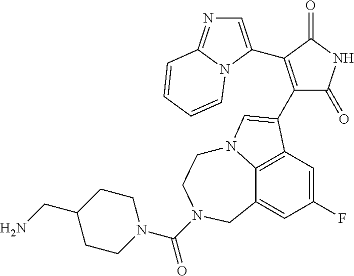 [SMILES: NCC1CCN(C(=O)N2CCn3cc(C4=C(c5cnc6ccccn56)C(=O)NC4=O)c4cc(F)cc(c43)C2)CC1] | 3-(2-(4-(aminomethyl)piperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H- pyrrole-2,5-dione |
| Compound I-20 | 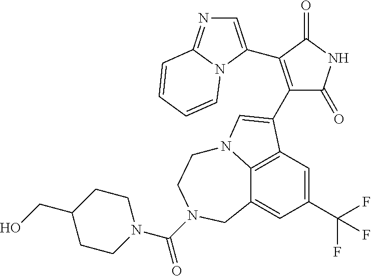 [SMILES: O=C1NC(=O)C(c2cnc3ccccn23)=C1c1cn2c3c(cc(C(F)(F)F)cc13)CN(C(=O)N1CCC(CO)CC1)CC2] | 3-(2-(4-(hydroxymethyl)piperidine-1- carbonyl)-9-(trifluoromethyl)-1,2,3,4- tetrahydro-(1,4]diazepino[6,7,1-hi]indol- 7-yl)-4-(imidazo[1,2-a]pyridin-3-yl)-1H- pyrrole-2,5-dione |
| Compound I-21 | 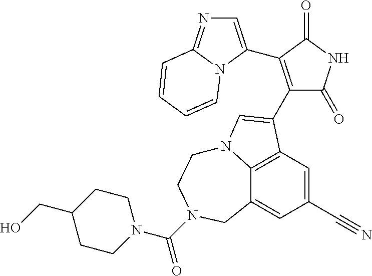 [SMILES: N#Cc1cc2c3c(c1)c(C1=C(c4cnc5ccccn45)C(=O)NC1=O)cn3CCN(C(=O)N1CCC(CO)CC1)C2] | 2-(4-(hydroxymethyl)piperidine-1- carbonyl)-7-(4-(imidazo[1,2-a]pyridin-3- yl)-2,5-dioxo-2,5-dihydro-1H-pyrrol- 3-yl)-1,2,3,4-tetrahydro-[1,4]diazepino [6,7,1-hi]indole-9-carbonitrile |
| Compound I-22 | 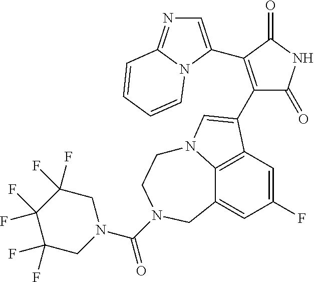 [SMILES: O=C1NC(=O)C(c2cnc3ccccn23)=C1c1cn2c3c(cc(F)cc13)CN(C(=O)N1CC(F)(F)C(F)(F)C(F)(F)C1)CC2] | 3-(9-fluoro-2-(3,3,4,4,5,5- hexafluoropiperidine-1-carbonyl)- 1,2,3,4-tetrahydro-[1,4]diazepino [6,7,1-hi]indol- 7-yl)-4-(imidazo[1,2-a]pyridin- 3-yl)-1H-pyrrole-2,5-dione |
| Compound I-23 | 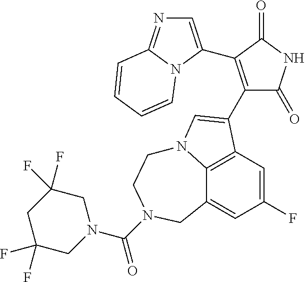 [SMILES: O=C1NC(=O)C(c2cnc3ccccn23)=C1c1cn2c3c(cc(F)cc13)CN(C(=O)N1CC(F)(F)CC(F)(F)C1)CC2] | 3-(9-fluoro-2-(3,3,5,5- tetrafluoropiperidine-1-carbonyl)- 1,2,3,4-tetrahydro-[1,4]diazepino [6,7,1-hi]indol-7-yl)-4-(imidazo[1,2-a] pyridin-3-yl)-1H-pyrrole-2,5-dione |
| Compound I-24 | 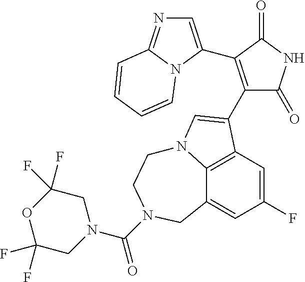 [SMILES: O=C1NC(=O)C(c2cnc3ccccn23)=C1c1cn2c3c(cc(F)cc13)CN(C(=O)N1CC(F)(F)OC(F)(F)C1)CC2] | 3-(9-fluoro-2-(2,2,6,6- tetrafluoromorpholine-4-carbonyl)- 1,2,3,4-tetrahydro-[1,4]diazepino[6,7,1- hi]indol-7-yl)-4-(imidazo[1,2-a] pyridin-3-yl)-1H-pyrrole-2,5-dione |
| Compound I-25 | 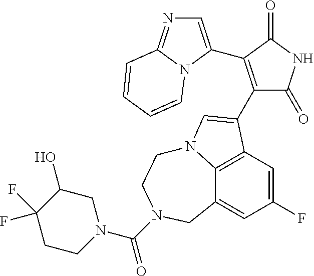 [SMILES: O=C1NC(=O)C(c2cnc3ccccn23)=C1c1cn2c3c(cc(F)cc13)CN(C(=O)N1CCC(F)(F)C(O)C1)CC2] | 3-(2-(4,4-difluoro-3-hydroxypiperidine- 1-carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole- 2,5-dione |
| Compound I-26 | 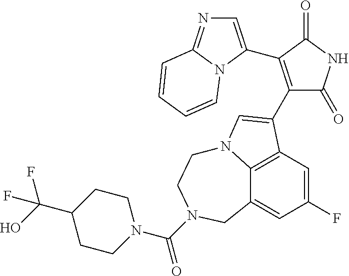 [SMILES: O=C1NC(=O)C(c2cnc3ccccn23)=C1c1cn2c3c(cc(F)cc13)CN(C(=O)N1CCC(C(O)(F)F)CC1)CC2] | 3-(2-(4- (difluoro(hydroxy)methyl)piperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole- 2,5-dione |
| Compound I-27 | 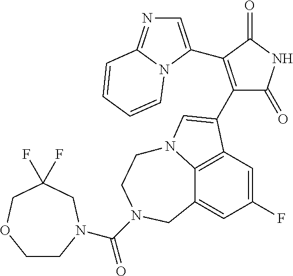 [SMILES: O=C1NC(=O)C(c2cnc3ccccn23)=C1c1cn2c3c(cc(F)cc13)CN(C(=O)N1CCOCC(F)(F)C1)CC2] | 3-(2-(6,6-difluoro-1,4-oxazepane-4- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole- 2,5-dione |
| Compound I-28 | 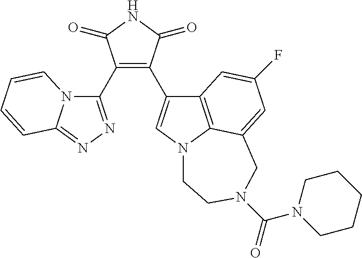 [SMILES: O=C1NC(=O)C(c2nnc3ccccn23)=C1c1cn2c3c(cc(F)cc13)CN(C(=O)N1CCCCC1)CC2] | 3-([1,2,4]triazolo[4,3-a]pyridin-3-yl)- 4-(9-fluoro-2-(piperidine-1-carbonyl)- 1,2,3,4-tetrahydro-[1,4]diazepino [6,7,l-hi]indol-7-yl)-1H-pyrrole- 2,5-dione |
| Compound I-29 | 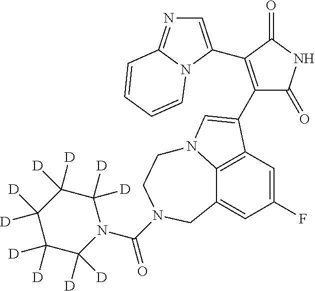 [SMILES: [2H]C1([2H])N(C(=O)N2CCn3cc(C4=C(c5cnc6ccccn56)C(=O)NC4=O)c4cc(F)cc(c43)C2)C([2H])([2H])C([2H])([2H])C([2H])([2H])C1([2H])[2H]] | 3-(9-fluoro-2-(piperidine-1-carbonyl- d10)-1,2,3,4-tetrahydro- [1,4]diazepino[6,7,1-hi]indol-7-yl)-4- (imidazo[1,2-a]pyridin-3-yl)- 1H-pyrrole-2,5-dione |
| Compound I-30 | 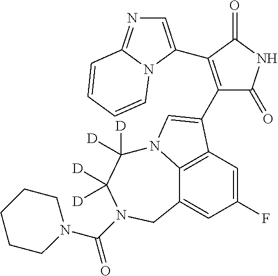 [SMILES: [2H]C1([2H])N(C(=O)N2CCCCC2)Cc2cc(F)cc3c(C4=C(c5cnc6ccccn56)C(=O)NC4=O)cn(c23)C1([2H])[2H]] | 3-(9-fluoro-2-(piperidine-1-carbonyl)- 1,2,3,4-tetrahydro-[1,4]diazepino[6,7,1- hi]indol-7-yl-3,3,4,4-d4)-4-(imidazo[1,2- a]pyridin-3-yl)-1H-pyrrole-2,5-dione |
| Compound I-31 | 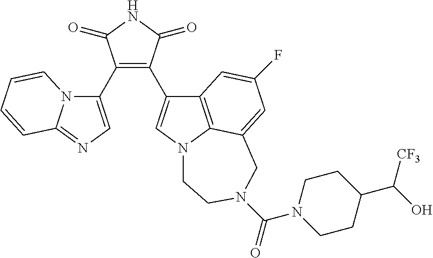 [SMILES: O=C1NC(=O)C(c2cnc3ccccn23)=C1c1cn2c3c(cc(F)cc13)CN(C(=O)N1CCC(C(O)C(F)(F)F)CC1)CC2] | 3-(9-fluoro-2-(4-(2,2,2-trifluoro-1- hydroxyethyl)piperidine-1-carbonyl)- 1,2,3,4-tetrahydro-[1,4]diazepino[6,7,1- hi]indol-7-yl)-4-(imidazo[1,2-a] pyridin-3-yl)-1H-pyrrole-2,5-dione |
| Compound I-32 | 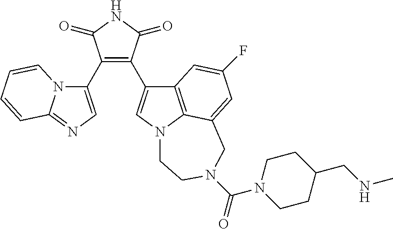 [SMILES: CNCC1CCN(C(=O)N2CCn3cc(C4=C(c5cnc6ccccn56)C(=O)NC4=O)c4cc(F)cc(c43)C2)CC1] | 3-(9-fluoro-2-(4- ((methylamino)methyl)piperidine-1- carbonyl)-1,2,3,4-tetrahyd ro- [1,4]diazepino[6,7,1-hi]indol-7-yl)-4- (imidazo[1,2-a]pyridin-3-yl)-1H- pyrrole-2,5-dione |
| Compound I-33 | 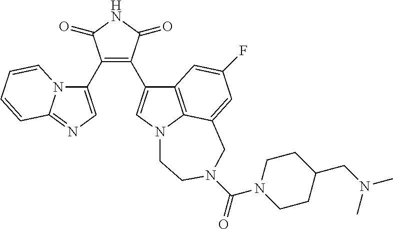 [SMILES: CN(C)CC1CCN(C(=O)N2CCn3cc(C4=C(c5cnc6ccccn56)C(=O)NC4=O)c4cc(F)cc(c43)C2)CC1] | 3-(2-(4- ((dimethylamino)methyl)piperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H- pyrrole-2,5-dione |
| Compound I-34 | 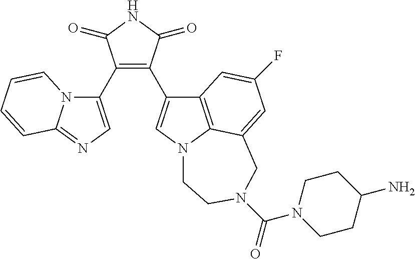 [SMILES: NC1CCN(C(=O)N2CCn3cc(C4=C(c5cnc6ccccn56)C(=O)NC4=O)c4cc(F)cc(c43)C2)CC1] | 3-(2-(4-aminopiperidine-1-carbonyl)- 9-fluoro-1,2,3,4-tetrahydro- [1,4]diazepino[6,7,1-hi]indol-7-yl)-4- (imidazo[1,2-a]pyridin-3-yl)- 1H-pyrrole-2,5-dione |
| Compound I-35 | 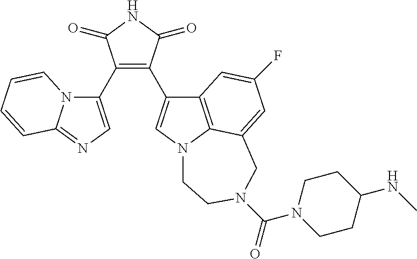 [SMILES: CNC1CCN(C(=O)N2CCn3cc(C4=C(c5cnc6ccccn56)C(=O)NC4=O)c4cc(F)cc(c43)C2)CC1] | 3-(9-fluoro-2-(4- (methylamino)piperidine-1-carbonyl)- 1,2,3,4-tetrahydro-[1,4]diazepino [6,7,1-hi]indol-7-yl)-4-(imidazo [1,2-a]pyridin-3- yl)-1H-pyrrole-2,5-dione |
| Compound I-36 | 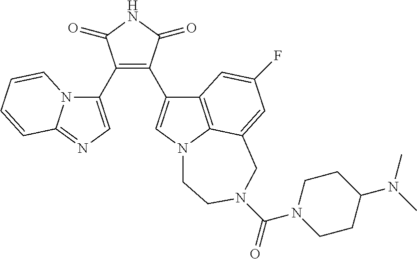 [SMILES: CN(C)C1CCN(C(=O)N2CCn3cc(C4=C(c5cnc6ccccn56)C(=O)NC4=O)c4cc(F)cc(c43)C2)CC1] | 3-(2-(4-(dimethylamino)piperidine-1- carbonyl)-9-fluoro-1,2,3,4-tetrahydro- [1,4]diazepino[6,7,1-hi]indol-7-yl)-4- (imidazo[1,2-a]pyridin-3-yl)- 1H-pyrrole-2,5-dione |
| Compound I-37 | 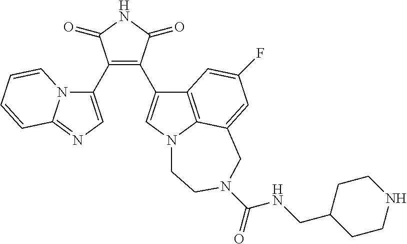 [SMILES: O=C1NC(=O)C(c2cnc3ccccn23)=C1c1cn2c3c(cc(F)cc13)CN(C(=O)NCC1CCNCC1)CC2] | 9-fluoro-7-(4-(imidazo[1,2-a]pyridin-3- yl)-2,5-dioxo-2,5-dihydro-1H- pyrrol-3-yl)-N-(piperidin-4-ylmethyl)- 3,4-dihydro-[1,4]diazepino [6,7,1-hi]indole-2(1H)-carboxamide |
| Compound I-38 | 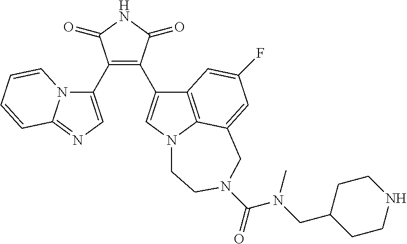 [SMILES: CN(CC1CCNCC1)C(=O)N1CCn2cc(C3=C(c4cnc5ccccn45)C(=O)NC3=O)c3cc(F)cc(c32)C1] | 9-fluoro-7-(4-(imidazo[1,2-a]pyridin-3- yl)-2,5-dioxo-2,5-dihydro-1H- pyrrol-3-yl)-N-methyl-N- (piperidin-4-ylmethyl)-3,4- dihydro-[1,4]diazepino[6,7,1-hi] indole-2(1H)-carboxamide |
| Compound I-39 | 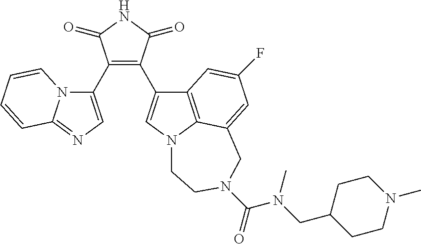 [SMILES: CN1CCC(CN(C)C(=O)N2CCn3cc(C4=C(c5cnc6ccccn56)C(=O)NC4=O)c4cc(F)cc(c43)C2)CC1] | 9-fluoro-7-(4-(imidazo[1,2-a]pyridin- 3-yl)-2,5-dioxo-2,5-dihydro- 1H-pyrrol-3-yl)- N-methyl-N-((1-methylpiperidin- 4-yl)methyl)-3,4-dihydro- [1,4]diazepino[6,7,1-hi]indole-2(1H)- carboxamide |
| Compound I-40 | 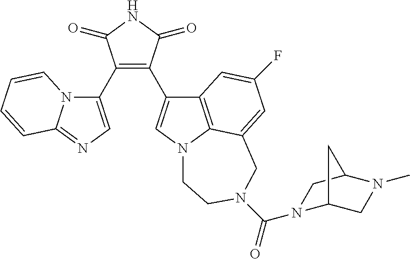 [SMILES: CN1CC2CC1CN2C(=O)N1CCn2cc(C3=C(c4cnc5ccccn45)C(=O)NC3=O)c3cc(F)cc(c32)C1] | 3-(9-fluoro-2-((1R,4R)-5-methyl-2,5- diazabicydo[2.2.1]heptane-2-carbonyl)- 1,2,3,4-tetrahydro-[1,4]diazepino [6,7,1-hi]indol-7-yl)-4-(imidazo[1,2-a] pyridin-3-yl)-1H-pyrrole-2,5-dione |
| Compound I-41 | 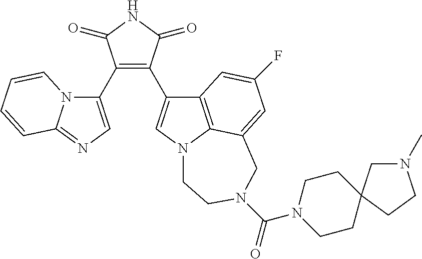 [SMILES: CN1CCC2(CCN(C(=O)N3CCn4cc(C5=C(c6cnc7ccccn67)C(=O)NC5=O)c5cc(F)cc(c54)C3)CC2)C1] | 3-(9-fluoro-2-(2-methyl-2,8- diazaspiro[4.5]decane-8-carbonyl)- 1,2,3,4-tetrahydro-[1,4]diazepino [6,7,1-hi]indol-7-yl)-4- (imidazo[1,2-a]pyridin-3- yl)-1H-pyrrole-2,5-dione |
| Compound I-42 | 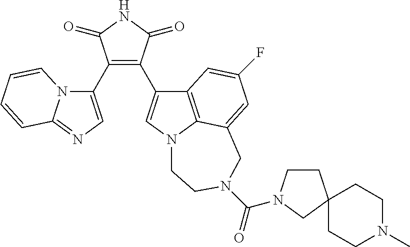 [SMILES: CN1CCC2(CC1)CCN(C(=O)N1CCn3cc(C4=C(c5cnc6ccccn56)C(=O)NC4=O)c4cc(F)cc(c43)C1)C2] | 3-(9-fluoro-2-(8-methyl-2,8- diazaspiro[4.5]decane-2-carbonyl)- 1,2,3,4-tetrahydro-[1,4]diazepino [6,7,1-hi]indol-7-yl)-4- (imidazo[1,2-a]pyridin-3- yl)-1H-pyrrole-2,5-dione |
| Compound I-43 | 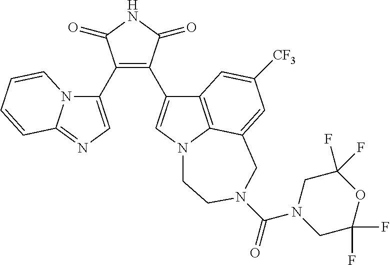 [SMILES: O=C1NC(=O)C(c2cnc3ccccn23)=C1c1cn2c3c(cc(C(F)(F)F)cc13)CN(C(=O)N1CC(F)(F)OC(F)(F)C1)CC2] | 3-(imidazo[1,2-a]pyridin-3-yl)-4-(2- (2,2,6,6-tetrafluoromorpholine-4- carbonyl)-9-(trifluoromethyl)-1,2,3,4- tetrahydro-[1,4]diazepino [6,7,1-hi]indol- 7-yl)-1H-pyrrole-2,5-dione |
| Compound I-44 | 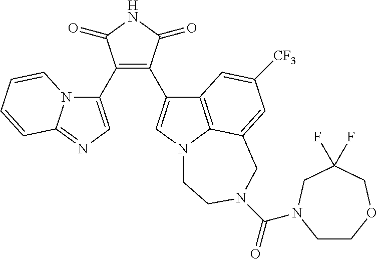 [SMILES: O=C1NC(=O)C(c2cnc3ccccn23)=C1c1cn2c3c(cc(C(F)(F)F)cc13)CN(C(=O)N1CCOCC(F)(F)C1)CC2] | 3-(2-(6,6-difluoro-1,4-oxazepane-4- carbonyl)-9-(trifluoromethyl)-1,2,3,4- tetrahydro-[1,4]diazepino[6,7,1-hi]indol- 7-yl)-4-(imidazo[1,2-a]pyridin-3-yl)-1H- pyrrole-2,5-dione |
| Compound I-45 | 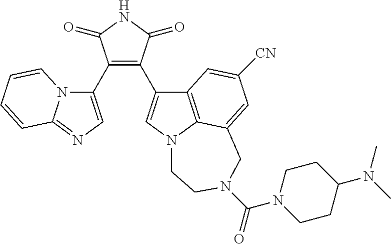 [SMILES: CN(C)C1CCN(C(=O)N2CCn3cc(C4=C(c5cnc6ccccn56)C(=O)NC4=O)c4cc(C#N)cc(c43)C2)CC1] | 2-(4-(dimethylamino)piperidine-1- carbonyl)-7-(4-(imidazo[1,2-a]pyridin- 3-yl)-2,5-dioxo-2,5-dihydro-1H- pyrrol-3-yl)-1,2,3,4-tetrahydro- [1,4]diazepino[6,7,1- hi]indole-9-carbonitrile |
| Compound I-46 | 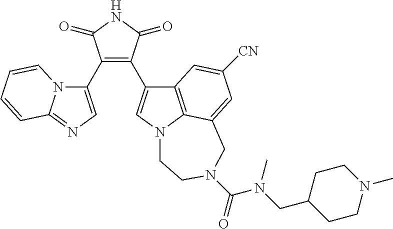 [SMILES: CN1CCC(CN(C)C(=O)N2CCn3cc(C4=C(c5cnc6ccccn56)C(=O)NC4=O)c4cc(C#N)cc(c43)C2)CC1] | 9-cyano-7-(4-(imidazo[1,2-a] pyridin-3-yl)- 2,5-dioxo-2,5-dihydro-1H-pyrrol- 3-yl)-N-methyl-N- ((1-methylpiperidin-4- yl)methyl)-3,4-dihydro- [1,4]diazepino[6,7,1-hi]indole- 2(1H)-carboxamide |
| Compound I-47 | 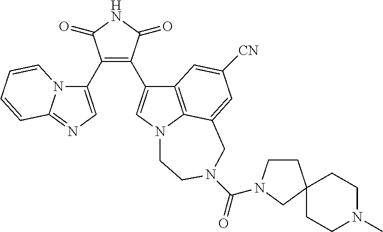 [SMILES: CN1CCC2(CC1)CCN(C(=O)N1CCn3cc(C4=C(c5cnc6ccccn56)C(=O)NC4=O)c4cc(C#N)cc(c43)C1)C2] | 7-(4-(imidazo[1,2-a]pyridin-3-yl)-2,5- dioxo-2,5-dihydro-1H-pyrrol-3-yl)- 2-(8-methyl-2,8-diazaspiro[4.5] decane-2-carbonyl)-1,2,3,4- tetrahydro-[1,4]diazepino [6,7,1-hi]indole-9-carbonitrile |
| Compound I-48 | 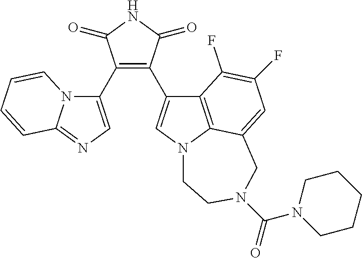 [SMILES: O=C1NC(=O)C(c2cnc3ccccn23)=C1c1cn2c3c(cc(F)c(F)c13)CN(C(=O)N1CCCCC1)CC2] | 3-(8,9-difluoro-2-(piperidine-1-carbonyl)- 1,2,3,4-tetrahydro-[1,4]diazepino[6,7,1- hi]indol-7-yl)-4-(imidazo[,2-a]pyridin-3- yl)-1H-pyrrole-2,5-dione |

The present disclosure also provides for the following compounds and pharmaceutically acceptable salts thereof for use in the methods and compositions disclosed herein.

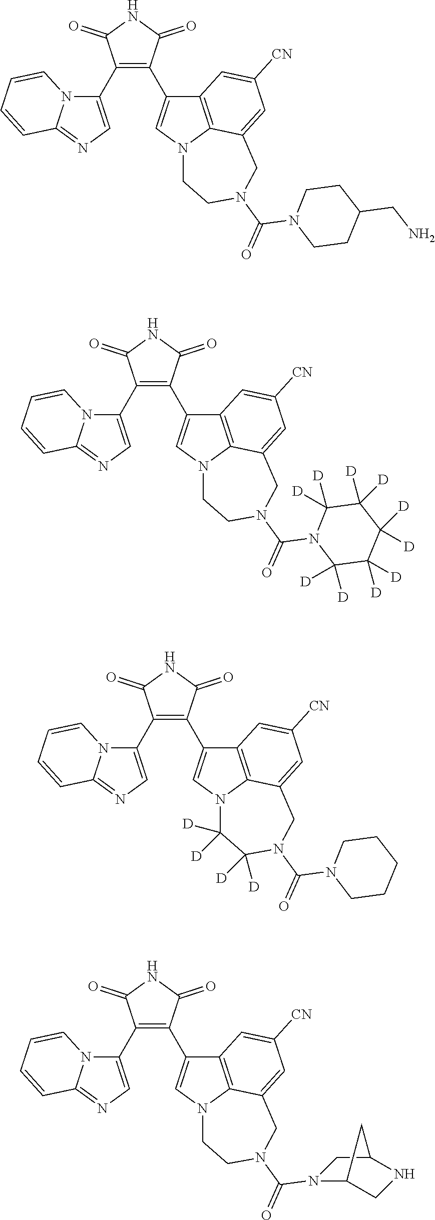 [SMILES: N#Cc1cc2c3c(c1)c(C1=C(c4cnc5ccccn45)C(=O)NC1=O)cn3CCN(C(=O)N1CCC(CN)CC1)C2 ; [2H]C1([2H])N(C(=O)N2CCn3cc(C4=C(c5cnc6ccccn56)C(=O)NC4=O)c4cc(C#N)cc(c43)C2)C([2H])([2H])C([2H])([2H])C([2H])([2H])C1([2H])[2H] ; [2H]C1([2H])N(C(=O)N2CCCCC2)Cc2cc(C#N)cc3c(C4=C(c5cnc6ccccn56)C(=O)NC4=O)cn(c23)C1([2H])[2H] ; N#Cc1cc2c3c(c1)c(C1=C(c4cnc5ccccn45)C(=O)NC1=O)cn3CCN(C(=O)N1CC3CC1CN3)C2]

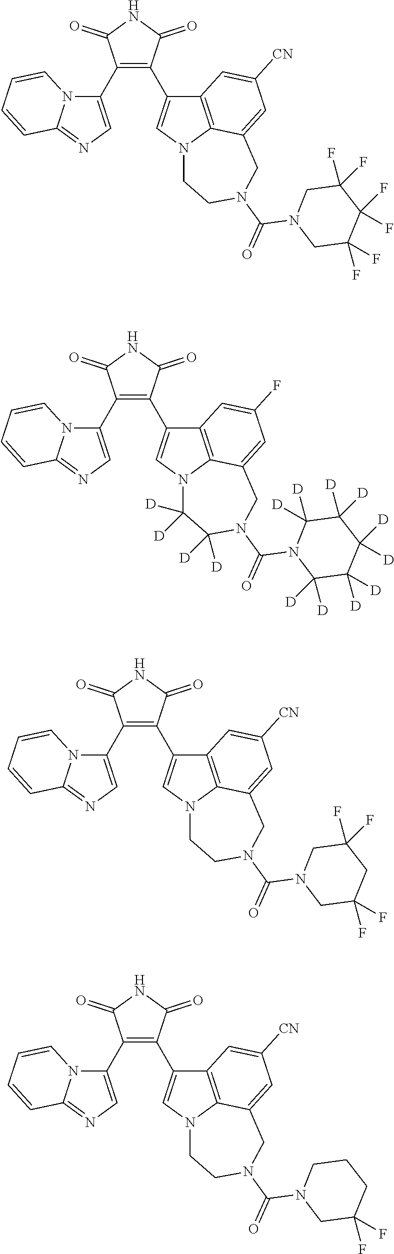 [SMILES: N#Cc1cc2c3c(c1)c(C1=C(c4cnc5ccccn45)C(=O)NC1=O)cn3CCN(C(=O)N1CC(F)(F)C(F)(F)C(F)(F)C1)C2 ; [2H]C1([2H])N(C(=O)N2C([2H])([2H])C([2H])([2H])C([2H])([2H])C([2H])([2H])C2([2H])[2H])Cc2cc(F)cc3c(C4=C(c5cnc6ccccn56)C(=O)NC4=O)cn(c23)C1([2H])[2H] ; N#Cc1cc2c3c(c1)c(C1=C(c4cnc5ccccn45)C(=O)NC1=O)cn3CCN(C(=O)N1CC(F)(F)CC(F)(F)C1)C2 ; N#Cc1cc2c3c(c1)c(C1=C(c4cnc5ccccn45)C(=O)NC1=O)cn3CCN(C(=O)N1CCCC(F)(F)C1)C2]

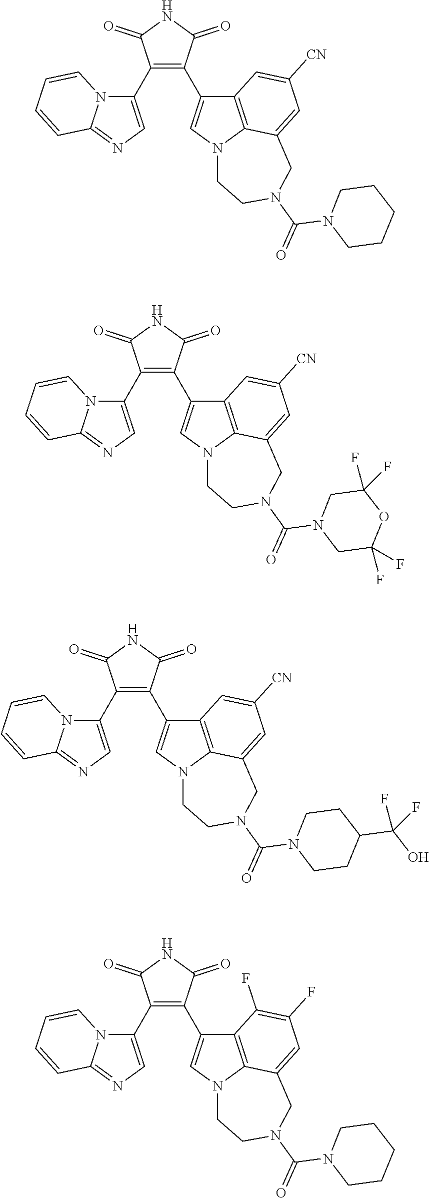 [SMILES: N#Cc1cc2c3c(c1)c(C1=C(c4cnc5ccccn45)C(=O)NC1=O)cn3CCN(C(=O)N1CCCCC1)C2 ; N#Cc1cc2c3c(c1)c(C1=C(c4cnc5ccccn45)C(=O)NC1=O)cn3CCN(C(=O)N1CC(F)(F)OC(F)(F)C1)C2 ; N#Cc1cc2c3c(c1)c(C1=C(c4cnc5ccccn45)C(=O)NC1=O)cn3CCN(C(=O)N1CCC(C(O)(F)F)CC1)C2 ; O=C1NC(=O)C(c2cnc3ccccn23)=C1c1cn2c3c(cc(F)c(F)c13)CN(C(=O)N1CCCCC1)CC2]

Unless otherwise stated, structures depicted herein are also meant to include compounds which differ only in the presence of one or more isotopically enriched atoms. For example, compounds having the present structure except for the replacement of a hydrogen atom by deuterium or tritium, or the replacement of a carbon atom by ¹³C or ¹⁴C, or the replacement of a nitrogen atom by ¹⁵N, or the replacement of an oxygen atom with ¹⁷O or ¹⁸O are within the scope of the present disclosure. Such isotopically labeled compounds are useful as research or diagnostic tools. In certain embodiments, deuteration can be used to slow metabolism and thus potentially improve the compound half-life. Any or all hydrogens in the compound can be replaced with deuterium.

In certain embodiments, the GSK inhibitor is Compound I-7 [3-(2-(4,4-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione].

In certain embodiments, the Wnt signaling pathway target (for example, for inhibition) is selected from the group consisting of AES (TLE/groucho), adenomatous polyposis coli (APC), ARHU, ARHV, AXIN1, AXIN2, -catenin, BMP4, BTRC (b-TrCP), CCND1, CCND2, CCND3, CD44, CDX1, CLD N1 (claudin-1), COL1A1, CTBP1, CTBP2, CTNNBI, CTNNB1P1 (ICAT), DKK 1, DKK2, DKK3, DKK4, Dsh, DVL2, EGR1, EFNB1 (ephrinB 1), ENPP2 (autotaxin), EP300, FBXW1B, FGF4, FO SL 1 (Fra-1), FRAT1, FRAT2, FRZB (FRP-3), FST (follistatin), FZD1, FZD2, FZD3, FZD4, FZD5, FZD6, FZD7, FZD8, FZD9, FZD10, GAS (gastrin), GIPC2, GIPC3, GJA1 (connexin43), GSK3A, glycogen synthase kinase-3B (GSK-313), ICAM1, ID2, ID3, JUN, LEFI, LRP5, LRP6, MFRP, MMP7, MMP26, MSX1, MSX2, MYC, N K D 1, NKD2, NOS2A (iNOS), PITX2, PLAUR (uPAR), serine/threonine protein phosphatase 2A (PP2A), PPN, PPP2R5D (B56 PP2A), PTGS2 (Cox-2), RET, SFRP1 (Secreted Frizzled Related Protein-1, including SFRP1 inhibitors), SFRP2 (FRP-2), SFRP4 (FRP-4), SFRP5, SMOH, SOX17, T (brachyury), TCF, VANGL1, VEGF, WIF1, WISP1, WISP2, WISP3, a Wnt protein, a Wnt receptor, and any analogue or homologue thereof.

Among other things, the methods presented here are useful for the preparation of pharmaceutical formulations for the prophylaxis and/or treatment of diseases associated with absence or lack of hair follicle epithelial cells, such as nonscarring alopecias (for example, telogen effluvium, anagen effluvium, androgenetic alopecia, and alopecia areata), scarring alopecias (for example, tinea capitis, lichen planopilaris, cicatricial alopecia, discoid lupus erythematosus, folliculitis decalvans, dissecting cellulitis of the scalp, frontal fibrosing alopecia and central centrifugal cicatricial alopecia), trichotillomania, traction alopecia, and hypotrichosis.

When stem cells in hair follicles are treated with minoxidil (or analogue thereof); the Shh pathway activators and optionally Wnt agonists of the present disclosure, whether the population is in vivo or in vitro, the treated cells exhibit stem-like behavior in that the treated cells have the capacity to proliferate and differentiate and, more specifically, differentiate into hair follicle epithelial cells. Preferably, the minoxidil (or analogue thereof) Shh pathway activators and optionally Wnt agonists induce and maintain the cells to produce daughter stem cells that can divide for many generations and maintain the ability to have a high proportion of the resulting cells differentiate into hair follicle epithelial cells. In certain embodiments, the proliferating stem cells express stem cell markers which may include Gli1, Krt15, CD34, Lgr5, Lgr6, Lrig1, Sox2, Versican, alkaline phosphatase (AP), Vimentin, CD133, CD200, ID2, DKK3, WIF1, FZD1, FZD2, PHLDA1, Follistatin and/or D102. AP activity has been used as a marker to detect the presence of DP (Dermal Papilla) and is regarded as an indicator for hair inductivity. Handjiski et al. (Br J Dermatol (1994) 131(3): 303-310) show that pelage DP of mice expressed strong and persistent AP activity throughout the entire hair cycle. CD133 (promininl) is a known hematopoetic iPSC marker. CD133 is expressed in DP cells of early anagen mouse skin. CD133 expression correlates with hair inductive properties (Yang and Cotsarelis. J Dermatol Sci (2010) 57(1): 2-11). Vimentin is a mesenchymal marker present in DP and dermal fibroblast cells (Rendl et al. PLOS Biol (2005) 3(11): e331). Versican is expressed in DP cells in follicles in anagen. Versican expression correlates with hair inductive properties (Yang and Cotsarelis. J Dermatol Sci (2010) 57(1): 2-11). Sox2 is a common marker of stem cells. Sox2-expressing cells are required for formation of awl/auchene follicles in a hair reconstitution assay (Driskell et al. Development (2009) 136(16): 2815-2823).

In some embodiments, the method of the present disclosure may be used to maintain, or even transiently increase stemness (i.e., self-renewal) of pre-existing stem cells in a hair follicle prior to significant hair follicle cell formation. In some embodiments, the pre-existing stem cells in a hair follicle comprise keratinocytes, melanocytes, dermal papilla cells and/or bulge cells. Morphological analyses with immunostaining (including cell counts) and lineage tracing across a Representative Microscopy Sample may be used to confirm expansion of one or more of these cell-types.

Advantageously, the methods of the present disclosure achieve these goals without the use of genetic manipulation. Germ-line manipulation is not a therapeutically desirable approach to treating hair loss. In general, the therapy preferably involves the administration of a small molecule, peptide, antibody, or other non-nucleic acid molecule or nucleic acid delivery vector unaccompanied by gene therapy. In certain embodiments, the therapy involves the administration of a small organic molecule. Preferably, hair maintenance or restoration is achieved through the use of a (non-genetic) therapeutic that is administered, for example, intradermally, transdermally, topically or orally.

In addition to expanding stem cells of hair follicles, the methods of the present disclosure have the capacity to maintain, in the daughter cells, the capacity to differentiate into may lineages of hair follicle epithelial cells. In in vivo populations, the maintenance of this capacity may be indirectly observed by an improvement in a subject's hair density, hair growth or ability of hair follicles to go through regenerative cycling. In in vitro populations, the maintenance of this capacity may be directly observed by an increase in the number of DP cells relative to a starting population or indirectly by measuring activity of one or more DP stem cell markers.

In certain embodiments, the stem cell population is of an in vivo subject, and the method is a treatment for hair loss (e.g., wherein the generation of hair follicle epithelial cells from the expanded population of stem cells results in partial or full recovery of hair loss).

In certain embodiments, the present disclosure is directed to a method of facilitating generation of dermal papilla cells, the method comprising: administering a composition of the present disclosure to expand the population of dermal papilla cells. In certain embodiments, the compounds can regenerate hair in a mammal. In certain embodiments, the dermal papilla cell population is of an in vivo subject. In certain embodiments, the dermal papilla cell population is of an in vivo subject for the treatment for alopecia. In certain embodiments, the present disclosure provides a method of generating dermal papilla cells using a composition of the present disclosure to proliferate dermal papilla cells in an initial population in vivo, resulting in an expanded population of dermal papilla cells.

In certain embodiments, the administering step is carried out by performing one or more injections into the dermis (e.g. intradermal administration). In some embodiments, the administration is topical and directed to the region of skin exhibiting hair loss. In some embodiments, the administration is oral. In some embodiments, the administration is transdermal and directed to the region of skin exhibiting hair loss.

In some embodiments, the skin is roughened or wounded before the administration of the therapy. In some embodiments, one or more needles are applied to the skin before the therapy is applied.

In certain embodiments, the administering step comprises administering minoxidil (or analogue thereof); the Shh pathway activators and optionally Wnt agonists, in a sustained manner. In certain embodiments the minoxidil (or analogue thereof); the Shh pathway activators and optionally Wnt agonists is administered daily. In certain embodiments minoxidil (or analogue thereof); the Shh pathway activators and optionally Wnt agonists is administered twice daily. In certain embodiments minoxidil (or analogue thereof); is administered twice daily and the the Shh pathway activators and optionally Wnt agonists is administered once daily.

In some embodiments, the stem cells are DP stem cells. In certain embodiments, the stem cells are hair follicle stem cells. In some embodiments, the stem cells comprise keratinocytes, melanocytes, dermal papilla cells and/or bulge cells.

In certain embodiments, the method further comprises performing high throughput screening using the generated hair follicle epithelial cells. In certain embodiments, the method comprises using the generated hair follicle epithelial cells to screen molecules for toxicity against hair follicle epithelial cells. In certain embodiments, the method comprises using the generated hair follicle epithelial cells to screen molecules for ability to improve survival of hair follicle epithelial cells (e.g., hair follicle epithelial cells exposed to said molecules).

In certain embodiments, the method further comprises performing high throughput screening using the generated expanded population of stem cells. In certain embodiments, the method further comprises using the generated stem cells to screen molecules for toxicity against stem cells and/or their progeny. In certain embodiments, the method comprises using the generated stem cells to screen molecules for ability to improve survival of stem cells and/or their progeny.

In another aspect, the disclosure is directed to a method of generating hair follicle epithelial cells, the method comprising: proliferating stem cells of hair follicles (e.g., of an in vitro, ex vivo, or in vivo sample/subject), resulting in an expanded population of stem cells of hair follicles (e.g., such that the expanded population is a factor of at least 1.25, 1.5, 1.75, 2, 3, 5, 10, or 20 greater than the initial stem cell population); and facilitating generation of hair follicle epithelial cells from the expanded population of hair follicle stem cells.

In another aspect, the disclosure is directed to a method of generating hair follicle epithelial cells, the method comprising administering a compound or composition provided herein to a cell population in one or more hair follicle of a subject, thereby facilitating generation of hair follicle epithelial cells.

In another aspect, the present disclosure provides pharmaceutical compositions comprising: a pharmaceutically-acceptable carrier and (i) menoxidil and (ii) a Sonic Hedgehog (Shh) activator, or a pharmaceutically-acceptable salt thereof. Optionally, the pharmaceutical compositions further include a Wnt agonist, or a pharmaceutically-acceptable salt thereof. In some embodiments, the Wnt agonist comprises a GSK3-alpha inhibitor. In other embodiments, the Wnt agonist comprises a GSK3-beta inhibitor. In some embodiments, the Shh pathway activator comprises a Smoothened agonist. In certain embodiments, the Shh pathway activator comprises Smoothen ciliary accumulation enhancers. In some embodiments, the composition is adapted for administration to the skin.

Certain embodiments relate to pharmaceutical compositions, comprising a pharmaceutically-acceptable carrier and (i) menoxidil (i) a Wnt agonist, a GSK3-alpha inhibitor, or a GSK3-beta inhibitor and (ii) a Sonic Hedgehog activator, Smoothened agonist, Smoothened ciliary accumulation enhancers, or a pharmaceutically-acceptable salt thereof. In some embodiments, the composition is adapted for administration to the skin.

In certain embodiments, the Shh pathway activator is selected from Purmorphamine, SAG, 20-alpha hydroxy cholesterol, and SAG HCl.

In certain embodiments of the compositions and methods provided herein, the minoxidil is at a concentration of about 0.1 mM to 500 mM, about, about about 1 mM to 500 mM, about 1 mM to 400 mM, about 1 mM to 300 mM, about 1 mM to 200 mM, about 1 mM to 100 mM. In some embodiments, the minoxidil is at a concentration of about 10 mM to 500 mM, about 10 mM to 400 mM 10 mM to 300 mM, about 10 mM to 250 mM, about 10 mM to 200 mM, about 10 mM to 150 mM, about 10 mM to 100 mM, or about 10 mM to 50 mM. In some embodiments, the minoxidil is at a concentration of about 20 mM to 500 mM, about 20 mM to 400 mM 20 mM to 300 mM, about 20 mM to 250 mM, about 20 mM to 200 mM, about 20 mM to 150 mM, about 20 mM to 100 mM, or about 20 mM to 50 mM.

In some embodiments, the minoxidil is at a concentration ratio of about 0.001 to 10 fold relative to an FDA approved minioxidil concentration, or about 0.1 to 50 fold relative to an FDA approved minoxidil Concentration, or about 0.1 to 5 fold relative to an FDA approved minoxidil Concentration, or about 1 to 5 fold relative to an FDA approved minoxidil Concentration, or about 0.001 to 1 fold relative to its Effective minoxidil Concentration, or about 0.01 to 1 fold relative to an FDA approved minioxidil, or about 0.1 to 1 fold relative to an FDA approved minoxidil Concentration,

In some embodiments, the minoxidil pathway activator is at about 0.01×. 0.1×, 2×, 3×, 5× or 10×, relative to an FDA approved minioxidil.

In some embodiments, the minoxidil is at a concentration of about 1 mM, about 2 mM, about 3 mM, about 4 mM, about 5 mM 10 mM, about 20 mM, about 30, mM, about 40 mM, about 50 mM, about 60 mM, 70 mM, about 80 mM, about 90, mM, about 100 mM, about 150 mM about 200 mM, about 250 mM, 300 mM, about 350 mM, about 400, mM, about 450 mM, or about 500 mM.

In certain embodiments of the compositions and methods provided herein, the Sonic Hedgehog (Shh) activator is at a concentration of about 0.01 μM to 1000 mM, about 0.1 μM to 1000 mM, about 1 μM to 100 mM, about 0.1 μM to 1 μM, about 1 μM to 10 μM, about 10 μM to 100 μM, about 100 μM to 1 mM, about 1 mM to 10 mM, or about 100 mM to 1000 mM, or about 10 mM to 100 mM, or about 100 mM to 1000 mM. In some embodiments, the Shh pathway activator is at a concentration ratio of about 0.1 to 1,000,000 fold relative to its Effective Shh Concentration, or about 1 to 100,000 fold relative to its Effective Shh Concentration, or about 10 to 10,000 fold relative to its Effective Shh Concentration, or about 100 to 1000 fold relative to its Effective Shh Concentration, or about 1000 fold relative to its Effective Shh Concentration. In some embodiments, the Shh pathway activator is at about 1×, 3×, 10×, 30×, 100×, 300×, 1000×, 3000×, 5000× relative to its Effective Shh Concentration. In some embodiments, the Shh pathway activator is at a concentration of about 0.01 nM to 1000 μM, or about 0.1 nM to 1000 μM, about 1 nM to 100 μM, about 10 nM to 10 μM, about 1 nM to 10 nM, about 10 nM to 100 nM, about 100 nM to 1000 nM, about 1 μM to 10 μM, about 10 μM to 100 μM, or about 100 μM to 1000 μM.

In certain embodiments of the compositions and methods provided herein, the Shh pathway activator is SAG (CAS 912545-86-9), which is at a concentration of about 0.01 μM to 1000 mM, about 0.1 μM to 10 mM, about 1 μM to 1 mM, about 10 μM to 100 μM, about M, about 20 μM, about 25 μM, about 50 μM. In some embodiments, the Shh pathway activator is SAG (CAS 912545-86-9), which is at a concentration ratio of about 0.1 to 1,000,000 fold relative to its Effective Shh Concentration, or about 1 to 100,000 fold relative to its Effective Shh Concentration, or about 10 to 10,000 fold relative to its Effective Shh Concentration, or about 100 to 1000 fold relative to its Effective Shh Concentration, or about 1000 fold relative to its Effective Shh Concentration. In some embodiments, the Shh pathway activator is SAG (CAS 912545-86-9), which is at about 1×, 3×, 10×, 30×, 100×, 300×, 1000×, 3000×, 5000× relative to its Effective Shh Concentration. In some embodiments, the Shh pathway activator is SAG (CAS 912545-86-9), which is at a concentration of about 0.001 nM to 1000 μM, about 0.01 nM to 10 μM, about 0.1 nM to 1 μM, about 1 nM to 100 nM, 1 nM to 10 nM, about 1 nM, about 2 nM, about 3 nM, about 4 nM, about 5 nM, about 6 nM, about 7 nM, about 9 nM, or about 10 nM.

In certain embodiments of the compositions and methods provided herein, the Shh pathway activator is 20-alpha hydroxy cholesterol, which is at a concentration of about 0.01 μM to 1000 mM, about 0.1 μM to 100 mM, about 1 μM to 10 mM, about 10 μM to 1 mM, about 125 μM, about 250 μM, about 500 μM, or about 1000 μM. In some embodiments, the Shh pathway activator is 20-alpha hydroxy cholesterol, which is at a concentration ratio of about 0.1 to 1,000,000 fold relative to its Effective Shh Concentration, or about 1 to 100,000 fold relative to its Effective Shh Concentration, or about 10 to 10,000 fold relative to its Effective Shh Concentration, or about 100 to 1000 fold relative to its Effective Shh Concentration, or about 1000 fold relative to its Effective Shh Concentration. In some embodiments, the Shh pathway activator is 20-alpha hydroxy cholesterol, which is at about 1×, 3×, 10×, 30×, 100×, 300×, 1000×, 3000×, 5000× relative to its Effective Shh Concentration. In some embodiments, the Shh pathway activator is 20-alpha hydroxy cholesterol, which is at a concentration of about 0.01 nM to 1000 μM, about 0.1 nM to 100 μM, about 1 nM to 10 μM, about 10 nM to 1 μM, about 25 nM, about 50 nM, about 100 nM, about 200 nM, or about 500 nM.

In certain embodiments of the compositions and methods provided herein, the Shh pathway activator is SAG HCl (CAS 912545-86-9), which is at a concentration of about 1 μM to 1000 mM, or about 10 μM to 1000 mM, or about 100 μM to 10 mM, or about 1 mM. In some embodiments, the Shh pathway activator is SAG HCl, which is at a concentration ratio of about 0.1 to 1,000,000 fold relative to its Effective Shh Concentration, or about 1 to 100,000 fold relative to its Effective Shh Concentration, or about 10 to 10,000 fold relative to its Effective Shh Concentration, or about 100 to 1000 fold relative to its Effective Shh Concentration, or about 1000 fold relative to its Effective Shh Concentration. In some embodiments, the Shh pathway activator is SAG HCl, which is at about 1×, 3×, 10×, 30×, 100×, 300×, 1000×, 3000×, 5000× relative to its Effective Shh Concentration. In some embodiments, the Shh pathway activator is SAG HCl, which is at a concentration of about 1 nM to 1000 μM, about 10 nM to 100 μM, about 100 nM to 10 μM, about 125 nM, about 250 nM, or about 500 nM, or about 750 nM.

In certain embodiments of the compositions and methods provided herein, the Shh pathway activator is Purmorphamine, which is at a concentration of about 1 μM to 1000 mM, or about 10 μM to 1000 mM, or about 100 μM to 10 mM, or about 2 mM. In some embodiments, the Shh pathway activator is Purmorphamine, which is at a concentration ratio of about 0.1 to 1,000,000 fold relative to its Effective Shh Concentration, or about 1 to 100,000 fold relative to its Effective Shh Concentration, or about 10 to 10,000 fold relative to its Effective Shh Concentration, or about 100 to 1000 fold relative to its Effective Shh Concentration, or about 1000 fold relative to its Effective Shh Concentration. In some embodiments, the Shh pathway activator is Purmorphamine, which is at about 1×, 3×, 10×, 30×, 100×, 300×, 1000×, 3000×, 5000× relative to its Effective Shh Concentration. In some embodiments, the Shh pathway activator is Purmorphamine, which is at a concentration of about 1 nM to 1000 μM, about 10 nM to 100 μM, about 100 nM to 10 μM, about 1 μM to 10 μM, about 1 μM, about 2 μM, about 3 μM, about 4 μM, about 5 μM, or about 6 μM.

In some embodiments of the methods and compositions described herein, the Wnt agonist, GSK3-alpha inhibitor, or GSK3-beta inhibitor is selected from CHIR99021, LY2090314, AZD1080, GSK3 inhibitor XXII, Compound I-6, Compound I-7, and Compound I-12.

In certain embodiments of the compositions and methods provided herein, the Wnt agonist, GSK3-alpha inhibitor, or GSK3-beta inhibitor is at a concentration of about 0.01 μM to 1000 mM, about 0.1 μM to 1000 mM, about 1 μM to 100 mM, about 10 μM to 10 mM, about 1 μM to 10 μM, about 10 μM to 100 μM, about 100 μM to 1000 μM, about 1 mM to 10 mM, or about 10 mM to 100 mM. In some embodiments, the Wnt agonist, GSK3-alpha inhibitor, or GSK3-beta inhibitor is at a concentration ratio of about 0.01 to 1,000,000 fold relative to its Effective Stemness Driver Concentration, or about 0.1 to 100,000 fold relative to its Effective Stemness Driver Concentration, or about 1 to 10,000 fold relative to its Effective Stemness Driver Concentration, or about 100 to 5000 fold relative to its Effective Stemness Driver Concentration, or about 50 to 2000 fold relative to its Effective Stemness Driver Concentration, or about 100 to 1000 fold relative to its Effective Stemness Driver Concentration. In further embodiments, the Wnt agonist, GSK3-alpha inhibitor, or GSK3-beta inhibitor is at about 1×, 3×, 10×, 30×, 100×, 300×, 1000×, 3000×, 5000× relative to its Effective Stemness Driver Concentration. In yet further embodiments, the Wnt agonist, GSK3-alpha inhibitor, or GSK3-beta inhibitor is at a concentration of about 0.01 nM to 1000 μM, about 0.1 nM to 1000 μM, about 1 nM to 100 μM, about 10 nM to 10 μM, about 1 nM to 10 nM, about 10 nM to 100 nM, about 100 nM to 1000 nM, about 1 μM to 10 μM, or about M to 100 μM.

In certain embodiments of the compositions and methods provided herein, the Wnt agonist, GSK3-alpha inhibitor, or GSK3-beta inhibitor is CHIR99021, which is at a concentration of about 1 μM to 1000 mM, about 10 μM to 100 mM, about 100 μM to 100 mM, about 1 mM to 10 mM, or about 10 mM. In some embodiments, the Wnt agonist, GSK3-alpha inhibitor, or GSK3-beta inhibitor is CHIR99021, which is at a concentration ratio of about 0.01 to 1,000,000 fold relative to its Effective Sternness Driver Concentration, or about 0.1 to 100,000 fold relative to its Effective Sternness Driver Concentration, or about 1 to 10,000 fold relative to its Effective Sternness Driver Concentration, or about 100 to 5000 fold relative to its Effective Sternness Driver Concentration, or about 50 to 2000 fold relative to its Effective Sternness Driver Concentration, or about 100 to 1000 fold relative to its Effective Sternness Driver Concentration. In some embodiments, the Wnt agonist, GSK3-alpha inhibitor, or GSK3-beta inhibitor is CHIR99021, which is at about 1×, 3×, 10×, 30×, 100×, 300×, 1000×, 3000×, 5000× relative to its Effective Sternness Driver Concentration. In some embodiments, the Wnt agonist, GSK3-alpha inhibitor, or GSK3-beta inhibitor is CHIR99021, which is at a concentration of about 1 nM to 1000 μM, about 10 nM to 100 μM, about 100 nM to 100 μM, about 100 nM to 1 μM, about 1 μM to 10 μM, or about 1, 2, 3, 4, 5, 6, 7, 8, 9, or 10 μM.

In certain embodiments of the compositions and methods provided herein, the Wnt agonist, GSK3-alpha inhibitor, or GSK3-beta inhibitor is LY2090314, which is at a concentration of about 0.01 μM to 1000 mM, about 0.1 μM to 10 mM, about 1 μM to 1 mM, about 10 μM, about 20 μM, about 30 μM, about 40 μM, or about 50 μM. In some embodiments, the Wnt agonist, GSK3-alpha inhibitor, or GSK3-beta inhibitor is LY2090314, which is at a concentration ratio of about 0.01 to 1,000,000 fold relative to its Effective Sternness Driver Concentration, or about 0.1 to 100,000 fold relative to its Effective Sternness Driver Concentration, or about 1 to 10,000 fold relative to its Effective Sternness Driver Concentration, or about 100 to 5000 fold relative to its Effective Sternness Driver Concentration, or about 50 to 2000 fold relative to its Effective Sternness Driver Concentration, or about 100 to 1000 fold relative to its Effective Sternness Driver Concentration. In some embodiments, the Wnt agonist, GSK3-alpha inhibitor, or GSK3-beta inhibitor is LY2090314, which is at about 1×, 3×, 10×, 30×, 100×, 300×, 1000×, 3000×, 5000× relative to its Effective Sternness Driver Concentration. In some embodiments, the Wnt agonist, GSK3-alpha inhibitor, or GSK3-beta inhibitor is LY2090314, which is at a concentration of about 0.01 nM to 1000 μM, about 0.1 nM to 10 μM, about 1 nM to 1 μM, about 1 nM to 100 nM, about 1 nM to 50 nM, or about 1, 2, 3, 4, 5, 6, 7, 8, 9, or 10 nM.

In certain embodiments of the compositions and methods provided herein, the Wnt agonist, GSK3-alpha inhibitor, or GSK3-beta inhibitor is AZD1080, which is at a concentration of about 0.1 μM to 1000 mM, about 1 μM to 1000 mM, about 10 μM to 100 mM, about 100 μM to 10 mM, about 1 mM to 10 mM, or about 1, 2, 3, 4, 5, 6, 7, 8, 9, or 10 mM. In some embodiments, the Wnt agonist, GSK3-alpha inhibitor, or GSK3-beta inhibitor is AZD1080, which is at a concentration ratio of about 0.01 to 1,000,000 fold relative to its Effective Stemness Driver Concentration, or about 0.1 to 100,000 fold relative to its Effective Stemness Driver Concentration, or about 1 to 10,000 fold relative to its Effective Stemness Driver Concentration, or about 100 to 5000 fold relative to its Effective Stemness Driver Concentration, or about 50 to 2000 fold relative to its Effective Stemness Driver Concentration, or about 100 to 1000 fold relative to its Effective Stemness Driver Concentration. In some embodiments, the Wnt agonist, GSK3-alpha inhibitor, or GSK3-beta inhibitor is AZD1080, which is at about 1×, 3×, 10×, 30×, 100×, 300×, 1000×, 3000×, 5000× relative to its Effective Stemness Driver Concentration. In some embodiments, the Wnt agonist, GSK3-alpha inhibitor, or GSK3-beta inhibitor is AZD1080, which is at a concentration of about 1 nM to 1000 μM, about 10 nM to 1000 μM, about 100 nM to 100 μM, about 1 μM to 10 μM, or about 1, 2, 3, 4, 5, 6, 7, 8, 9, or 10 μM.

In certain embodiments of the compositions and methods provided herein, the Wnt agonist, GSK3-alpha inhibitor, or GSK3-beta inhibitor is GSK3 inhibitor XXII, which is at a concentration of about 0.1 μM to 1000 mM, about 1 μM to 100 mM, about 10 μM to 10 mM, about 100 μM to 10 mM, about 100 μM to 1 mM, or about 1, 2, 3, 4, 5, 6, 7, 8, 9, or 10 mM. In some embodiments, the Wnt agonist, GSK3-alpha inhibitor, or GSK3-beta inhibitor is GSK3 inhibitor XXII, which is at a concentration ratio of about 0.01 to 1,000,000 fold relative to its Effective Sternness Driver Concentration, or about 0.1 to 100,000 fold relative to its Effective Sternness Driver Concentration, or about 1 to 10,000 fold relative to its Effective Stemness Driver Concentration, or about 100 to 5000 fold relative to its Effective Stemness Driver Concentration, or about 50 to 2000 fold relative to its Effective Stemness Driver Concentration, or about 100 to 1000 fold relative to its Effective Stemness Driver Concentration. In some embodiments, the Wnt agonist, GSK3-alpha inhibitor, or GSK3-beta inhibitor is GSK3 inhibitor XXII, which is at about 1×, 3×, 10×, 30×, 100×, 300×, 1000×, 3000×, 5000× relative to its Effective Stemness Driver Concentration. In some embodiments, the Wnt agonist, GSK3-alpha inhibitor, or GSK3-beta inhibitor is GSK3 inhibitor XXII, which is at a concentration of about 0.1 nM to 1000 μM, about 1 nM to 100 μM, about 10 nM to 10 μM, about 100 nM to 1 μM, or about 0.5 μM.

In certain embodiments of the compositions and methods provided herein, the Wnt agonist is Compound I-6, which is at a concentration of about 0.01 μM to 1000 mM, about 0.1 μM to 10 mM, about 1 μM to 1 mM, about 10 μM to 1 mM, about 125 μM, about 250 μM, about 500 μM, or about 750 μM. In some embodiments, the Wnt agonist is Compound I-6, which is at a concentration ratio of about 0.01 to 1,000,000 fold relative to its Effective Sternness Driver Concentration, or about 0.1 to 100,000 fold relative to its Effective Sternness Driver Concentration, or about 1 to 10,000 fold relative to its Effective Sternness Driver Concentration. In some embodiments, the Wnt agonist is Compound I-6, which is about 100 to 5000 fold relative to its Effective Sternness Driver Concentration, or about 50 to 2000 fold relative to its Effective Sternness Driver Concentration, or about 100 to 1000 fold relative to its Effective Sternness Driver Concentration. In some embodiments, the Wnt agonist is Compound I-6, which is at about 1×, 3×, 10×, 30×, 100×, 300×, 1000×, 3000×, 5000× relative to its Effective Sternness Driver Concentration. In some embodiments, the Wnt agonist is Compound I-6, which is at a concentration of about 0.01 nM to 1000 μM, about 0.1 nM to 10 μM, about 1 nM to 1 μM, about 10 nM to 100 nM, about 25 nM, about 25 nM, or about 75 nM.

In certain embodiments of the compositions and methods provided herein, the Wnt agonist is Compound I-7, which is at a concentration of about 0.01 μM to 1000 mM, about 0.1 μM to 10 mM, about 1 μM to 1 mM, about 10 μM, about 20 μM, about 30 μM, about 40 μM, or about 50 μM. In some embodiments, the Wnt agonist is Compound I-7, which is at a concentration ratio of about 0.01 to 1,000,000 fold relative to its Effective Sternness Driver Concentration, or about 0.1 to 100,000 fold relative to its Effective Sternness Driver Concentration, or about 1 to 10,000 fold relative to its Effective Sternness Driver Concentration, or about 100 to 5000 fold relative to its Effective Sternness Driver Concentration, or about 50 to 2000 fold relative to its Effective Sternness Driver Concentration, or about 100 to 1000 fold relative to its Effective Sternness Driver Concentration. In some embodiments, the Wnt agonist is Compound I-7, which is at about 1×, 3×, 10×, 30×, 100×, 300×, 1000×, 3000×, 5000× relative to its Effective Sternness Driver Concentration. In some embodiments, the Wnt agonist is Compound I-7, which is at a concentration of about 0.01 nM to 1000 μM, about 0.1 nM to 10 μM, about 1 nM to 1 μM, about 1 nM to 100 nM, about 5 nM, about 10 nM or about 20 nM.

In certain embodiments of the compositions and methods provided herein, the Wnt agonist is Compound I-12, which is at a concentration of about 0.01 μM to 1000 mM, about 0.1 μM to 10 mM, about 1 μM to 1 mM, about 10 μM to 1 mM, about 125 μM, about 250 μM, about 500 μM, or about 750 μM. In some embodiments, the Wnt agonist is Compound I-12, which is at a concentration ratio of about 0.01 to 1,000,000 fold relative to its Effective Sternness Driver Concentration, or about 0.1 to 100,000 fold relative to its Effective Sternness Driver Concentration, or about 1 to 10,000 fold relative to its Effective Sternness Driver Concentration, or about 100 to 5000 fold relative to its Effective Sternness Driver Concentration, or about 50 to 2000 fold relative to its Effective Sternness Driver Concentration, or about 100 to 1000 fold relative to its Effective Sternness Driver Concentration. In some embodiments, the Wnt agonist is Compound I-12, which is at about 1×, 3×, 10×, 30×, 100×, 300×, 1000×, 3000×, 5000× relative to its Effective Sternness Driver Concentration. In some embodiments, the Wnt agonist is Compound I-12, which is at a concentration of about 0.01 nM to 1000 μM, about 0.1 nM to 10 μM, about 1 nM to 1 μM, about 10 nM to 100 nM, about 25 nM about 50 nM or about 75 nM.

In certain embodiments of the compositions and methods described herein, minoxidil is at a concentration of 10 mM to about 250 mM in combination with Purmorphamine at a concentration of about 100 nM to about 10 μM. In certain embodiments, minoxidil is at a concentration of 10 mM to about 250 mM in combination with SAG at a concentration of about 1 nM to about 100 nM. In certain embodiments, minoxidil is at a concentration of 10 mM to about 250 mM in combination with 20-alpha hydroxy cholesterol at a concentration of about 1 μM to about 100 μM. In certain embodiments, minoxidil is at a concentration of 10 mM to about 250 mM in combination with SAG HCl at a concentration of about 10 nM to about 1 μM.

In certain embodiments of the compositions and methods described herein, minoxidil is at a concentration of 10 mM to about 250 mM in combination with CHIR99021 is at a concentration of about 100 nM to about 10 μM in combination with Purmorphamine at a concentration of about 100 nM to about 10 μM. In certain embodiments, minoxidil is at a concentration of 10 mM to about 250 mM in combination with CHIR99021 is at a concentration of about 100 nM to about 10 μM in combination with SAG at a concentration of about 1 nM to about 100 nM. In certain embodiments, minoxidil is at a concentration of 10 mM to about 250 mM in combination with CHIR99021 is at a concentration of about 100 nM to about 10 μM in combination with 20-alpha hydroxy cholesterol at a concentration of about 1 μM to about 100 μM. In certain embodiments, minoxidil is at a concentration of 10 mM to about 250 mM in combination with CHIR99021 is at a concentration of about 100 nM to about 10 μM in combination with SAG HCl at a concentration of about 10 nM to about 1 μM.

In certain embodiments of the compositions and methods described herein, minoxidil is at a concentration of 10 mM to about 250 mM in combination with LY2090314 is at a concentration of about 1 nM to about 100 nM in combination with Purmorphamine at a concentration of about 100 nM to about 10 μM. In certain embodiments, minoxidil is at a concentration of 10 mM to about 250 mM in combination with LY2090314 is at a concentration of about 1 nM to about 100 nM in combination with SAG at a concentration of about 1 nM to about 100 nM. In certain embodiments, minoxidil is at a concentration of 10 mM to about 250 mM in combination with LY2090314 is at a concentration of about 1 nM to about 100 nM in combination with 20-alpha hydroxy cholesterol at a concentration of about 1 μM to about 100 μM. In certain embodiments, minoxidil is at a concentration of 10 mM to about 250 mM in combination with LY2090314 is at a concentration of about 1 nM to about 100 nM in combination with SAG HCl at a concentration of about 10 nM to about 1 μM.

In certain embodiments of the compositions and methods described herein, minoxidil is at a concentration of 10 mM to about 250 mM in combination with AZD1080 is at a concentration of about 1 μM to about 100 μM in combination with Purmorphamine at a concentration of about 100 nM to about 10 μM. In certain embodiments, minoxidil is at a concentration of 10 mM to about 250 mM in combination with AZD1080 is at a concentration of about 1 μM to about 100 μM in combination with SAG at a concentration of about 1 nM to about 100 nM. In certain embodiments, minoxidil is at a concentration of 10 mM to about 250 mM in combination with AZD1080 is at a concentration of about 1 μM to about 100 μM in combination with 20-alpha hydroxy cholesterol at a concentration of about 1 μM to about 100 μM. In certain embodiments, minoxidil is at a concentration of 10 mM to about 250 mM in combination with AZD1080 is at a concentration of about 1 μM to about 100 μM in combination with SAG HCl at a concentration of about 10 nM to about 1 μM.

In certain embodiments of the compositions and methods described herein, minoxidil is at a concentration of 10 mM to about 250 mM in combination with GSK3 inhibitor XXII is at a concentration of about 100 nM to about 10 μM in combination with Purmorphamine at a concentration of about 100 nM to about 10 μM. In certain embodiments, minoxidil is at a concentration of 10 mM to about 250 mM in combination with GSK3 inhibitor XXII is at a concentration of about 100 nM to about 10 μM in combination with SAG at a concentration of about 1 nM to about 100 nM. In certain embodiments, minoxidil is at a concentration of 10 mM to about 250 mM in combination with GSK3 inhibitor XXII is at a concentration of about 100 nM to about 10 μM in combination with 20-alpha hydroxy cholesterol at a concentration of about 1 μM to about 100 μM. In certain embodiments, minoxidil is at a concentration of 10 mM to about 250 mM in combination with GSK3 inhibitor XXII is at a concentration of about 100 nM to about 10 μM in combination with SAG HCl at a concentration of about 10 nM to about 1 μM.

In certain embodiments of the compositions and methods described herein, minoxidil is at a concentration of 10 mM to about 250 mM in combination with Compound I-6 is at a concentration of about 1 nM to about 100 nM in combination with Purmorphamine at a concentration of about 100 nM to 10 μM. In certain embodiments, minoxidil is at a concentration of 10 mM to about 250 mM in combination with Compound I-6 is at a concentration of about 1 nM to about 100 nM in combination with SAG at a concentration of about 1 nM to about 100 nM. In certain embodiments, minoxidil is at a concentration of 10 mM to about 250 mM in combination with Compound I-6 is at a concentration of about 1 nM to about 100 nM in combination with 20-alpha hydroxy cholesterol at a concentration of about 1 μM to about 100 μM. In certain embodiments, minoxidil is at a concentration of 10 mM to about 250 mM in combination with Compound I-6 is at a concentration of about 1 nM to about 100 nM in combination with SAG HCl at a concentration of about 10 nM to about 1 μM.

In certain embodiments of the compositions and methods described herein, minoxidil is at a concentration of 10 mM to about 250 mM in combination with Compound I-7 is at a concentration of about 1 nM to about 100 nM in combination with Purmorphamine at a concentration of about 100 nM to about 10 μM. In certain embodiments, minoxidil is at a concentration of 10 mM to about 250 mM in combination with Compound I-7 is at a concentration of about 1 nM to about 100 nM in combination with SAG at a concentration of about 1 nM to about 100 nM. In certain embodiments, minoxidil is at a concentration of 10 mM to about 250 mM in combination with Compound I-7 is at a concentration of about 1 nM to about 100 nM in combination with 20-alpha hydroxy cholesterol at a concentration of about 1 μM to about 100 μM. In certain embodiments, minoxidil is at a concentration of 10 mM to about 250 mM in combination with Compound I-7 is at a concentration of about 1 nM to about 100 nM in combination with SAG HCl at a concentration of about 10 nM to about 1 μM.

In certain embodiments of the compositions and methods described herein, minoxidil is at a concentration of 10 mM to about 250 mM in combination with Compound I-12 is at a concentration of about 10 nM to about 1000 nM in combination with Purmorphamine at a concentration of about 100 nM to about 10 μM. In certain embodiments, minoxidil is at a concentration of 10 mM to about 250 mM in combination with Compound I-12 is at a concentration of about 10 nM to about 1000 nM in combination with SAG at a concentration of about 1 nM to about 100 nM. In certain embodiments, minoxidil is at a concentration of 10 mM to about 250 mM in combination with Compound I-12 is at a concentration of about 10 nM to about 1000 nM in combination with 20-alpha hydroxy cholesterol at a concentration of about 1 μM to about 100 μM. In certain embodiments, minoxidil is at a concentration of 10 mM to about 250 mM in combination with Compound I-12 is at a concentration of about 10 nM to about 1000 nM in combination with SAG HCl at a concentration of about 10 nM to about 1 μM.

In certain embodiments of the compositions and methods described herein, minoxidil is at a concentration of 10 mM to about 250 mM in combination with CHIR99021 is at a concentration of about 100 μM to about 10 mM in combination with Purmorphamine at a concentration of about 100 μM to about 10 mM. In certain embodiments, minoxidil is at a concentration of 10 mM to about 250 mM in combination with CHIR99021 is at a concentration of about 100 μM to about 10 mM in combination with SAG at a concentration of about 1 μM to about 100 μM. In certain embodiments, minoxidil is at a concentration of 10 mM to about 250 mM in combination with CHIR99021 is at a concentration of about 100 μM to about 10 mM in combination with 20-alpha hydroxy cholesterol at a concentration of about 1 mM to about 100 mM. In certain embodiments, minoxidil is at a concentration of 10 mM to about 250 mM in combination with CHIR99021 is at a concentration of about 100 μM to about 10 mM in combination with SAG HCl at a concentration of about 10 μM to about 1 mM.

In certain embodiments of the compositions and methods described herein, minoxidil is at a concentration of 10 mM to about 250 mM in combination with LY2090314 is at a concentration of about 1 μM to about 100 μM in combination with Purmorphamine at a concentration of about 100 μM to about 10 mM. In certain embodiments, minoxidil is at a concentration of 10 mM to about 250 mM in combination with LY2090314 is at a concentration of about 1 μM to about 100 μM in combination with SAG at a concentration of about 1 μM to about 100 μM. In certain embodiments, minoxidil is at a concentration of 10 mM to about 250 mM in combination with LY2090314 is at a concentration of about 1 μM to about 100 μM in combination with 20-alpha hydroxy cholesterol at a concentration of about 1 mM to about 100 mM. In certain embodiments, minoxidil is at a concentration of 10 mM to about 250 mM in combination with LY2090314 is at a concentration of about 1 μM to about 100 μM in combination with SAG HCl at a concentration of about 10 μM to about 1 mM.

In certain embodiments of the compositions and methods described herein, minoxidil is at a concentration of 10 mM to about 250 mM in combination with AZD1080 is at a concentration of about 1 mM to about 100 mM in combination with Purmorphamine at a concentration of about 100 μM to about 10 mM. In certain embodiments, minoxidil is at a concentration of 10 mM to about 250 mM in combination with AZD1080 is at a concentration of about 1 mM to about 100 mM in combination with SAG at a concentration of about 1 μM to about 100 μM. In certain embodiments, minoxidil is at a concentration of 10 mM to about 250 mM in combination with AZD1080 is at a concentration of about 1 mM to about 100 mM in combination with 20-alpha hydroxy cholesterol at a concentration of about 1 mM to about 100 mM. In certain embodiments, minoxidil is at a concentration of 10 mM to about 250 mM in combination with AZD1080 is at a concentration of about 1 mM to about 100 mM in combination with SAG HCl at a concentration of about 10 μM to about 1 mM.

In certain embodiments of the compositions and methods described herein, minoxidil is at a concentration of 10 mM to about 250 mM in combination with GSK3 inhibitor XXII is at a concentration of about 100 μM to about 10 mM in combination with Purmorphamine at a concentration of about 100 μM to about 10 mM. In certain embodiments, minoxidil is at a concentration of 10 mM to about 250 mM in combination with GSK3 inhibitor XXII is at a concentration of about 100 μM to about 10 mM in combination with SAG at a concentration of about 1 μM to about 100 μM. In certain embodiments, minoxidil is at a concentration of 10 mM to about 250 mM in combination with GSK3 inhibitor XXII is at a concentration of about 100 μM to about 10 mM in combination with 20-alpha hydroxy cholesterol at a concentration of about 1 mM to about 100 mM. In certain embodiments, minoxidil is at a concentration of 10 mM to about 250 mM in combination with GSK3 inhibitor XXII is at a concentration of about 100 μM to about 10 mM in combination with SAG HCl at a concentration of about 10 μM to about 1 mM.

In certain embodiments of the compositions and methods described herein, minoxidil is at a concentration of 10 mM to about 250 mM in combination with Compound I-6 is at a concentration of about 1 μM to about 100 μM in combination with Purmorphamine at a concentration of about 100 μM to about 10 mM. In certain embodiments, minoxidil is at a concentration of 10 mM to about 250 mM in combination with Compound I-6 is at a concentration of about 1 μM to about 100 μM in combination with SAG at a concentration of about 1 μM to about 100 μM. In certain embodiments, minoxidil is at a concentration of 10 mM to about 250 mM in combination with Compound I-6 is at a concentration of about 1 μM to about 100 μM in combination with 20-alpha hydroxy cholesterol at a concentration of about 1 mM to about 100 mM. In certain embodiments, minoxidil is at a concentration of 10 mM to about 250 mM in combination with Compound I-6 is at a concentration of about 1 μM to about 100 μM in combination with SAG HCl at a concentration of about 10 μM to about 1 mM.

In certain embodiments of the compositions and methods described herein, minoxidil is at a concentration of 10 mM to about 250 mM in combination with Compound I-7 is at a concentration of about 1 μM to about 100 μM in combination with Purmorphamine at a concentration of about 100 μM to about 10 mM. In certain embodiments, minoxidil is at a concentration of 10 mM to about 250 mM in combination with Compound I-7 is at a concentration of about 1 μM to about 100 μM in combination with SAG at a concentration of about 1 μM to about 100 μM. In certain embodiments, minoxidil is at a concentration of 10 mM to about 250 mM in combination with Compound I-7 is at a concentration of about 1 μM to about 100 μM in combination with 20-alpha hydroxy cholesterol at a concentration of about 1 mM to about 100 mM. In certain embodiments, minoxidil is at a concentration of 10 mM to about 250 mM in combination with Compound I-7 is at a concentration of about 1 μM to about 100 μM in combination with SAG HCl at a concentration of about 10 μM to about 1 mM.

In certain embodiments of the compositions and methods described herein, minoxidil is at a concentration of 10 mM to about 250 mM in combination with Compound I-12 is at a concentration of about 10 μM to about 1000 μM in combination with Purmorphamine at a concentration of about 100 μM to about 10 mM. In certain embodiments, minoxidil is at a concentration of 10 mM to about 250 mM in combination with Compound I-12 is at a concentration of about 10 μM to about 1000 μM in combination with SAG at a concentration of about 1 μM to about 100 μM. In certain embodiments, minoxidil is at a concentration of 10 mM to about 250 mM in combination with Compound I-12 is at a concentration of about 10 μM to about 1000 μM in combination with 20-alpha hydroxy cholesterol at a concentration of about 1 mM to about 100 mM. In certain embodiments, minoxidil is at a concentration of 10 mM to about 250 mM in combination with Compound I-12 is at a concentration of about 10 μM to about 1000 μM in combination with SAG HCl at a concentration of about 10 μM to about 1 mM.

In certain embodiments of the compositions and methods provided herein, include minoxidil and the Shh pathway activator is SAG (CAS 912545-86-9) or SAG-HCl. In some embodiments, SAG is at a concentration of about 1 μM to 1000 mM, or about 10 μM to 1000 mM, or about 100 μM to 10 mM, or about 1 mM. In some embodiments, SAG HCl is at a concentration ratio of about 0.1 to 1,000,000 fold relative to its Effective Shh Concentration, or about 1 to 100,000 fold relative to its Effective Shh Concentration, or about 10 to 10,000 fold relative to its Effective Shh Concentration, or about 100 to 1000 fold relative to its Effective Shh Concentration, or about 1000 fold relative to its Effective Shh Concentration. In some embodiments, SAG HCl is at about 1×, 3×, 10×, 30×, 100×, 300×, 1000×, 3000×, 5000× relative to its Effective Shh Concentration. In some embodiments, SAG HCl is at a concentration of about 1 nM to 1000 μM, about 10 nM to 100 μM, about 100 nM to 10 μM, about 125 nM, about 250 nM, or about 500 nM, or about 750 nM.

In certain embodiments of the compositions and methods provided herein, include minoxidil and the Shh pathway activator is SAG (CAS 912545-86-9) or SAG-HCl in combination with a Wnt agonist selected from one or more of an SFRP1 inhibitor (for example, WAY-316606), a SFRP2 inhibitor, a SFRP3 inhibitor, a SFRP4 inhibitor, a SFRP5 inhibitor, a cyclosporine or an analog thereof (for example, cyclosporine A (CsA), PSC833 (Valspodar)), a DKK1 inhibitor (for example, WAY-262611), and a WIF1 inhibitor. In some embodiments, SAG is at a concentration of about 1 μM to 1000 mM, or about 10 μM to 1000 mM, or about 100 μM to 10 mM, or about 1 mM. In some embodiments, SAG HCl is at a concentration ratio of about 0.1 to 1,000,000 fold relative to its Effective Shh Concentration, or about 1 to 100,000 fold relative to its Effective Shh Concentration, or about 10 to 10,000 fold relative to its Effective Shh Concentration, or about 100 to 1000 fold relative to its Effective Shh Concentration, or about 1000 fold relative to its Effective Shh Concentration. In some embodiments, SAG HCl is at about 1×, 3×, 10×, 30×, 100×, 300×, 1000×, 3000×, 5000× relative to its Effective Shh Concentration. In some embodiments, SAG HCl is at a concentration of about 1 nM to 1000 μM, about 10 nM to 100 μM, about 100 nM to 10 μM, about 125 nM, about 250 nM, or about 500 nM, or about 750 nM.

Hair Follicle Growth

Hair follicles develop through complex morphogenetic processes resulting from reciprocal molecular interactions between epithelium and underlying mesenchyme during embryonic development. Each hair follicle goes through regenerative cycling. The hair cycle consists of phases of growth (anagen), degeneration (catagen) and rest (telogen). In catagen, hair follicle stem cells are maintained in the bulge. Then the resting follicle re-enters anagen (regeneration) when proper molecular signals are provided. During late telogen to early anagen transition, signals from the dermal papilla (DP) stimulate the hair germ and quiescent bulge stem cells to become activated. In anagen, stem cells in the bulge give rise to hair germs, then the transient amplifying cells in the matrix of the new follicle proliferate rapidly to form a new hair filament. After catagen, follicles undergo apoptosis. The hair filament remains in the telogen follicle to become a club hair, which later is detached during exogen. These regenerative cycles continue repetitively throughout the lifetime of an organism.

Minoxidil

Minoxidil is an antihypertensive vasodilator medication and is used to treat hair loss. It is effective in helping promote hair growth in people with androgenic alopecia regardless of sex. The mechanism by which minoxidil promotes hair growth is not fully understood. Minoxidil is a potassium channel opener, causing hyperpolarization of cell membranes. Hypothetically, by widening blood vessels and opening potassium channels, it allows more oxygen, blood, and nutrients to the follicles. This may cause follicles in the telogen phase to shed, which are then replaced by thicker hairs in a new anagen phase. Minoxidil is a prodrug that is converted by sulfation via the sulfotransferase enzyme SULTIA1 to its active form, minoxidil sulfate. Several studies demonstrated that the activity of sulfotransferase in hair follicles predict minoxidil response in the treatment of hair loss.

Hedgehog Pathway

The evolutionarily conserved Hedgehog (Hh) pathway is essential for normal embryonic development and plays critical roles in adult tissue maintenance, renewal and regeneration. Secreted Hh proteins act in a concentration- and time-dependent manner to initiate a series of cellular responses that range from survival and proliferation to cell fate specification and differentiation. Proper levels of Hh signaling require the regulated production, processing, secretion and trafficking of Hh ligands—in mammals this includes Sonic (Shh), Indian (Ihh) and Desert (Dhh). All Hh ligands are synthesized as precursor proteins that undergo autocatalytic cleavage and concomitant cholesterol modification at the carboxy terminus and palmitoylation at the amino terminus, resulting in a secreted, dually-lipidated protein. Hh ligands are released from the cell surface through the combined actions of Dispatched and Scube2, and subsequently trafficked over multiple cells through interactions with the cell surface proteins LRP2 and the Glypican family of heparan sulfate proteoglycans (GPC1-6). Hh proteins initiate signaling through binding to the canonical receptor Patched (PTCH1) and to the co-receptors GAS1, CDON and BOC. Hh binding to PTCH1 results in derepression of the GPCR-like protein Smoothened (SMO) that results in SMO accumulation in cilia and phosphorylation of its cytoplasmic tail. SMO mediates downstream signal transduction that includes dissociation of GLI proteins (the transcriptional effectors of the Hh pathway) from kinesin-family protein, Kif7, and the key intracellular Hh pathway regulator SUFU. GLI proteins also traffic through cilia and in the absence of Hh signaling are sequestered by SUFU and Kif7, allowing for GLI phosphorylation by PKA, GSK30 and CK1, and subsequent processing into transcriptional repressors (through cleavage of the carboxy-terminus) or targeting for degradation (mediated by the E3 ubiquitin ligase 3-TrCP). In response to activation of Hh signaling, GLI proteins are differentially phopshorylated and processed into transcriptional activators that induce expression of Hh target genes, many of which are components of the pathway (e.g. PTCH1 and GLI1). Feedback mechanisms include the induction of Hh pathway antagonists (PTCH1, PTCH2 and Hhip1) that interfere with Hh ligand function, and GLI protein degradation mediated by the E3 ubiquitin ligase adaptor protein, SPOP. In addition to vital roles during normal embryonic development and adult tissue homeostasis, aberrant Hh signaling is responsible for the initiation of a growing number of cancers including, classically, basal cell carcinoma, edulloblastoma, and rhabdomyosarcoma; more recently overactive Hh signaling has been implicated in pancreatic, lung, prostate, ovarian, and breast cancer.

Wnt Pathway

Three Wnt signaling pathways have been characterized: the canonical Wnt pathway, the noncanonical planar cell polarity pathway, and the noncanonical Wnt/calcium pathway. All three pathways are activated by the binding of a Wnt-protein ligand to a Frizzled family receptor, which passes the biological signal to the Dishevelled protein inside the cell.

The canonical Wnt pathway leads to regulation of gene transcription, and is thought to be negatively regulated in part by the SPATS 1 gene.

The noncanonical planar cell polarity pathway regulates the cytoskeleton that is responsible for the shape of the cell.

The noncanonical Wnt/calcium pathway regulates calcium inside the cell.

Wnt signaling pathways use either nearby cell-cell communication (paracrine) or same-cell communication (autocrine). They are highly evolutionarily conserved in animals, which means they are similar across animal species from fruit flies to humans.

Wnt signaling was first identified for its role in carcinogenesis, then for its function in embryonic development. The embryonic processes it controls include body axis patterning, cell fate specification, cell proliferation and cell migration. These processes are necessary for proper formation of important tissues including bone, heart and muscle. Its role in embryonic development was discovered when genetic mutations in Wnt pathway proteins produced abnormal fruit fly embryos. Wnt signaling also controls tissue regeneration in adult bone marrow, skin and intestine. Research found that the genes responsible for these abnormalities also influenced breast cancer development in mice.

This pathway's clinical importance was demonstrated by mutations that lead to various diseases, including breast and prostate cancer, glioblastoma, type II diabetes and others. Encouragingly, in recent years researchers reported first successful use of Wnt pathway inhibitors in mouse models of disease.

Administration

In certain embodiments, pharmaceutical formulations are adapted to administer the drug locally to an area of skin where hair loss is occurring. Liquid, gel or foam formulations may be used. It is also possible to apply the active ingredient topically or to employ a combination of delivery approaches.

Injection approaches include by osmotic pump, or, by combination with implanted biomaterial, and more preferably, by injection or infusion. Biomaterials that can aid in controlling release kinetics and distribution of drug include hydrogel materials, degradable materials. One class of materials that is most preferably used includes in situ gelling materials. Other materials include collagen or other natural materials including fibrin, gelatin, and decelluarized tissues. Gelfoam may also be suitable.

Delivery may also be enhanced via alternate means including but not limited to agents added to the delivered composition such as penetration enhancers, or could be through devices via ultrasound, electroporation, or high speed jet.

With regard to human and veterinary treatment, the amount of a particular agent(s) that is administered may be dependent on a variety of factors, including the disorder being treated and the severity of the disorder; activity of the specific agent(s) employed; the age, body weight, general health, sex and diet of the patient; the time of administration, route of administration, and rate of excretion of the specific agent(s) employed; the duration of the treatment; drugs used in combination or coincidental with the specific agent(s) employed; the judgment of the prescribing physician or veterinarian; and like factors known in the medical and veterinary arts.

The agents described herein may be administered in a therapeutically effective amount to a subject in need of treatment. Administration of compounds described herein can be via any of suitable route of administration, particularly intradermally, orally or topically. Other routes include ingestion, or alternatively parenterally, for example intravenously, intra-arterially, intraperitoneally, intrathecally, intraventricularly, intraurethrally, intrasternally, intracranially, intramuscularly, intranasally, subcutaneously, sublingually, transdermally, or by inhalation or insufflations, or topical for absorption through the skin. Such administration may be as a single or multiple oral dose, defined number of ear drops, or a bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, the compounds are preferably formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives.

Compounds and compositions described herein can be administered by a number of methods sufficient to deliver the compound to an area of skin with hair loss.

“Contacting” as used herein for administration of the compositions of the present disclosure, refers, in some embodiments, to bringing skin, in one embodiment, scalp, eyebrow, etc., into contact with one or more compound, factor, cell, etc. In another embodiment, the term refers to embedding the one or more compound, factor, cell, etc. into the skin region of interest. In another embodiment, the term refers to injecting the one or more compound, factor, cell, etc. into the skin region of interest. In another embodiment, the term refers to any other type of contacting known in the art. Each possibility represents a separate embodiment of the present disclosure.

In another embodiment, the step of contacting in methods of administering one or more compounds of the present disclosure comprises directly contacting the skin region of interest with the compound, RNA, protein, etc. In another embodiment, the step of contacting comprises indirectly contacting the skin region of interest via contacting another site or tissue of the subject, after which the compound, RNA, or protein is transported to the skin region of interest by a biological process; e.g, diffusion, active transport, or circulation in a fluid such as the blood, lymph, interstitial fluid, etc. Each possibility represents a separate embodiment of the present disclosure.

In particular embodiments, the compounds, compositions and formulations of the disclosure are locally administered, meaning that they are not administered systemically.

In some embodiments, composition provided herein is administered to a subject in need thereof once. In some embodiments, composition provided herein is administered to a subject in need thereof more than once. In some embodiments, a first administration of composition provided herein is followed by a second, third, fourth, or fifth administration of composition provided herein.

The number of times a compound is administered to an subject in need thereof depends on the discretion of a medical professional, the disorder, the severity of the disorder, and the subject's response to the formulation. In some embodiments, the compound disclosed herein is administered once to a subject in need thereof with a mild acute condition. In some embodiments, the compound disclosed herein is administered more than once to a subject in need thereof with a moderate or severe acute condition. In the case wherein the subject's condition does not improve, upon the doctor's discretion the compound may be administered chronically, that is, for an extended period of time, including throughout the duration of the subject's life in order to ameliorate or otherwise control or limit the symptoms of the subject's disease or condition.

In the case wherein the subject's status does improve, upon the doctor's discretion the compound may administered continuously; alternatively, the dose of drug being administered may be temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once the subject's hair density or hair growth has improved, a maintenance dose can be administered, if necessary. Subsequently, the dosage or the frequency of administration, or both, is optionally reduced, as a function of the symptoms, to a level at which the improved disease, disorder or condition is retained. In certain embodiments, subjects require intermittent treatment on a long-term basis upon any recurrence of symptoms.

In certain embodiments, the pharmaceutical formulations may also contain an additional agent selected from a Notch activator, HDAC inhibitor, a BMP4 antagonist, Noggin (Inhibits BMP4), Sox2, Vitamin D (calcitriol), Vitamin B (nicotinomide), Vitamin A, Vitamin C (pVc). Lgr4, p38/MAPK inhibition, ROCK inhibition, and/or Alk4/7 inhibition. In certain embodiments, the pharmaceutical formulations may also contain an epidermal growth factor (EGF), fibroblast growth factor (FGF), insulin-like growth factor (IGF), or a combination thereof.

Pharmaceutical Compositions

The present disclosure provides pharmaceutical preparations comprising the present compounds. The present compounds may be conveniently formulated for administration with a biologically acceptable medium, such as water, buffered saline, polyol (for example, glycerol, propylene glycol, liquid polyethylene glycol and the like) or suitable mixtures thereof. The optimum concentration of the active ingredient(s) in the chosen medium may be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the present compound, its use in the pharmaceutical preparation of the present invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”.

Pharmaceutical formulations of the present invention may also include veterinary compositions, e.g., pharmaceutical preparations of the present compounds suitable for veterinary uses, e.g., for the treatment of livestock, such as goats, horses, sheep, etc., or domestic animals, e.g., dogs, cats, rabbits, etc., or other animals, such as apes, monkeys, and chimpanzees.

The present compounds may be formulated for administration in any convenient way for use in human or veterinary medicine. In certain embodiments, the compound included in the pharmaceutical preparation may be active itself, or may be a prodrug, e.g., capable of being converted to an active compound in a physiological setting.

The present disclosure provides pharmaceutically acceptable compositions comprising a therapeutically effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments, the present compounds may be simply dissolved or suspended in sterile water. In certain embodiments, the pharmaceutical preparation is non-pyrogenic, i.e., does not elevate the body temperature of a patient. The phrase “therapeutically effective amount” as used herein means that amount of a compound, material, or composition comprising a compound of the present invention which is effective for producing some desired therapeutic effect in at least a sub-population of cells in an animal and thereby blocking the biological consequences of that pathway in the treated cells, at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, useful for preparing a medically or therapeutically useful composition of the present compounds. Each carrier must be “acceptable” in the sense of being compatible with the other ingredients of the formulation and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, certain embodiments of the present compounds may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts may be prepared in situ during the final isolation and purification of the compounds of the present invention, or by separately reacting a purified compound of the present invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the present compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present disclosure may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts may likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethyl enedi amine, ethanolamine, diethanol amine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants may also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which may be combined with a carrier material to produce a single dosage form will vary depending upon the host being treated, the particular mode of administration. The amount of active ingredient which may be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present disclosure with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the present disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present disclosure may also be administered as a bolus, electuary or paste.

In solid dosage forms of the present disclosure for oral administration (capsules, tablets, pills, dragees, powders, granules and the like), the active ingredient may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, silicon dioxide, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent. The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which may be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which may be used include polymeric substances and waxes. The active ingredient may also be in microencapsulated form, if appropriate, with one or more of the above-described excipients.

Liquid dosage forms for oral administration of the compounds of the present invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions may also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the present disclosure for rectal or vaginal administration may be presented as a suppository, which may be prepared by mixing one or more of the present compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active present compound.

Formulations of the present disclosure which may be useful for vaginal administration also include pessaries, tampons, creams, gels, pastes, foams or spray formulations containing such carriers as are known in the art to be appropriate.

Dosage forms for the topical or transdermal administration of a compound of this invention include liquids, powders, sprays, foams, ointments, pastes, creams, lotions, gels, solutions, patches and inhalants. The active compound may be mixed under sterile conditions with a pharmaceutically acceptable carrier, and with any preservatives, buffers, or propellants which may be required.

In addition to the active ingredient, the topical liquid forms may contain inert diluents commonly used in the art, such as, for example, water or other solvents, solubilizing agents and emulsifiers, such as stearyl alcohol, cetyl alcohol, ethyl alcohol, isopropyl alcohol, ethyl carbonate, ethyl acetate, benzyl alcohol, benzyl benzoate, 1,3-butylene glycol, oils (in particular, cottonseed, groundnut, corn, germ, olive, castor and sesame oils), glycerol, tetrahydrofuryl alcohol, propylene glycol, polyethylene glycols and fatty acid esters of sorbitan, and mixtures thereof.

In addition to the active ingredient, the topical gel forms may contain inert diluents commonly used in the art, such as, for example, water or other solvents, solubilizing agents and emulsifiers, such as carbopol (such as carbopol 934) hydroxypropyl cellulose, hydroxyl Pr cellulose, hydroxypropyl methylcellulose methyl-3-cyclodextrin calcium alginate, sodium alginate, carbopol 934 and hydroxyethylcellulose.

Minoxidil was dissolved in solvent system comprising water and propylene glycol in ratio 35:15 with liq. paraffin as oil phase. The prepd. w/o emulsion was then mixed with carbopol gel soln. in 1:1 ratio and finally neutralized with triethanolamine to form emulgel.

Gel prepd. by using Natrosol, Carbopol 974 and HPMC K100 (hydroxyl Pr Me cellulose) as viscosity enhancing agents.

Gel prepd. using these polymers such as Carbopol-934, Carbopol-941, Poloxamer 188 in different concentrations

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays may contain, in addition to a compound of this invention, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms may be made by dissolving or dispersing the present compounds in the proper medium. Absorption enhancers may also be used to increase the flux of the present compounds across the skin. The rate of such flux may be controlled by either providing a rate controlling membrane or dispersing the compound in a polymer matrix or gel.

Examples of suitable aqueous and nonaqueous carriers which may be employed in the pharmaceutical compositions of the present disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants. These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms may be made by forming microencapsulated matrices of the present compounds in biodegradable polymers such as polylactide-polyglycolide. Depending on the ratio of drug to polymer, and the nature of the particular polymer employed, the rate of drug release may be controlled. Examples of other biodegradable polymers include poly(orthoesters) and poly(anhydrides). Depot injectable formulations may also be prepared by entrapping the drug in liposomes or microemulsions which are compatible with body tissue.

When the compounds of the present disclosure may be administered as pharmaceuticals, to humans and animals, they may be given per se or as a pharmaceutical composition containing, for example, about 0.1 to 99.5% (more preferably, about 0.5 to 90%) of active ingredient in combination with a pharmaceutically acceptable carrier.

The addition of one or more of the present compounds of the present disclosure to animal feed may be accomplished by preparing an appropriate feed premix containing the active compound in an effective amount and incorporating the premix into the complete ration.

Alternatively, an intermediate concentrate or feed supplement containing the active ingredient may be blended into the feed. The way in which such feed premixes and complete rations may be prepared and administered are described in reference books (such as “Applied Animal Nutrition”, W.H. Freedman and CO., San Francisco, U.S.A., 1969 or “Livestock Feeds and Feeding” O and B books, Corvallis, Ore., U.S.A., 1977).

Compositions with Poloxamers

In certain embodiments, the present disclosure provides a pharmaceutical composition comprising: a) a compound(s) of the present disclosure and b) a poloxamer.

In certain embodiments, the pH of the pharmaceutical composition is between about 5 and about 9. In certain embodiments, the pH of the pharmaceutical composition is about 5, 6, 7, 8, or 9.

In certain embodiments, the solubility of the compound in the presence of the poloxamer is about 3-fold higher than the solubility of the compound at the same pH in the absence of poloxamer. In certain embodiments, the solubility of the compound in the presence of the poloxamer is about 2-, 3-, 4- or 5-fold higher than the solubility of the compound at the same pH in the absence of poloxamer.

In certain embodiments, the pharmaceutical formulations may also contain a poloxamer. Poloxamers are nonionic triblock copolymers composed of a central hydrophobic chain of polyoxypropylene (poly(propylene oxide)) flanked by two hydrophilic chains of polyoxyethylene (poly(ethylene oxide)). Poloxamers are often considered “functional excipients” because they are essential components and play an important role in a formulation.

In some embodiments, the poloxamer comprises at least one of Poloxamer 124, Poloxamer 188, Poloxamer 237, Poloxamer 338 or Poloxamer 407. In some embodiments, the poloxamer comprises mixtures of two or more of Poloxamer 124, Poloxamer 188, Poloxamer 237, Poloxamer 338 or Poloxamer 407. In some embodiments, the mixture of two or more poloxamers comprise Poloxamer 407 and Poloxamer 124. In another embodiment the Poloxamer comprises at least one of Poloxamer 188 and Poloxamer 407 or mixtures thereof. In some embodiments, the poloxamer is Poloxamer 407.

In some embodiments, the poloxamer is in a concentration between about 5 wt % and about 25 wt % relative to the composition. In some embodiments, the poloxamer is in a concentration between about 10 wt % and about 23 wt % relative to the composition. In some embodiments, the poloxamer is in a concentration between about 15 wt % and about 20 wt % relative to the composition. In some embodiments, the poloxamer is in a concentration is approximately 17 wt % relative to the composition. In some embodiments, the poloxamer is in a concentration is approximately 21 wt % relative to the composition.

In some embodiments, the poloxamer can be in a concentration between 21 wt % and 40 wt % relative to the composition. In another embodiment the poloxamer is in a concentration between 21 wt % and 30 wt % relative to the composition. In another embodiment the poloxamer is in a concentration between 23 wt % and 29 wt % relative to the composition. In another embodiment the poloxamer is in a concentration between 23 wt % and 27 wt % relative to the composition. In another embodiment the poloxamer is in a concentration of 25 wt % relative to the composition.

In some embodiments, the gelation temperature of the pharmaceutical composition is greater than about 10° C. In some embodiments, the gelation temperature of the pharmaceutical composition is between about 11° C. and about 32° C. In some embodiments, the gelation temperature of the pharmaceutical composition is between about 15° C. and about 30° C. In some embodiments, the gelation temperature of the pharmaceutical composition is between about 20° C. and about 28° C. In some embodiments, the gelation temperature of the pharmaceutical composition is between about 24° C. and about 26° C.

In some embodiments, the gelation temperature of the pharmaceutical composition is about 15° C. In some embodiments, the gelation temperature of the pharmaceutical composition is about 20° C. In some embodiments, the gelation temperature of the pharmaceutical composition is about 24° C. In some embodiments, the gelation temperature of the pharmaceutical composition is about 26° C. In some embodiments, the gelation temperature of the pharmaceutical composition is about 28° C. In some embodiments, the gelation temperature of the pharmaceutical composition is about 30° C. In some embodiments, the gelation temperature of the pharmaceutical composition is about 32° C.

Methods of Making Compounds

The compounds described herein may be made from commercially available starting materials or synthesized using known organic, inorganic, and/or enzymatic processes.

The compounds of the present invention can be prepared in a number of ways well known to those skilled in the art of organic synthesis. By way of example, compounds of the disclosure can be synthesized using the methods described below, together with synthetic methods known in the art of synthetic organic chemistry, or variations thereon as appreciated by those skilled in the art. These methods include but are not limited to those methods described below.

A representative synthesis for subject compounds is shown in Scheme 1.

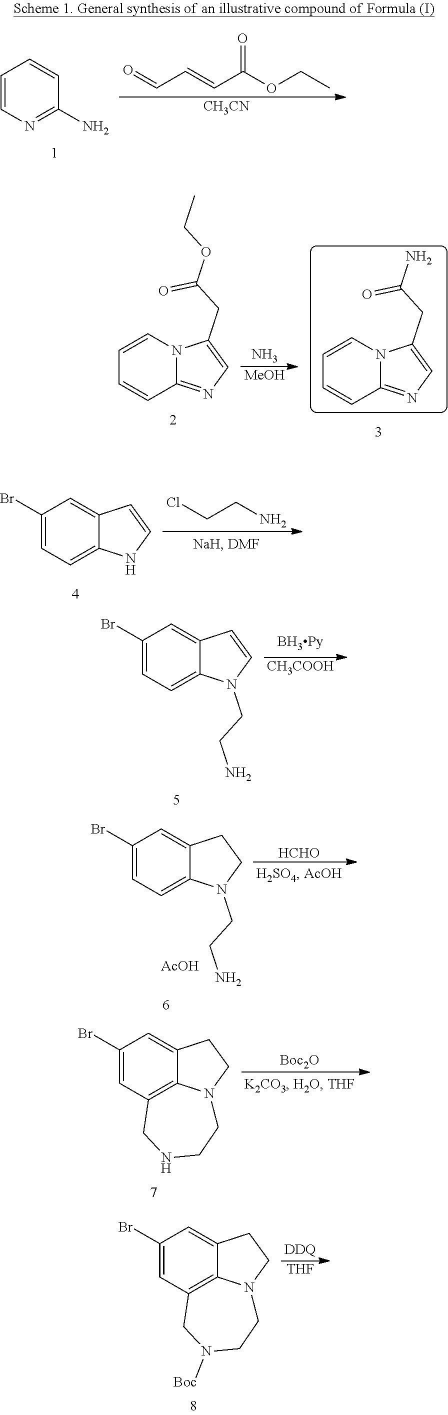

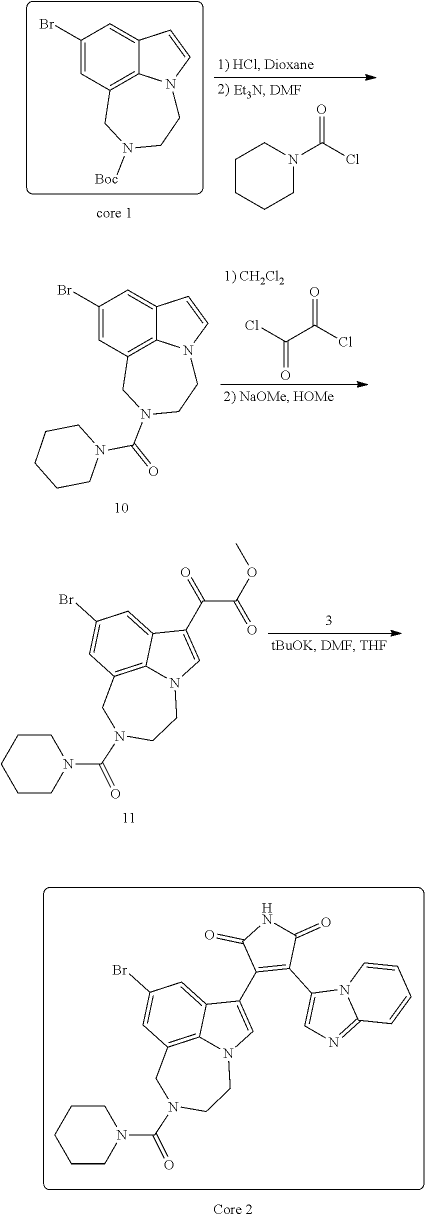 [SMILES: CC(C)(C)OC(=O)N1CCn2ccc3cc(Br)cc(c32)C1 ; O=C(Cl)N1CCCCC1 ; O=C(N1CCCCC1)N1CCn2ccc3cc(Br)cc(c32)C1 ; O=C(Cl)C(=O)Cl ; COC(=O)C(=O)c1cn2c3c(cc(Br)cc13)CN(C(=O)N1CCCCC1)CC2 ; O=C1NC(=O)C(c2cnc3ccccn23)=C1c1cn2c3c(cc(Br)cc13)CN(C(=O)N1CCCCC1)CC2]

In Scheme 1, compound I-11 is an embodiment wherein Q¹ is CH; Q² is N; and Q³ is C; R² is bromo; Ar is

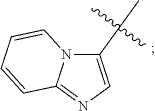 [SMILES: *c1cnc2ccccn12]

### and —Z—W—X—Y— is.

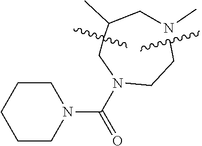

Compounds of formula 1 and the allylic aldehyde are commercially available starting materials. Alternatively, compounds of formula 1 and the allylic aldehyde can be synthesized via a variety of different synthetic routes using commercially available starting materials and/or starting materials prepared by conventional synthetic methods.

With continued reference to Scheme 1, compound 1 and an allylic aldehyde are reacted to form compound 2 in a condensation reaction in a suitable solvent such as acetonitrile at a temperature, for example, from about 40° C. to 100° C. Compound 2 is reacted with ammonia to form compound 3 in a suitable solvent such as methanol at a temperature, for example, in the range from 0° C. to room temperature. Compound 3 can be used in a coupling reaction to be discussed below.

With continued reference to Scheme 1, compound 5 may be prepared from an alkylation of compound 4 with an alkyl halide in the presence of a base, such as an alkali metal hydride, such as sodium hydride. The reaction can be run in a suitable solvent, such as dimethylformamide (DMF) at a temperature, for example, in the range from 0° C. to room temperature.

Compound 6 may be prepared from the reduction of compound 5. Suitable reduction reagents include borane pyridine complex. The reaction can be run in a suitable solvent, such as acetic acid at a temperature, for example, in the range from 0° C. to room temperature.

Compound 7 may be prepared from reaction of compound 6. The reaction is carried with formaldehyde in the presence of acid such as sulfuric acid and acetic acid.

Compound 8 may be prepared from protection of the amino group of compound 7. Suitable reagents include BOC anhydride. The reaction can be run in a suitable solvent, such as tetrahydrofuran (THF) at a temperature, for example, in the range from 0° C. to room temperature.

Core 1 may be prepared from the dehydrogenation of compound 8. Suitable reagents include DDQ (2,3-dichloro-5,6-dicyano-1,4-benzoquinone). The reaction can be run in a suitable solvent, such as tetrahydrofuran (THF) at a temperature, for example, in the range from 0° C. to room temperature.

Compound 10 may be prepared from core 1 by deprotection of the amino group and then the subsequent reaction with acyl halide. The deprotection of the amino group can be performed under acidic conditions, if the protecting group is BOC. Then reaction with an acyl halide can result in compound 10. The reaction can be run in a suitable solvent, such as dimethylformamide (DMF) at a temperature, for example, in the range from 0° C. to room temperature.

Compound 11 may be prepared from compound 10 by an acylation reaction, such as a Friedel Crafts acylation reaction. In this reaction, an acyl halide is reacted with compound 10 in a suitable solvent, such as methylene chloride at a temperature, for example, in the range from 30° C. to 100° C. The product is then reacted an alcohol and base to form an ester, as in compound 11.

Compound 11 and Compound 3 are reacted to form the 1H-pyrrole-2,5-dione compound. The reaction is carried out in an inert organic solvent such as dimethylformalnide, tetrahy drofuran, and the like and in the presence of a base such as potassium tert-butoxide.

### Synthetic Schemes

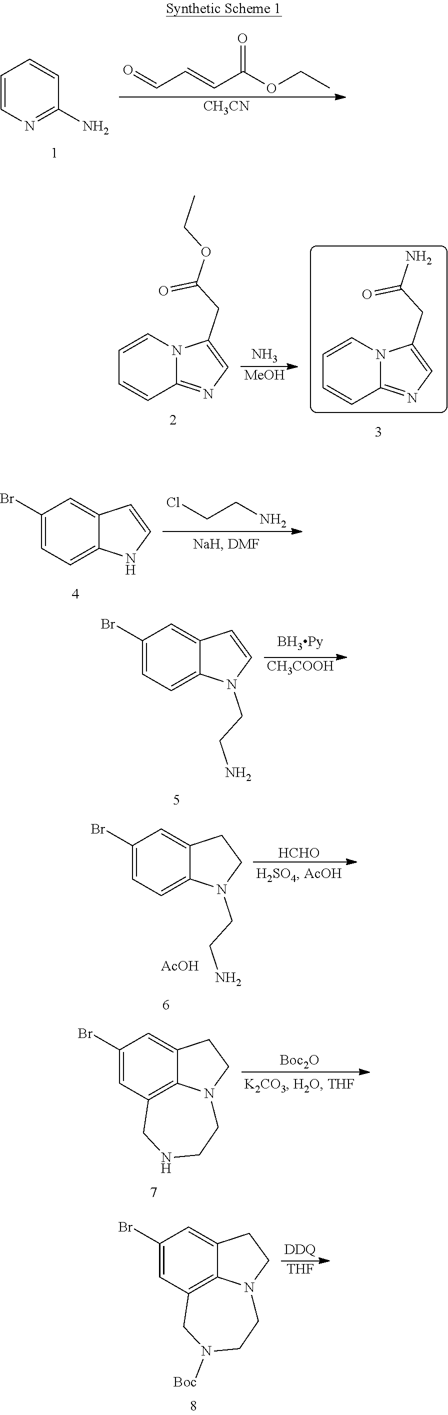

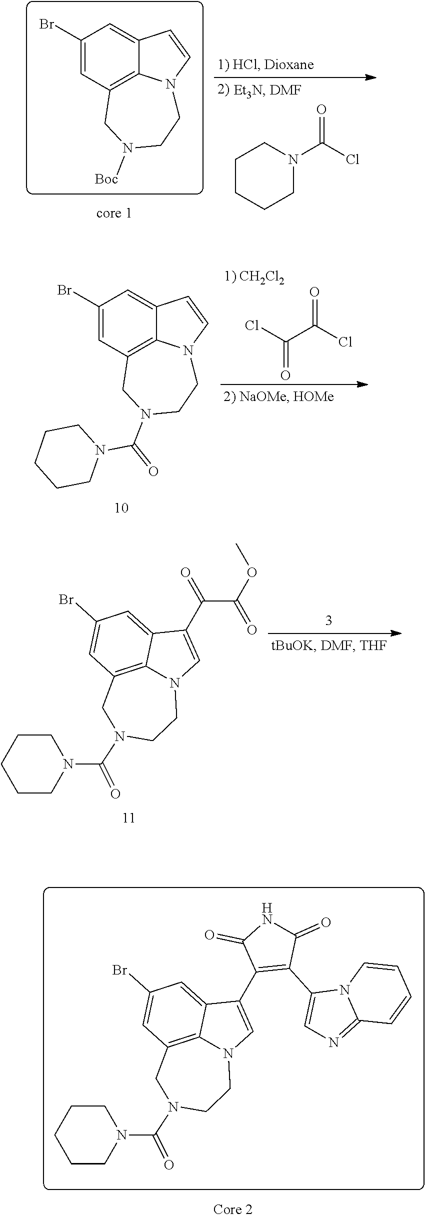 [SMILES: CC(C)(C)OC(=O)N1CCn2ccc3cc(Br)cc(c32)C1 ; O=C(Cl)N1CCCCC1 ; O=C(N1CCCCC1)N1CCn2ccc3cc(Br)cc(c32)C1 ; O=C(Cl)C(=O)Cl ; COC(=O)C(=O)c1cn2c3c(cc(Br)cc13)CN(C(=O)N1CCCCC1)CC2 ; O=C1NC(=O)C(c2cnc3ccccn23)=C1c1cn2c3c(cc(Br)cc13)CN(C(=O)N1CCCCC1)CC2]

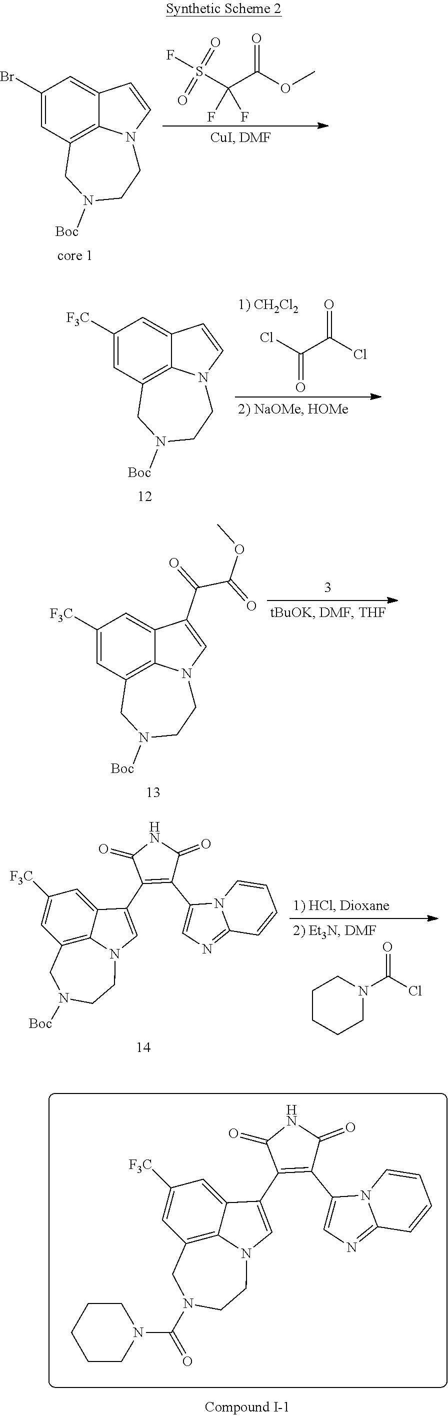

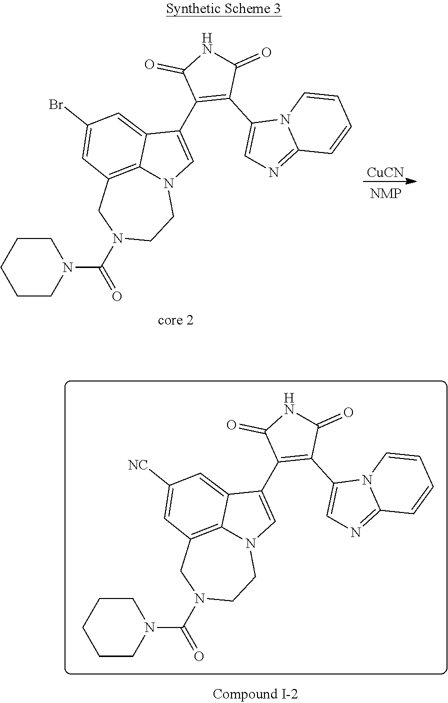

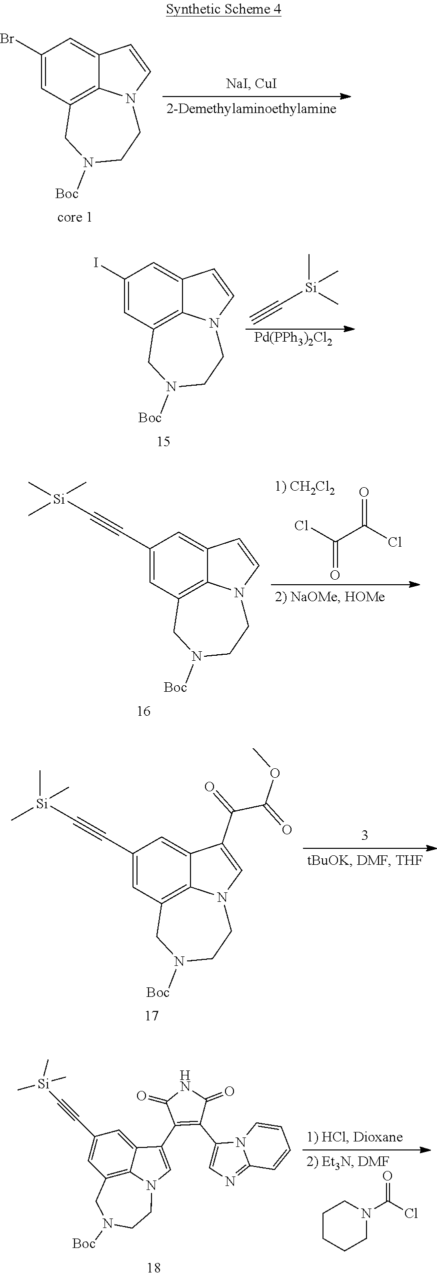

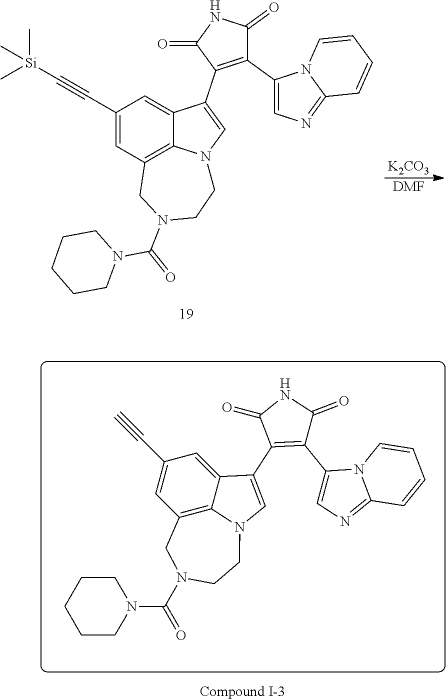

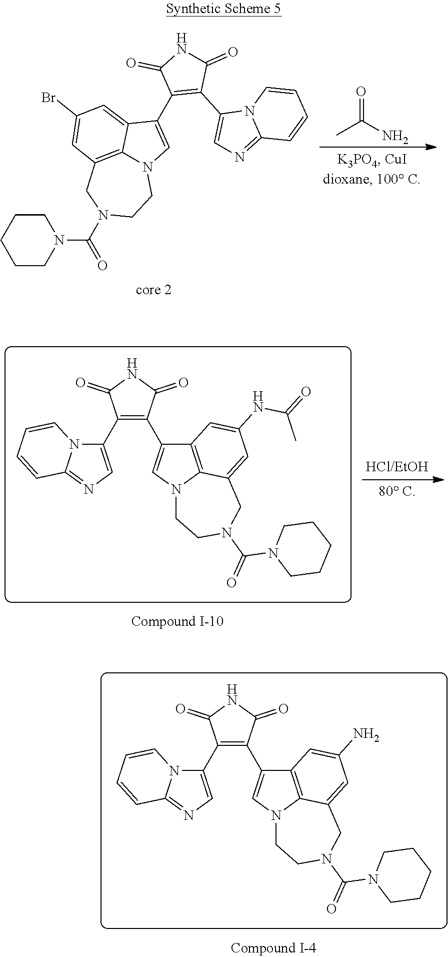

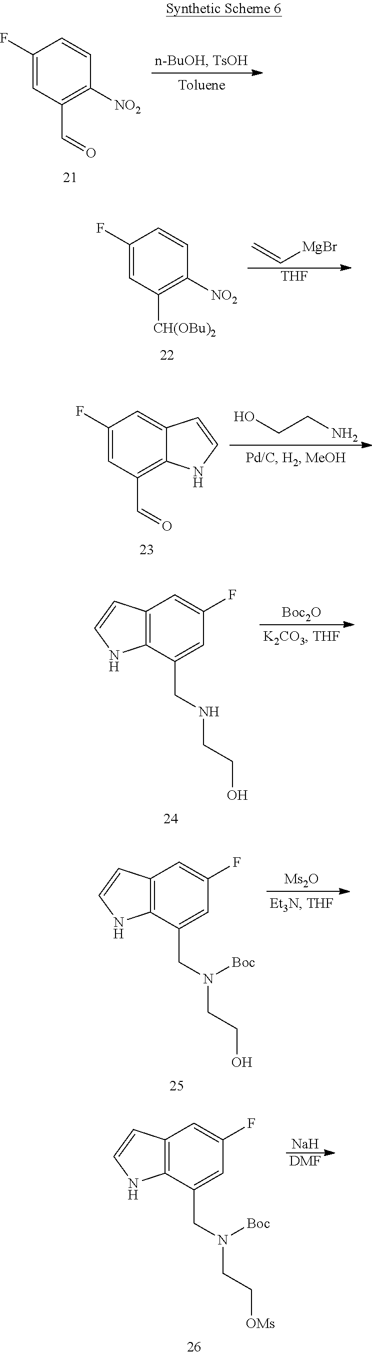

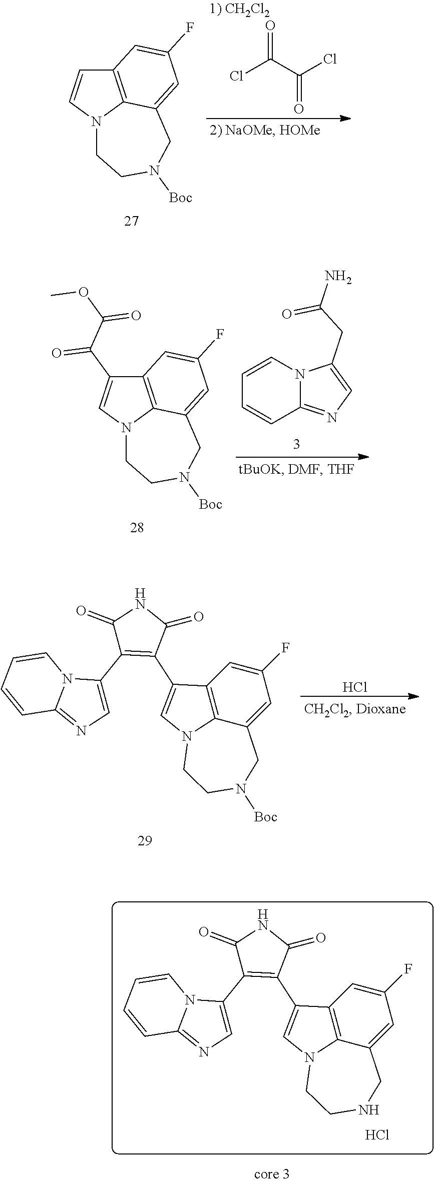

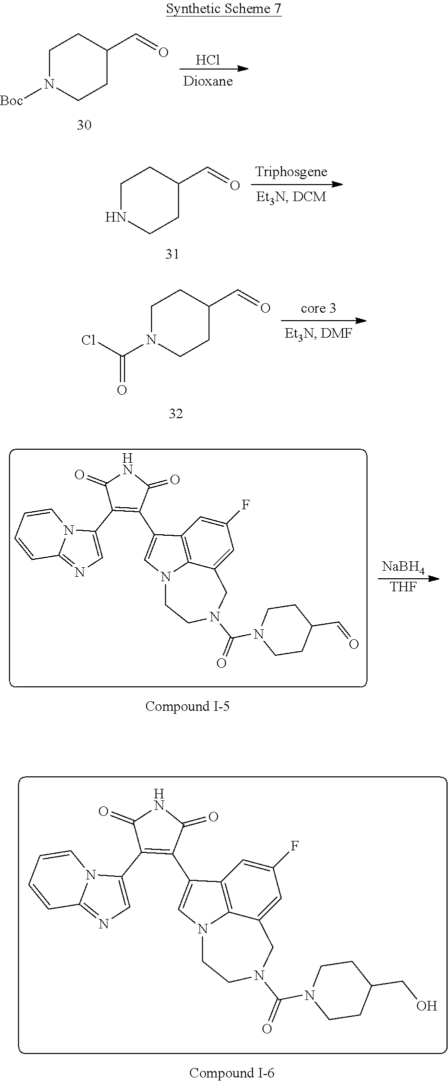

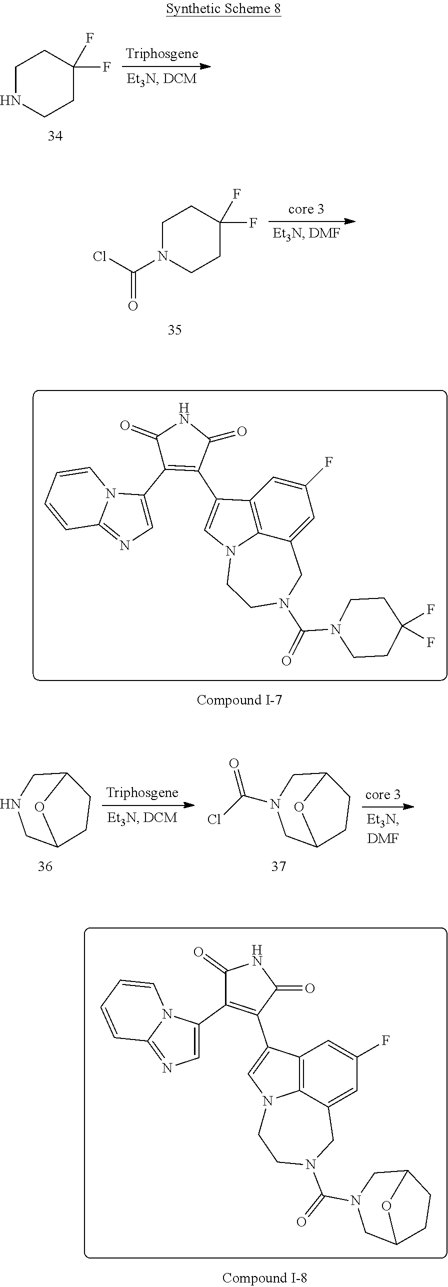 [SMILES: FC1(F)CCNCC1 ; O=C(Cl)N1CCC(F)(F)CC1 ; O=C1NC(=O)C(c2cnc3ccccn23)=C1c1cn2c3c(cc(F)cc13)CN(C(=O)N1CCC(F)(F)CC1)CC2 ; C1CC2CNCC1O2 ; O=C(Cl)N1CC2CCC(C1)O2 ; O=C1NC(=O)C(c2cnc3ccccn23)=C1c1cn2c3c(cc(F)cc13)CN(C(=O)N1CC3CCC(C1)O3)CC2]

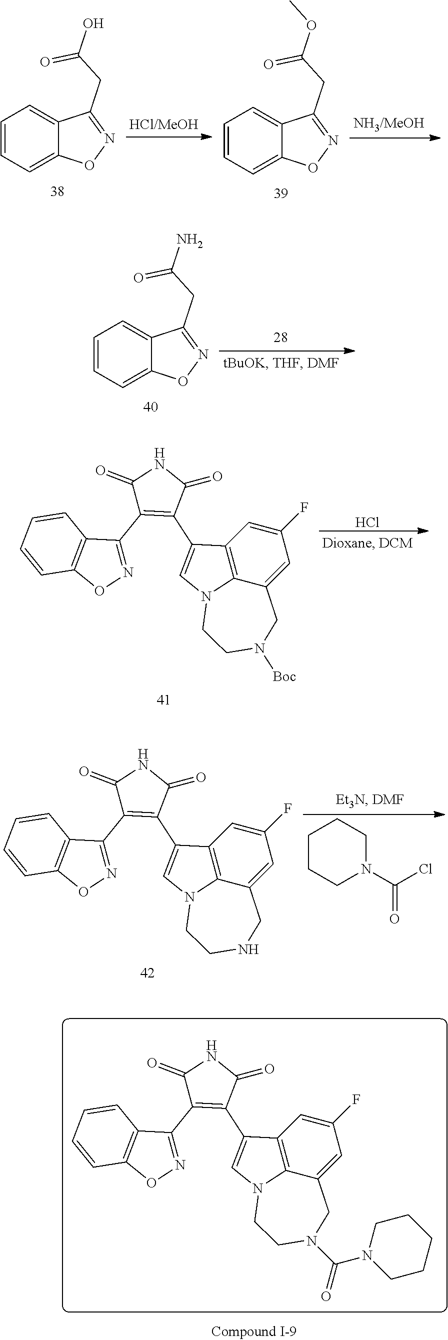

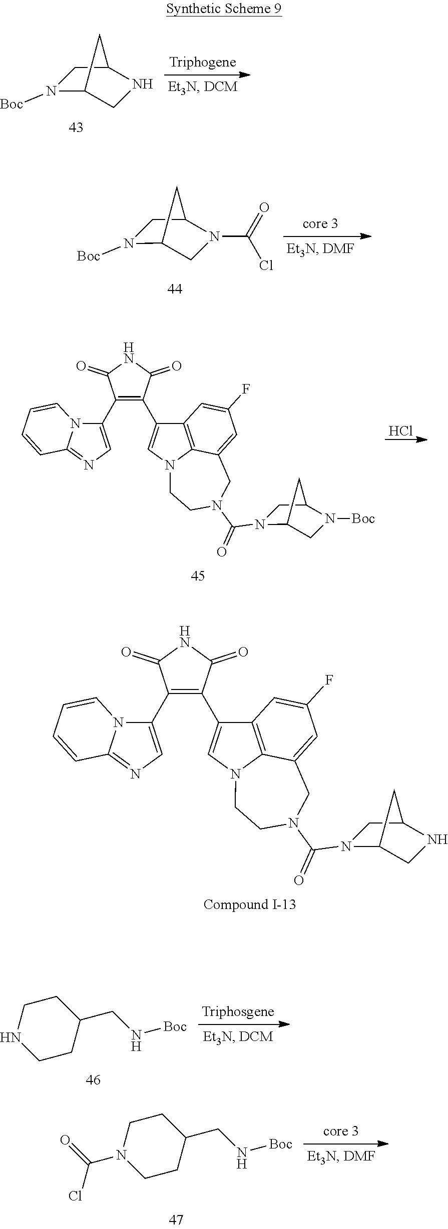

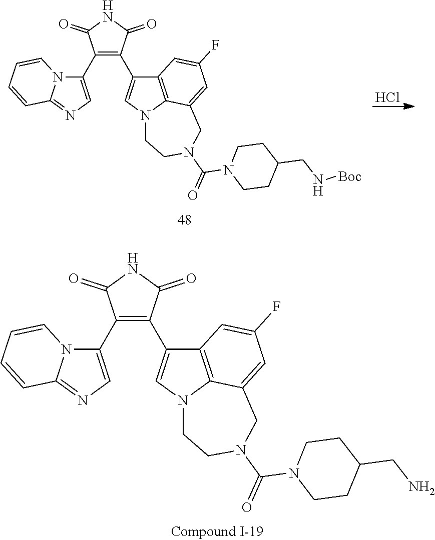 [SMILES: CC(C)(C)OC(=O)NCC1CCN(C(=O)N2CCn3cc(C4=C(c5cnc6ccccn56)C(=O)NC4=O)c4cc(F)cc(c43)C2)CC1 ; Cl ; NCC1CCN(C(=O)N2CCn3cc(C4=C(c5cnc6ccccn56)C(=O)NC4=O)c4cc(F)cc(c43)C2)CC1]

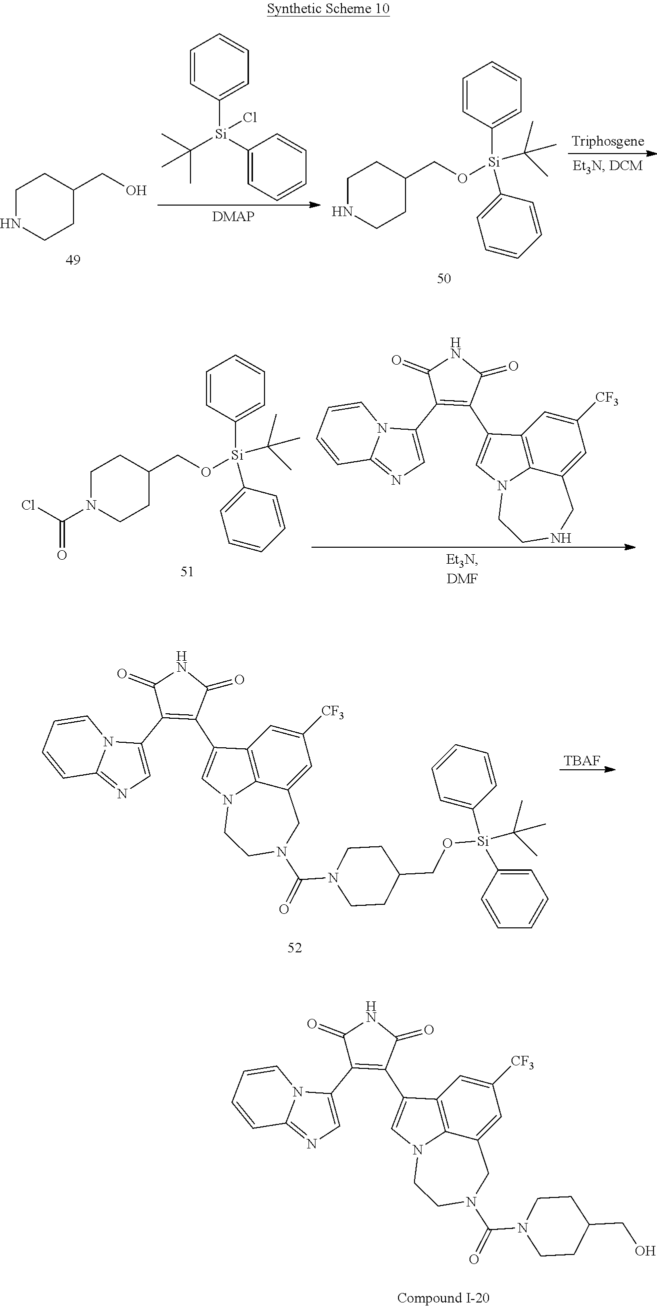

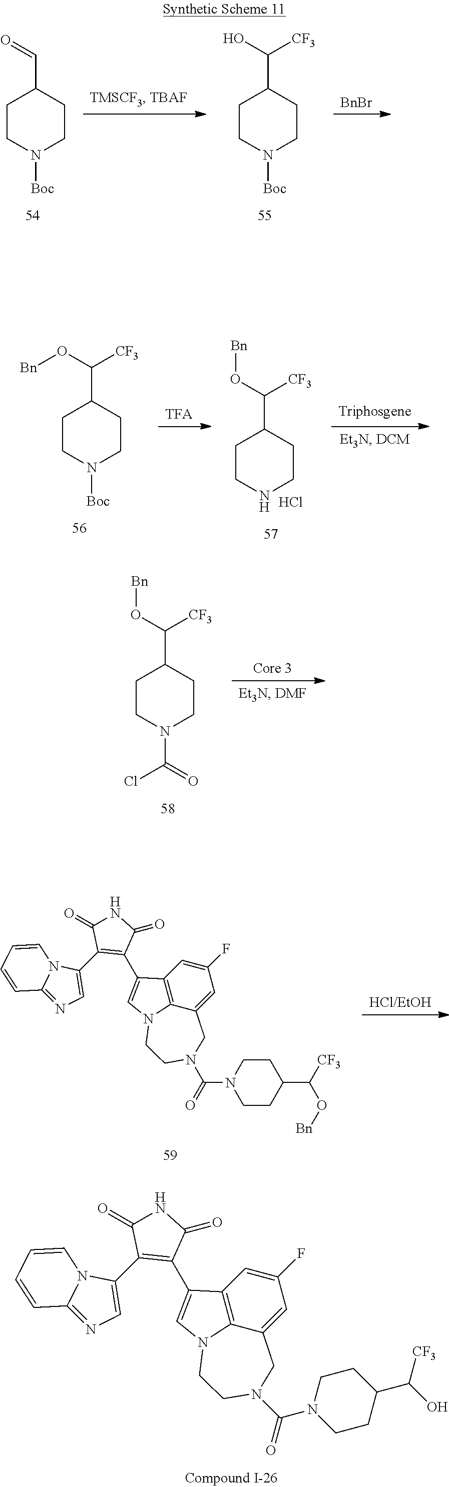

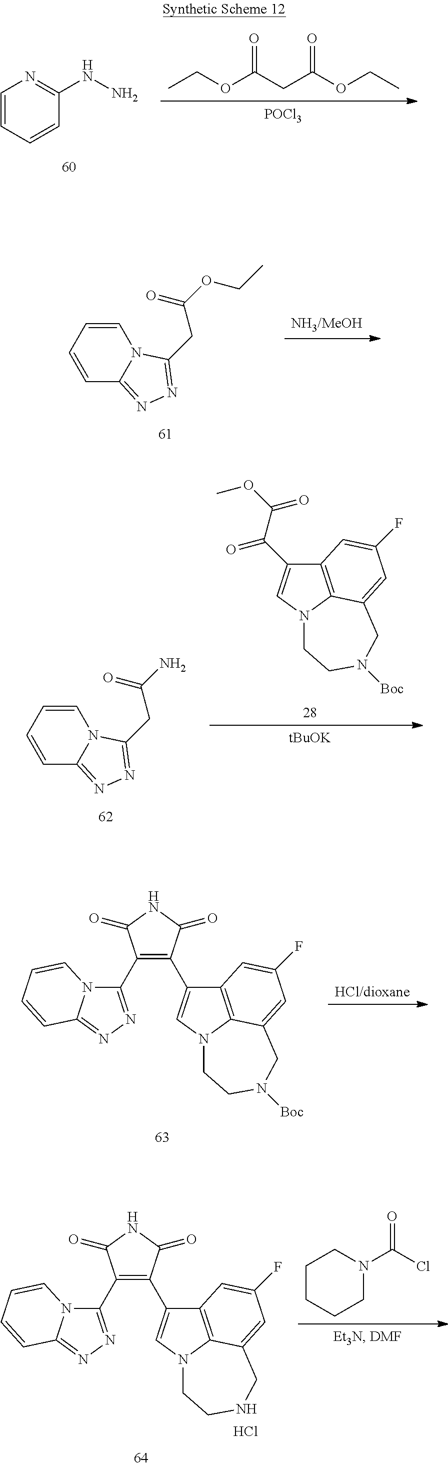

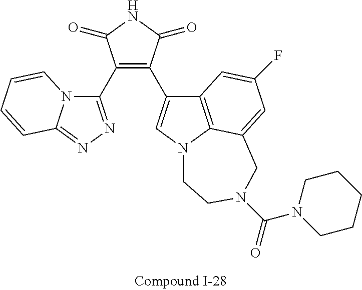 [SMILES: O=C1NC(=O)C(c2nnc3ccccn23)=C1c1cn2c3c(cc(F)cc13)CN(C(=O)N1CCCCC1)CC2]

### EXPERIMENTAL PROCEDURES

### Synthesis of Intermediate 2

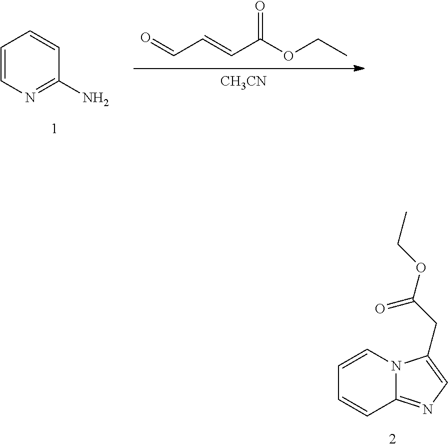

To a solution of intermediate 1 (20 g, 213 mmol) in MeCN (540 mL) was ethyl (E)-4-oxo-butenoate (28.6 g, 223 mmol). The reaction mixture was heated to 80° C. and stirred for 6 hrs. The reaction mixture was concentrated under reduced pressure, the residue was purified by flash column chromatography (eluted with Dichloromethane/MeOH from 1:0 to 200:1) to give the crude intermediate 2 (25 g) as brown solid.

### Synthesis of Intermediate 3

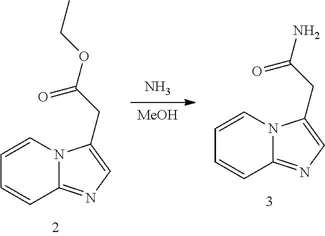

To a solution of crude intermediate 2 (25 g) in MeOH (100 mL) was added NH₃/MeOH (6 M, 100 mL). The reaction mixture was stirred at room temperature overnight. The mixture was poured into EtOAc (500 mL), and then filtered. The filter cake was dried in vacuo to give intermediate 3 (13 g, 35% for two steps) as a brown solid.

¹H NMR (DMSO-d₆, 400 MHz): δ (ppm) 8.30 (d, J=6.8 Hz, 1H), 7.60 (s br, 1H), 7.54 (d, J=5.2 Hz, 1H), 7.41 (s, 1H), 7.19-7.23 (m, 1H), 7.06 (s br, 1H), 6.89-6.93 (m, 1H), 3.80 (s, 2H).

### Synthesis of Intermediate 5

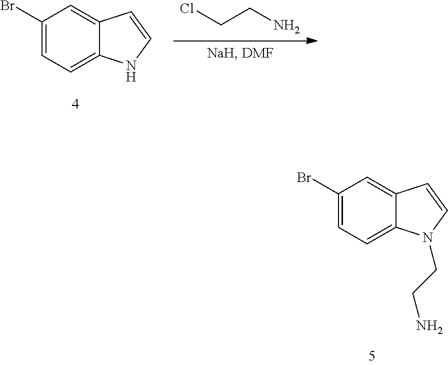

To a solution of intermediate 4 (100 g, 0.51 mol) in DMF (1000 mL) was added NaH (60%, 61 g, 1.53 mol) at 0° C. The mixture was stirred at room temperature for 20 mins. 2-chloroethylamine hydrochloride (89.2 g, 0.77 mol) was added to the mixture in portions at 0° C. The mixture was stirred at room temperature for 2 hrs. TLC (Petroleum Ether/EtOAc=5/1) showed the reaction was complete. The mixture was poured into ice-water and extracted with EtOAc (600 mL×3). The combined organic phases were washed with water, brine, dried over Na₂SO₄ and concentrated in vacuum to give intermediate 5 (110 g, 90%) as yellow oil.

¹H NMR (CDCl₃, 400 MHz): δ (ppm) 7.74-7.75 (d, 1H, J=1.2 Hz), 7.21-7.29 (m, 2H), 7.13-7.14 (d, 1H. J=3.2 Hz), 6.44-6.45 (d, 1H, J=2.8 Hz), 4.14-4.17 (t, 2H, J=6 Hz), 3.08-3.11 (t, 2H, J=6 Hz).

### Synthesis of Intermediate 6

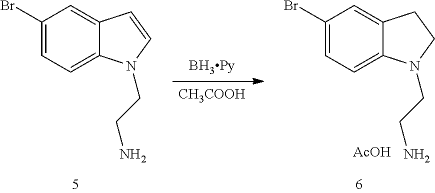

To a solution of intermediate 5 (150 g, 0.63 mol) in AcOH (720 mL) was added Borane pyridine complex (9.3 M, 135.5 mL, 1.26 mol) at room temperature under N2. The mixture was stirred at room temperature for overnight. Then the mixture was adjusted pH=9-10 with aqueous NaOH, extracted with EtOAc (800 mL×3). The combined organic phases were concentrated in vacuum to give crude compound. Water (720 mL) was added to the crude compound, followed by the slow addition of concentrated HCl (240 mL). The mixture was stirred at room temperature for 30 mins, adjusted pH=10-11 with aqueous NaOH, extracted with EtOAc (800 mL×3), concentrated to give crude compound. To a solution of the crude compound in methyl tertiary butyl ether (500 mL) was added AcOH (28 mL) at room temperature. The mixture was stirred at room temperature for 30 mins, then filtered, the filter cake was washed with methyl tertiary butyl ether, dried to give intermediate 6 (120 g, 63.5%) as white solid.

¹H NMR (DMSO-d₆, 400 MHz): δ (ppm) 7.14 (s, 1H), 7.09-7.12 (d, 1H, J=8.4 Hz), 6.43-6.45 (d, 1H, J=8.4 Hz), 3.33-3.37 (t, 2H, J=8.4 Hz), 3.06-3.09 (t, 2H, J=6.6 Hz), 2.87-2.92 (t, 2H, J=8.4 Hz), 2.76-2.79 (t, 2H, J=6.6 Hz).

### Synthesis of Intermediate 7

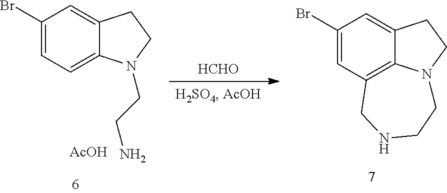

To a solution of H₂SO₄ (12.6 mL) in AcOH (80 mL) and HCHO (37% aqueous, 660 mL) was added intermediate 6 (100 g, 0.33 mol) in portions at room temperature. The mixture was stirred at 50° C. for 20 mins. Then the mixture was adjusted pH=9-10 with aqueous NaOH, extracted with EtOAc (800 mL×3), concentrated to give crude intermediate 7 (100 g) as yellow solid, which was used directly for the next step without purification.

### Synthesis of Intermediate 8

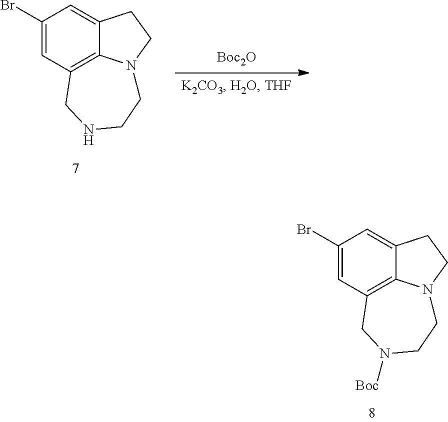

A mixture of intermediate 7 (100 g, crude) and aqueous K₂CO₃ (300 mL, 1 M) in THF (700 mL) was added (Boc)₂O (94.4 g) at room temperature. The mixture was stirred at room temperature for overnight. TLC (dichloromethane/MeOH=10/1) showed the reaction was complete. Then H₂O was added, extracted with EtOAc (500 mL×3). The combined organic phases were washed with brine, dried over Na₂SO₄ and concentrated in vacuum. The residue was purified by flash column chromatography (eluted with petroleum ether/EtOAc from 50:1 to 5:1) to give intermediate 8 (75 g, 64.6% for two steps) as yellow solid.

¹H NMR (CDCl₃, 400 MHz): δ (ppm) 7.11 (bs, 1H), 6.99 (bs, 1H), 4.30-4.37 (m, 2H), 3.68 (m, 2H), 3.36-3.40 (m, 2H), 2.96-3.01 (m, 4H), 1.41 (s, 9H).

### Synthesis of Core 1

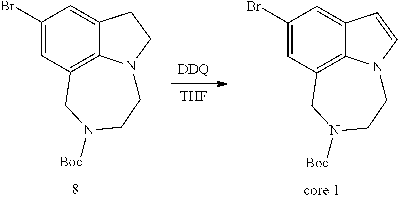

To a solution of intermediate 8 (49 g, 0.14 mol) in THF (490 mL) was added a solution of DDQ (37.9 g, 0.17 mol) in THF (490 mL) at 0° C. under N2. The mixture was stirred at 0° C. for 15 mins. TLC (petroleum ether/EtOAc=5/1) showed the reaction was complete. Then the mixture was poured into aq. Na₂CO₃ and extracted with EtOAc (400 mL×2). The combined organic phases were washed with brine, dried over Na₂SO₄ and concentrated in vacuum. The residue was purified by flash column chromatography (eluted with petroleum ether/EtOAc from 50:1 to 10:1) to give core 1 (24 g, 49%) as yellow solid.

¹H NMR (CDCl₃, 400 MHz): δ (ppm) 7.63 (bs, 1H), 7.04-7.15 (m, 2H), 6.46-6.47 (d, 1H, J=3.2 Hz), 4.76-4.83 (m, 2H), 4.25 (m, 2H), 3.92 (m, 2H), 1.42-1.45 (m, 9H).

### Synthesis of Intermediate 10

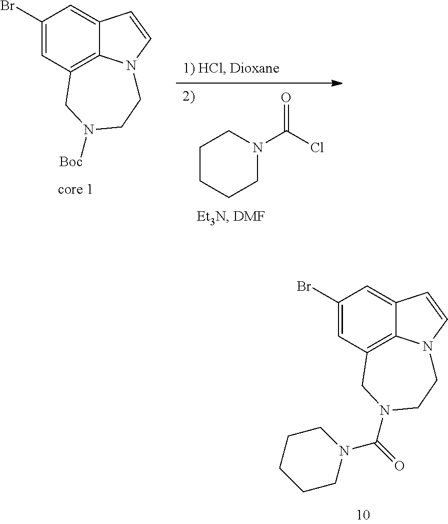

To a solution of core 1 (10 g, 28.5 mmol) in dichloromethane (100 mL) was added HCl/dioxane (7 M, 50 mL) at room temperature. The mixture was stirred at room temperature for 1 hr. TLC (petroleum ether/EtOAc=5/1) showed the reaction was complete. The solvent was concentrated in vacuum to give white solid.

To a solution of the white solid and 1-Piperidinecarbonyl chloride (4.6 g, 31.3 mmol) in DMF (100 mL) was added Et₃N (8.6 g, 85.5 mmol) below 5° C. The mixture was stirred at room temperature for 1 hr. TLC (dichloromethane/MeOH=10/1) showed the reaction was complete. Then the mixture was poured into ice-water and extracted with EA (200 mL×4). The combined organic phases were washed with water, brine, dried over Na₂SO₄ and concentrated in vacuum. The residue was purified by flash column chromatography (eluted with petroleum ether/EtOAc from 20:1 to 1:1) to give intermediate 10 (7.5 g, 72.8%) as yellow solid.

¹H NMR (CDCl₃, 400 MHz): δ (ppm) 7.86 (d, J=1.6 Hz, 1H), 7.26 (s, 1H), 7.02 (t, J=1.6 Hz, 1H), 6.47 (d, J=3.2 Hz, 1H), 4.63 (s, 2H), 4.22-4.20 (t, J=4.8 Hz, 2H), 3.98-4.00 (t, J=4.8 Hz, 2H), 3.18-3.19 (m, 4H), 1.58-1.40 (m, 6H).

### Synthesis of Intermediate 11

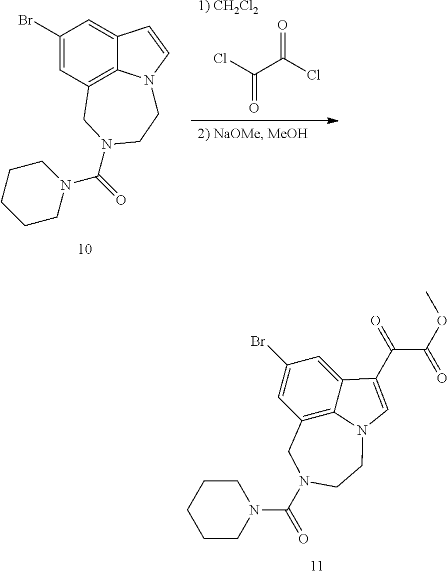

To a solution of intermediate 10 (10 g, 27.6 mmol) in dichloromethane (100 mL) was added (COCl)₂ (8.8 g, 69 mmol) at 0° C. under N2. The mixture was stirred at 40° C. for 1 hr. TLC (petroleum ether/EtOAc=1/1) showed the reaction was complete. Then a solution of NaOMe (3.7 g, 69 mmol) in MeOH (10 mL) was added at −60° C. under N2. The mixture was stirred at room temperature for 1 hr. Water was added, extracted with dichloromethane (100 mL×3). The combined organic phases were washed with brine, dried over Na₂SO₄ and concentrated in vacuum. The residue was purified by flash column chromatography (eluted with petroleum ether/EtOAc from 5:1 to 1:2) to give intermediate 11 (6 g, 48.5%) as yellow solid.

¹H NMR (CDCl₃, 400 MHz): δ (ppm) 8.70 (d, J=1.6 Hz, 1H), 8.29 (s, 1H), 7.41 (s, 1H), 4.67 (s, 2H), 4.41-4.39 (m, 2H), 4.01-3.98 (m, 2H), 3.95 (s, 3H), 3.15-3.01 (m, 4H), 1.60-1.40 (m, 6H).

### Synthesis of Core 2

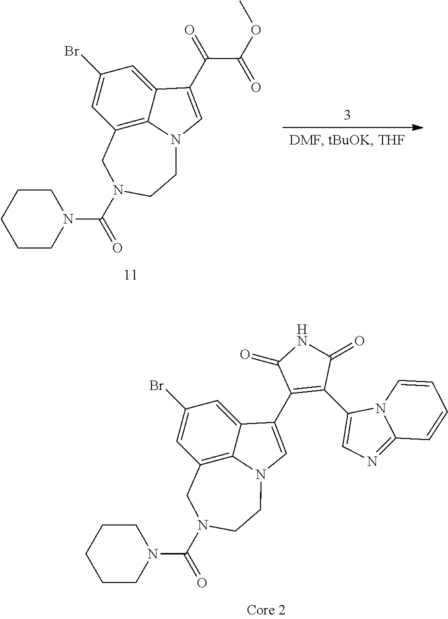

A solution of intermediate 11 (10 g, 22.3 mmol) and intermediate 3 (3.9 g, 22.3 mmol) in DMF (180 mL) was added a solution of t-BuOK (6.4 g, 19.0 mmol) in THF (100 mL) at 0-10° C. The mixture was stirred at 0-10° C. for 15 mins. TLC (dichloromethane/MeOH=15/1) showed the reaction was complete. Then the mixture was poured into ice-water and extracted with EtOAc (100 mL×4). The combined organic phases were washed with water, brine, dried over Na₂SO₄ and concentrated in vacuum. The residue was purified by flash column chromatography (eluted with petroleum ether/EtOAc/THF from 10:5:1 to 1:1:1) to give core 2 (6.5 g, 50.7%) as orange solid.

¹H NMR (DMSO-d₆, 400 MHz): δ (ppm) 11.25 (s, 1H), 8.02 (s, 1H), 7.91 (s, 1H), 7.65-7.68 (d, J=6.8 Hz, 1H), 7.59-7.61 (d, J=6.8 Hz, 1H), 7.19-7.23 (t, J=7.6 Hz, 1H), 7.04 (s, 1H), 6.55-6.58 (t, J=6.4 Hz, 1H), 6.08 (s, 1H), 4.63 (s, 2H), 4.50-4.62 (m, 2H), 3.82-3.86 (m, 2H), 2.94-3.06 (m, 4H), 1.40-1.60 (m, 6H).

LC/MS M+1=573.1

### Synthesis of Intermediate 12

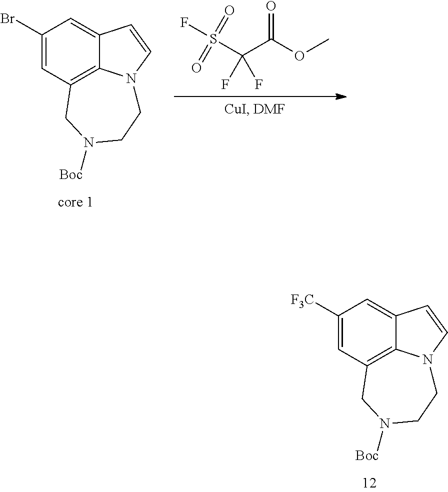

To a solution of core 1 (10 g, 28.5 mmol) in DMF (200 mL) was added CuI (5.4 g, 28.5 mmol) and methyl 2,2-difluoro-2-(fluorosulfonyl)acetate (19.2 g, 100 mmol) at room temperature under N2. The mixture is stirred at 80° C. for 2.5 hrs. TLC (petroleum ether/EtOAc=5/1) showed that the reaction was complete. The mixture was cooled to room temperature, and filtered. The filtrate was added water and extracted with EtOAc (100 mL×4). The combined organic phases were washed with water, brine, dried over Na₂SO₄ and concentrated in vacuum. The residue was purified by flash column chromatography (eluted with petroleum ether/EtOAc from 20:1 to 5:1) to give intermediate 12 (5.5 g, 56.7%) as yellow solid.

¹H NMR (CDCl₃, 400 MHz): δ (ppm) 7.81 (s, 1H), 7.13-7.28 (m, 2H), 6.62 (s, 1H), 4.92-4.84 (m, 2H), 4.22 (s, 2H), 3.98-3.97 (m, 2H), 1.40 (s, 9H).

### Synthesis of Intermediate 13

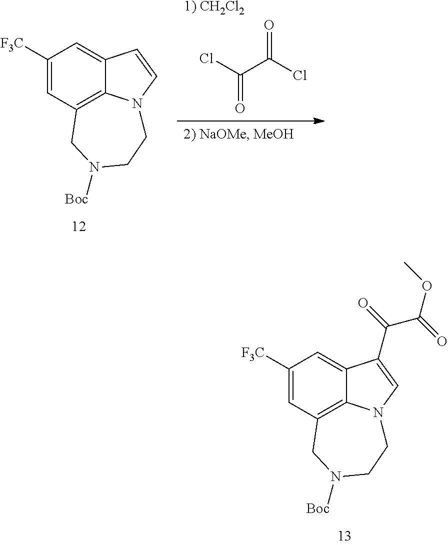

To a solution of intermediate 12 (5.0 g, 14.7 mmol) in dichloromethane (75 mL) was added (COCl)₂ (4.6 g, 36.7 mmol) under N2. The mixture was stirred at 40° C. for 1 hr. TLC (petroleum ether/EtOAc=1/1) showed the reaction was complete. Then a solution of NaOMe (1.98 g, 36.7 mmol) in MeOH (10 mL) was added at −60° C. under N2. The mixture was stirred at room temperature for 1 hr. Water was added, extracted with dichloromethane (100 mL×3). The combined organic phases were washed with brine, dried over Na₂SO₄ and concentrated in vacuum. The residue was purified by flash column chromatography (eluted with petroleum ether/EtOAc from 10:1 to 5:1) to give intermediate 13 (3.8 g, 60.7%) as a yellow solid.

¹H NMR (CDCl₃, 400 MHz): δ (ppm) 8.64 (s, 1H), 8.44 (s, 1H), 7.40-7.31 (m, 1H), 4.98-4.88 (m, 2H), 4.51-4.50 (m, 2H), 4.04-4.01 (m, 2H), 3.96 (s, 3H), 1.40 (s, 9H).

### Synthesis of Intermediate 14

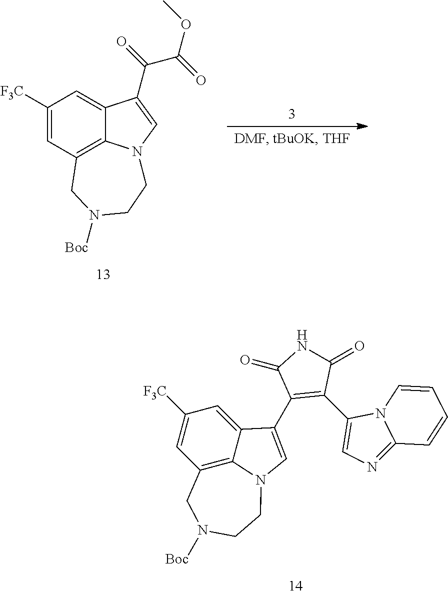

To a solution of intermediate 13 (5.5 g, 12.9 mmol) and intermediate 3 (2.25 g, 12.9 mmol) in DMF (110 mL) was added a solution of tBuOK (3.6 g, 32.2 mmol) in THF (10 mL) at 0-10° C. The mixture was stirred at 0-10° C. for 15 min. TLC (petroleum ether/EtOAc=1/1) showed the reaction was complete. Then the mixture was poured into ice-water and extracted with EtOAc (100 mL×4). The combined organic phases were washed with water, brine, dried over Na₂SO₄ and concentrated in vacuum. The residue was purified by flash column chromatography (eluted with dichloromethane/MeOH from 100:1 to 30:1) to give intermediate 14 (3.5 g, 58.3%) as orange solid.

¹H NMR (CDCl₃, 400 MHz): δ (ppm) 8.16 (s, 1H), 8.02 (s, 1H), 7.96 (s, 1H), 7.35 (s, 1H), 7.27 (s, 1H), 7.12-7.08 (m, 2H)), 6.40-6.38 (m, 2H, 4.89-4.77 (m, 2H), 4.50 (s, 2H), 4.04 (s, 2H), 1.48-1.37 (m, 9H).

### Synthesis of Compound I-1

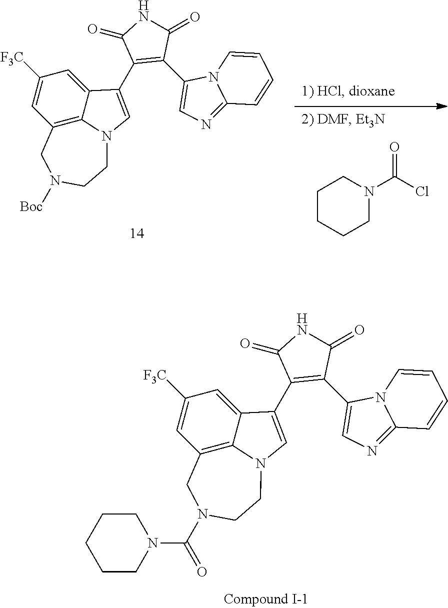

To a solution of intermediate 14 (5 g, 9.1 mmol) in dichloromethane (50 mL) was added HCl/dioxane (50 mL, 7M) at room temperature. The mixture was stirred at room temperature for 1 hr. TLC (dichloromethane/MeOH=15/1) showed the reaction was complete. The solvent was concentrated in vacuum to give white solid.

To a solution of the white solid and 1-Piperidinecarbonyl chloride (1.8 g, 12.3 mmol) in DMF (40 mL) was added Et₃N (2.49 g, 24.6 mmol) at room temperature. The mixture was stirred at room temperature for 1 hr. TLC (dichloromethane/MeOH=10/1) showed the reaction was complete. Then the mixture was poured into ice-water and extracted with EtOAc (100 mL×4). The combined organic phases were washed with water, brine, dried over Na₂SO₄ and concentrated in vacuum. The residue was purified by flash column chromatography (eluted with dichloromethane/THF from 20:1 to 10:1) to give compound I-1 (3.0 g, 70%) as red solid.

¹H NMR (DMSO-d₆, 400 MHz): δ (ppm) 11.30 (s, 1H), 8.17 (s, 1H), 7.93 (s, 1H), 7.64-7.60 (m, 2H), 7.19 (s, 1H), 6.56 (m, 1H), 6.31 (s, 1H), 4.77-4.69 (m, 2H), 4.68-4.60 (m, 2H), 3.95-3.85 (m, 2H), 3.10-2.90 (m, 4H), 1.55-1.35 (m, 6H).

LC/MS M+1 563.1

### Synthesis of Compound I-2

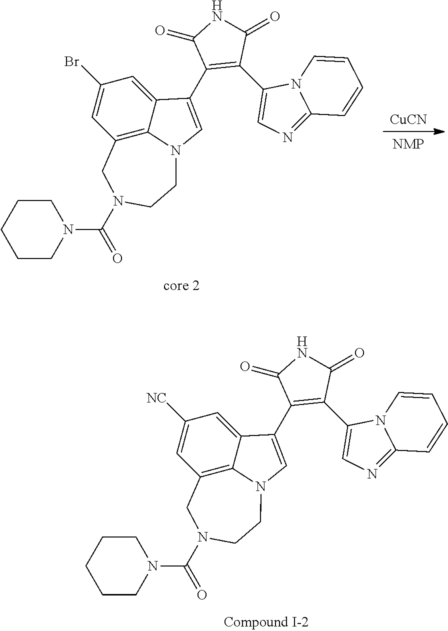

A solution of core 2 (5 g, 8.7 mmol) in N-Methyl-2-Pyrrolidone (50 mL) was added CuCN (2.5 g, 28 mol) at room temperature. The mixture was stirred at 150° C. for 6 hr under N2. Then the mixture was poured into ice-water and extracted with EtOAc (100 mL×4). The combined organic phases were washed with brine, dried over Na₂SO₄ and concentrated in vacuum. The residue was purified by flash column chromatography (eluted with dichloromethane/MeOH from 1:0 to 50:1) to give compound I-2 (2.2 g, 49%) as orange solid.

¹H NMR (DMSO-d₆): δ (ppm) 11.30 (s, 1H), 8.07 (s, 1H), 7.97 (s, 1H), 7.66-7.68 (d, 1H, J=9.2 Hz), 7.54-7.56 (d, 1H, J=6.8 Hz), 7.29 (s, 1H), 7.19-7.23 (t, J=8 Hz, 1H), 6.53-6.57 (t, 1H, J=6.8 Hz), 6.44 (s, 1H), 4.68 (S, 4H), 4.60-4.63 (m, 4H), 3.84-3.88 (m, 2H), 2.96-3.05 (m, 4H), 1.43-1.47 (m, 6H).

LC/MS M+1 520.1

### Synthesis of Intermediate 15

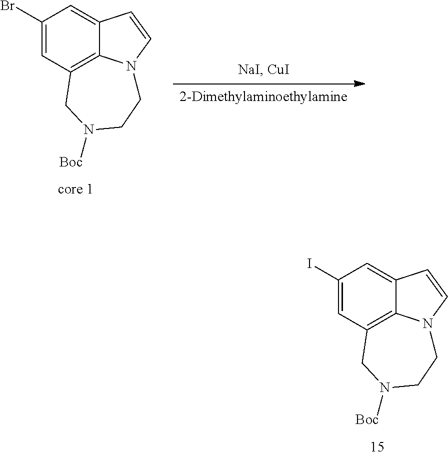

To a solution of core 1 (50 g, 0.14 mol) in 1,4-dioxane (500 mL) was added NaI (42.7 g, 0.28 mol) and CuI (2.7 g) and 2-Dimethylaminoethylamine (2.5 g). The mixture was stirred at 140° C. overnight under N2. The mixture was filtered, the filter cake was washed with dichloromethane. The combined organic phases were concentrated in vacuum. The residue was washed with methyl tertiary butyl ether to give intermediate 15 (50 g, 88.0%) as yellow solid.

### Synthesis of Intermediate 16

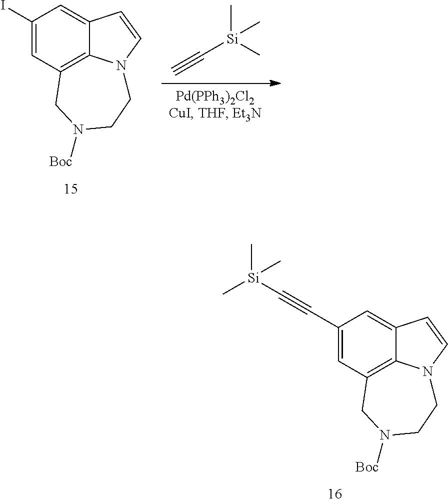

To a solution of intermediate 15 (10 g, 25.1 mmol) in Et₃N (120 mL) and THF (60 mL) at room temperature, then Pd(PPh₃)₂Cl₂ (1.2 g, 2.4 mmol) and CuI (1.2 g, 2.4 mmol) was added under N2. Then ethynyltrimethylsilane (4.75 g, 48.4 mmol) was added drop wise. The mixture was stirred at 60° C. overnight. The mixture was concentrated in vacuum to remove Et₃N. Then the mixture was poured into ice-water, extracted with CH₂Cl₂ (300 mL×2). The combined organic phases were washed with water, brine, dried over Na₂SO₄ and concentrated in vacuum. The residue was purified by flash column chromatography (eluted with petroleum ether/EtOAc from 100:1 to 50:1) to give intermediate 16 (6 g, 64.8%) as yellow solid.

¹H NMR (CDCl₃, 400 MHz): δ (ppm) 7.63-7.67 (m, 1H), 7.04-7.41 (m, 2H), 6.46-6.49 (m, 1H), 4.77-4.84 (m, 2H), 4.23-4.29 (m, 2H), 3.90-3.97 (m, 2H), 1.43 (s, 9H), 0.26 (s, 9H).

### Synthesis of Intermediate 17

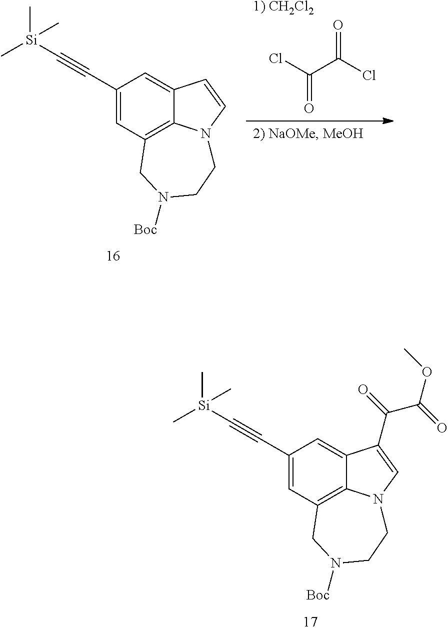

A solution of intermediate 16 (10 g, 27 mmol) in dichloromethane (110 mL) was added (COCl)₂ (8.5 g, 67.4 mmol) at 0° C. under N2. The mixture was stirred at 40° C. for 1 hr. TLC (petroleum ether/EtOAc=5/1) showed the reaction was complete. Then a solution of NaOMe (3.64 g, 67.4 mmol) in MeOH (10 mL) was added at −60° C. under N2, The mixture was stirred at room temperature for 1 hr. Water was added, extracted with dichloromethane (100 mL×3). The combined organic phases were washed with water, brine, dried over Na₂SO₄ and concentrated in vacuum. The residue was purified by flash column chromatography (eluted with petroleum ether/EtOAc from 100:1 to 5:1) to give intermediate 17 (7 g, 56.7%) as yellow solid.

### Synthesis of Intermediate 18

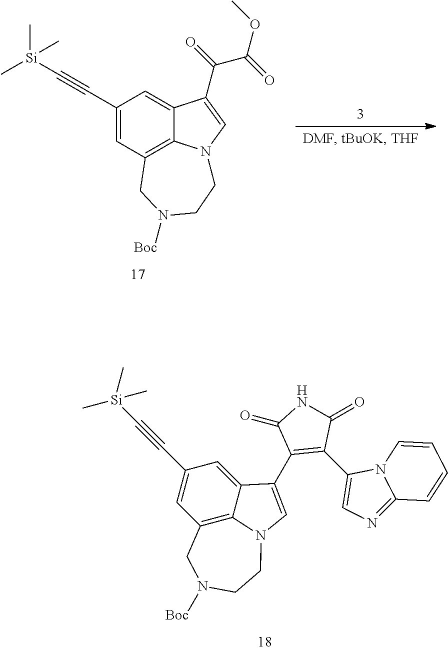

To a solution of intermediate 17 (8 g, 17.6 mmol) and intermediate 3 (3.1 g, 17.6 mmol) in DMF (80 mL) was added a solution of tBuOK (4.9 g, 44 mmol) in THF (30 mL) at 0-10° C. The mixture was stirred at 0-10° C. for 15 min. TLC (petroleum ether/EtOAc=1/1) showed the reaction was complete. Then the mixture was poured into ice-water and extracted with EtOAc (100 mL×4). The combined organic phases were washed with water, brine, dried over Na₂SO₄ and concentrated in vacuum. The residue was purified by flash column chromatography (eluted with dichloromethane/MeOH from 100:1 to 50:1) to give intermediate 18 (5 g, 49%) as orange solid.

### Synthesis of Intermediate 19

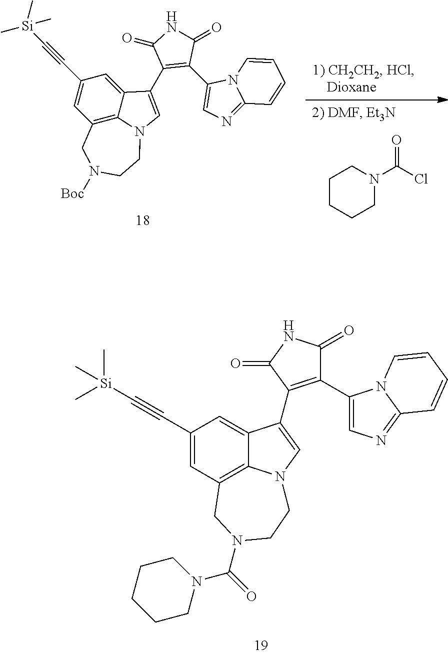

To a solution of intermediate 18 (5 g, 8.6 mmol) in dichloromethane (50 mL) was added HCl/dioxane (50 mL, 7M) at room temperature. The mixture was stirred at room temperature for 1 hr. TLC (dichloromethane/MeOH=15/1) showed the reaction was complete. The solvent was concentrated in vacuum to give white solid.

To a solution of the white solid and 1-Piperidinecarbonyl chloride (1.4 g, 9.5 mmol) in DMF (50 mL) was added Et₃N (2.6 g, 25.8 mmol) at room temperature. The mixture was stirred at room temperature for 1 hr. TLC (dichloromethane/MeOH=10/1) showed the reaction was complete. Then the mixture was poured into ice-water and extracted with EtOAc (100 mL×4). The combined organic phases were washed with water, brine, dried over Na₂SO₄ and concentrated in vacuum to give the crude intermediate 19 (5.2 g), which was used directly for the next step without purification.

### Synthesis of Compound I-3

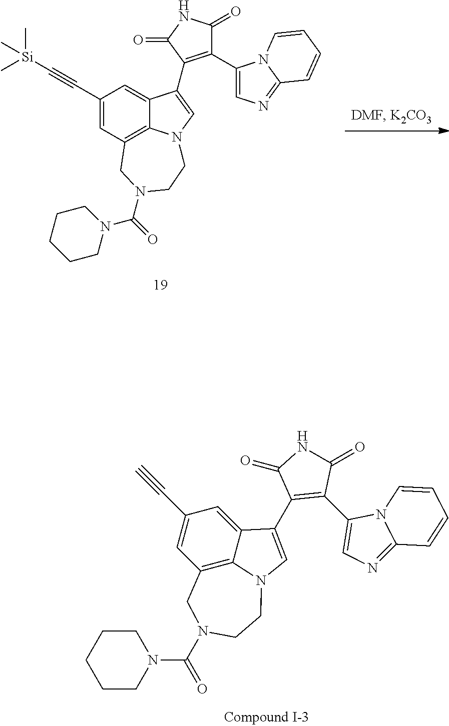

To a solution of the crude intermediate 19 (5.2 g) in DMF (100 mL) was added K₂CO₃ (2 g). The mixture was heated to 50° C. and stirred for 1 hr. Then the mixture was poured into ice-water and extracted with EtOAc (100 mL×4). The combined organic phases were washed with water, brine, dried over Na₂SO₄ and concentrated in vacuum. The residue was purified by flash column chromatography (eluted with dichloromethane/MeOH from 100:1 to 30:1) to give compound I-3 (3 g, 18.6% for two steps) as red solid.

¹H NMR (DMSO-d₆, 400 MHz): δ (ppm) 11.26 (s, 1H), 8.02 (s, 1H), 7.92 (s, 1H), 7.65-7.59 (m, 2H), 7.20 (t, 1H, J=8 Hz), 6.97 (s, 1H), 6.58-6.55 (t, J=6.8 Hz, 1H), 6.18 (s, 1H), 4.63 (s, 2H), 4.50-4.60 (m, 2H), 3.80-3.90 (m, 2H), 3.75 (s, 1H), 2.95-3.05 (m, 4H), 1.46-1.35 (m, 6H).

LC/MS M+1 519.2

### Synthesis of Compound I-10

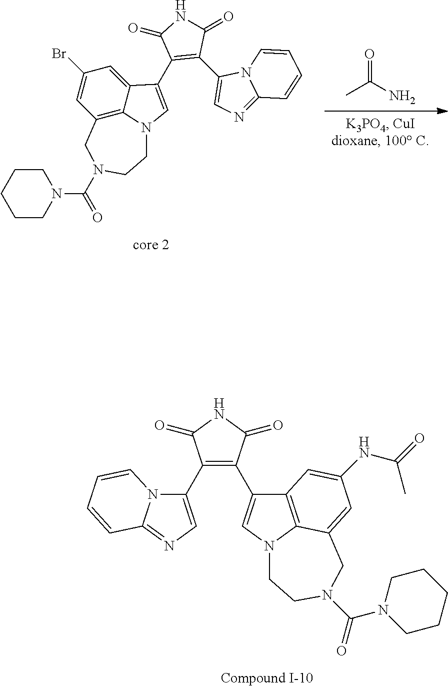

A solution of core 2 (5 g, 8.8 mmol) in dioxane (50 mL) was added acetamide (3.1 g, 53.3 mmol), CuI (1.1 g, 5.8 mmol), K₃PO₃ (5.5 g, 26.4 mmol) at RT under N2. The mixture was stirred at room temperature for 20 mins. N¹,N²-dimethylethane-1,2-diamine (1.56 g, 17.8 mmol) was added, and then the mixture was stirred at 115° C. for 5 hrs under N₂. TLC (dichloromethane/MeOH=15/1) showed the reaction was complete. Then the mixture was poured into ice-water and extracted with EtOAc/THF (3/1, 100 mL×4). The combined organic phases were washed with water, brine, dried over Na₂SO₄ and concentrated in vacuum. The residue was purified by flash column chromatography (eluted with dichloromethane/MeOH from 200:1 to 50:1) to give compound I-10 (3 g, 62%) as red solid.

¹H NMR (DMSO-d₆, 400 MHz): δ (ppm) 11.21 (s, 1H), 9.43 (s, 1H), 7.86 (s, 1H), 7.78 (s, 1H), 7.58-7.64 (m, 2H), 7.17-7.21 (t, 1H, J=8.0 Hz), 7.00 (s, 1H), 6.77 (s, 1H), 6.56-6.58 (t, 1H, J=7.2 Hz), 4.60 (s, 2H), 4.40-4.50 (m, 2H), 3.80-3.88 (m, 2H), 2.88-3.12 (m, 4H), 1.85 (s, 3H), 1.15-1.20 (m, 6H).

LC/MS M+1 552.2

### Synthesis of Compound I-4

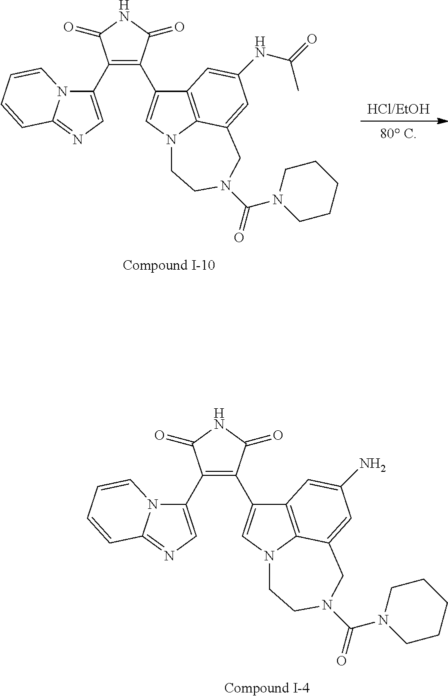 [SMILES: CC(=O)Nc1cc2c3c(c1)c(C1=C(c4cnc5ccccn45)C(=O)NC1=O)cn3CCN(C(=O)N1CCCCC1)C2 ; "80° C." ; "HCl/EtOH" ; Nc1cc2c3c(c1)c(C1=C(c4cnc5ccccn45)C(=O)NC1=O)cn3CCN(C(=O)N1CCCCC1)C2]

A solution of compound I-10 (3 g, 5.4 mmol) in EtOH (10 mL) was added HCl (28 mL, 6 N) at room temperature. The mixture was stirred at 80° C. for 3 hrs. TLC (dichloromethane/MeOH=15/1) showed the reaction was complete. Then the mixture was poured into ice-water and extracted with dichloromethane (100 mL×2), then the water phase was adjusted pH=9-10 with aq.Na₂CO₃, extracted with dichloromethane (100 mL×6), concentrated in vacuum. The residue was washed with methyl tertiary butyl ether, filtered. The filter cake was dried in vacuo to give compound I-4 (2.1 g, 75%) as red solid.

¹H NMR (DMSO-d₆, 400 MHz): δ (ppm) 11.19 (s, 1H), 7.86 (s, 1H), 7.84 (s, 1H), 7.62 (bs, 2H), 7.18-7.22 (t, 1H, J=7.2 Hz), 6.57-6.61 (t, 1H, J=6.8 Hz), 6.24 (s, 1H), 5.27 (s, 1H), 4.50 (s, 2H), 4.36-4.39 (m, 2H), 4.14-4.17 (m, 2H), 3.75-3.78 (m, 2H), 2.88-3.05 (m, 4H), 1.44-1.48 (m, 6H).

MS/LC M+1 511.1

### Synthesis of Intermediate 22

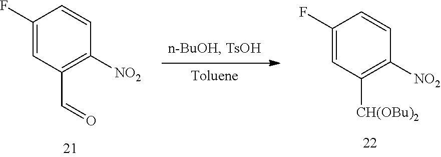

To a solution of intermediate 21 (100 g, 0.59 mol) in dry toluene (890 mL) was added n-BuOH (131.6 g, 1.78 mol) and TsOH (10 g) at room temperature. The mixture was stirred at 120° C. for overnight, and removed the water using a Dean-stark apparatus. TLC (petroleum ether/EtOAc=5/1) showed the reaction was completed. The mixture was concentrated in vacuum to give crude intermediate 22. The crude intermediate 22 was purified by flash column chromatography (eluted with petroleum ether/EtOAc from 100:1 to 20:1) to give intermediate 22 (120 g, 67.8%) as a yellow oil.

¹H NMR (CDCl₃, 400 MHz): δ (ppm) 7.89-7.92 (dd, 1H, J=4.8 Hz, 8.8 Hz), 7.51-7.54 (dd, 1H, J=2.8 Hz, 9.6 Hz), 7.09-7.14 (m, 1H), 6.04 (s, 1H), 3.50-3.56 (m, 4H), 1.55-1.62 (m, 4H), 1.33-1.42 (m, 4H), 0.83-0.93 (m, 6H).

### Synthesis of Intermediate 23

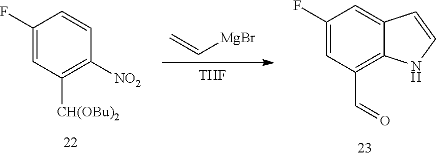

To a solution of intermediate 22 (50 g, 0.17 mol) in dry THF (1500 mL) was added vinylmagnesium bromide solution (1 M, 668.8 mL, 668.8 mmol) drowpwised at −40° C. The mixture was stirred at −40° C. for 1 hr. TLC (petroleum ether/EtOAc=5/1) showed the reaction was completed Then the mixture was poured into aq.NH₄Cl, extracted with EtOAc (300 mL×3), the organic phases were concentrated to give crude compound. The crude compound was purified by flash column chromatography (eluted with petroleum ether/EtOAc from 100:1 to 20:1) to give the compound (24 g) as yellow oil. To a solution of the compound (24 g) in THF (100 mL) was added HCl (0.5 N, 80 mL) drop wised at room temperature. The mixture was stirred at room temperature for 1 hr. TLC (petroleum ether/EtOAc=5/1) showed the reaction was completed. The mixture was adjusted pH=10 with aq.NaOH, extracted with EtOAc (300 mL×3), concentrated in vacuum to give intermediate 23 (16 g, 58.8%) as yellow solid.

¹H NMR (CDCl₃, 400 MHz): δ (ppm) 10.06 (s, 1H), 7.62 (dd, 1H, J=2 Hz, 9.2 Hz), 7.38-7.41 (m, 2H), 6.60 (t, 1H, J=2.4 Hz).

### Synthesis of Intermediate 24

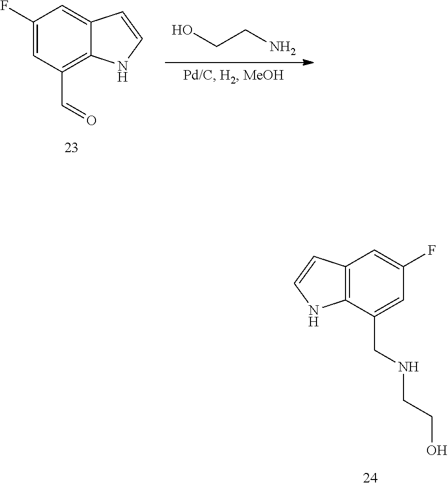

To a solution of intermediate 23 (140 g, 0.86 mol) in MeOH (2100 mL) was added 2-aminoethanol (78 g, 1.3 mol) and Pd/C (14 g) at room temperature under N2. The mixture was stirred at room temperature for 2 hrs under N2. Then the mixture was stirred at room temperature for overnight under H₂. The mixture was filtered, concentrated to give intermediate 24 (200 g, crude) as a yellow oil, which was used directly for the next step without purification.

### Synthesis of Intermediate 25

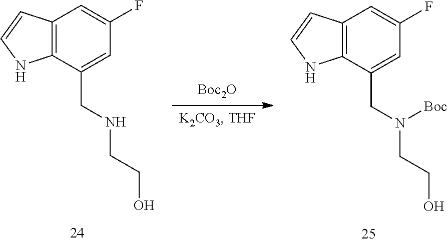

A mixture of intermediate 24 (90 g, crude) and K₂CO₃ (467 mL, 1 M) in THF (1300 mL) was added Boc₂O (141 g) at room temperature. The mixture was stirred at room temperature for overnight. TLC (dichloromethane/MeOH=10/1) showed the reaction was completed Then H₂O was added, extracted with EtOAc (500 mL×3). The combined organic phases were washed with brine, dried over Na₂SO₄ and concentrated in vacuum. The residue was purified by flash column chromatography (eluted with petroleum ether/EtOAc from 10:1 to 1:1) to give intermediate 25 (66.6 g, 56% for two steps) as yellow oil.

¹H NMR (CDCl₃, 400 MHz): δ (ppm) 10.17 (s, 1H), 7.23-7.26 (m, 2H), 6.78-6.82 (dd, 1H, J=9.2 Hz, J=2 Hz), 6.48-6.50 (t, 1H, J=2.4 Hz), 4.67 (s, 2H), 3.66-3.71 (m, 2H), 3.25-35 (m, 2H), 1.40 (s, 9H).

### Synthesis of Intermediate 26

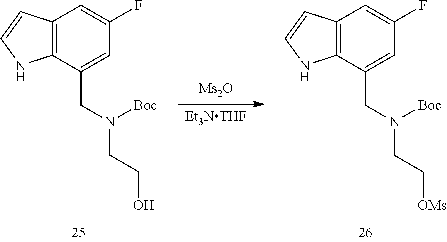 [SMILES: "Ms2O" ; CC(C)(C)OC(=O)N(CCO)Cc1cc(F)cc2cc[nH]c12 ; "Et3N·THF" ; CC(C)(C)OC(=O)N(CCOS(C)(=O)=O)Cc1cc(F)cc2cc[nH]c12]

To a solution of intermediate 25 (50 g, 0.26 mol) in THF (1000 mL) was added Et₃N (79 g, 0.79 mol) and Ms₂O (55 g, 0.32 mol) at 0° C. under N2. The mixture was stirred at 0° C. for 2 hrs. TLC (petroleum ether/EtOAc=3/1) showed the reaction was completed. Then it was poured into ice-water and extracted with EtOAc (400 mL×2). The combined organic phases were washed with brine, dried over Na₂SO₄ and concentrated in vacuum to give intermediate 26 (50 g, 79%) as yellow oil.

¹H NMR (CDCl₃, 400 MHz): δ (ppm) 10.08 (bs, 1H), 7.24-7.29 (m, 2H), 6.81-6.84 (m, 1H), 6.49-6.50 (d, 1H, J=2.4 Hz), 4.67 (s, 2H), 4.28-4.31 (m, 2H), 3.48-3.52 (m, 2H), 2.79 (s, 3H), 1.51 (s, 9H).

### Synthesis of Intermediate 27

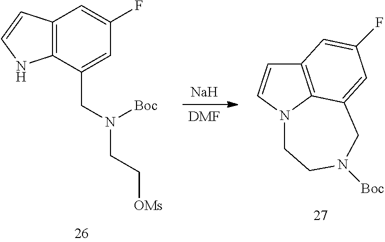

A solution of intermediate 26 (65 g, 0.19 mol) in DMF (722 mL) was added NaH (60%, 11.5 g, 0.29 mol) at 0° C. The mixture was stirred at 0° C. for 1 hr under N2. TLC (petroleum ether/EtOAc=3/1) showed the reaction was completed. Then the mixture was poured into ice-water and extracted with EtOAc (500 mL×4). The combined organic phases were washed with water, brine, dried over Na₂SO₄ and concentrated in vacuum. The residue was purified by flash column chromatography (eluted with petroleum ether/EtOAc from 50:1 to 10:1) to give intermediate 27 (26 g, 53.2%) as yellow solid.

¹H NMR (CDCl₃, 400 MHz): δ (ppm) 7.14-7.16 (d, 1H, J=8 Hz), 7.07 (s, 1H), 6.72-6.83 (m, 1H), 6.48-6.49 (d, 1H, J=2.8 Hz), 4.84-4.76 (s, 2H), 4.24-4.25 (m, 2H), 3.94 (m, 2H), 1.45-1.48 (m, 9H).

### Synthesis of Intermediate 28

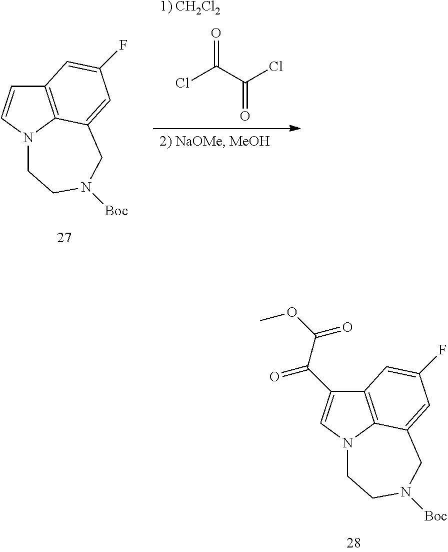

A solution of intermediate 27 (27.5 g, 95.0 mmol) in dichloromethane (900 mL) was added (COCl)₂ (18 g, 142 mmol) at 0° C. under N2. The mixture was stirred at 0° C. for 2 hrs. TLC (petroleum ether/EtOAc=1/1) showed the reaction was completed. Then a solution of NaOMe (13.4 g, 247 mmol) in MeOH (40.8 mL) was added at −60° C. under N2. The mixture was stirred at room temperature for 1 hr. Water was added, extracted with dichloromethane (200 mL×3). The combined organic phases were washed with water, brine, dried over Na₂SO₄ and concentrated in vacuum. The residue was purified by flash column chromatography (eluted with petroleum ether/EtOAc from 20:1 to 1:1) to give intermediate 28 (20 g, 56%) as white solid.

¹H NMR (CDCl₃, 400 MHz): δ (ppm) 8.37 (s, 1H), 8.02-8.04 (d, 1H, J=8.4 Hz), 6.84-6.91 (m, 1H), 4.80-4.90 (m, 2H), 4.44 (bs, 2H), 3.91-3.98 (m, 5H), 1.41-1.46 (m, 9H).

### Synthesis of Intermediate 29

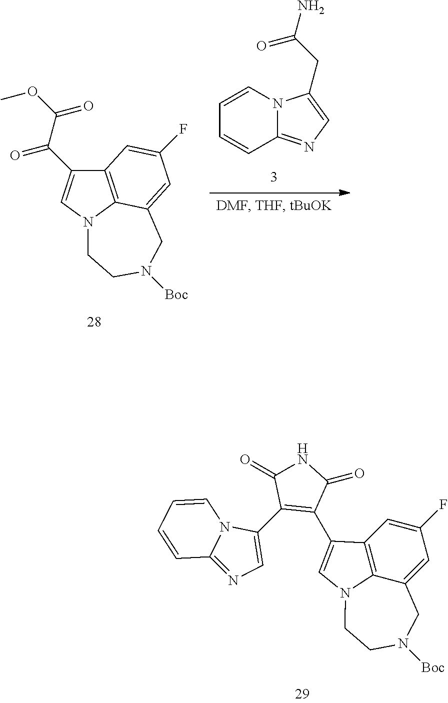

A solution of intermediate 28 (10 g, 26.5 mmol) and intermediate 3 (4.6 g, 26.5 mmol) in DMF (120 mL) was added A solution of tBuOK (7.4 g, 66.2 mmol) in THF (100 mL) at 0-10° C. The mixture was stirred at 0-10° C. for 15 min. TLC (petroleum ether/EtOAc=1/1) showed the reaction was completed. Then the mixture was poured into ice-water and extracted with EtOAc (100 mL×4). The combined organic phases were washed with water, brine, dried over Na₂SO₄ and concentrated in vacuum. The residue was purified by flash column chromatography (eluted with petroleum ether/EtOAc from 20:1 to 1:1) to give intermediate 29 (7 g, 52.5%) as red solid.

¹H NMR (DMSO-d₆, 400 MHz): δ (ppm) 11.07 (s, 1H), 8.02-8.01 (d, 1H, J=4 Hz), 8.96 (s, 1H), 7.61-7.63 (t, 1H, J=8.8 Hz), 7.45 (s, 1H), 7.17 (s, 1H), 6.57-6.64 (m, 1H), 6.47-6.49 (d, 1H, J=8 Hz), 5.77-5.80 (d, 1H J=8.4 Hz), 4.73 4.78 (2H), 4.52 (bs, 2H), 3.96 (bs, 2H), 1.25-1.44 (m, 9H).

### Synthesis of Core 3

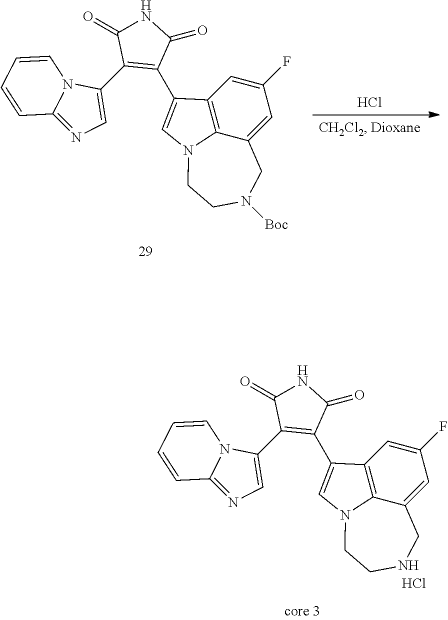

A solution of intermediate 29 (5 g, 9.9 mmol) in dichloromethane (50 mL) was added HCl/dioxane (50 mL, 7M) at room temperature. The mixture was stirred at room temperature for 1 hr. TLC (dichloromethane/MeOH=15/1) showed the reaction was completed. The solvent was concentrated in vacuum. The residue was washed with methyl tertiary butyl ether, filtered. The filter cake was dried in vacuo to give core 3 (4 g, 91.9%) as orange solid.

¹H NMR (DMSO-d₆, 400 MHz): δ (ppm) 11.59 (s, 1H), 10.46 (bs, 2H), 8.49 (s, 1H), 8.18 (s, 1H), 8.08-8.12 (m, 2H, J=14 Hz), 7.88-7.92 (t, 1H, J=8.0 Hz), 7.25-7.29 (t, 1H, J=6.8 Hz), 7.04-7.07 (m, 1H), 6.22-6.25 (m, 1H), 4.76 (bs, 2H), 4.62 (bs, 2H), 3.68 (bs, 2H).

### Synthesis of Intermediate 31

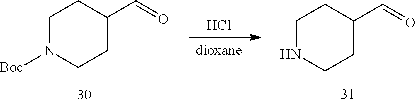

A solution of intermediate 30 (20 g, 94 mmol) in dioxane (100 mL) was added HCl/dioxane (100 mL, 7M) at room temperature. The mixture was stirred at room temperature for 2 hrs. TLC (petroleum ether/EtOAc=3/1) showed the reaction was completed. The mixture was poured onto methyl tertiary butyl ether (300 mL), and filtered. The filter cake was dried in vacuo to give intermediate 31 hydrochloride (8 g, 57%) as white solid.

### Synthesis of Intermediate 32

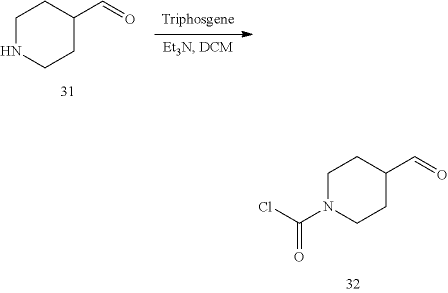

A solution of intermediate 31 hydrochloride (7 g, 47 mmol) in dichloromethane (180 mL) was added Et₃N (14.2 g, 141 mmol) at room temperature. The mixture was stirred at room temperature for 10 min. Then a solution of Triphosgene (5.6 g, 19 mmol) in dichloromethane (20 mL) was added to the mixture at 0° C.˜10° C. The mixture was stirred at room temperature for 2 hrs. TLC (dichloromethane/MeOH=10/1) showed the reaction was completed. Then the mixture was washed with aq. NaHCO₃, water, brine, dried over Na₂SO₄ and concentrated in vacuum. The crude product was distilled in vacuum to afford intermediate 32 (2.5 g, 30.5%) as colorless oil.

### Synthesis of Compound I-5

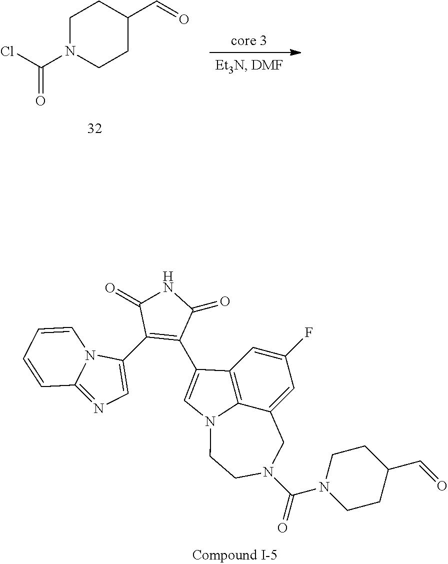

A solution of core 3 (5 g, 11.45 mmol) in DMF (70 mL) was added Et₃N (3.5 g, 34.35 mmol) at room temperature. The mixture was stirred at room temperature for 10 min. Then a solution of intermediate 32 (3.6 g, 20.5 mmol) in DMF (5 mL) was added to the mixture at 0° C.˜10° C. The mixture was stirred at room temperature for 2 hrs. TLC (dichloromethane/MeOH=15/1) showed the reaction was completed. The mixture was poured into ice-water and extracted with methyl tertiary butyl ether to remove impurities, then extracted with EtOAc (100 mL×5). The combined EtOAc phases were washed with water, brine, dried over Na₂SO₄ and concentrated in vacuum to give the crude Compound I-5 (4.5 g, crude) as an orange solid.

¹H NMR (DMSO-d₆, 400 MHz): δ (ppm) 11.3 (s, 1H), 9.58 (s, 1H), 8.05 (s, 1H), 7.92 (s, 1H), 7.65 (d, 1H, J=8.8 Hz), 7.60 (d, 1H, J=6.8 Hz), 7.21 (t, 1H, J=8 Hz), 6.78 (d, 1H, J=9.6 Hz), 6.58 (t, 1H, J=6.8 Hz), 5.62 (d, 1H, J=2 Hz), 4.64 (s, 2H), 4.55 (s, 2H), 3.85 (s, 2H), 3.21-3.37 (m, 2H), 2.73-2.76 (m, 2H), 1.75-1.76 (m, 2H), 1.33-1.47 (m, 3H).

### Synthesis of Compound I-6

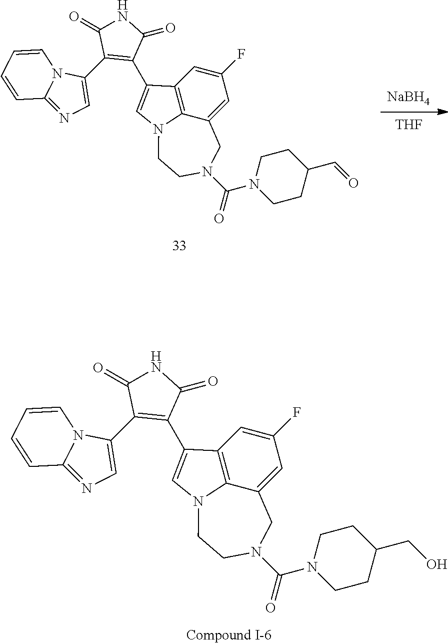 [SMILES: O=CC1CCN(C(=O)N2CCn3cc(C4=C(c5cnc6ccccn56)C(=O)NC4=O)c4cc(F)cc(c43)C2)CC1 ; "NaBH4" ; O=C1NC(=O)C(c2cnc3ccccn23)=C1c1cn2c3c(cc(F)cc13)CN(C(=O)N1CCC(CO)CC1)CC2 ; "THF"]

To solution of crude intermediate 33 (4.5 g) in THF (100 mL) was added NaBH₄ (0.16 g, 4.2 mmol) in portions below 5° C. After addition, the reaction mixture was stirred below 5° C. for 0.5 hr. TLC (dichloromethane/MeOH=15/1) showed the reaction was completed. Then the mixture was poured into water and extracted with EtOAc (100 mL×4). The combined organic phases were washed with water, brine, dried over Na₂SO₄ and concentrated in vacuum. The residue was purified by flash column chromatography (eluted with dichloromethane/MeOH from 200:1 to 30:1) to give compound I-6 (3 g, 48% for two steps) as orange solid.

¹H NMR (DMSO-d₆, 400 MHz): δ (ppm) 11.24 (s, 1H), 8.06 (s, 1H), 7.92 (s, 1H), 7.64-7.66 (d, 1H, J=8.8 Hz), 7.59-7.61 (d, 1H, J=6.4 Hz), 7.18-7.22 (t, 1H J=8 Hz), 6.78-6.80 (d, 1H, J=9.6 Hz), 6.56-6.59 (t, 1H J=6.8 Hz), 5.62-5.65 (d,d, 1H J=2 Hz J=10 Hz), 4.63 (s, 2H), 4.55 (s, 2H), 4.45-4.47 (t, 2H, J=5.2 Hz), 3.85 (s, 2H), 3.40-3.43 (d, 2H J=12.4 Hz), 3.23-3.26 (t, 2H, J=4.8 Hz), 2.58-2.65 (t, 2H, J=12 Hz), 1.55-1.58 (d, 2H J=12.8 Hz), 1.47-1.48 (d, 1H J=6.4 Hz), 1.05-1.13 (m, 2H).

LC/MS M+1 543.1

### Synthesis of Intermediate 35

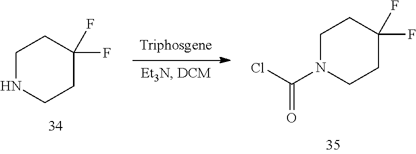

A solution of intermediate 34 hydrochloride (8.6 g, 55.0 mmol) in dichloromethane (240 mL) was added Et₃N (16.7 g, 165.0 mmol) at room temperature. The mixture was stirred at room temperature for 10 min. Then a solution of Triphosgene (6.5 g 22.0 mmol) in dichloromethane (20 mL) was added to the mixture at 0° C.˜10° C. The mixture was stirred at room temperature for 2 hrs. TLC (dichloromethane/MeOH=10/1) showed the reaction was completed. Then the mixture was washed with aq. NaHCO₃, water, brine, dried over Na₂SO₄ and concentrated in vacuum. The crude product was distilled in vacuum to afford intermediate 35 (4.2 g, 42%) as colorless oil.

¹H NMR (CDCl₃, 400 MHz): δ (ppm) 3.80-3.85 (bs, 2H), 3.71-3.75 (bs, 2H), 2.01-2.11 (m, 4H).

### Synthesis of Compound I-7

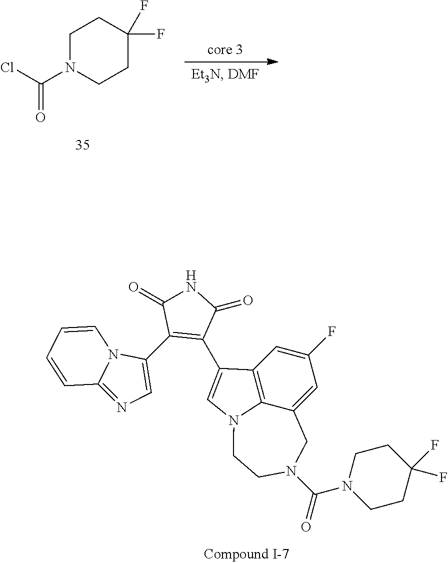

A solution of core 3 (3 g, 6.8 mmol) in DMF (40 mL) was added Et₃N (2.1 g, 20.6 mmol) at room temperature. The mixture was stirred at room temperature for 10 min. Then a solution of intermediate 35 (1.4 g 7.5 mmol) in DMF (5 mL) was added to the mixture at 0° C.˜10° C. The mixture was stirred at room temperature for 2 hrs. TLC (dichloromethane/MeOH=10/1) showed the reaction was completed. Then the mixture was poured into ice-water and extracted with methyl tertiary butyl ether to remove impurities, filtered. The filter cake was washed with water (100 mL×3), dissolved by dichloromethane (200 mL), washed with brine, dried over Na₂SO₄ and concentrated in vacuum to give Compound I-7 (2.2 g, 58%) as orange solid.

¹H NMR (DMSO-d₆, 400 MHz): δ (ppm) 11.24 (s, 1H), 8.06 (s, 1H), 7.91 (s, 1H), 7.62-7.66 (m, 2H), 7.19-7.23 (t, 1H, J=8 Hz), 6.84-6.86 (d, 1H, J=8.0 Hz), 6.58-6.61 (t, 1H, J=6.4 Hz), 5.63-5.66 (d, 1H, J=8.0 Hz), 4.70 (s, 2H), 4.58 (bs, 2H), 3.90 (bs, 2H), 3.15 (bs, 4H), 1.93-1.96 (m, 4H).

LC/MS M+1 549.1

### Synthesis of Intermediate 37

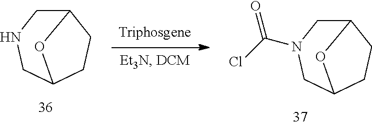

A solution of intermediate 36 hydrochloride (15 g, 100 mmol) in dichloromethane (430 mL) was added Et₃N (30.6 g, 300 mmol) at room temperature. The mixture was stirred at room temperature for 10 min. Then a solution of Triphosgene (11.9 g 40 mmol) in dichloromethane (20 mL) was added to the mixture at 0° C.˜10° C. The mixture was stirred at room temperature for 2 hrs. TLC (dichloromethane/MeOH=10/1) showed the reaction was completed. Then the mixture was washed with aq. NaHCO₃, water, brine, dried over Na₂SO₄ and concentrated in vacuum. The crude product was distilled in vacuum to afford intermediate 37 (9.2 g, 52%) as colorless oil.

¹H NMR (CDCl₃, 400 MHz): δ (ppm) 4.40 (s, 2H), 3.90-3.94 (d, 2H, J=13.2 Hz), 3.41-3.44 (d, 1H, J=13.2 Hz), 3.23-3.36 (d, 1H, J=12.8 Hz), 1.90-2.04 (m, 2H), 1.80-1.86 (m, 2H).

### Synthesis of Compound I-8

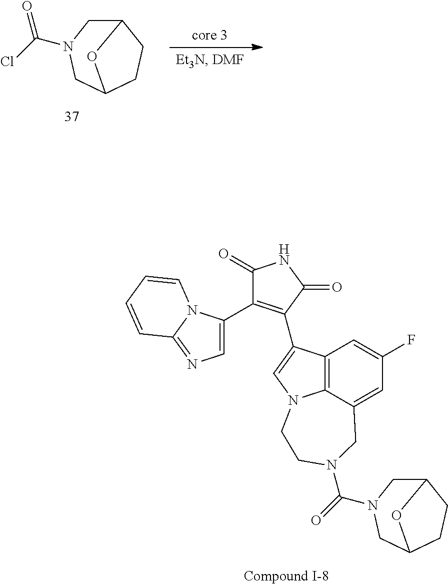

A solution of core 3 (3 g, 6.8 mmol) in DMF (40 mL) was added Et₃N (2.1 g, 20.6 mmol) at room temperature. The mixture was stirred at room temperature for 10 min. Then a solution of intermediate 37 (1.3 g 7.5 mmol) in DMF (5 mL) was added to the mixture at 0° C.˜10° C. The mixture was stirred at room temperature for 2 hrs. TLC (dichloromethane/MeOH=10/1) showed the reaction was completed. Then the mixture was poured into ice-water and extracted with EtOAc (100 mL×4). The combined organic phases were washed with water, brine, dried over Na₂SO₄ and concentrated in vacuum. The residue was purified by flash column chromatography (eluted with dichloromethane/MeOH from 200:1 to 50:1) to give compound I-8 (2.3 g, 62%) as orange solid.

¹H NMR (DMSO-d₆, 400 MHz): δ (ppm) 11.24 (s, 1H), 8.05 (s, 1H), 7.92 (s, 1H), 7.60-7.66 (m, 2H), 7.20-7.24 (t, 1H, J=7.6 Hz), 6.80-6.82 (d, 1H, J=9.2 Hz), 6.57-6.59 (t, 1H J=6.8 Hz), 5.64-5.67 (d, 1H J=10 Hz), 4.60 (s, 2H), 4.54 (bs, 2H), 4.19 (s, 2H), 3.84 (bs, 2H), 3.20-3.24 (d, 2H J=12.8 Hz), 3.01-3.04 (d, 2H, J=12 Hz), 1.74 (s, 4H).

LC/MS M+1 541.1

### Synthesis of Intermediate 39

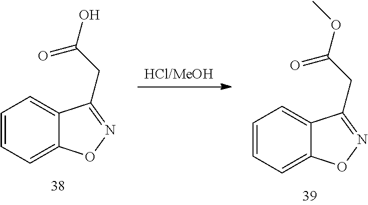

To a solution of intermediate 38 (20 g, 113 mmol) in MeOH (100 mL) was added HCl/MeOH (4 M, 100 mL), then stirred at room temperature overnight. The mixture was concentrated in vacuum. To the residue was added water (500 mL), then extracted with EtOAc (200 mL×4). The combined organic phases were washed with brine, dried over Na₂SO₄ and concentrated in vacuum to give the crude intermediate 39 (21 g) as brown solid, which was used directly for the next step without purification.

### Synthesis of Intermediate 40

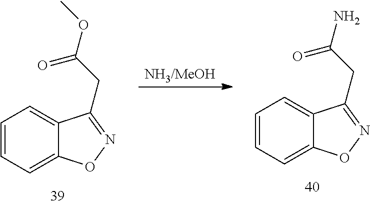

To a solution of crude intermediate 39 (21 g) in MeOH (100 mL) was added NH₃/MeOH (6 M, 100 mL). The reaction mixture was stirred at room temperature overnight. The mixture was poured into EtOAc (500 mL), and then filtered. The filter cake was dried in vacuo to give intermediate 40 (8 g, 40% for two steps) as off-white solid.

¹H NMR (DMSO-d₆, 400 MHz): δ (ppm) 7.86 (d, J=8.0 Hz, 1H), 7.80 (s br, 1H), 7.72 (d, J=8.4 Hz, 1H), 7.62-7.66 (m, 1H), 7.37-7.41 (m, 1H), 7.22 (s br, 1H), 3.88 (s, 2H).

### Synthesis of Intermediate 41

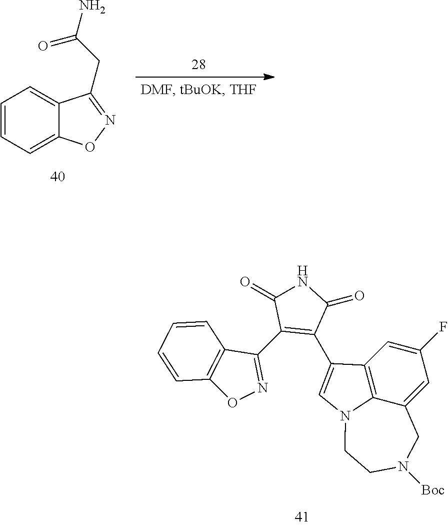

To a solution of intermediate 28 (10 g, 26.5 mmol) and intermediate 40 (4.6 g, 26.5 mmol) in DMF (120 mL) was added a solution of tBuOK (7.4 g, 66.2 mmol) in THF (100 mL) at 0-10° C. The mixture was stirred at 0-10° C. for 15 min. TLC (petroleum ether/EtOAc=1/1) showed the reaction was complete. Then the mixture was poured into ice-water and extracted with EtOAc (100 mL×4). The combined organic phases were washed with water, brine, dried over Na₂SO₄ and concentrated in vacuum. The residue was purified by flash column chromatography (eluted with petroleum ether/EtOAc from 10:5 to 1:2) to give intermediate 41 (5 g, 37.5%) as red solid.

¹H NMR (DMSO-d₆, 400 MHz): δ (ppm) 11.07 (s, 1H), 8.02-8.01 (d, 1H, J=4 Hz), 8.96 (s, 1H), 7.61-7.63 (t, 1H, J=8.8 Hz), 7.45 (s, 1H), 7.17 (s, 1H), 6.57-6.64 (m, 1H), 6.47-6.49 (d, 1H, J=8 Hz), 5.77-5.80 (d, 1H, J=8.4 Hz), 4.73-4.78 (d, 2H), 4.52 (bs, 2H), 3.96 (bs, 2H), 1.25-1.44 (m, 9H).

### Synthesis of Intermediate 42

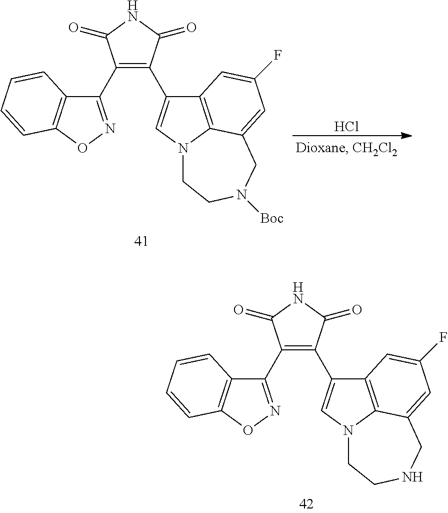 [SMILES: CC(C)(C)OC(=O)N1CCn2cc(C3=C(c4noc5ccccc45)C(=O)NC3=O)c3cc(F)cc(c32)C1 ; Cl ; O=C1NC(=O)C(c2cn3c4c(cc(F)cc24)CNCC3)=C1c1noc2ccccc12]

A solution of intermediate 41 (5 g, 9.9 mmol) in dichloromethane (50 mL) was added HCl/dioxane (50 mL, 7M) at room temperature. The mixture was stirred at room temperature for 1 hr. TLC (dichloromethane/MeOH=15/1) showed the reaction was complete. The solvent was concentrated to give intermediate 42 (4 g, 92%) as orange solid.

¹H NMR (DMSO-d₆, 400 MHz): δ (ppm) 11.59 (s, 1H), 10.46 (s, 1H), 8.49 (s, 1H), 8.17 (s, 1H), 8.08-8.12 (m, 2H, J=14 Hz), 7.88-7.92 (t, 1H, J=8.4 Hz), 7.25-7.29 (t, 1H, J=13.6 Hz), 7.04-7.07 (t, 1H, J=10 Hz), 6.22-6.25 (m, 1H), 4.76 (s, 2H), 4.62 (s, 2H), 4.45 (s, 2H), 3.68 (s, 2H).

### Synthesis of Compound I-9

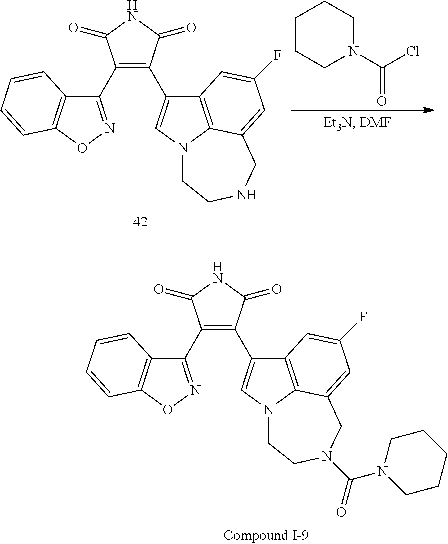

To A solution of intermediate 42 (2 g, 4.9 mmol) and 1-Piperidinecarbonyl chloride (1.1 g, 7.4 mmol) in DMF (20 mL) was added Et₃N (1.5 g, 14.9 mmol) at room temperature. The mixture was stirred at room temperature for 30 min. TLC (dichloromethane/MeOH=15/1) showed the reaction was complete. The mixture was poured into ice-water and extracted with EtOAc (100 mL×4). The combined organic phases were washed with water, brine, dried over Na₂SO₄ and concentrated in vacuum. The residue was washed with methyl tertiary butyl ether, filtered. The filter cake was dried in vacuum to give Compound I-9 (1.1 g, 43.1%) as an orange solid.

¹H NMR (DMSO-d₆, 400 MHz): δ (ppm) 11.48 (s, 1H), 8.23 (s, 1H), 7.83-7.86 (d, 1H, J=8.8 Hz), 7.68-7.76 (m, 1H), 7.35-7.39 (t, 1H, J=7.6 Hz), 6.89-6.92 (m, 1H), 6.04-6.07 (m, 1H), 4.66 (s, 2H), 4.57-4.65 (bs, 2H), 3.83-3.90 (m, 2H), 3.01-3.05 (m, 4H), 1.46-1.49 (m, 6H).

LC/MS M+514.1

### Synthesis of Compound I-12

Compound I-12 can be synthesized in a similar manner to Compound I-7 using 3,3-difluropiperdine as the starting material.

¹H NMR (DMSO-d₆, 400 MHz): δ (ppm) 11.25 (s, 1H), 8.06 (s, 1H), 7.93 (s, 1H), 7.58-7.66 (m, 2H), 7.18-7.23 (t, 1H, J=8 Hz), 6.81-6.79 (d, 1H, J=8.0 Hz), 6.58-6.61 (t, 1H, J=6.4 Hz), 5.65-5.67 (d, 1H, J=8.0 Hz), 4.66 (s, 2H), 4.56 (bs, 2H), 3.88 (bs, 2H), 3.31-3.64 (bs, 2H), 3.07 (bs, 2H), 1.96-2.01 (bs, 2H), 1.69 (bs, 2H).

LC/MS M+1 549.1

### Synthesis of Compound I-13

Compound I-13 as shown in Synthetic Scheme 9.

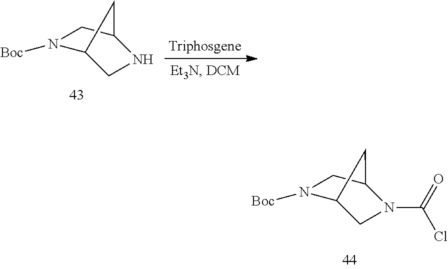 [SMILES: CC(C)(C)OC(=O)N1CC2CC1CN2 ; CC(C)(C)OC(=O)N1CC2CC1CN2C(=O)Cl]

The synthesis of Intermediate 44 is a similar manner as Intermediate 47, yield 43.1%.

¹H NMR (400 MHz, CDCl₃) δ 1.47 (9H, s), 1.91-1.94 (2H, m), 3.37-3.56 (4H, m), 4.40-4.70 (1H, m), 4.71-4.74 (1H, m)

### Synthesis of Intermediate 45

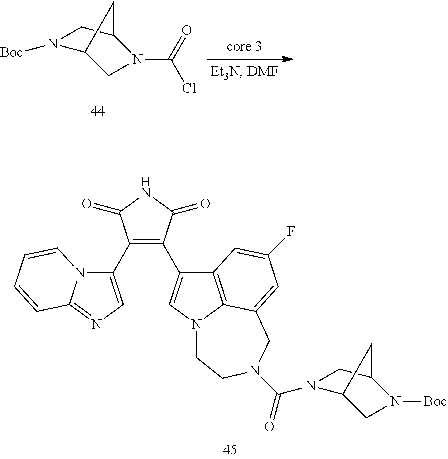 [SMILES: CC(C)(C)OC(=O)N1CC2CC1CN2C(=O)Cl ; CC(C)(C)OC(=O)N1CC2CC1CN2C(=O)N1CCn2cc(C3=C(c4cnc5ccccn45)C(=O)NC3=O)c3cc(F)cc(c32)C1]

The synthesis of Intermediate 45 is a similar manner as Intermediate 48, crude.

### Synthesis of Compound I-13

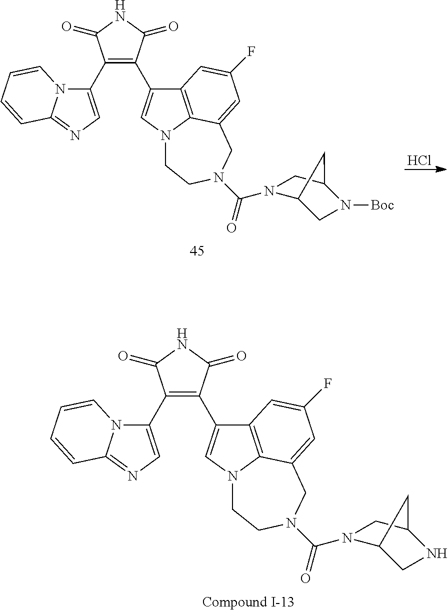 [SMILES: CC(C)(C)OC(=O)N1CC2CC1CN2C(=O)N1CCn2cc(C3=C(c4cnc5ccccn45)C(=O)NC3=O)c3cc(F)cc(c32)C1 ; Cl ; O=C1NC(=O)C(c2cnc3ccccn23)=C1c1cn2c3c(cc(F)cc13)CN(C(=O)N1CC3CC1CN3)CC2]

The synthesis of Compound I-13 in a similar manner as Compound I-19, yield 20.7% for two steps.

¹H NMR (400 MHz, DMSO-d₆) δ1.45-1.48 (1H, d, J=9.2 Hz), 1.54-1.56 (1H, d, J=9.2 Hz), 2.71-2.73 (1H, d, J=9.2 Hz), 2.88-2.90 (1H, d, J=8.4 Hz), 2.97-2.99 (1H, d, J=9.6 Hz), 3.35-3.37 (1H, d, J=8.0 Hz), 3.49 (1H, s), 3.75-3.76 (1H, m), 3.90-3.92 (1H, m), 4.04 (1H, s), 4.46-4.50 (1H, m), 4.58-4.74 (3H, m), 5.60-5.62 (1H, d, J=8.4 Hz), 6.60-6.63 (1H, m), 6.78-6.80 (1H, d, J=9.6 Hz), 7.20-7.24 (1H, m), 7.63-7.66 (2H, m), 7.90 (1H, s), 8.06 (1H, s).

LCMS purity is >95%, Rt=2.55 min; MS Calcd: 525.5; MS Found: 526.2 [M+1]+.

### Synthesis of Compound I-14

Compound I-14 can be synthesized in a similar manner to Compound I-8.

LC/MS M+1 548.2

### Synthesis of Compound I-15

Compound I-15 can be synthesized in a similar manner to Compound I-7.

LC/MS M+1 556.2

### Synthesis of Compound I-16

Compound I-16 can be synthesized in a similar manner to Compound I-7.

LC/MS M+1 556.2

### Synthesis of Compound I-17

Compound I-17 can be synthesized in a similar manner to Compound I-7.

LC/MS M+1 599.2

### Synthesis of Compound I-18

Compound I-18 can be synthesized in a similar manner to Compound I-8.

LC/MS M+1 591.2

### Synthesis of Compound I-19

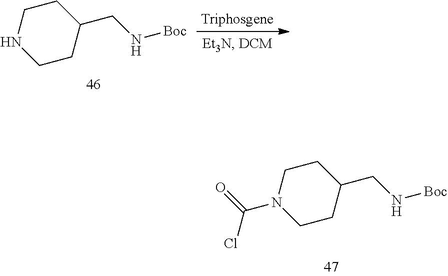 [SMILES: CC(C)(C)OC(=O)NCC1CCNCC1 ; CC(C)(C)OC(=O)NCC1CCN(C(=O)Cl)CC1]

To a solution, Intermediate 46 (1.0 g, 4.67 mmol) in DCM (25 mL) was added Et₃N (1.41 g, 14.0 mmol). The suspension was stirred and treated dropwise with Triphosgene (0.55 g, 1.87 mmol) in DCM (5 mL) at 0˜10° C. After addition was completed, the suspension was stirred for 2 hrs at room temperature. The reaction mixture poured into iced water (20 mL), extracted with DCM, and the organic layer was washed with NaHCO₃(aq), brine, dried over Na₂SO₄, filtered and concentrated in vacuum. The residue was purified by silica gel column chromatography to give Intermediate 47 (0.6 g, 46.5%) as colorless oil.

¹H NMR (400 MHz, CDCl₃) δ 1.20-1.27 (2H, m), 1.41 (9H, s), 1.72-1.79 (3H, m), 2.84-2.90 (1H, m), 3.03-3.09 (3H, m), 4.32-4.35 (2H, m), 4.66 (1H, br s).

### Synthesis of Compound I-19

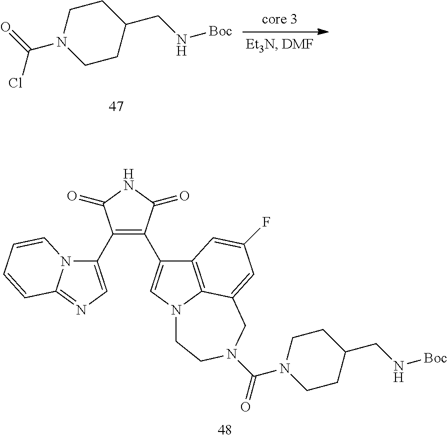 [SMILES: CC(C)(C)OC(=O)NCC1CCN(C(=O)Cl)CC1 ; CC(C)(C)OC(=O)NCC1CCN(C(=O)N2CCn3cc(C4=C(c5cnc6ccccn56)C(=O)NC4=O)c4cc(F)cc(c43)C2)CC1]

To a solution of Core 3 (1.0 g, 2.29 mmol) in DMF (13 mL) was added Et₃N (0.70 g, 6.87 mmol). The suspension was stirred and treated dropwise with Intermediate 47 (0.70 g, 2.52 mmol) in DMF (2 mL) at 0˜10° C. After addition was completed, the suspension was stirred for 2 hrs at room temperature. The reaction mixture was poured into iced water (60 mL), filtered and concentrated in vacuo to give Intermediate 48 (1.0 g, crude) as red solid

### Synthesis of Compound I-19

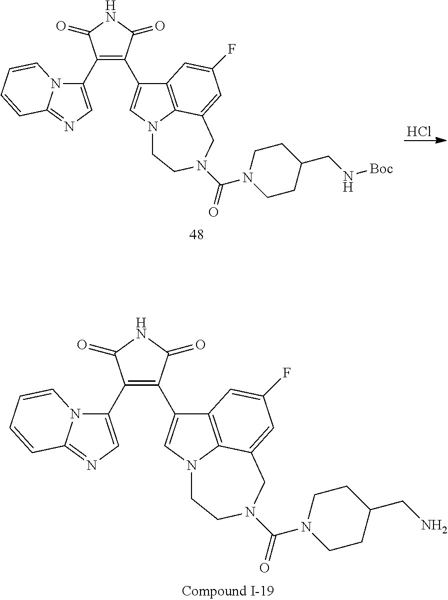 [SMILES: CC(C)(C)OC(=O)NCC1CCN(C(=O)N2CCn3cc(C4=C(c5cnc6ccccn56)C(=O)NC4=O)c4cc(F)cc(c43)C2)CC1 ; Cl ; NCC1CCN(C(=O)N2CCn3cc(C4=C(c5cnc6ccccn56)C(=O)NC4=O)c4cc(F)cc(c43)C2)CC1]

To a solution of Intermediate 48 (1.0 g, crude) in DCM (30 mL) was added HCl/dioxane (10 mL, 8 mol/L) at room temperature, and the mixture was stirred at room temperature for 2 hrs. The reaction mixture was filtered, and the filter cake was dissolved in water, adjusted to pH=8-9 with Na₂CO₃ (aq), and filtered again, washed with water and concentrated in vacuo to give Compound I-19 (130 mg, 10.5%, two steps) as red solid.

¹H NMR (400 MHz, DMSO-d₆) δ1.03-1.11 (2H, m), 1.13-1.62 (3H, m), 2.44-2.82 (4H, m), 3.41-3.44 (2H, m), 3.85 (2H, m), 4.54 (2H, m), 4.64 (2H, m), 5.64-5.66 (1H, d, J=9.6 Hz), 6.56-6.58 (1H, m), 6.78-6.81 (1H, m), 7.19-7.23 (1H, m), 7.60-7.66 (2H, m), 7.92 (1H, s), 8.06 (1H, s).

LCMS purity is >95%, Rt=2.77 min; MS Calcd: 541.6; MS Found: 542.2 ([M+1]⁺.

### Synthesis of Compound I-20

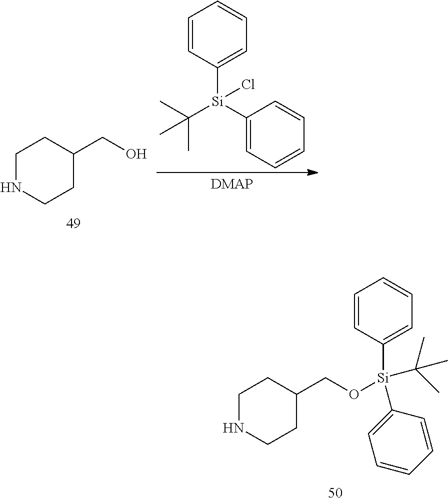

To a solution of Intermediate 49 (15 g, 130.2 mmol) in DCM (1.5 L) was added Et₃N (19.8 g, 195.3 mmol) and DMAP (0.8 g, 6.5 mmol). The suspension was stirred and treated dropwise with tert-butylchlorodiphenylsilane (53.7 g, 195.3 mmol) at 0˜10° C. After addition was completed, the suspension was stirred for 5 hrs at room temperature. The reaction mixture poured into iced water (500 mL), extracted with DCM (300 mL×2), and the organic layer was washed with brine (300 mL×2), dried over Na₂SO₄, filtered and concentrated in vacuum to give Intermediate 50 (30 g, crude) as yellow oil.

### Synthesis of Intermediate 51

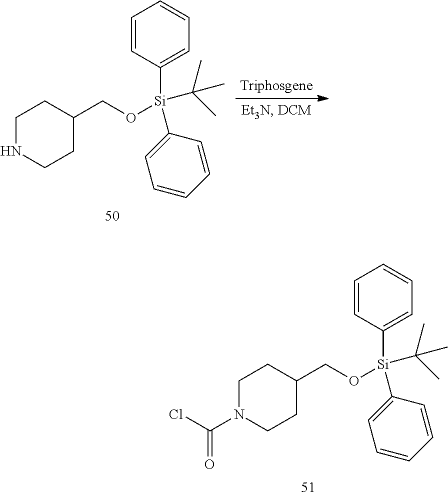 [SMILES: CC(C)(C)[Si](OCC1CCNCC1)(c1ccccc1)c1ccccc1 ; CC(C)(C)[Si](OCC1CCN(C(=O)Cl)CC1)(c1ccccc1)c1ccccc1]

To a solution of Intermediate 50 (30 g, crude, 84.8 mmol) in DCM (900 mL) was added Et₃N (25.8 g, 254.5 mmol). The suspension was stirred and treated dropwise with Triphosgene (10.1 g, 33.9 mmol) in DCM (50 mL) at 0˜10° C. After addition was completed, the suspension was stirred for 2 hrs at room temperature. The reaction mixture poured into iced water (300 mL), extracted with DCM, and the organic layer was washed with NaHCO₃(aq), brine, dried over Na₂SO₄, filtered and concentrated in vacuum. The residue was purified by silica gel column chromatography to give Intermediate 51 (3.1 g, 8.8%) as colorless oil.

¹H NMR (400 MHz, CDCl₃) δ1.05 (9H, s), 1.22-1.29 (2H, m), 1.78-1.83 (3H, m), 2.84-2.90 (1H, m), 3.01-3.07 (1H, m), 3.50-3.51 (2H, d, J=5.6 Hz), 4.30-4.34 (2H, m), 7.36-7.45 (6H, m), 7.62-7.64 (4H, m)

### Synthesis of Intermediate 52

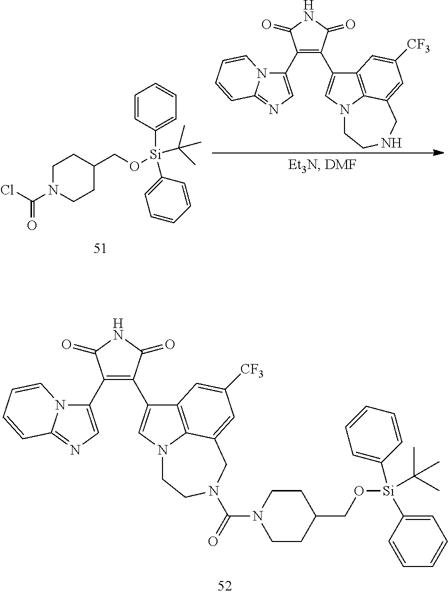 [SMILES: CC(C)(C)[Si](OCC1CCN(C(=O)Cl)CC1)(c1ccccc1)c1ccccc1 ; O=C1NC(=O)C(c2cnc3ccccn23)=C1c1cn2c3c(cc(C(F)(F)F)cc13)CNCC2 ; CC(C)(C)[Si](OCC1CCN(C(=O)N2CCn3cc(C4=C(c5cnc6ccccn56)C(=O)NC4=O)c4cc(C(F)(F)F)cc(c43)C2)CC1)(c1ccccc1)c1ccccc1]

To a solution of Maleimide Core (1.6 g, 3.28 mmol) in DMF (15 mL) was added Et₃N (1.0 g, 9.84 mmol). The suspension was stirred and treated dropwise with Intermediate 51 (1.5 g, 3.60 mmol) in DMF (5 mL) at 0˜10° C. After addition was completed, the suspension was stirred for 2 hrs at room temperature. The reaction mixture poured into iced water (80 mL), filtered and the filter cake was washed with water and MTBE concentrated in vacuo to give Intermediate 52 (0.98 g, crude) as red solid.

### Synthesis of Compound I-20

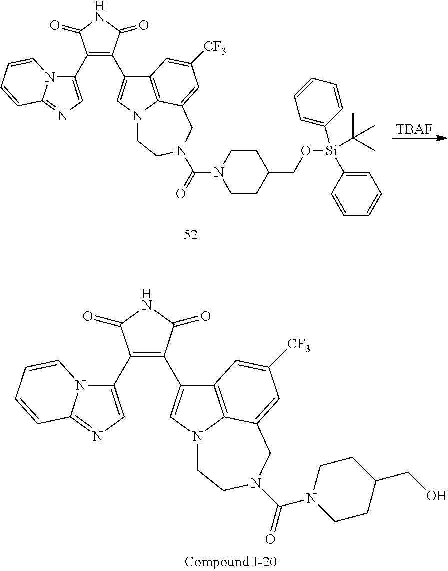

To a solution of Intermediate 52 (0.98 g, crude) in THF (20 mL) was added TBAF (0.56 g, 1.77 mmol) in THF (10 mL) at room temperature, and the mixture was stirred at room temperature for 2 hrs at room temperature. The reaction mixture was poured into iced water (40 mL), filtered, and the filter cake was concentrated in vacuo, purified by silica gel column chromatography to give to Compound I-20 (160 mg, 22.8%) as orange solid.

¹H NMR (400 MHz, DMSO-d₆) δ1.05-1.13 (2H, m), 1.48-1.58 (3H, m), 2.54-2.67 (2H, m), 3.23-3.26 (2H, m), 3.40-3.44 (2H, m), 3.87-3.90 (2H, m) 4.44-4.47 (1H, m) 4.62-4.69 (2H, m), 4.72 (2H, s), 6.31 (1H, s), 6.53-6.56 (1H, m), 7.14-7.20 (2H, m), 7.60-7.64 (2H, m), 7.92 (1H, s), 8.16 (1H, s), 11.29 (1H, s).

LCMS purity is >95%, Rt=2.87 min; MS Calcd: 592.6; MS Found: 593.2 ([M+1]⁺.

### Synthesis of Compound I-21

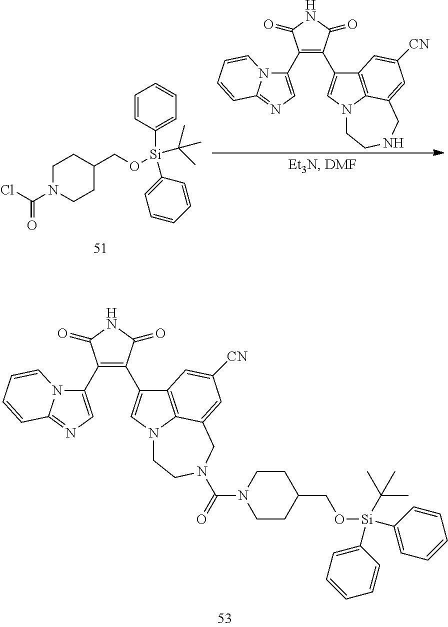 [SMILES: CC(C)(C)[Si](OCC1CCN(C(=O)Cl)CC1)(c1ccccc1)c1ccccc1 ; N#Cc1cc2c3c(c1)c(C1=C(c4cnc5ccccn45)C(=O)NC1=O)cn3CCNC2 ; CC(C)(C)[Si](OCC1CCN(C(=O)N2CCn3cc(C4=C(c5cnc6ccccn56)C(=O)NC4=O)c4cc(C#N)cc(c43)C2)CC1)(c1ccccc1)c1ccccc1]

The synthesis of Intermediate 53 is a similar manner as Intermediate 52, crude.

### Synthesis of Compound I-21

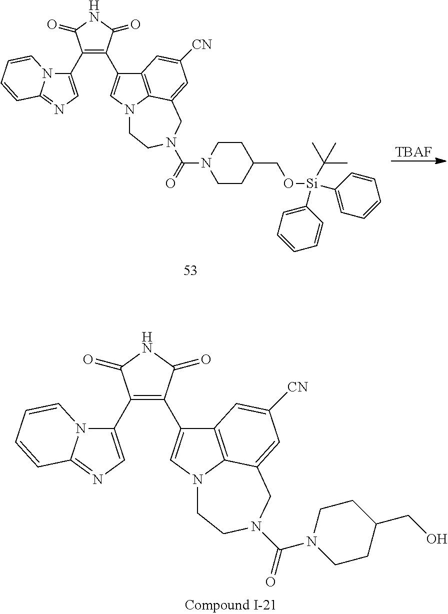 [SMILES: CC(C)(C)[Si](OCC1CCN(C(=O)N2CCn3cc(C4=C(c5cnc6ccccn56)C(=O)NC4=O)c4cc(C#N)cc(c43)C2)CC1)(c1ccccc1)c1ccccc1 ; "TBAF" ; N#Cc1cc2c3c(c1)c(C1=C(c4cnc5ccccn45)C(=O)NC1=O)cn3CCN(C(=O)N1CCC(CO)CC1)C2]

The synthesis of Compound I-21 is the same as Compound I-20, yield 28.6%.

¹H NMR (400 MHz, DMSO-d₆) δ1.08-1.13 (2H, m), 1.47-1.56 (1H, m), 1.56-1.59 (2H, m), 2.58-2.65 (2H, m), 3.23-3.26 (2H, m), 3.40-3.43 (2H, m), 3.86 (2H, s), 4.45-4.61 (1H, m), 4.61 (2H, s), 4.68 (2H, s), 6.44 (1H, s), 6.53-6.57 (1H, m), 7.19-7.24 (1H, m), 7.28 (1H, s), 7.54-7.56 (1H, d, J=6.8 Hz), 7.67-7.69 (2H, d, J=9.2 Hz), 7.98 (1H, s), 8.08 (1H, s), 11.32 (1H, s).

LCMS purity is >95%, Rt=3.41 min; MS Calcd: 549.6; MS Found: 550.2 [M+1]⁺.

Synthesis of Compounds I-22, 23, 24, 25, 27 can be synthesized in a similar manner to Compound I-7.

### Synthesis of Compound I-26

Compound I-26 can be synthesized as shown in Synthetic Scheme 11.

### Synthesis of Compound I-28

Compound I-28 can be synthesized as shown in Synthetic Scheme 12.

Synthesis of Compounds I-29, 30 can be synthesized in a similar manner as the undeuterated material by utilizing the appropriate deuterated starting materials.

### Definitions

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

An “additive effect” refers to an effect wherein two or more substances or actions used in combination produce a total effect, the same as the arithmetic sum of the individual effects.

“Administration” refers to introducing a substance into a subject. In some embodiments, administration is intradermal injection, topical, transdermal or oral. In some embodiments, administration is directly to the scalp. In some embodiments, administration is directly to the skin via an implant delivery system. In certain embodiments “causing to be administered” refers to administration of a second component after a first component has already been administered (e.g., at a different time and/or by a different actor).

An “antibody” refers to an immunoglobulin polypeptide, or fragment thereof, having immunogen binding ability.

As used herein, an “agonist” is an agent that causes an increase in the expression or activity of a target gene, protein, or a pathway, respectively. Therefore, an agonist can bind to and activate its cognate receptor in some fashion, which directly or indirectly brings about this physiological effect on the target gene or protein. An agonist can also increase the activity of a pathway through modulating the activity of pathway components, for example, through inhibiting the activity of negative regulators of a pathway. Therefore, a “Wnt agonist” can be defined as an agent that increases the activity of Wnt pathway, which can be measured by increased TCF/LEF-mediated transcription in a cell. Therefore, a “Wnt agonist” can be a true Wnt agonist that bind and activate a Frizzled receptor family member, including any and all of the Wnt family proteins, an inhibitor of intracellular beta-catenin degradation, and activators of TCF/LEF.

An “antagonist” refers to an agent that binds to a receptor or protein, and which in turn decreases or eliminates binding by other molecules.

“Anti-sense” refers to a nucleic acid sequence, regardless of length, that is complementary to the coding strand or mRNA of a nucleic acid sequence. Antisense RNA can be introduced to an individual cell, tissue or organanoid. An anti-sense nucleic acid can contain a modified backbone, for example, phosphorothioate, phosphorodithioate, or other modified backbones known in the art, or may contain non-natural internucleoside linkages.

“Biocompatible Matrix” as used herein is a polymeric carrier that is acceptable for administration to humans for the release of therapeutic agents. A Biocompatible Matrix may be a biocompatible gel or foam.

“Cell Density” as used herein in connection with a specific cell type is the mean number of that cell type per area in a Representative Microscopy Sample. The cell types may include but are not limited to hair follicle stem cells, dermal papilla stem cells, keratinocytes, melanocytes, and bulge cells. The Cell Density may be assessed with a given cell type in a given organ or tissue, including but not limited to, a hair follicle.

“Complementary nucleic acid sequence” refers to a nucleic acid sequence capable of hybridizing with another nucleic acid sequence comprised of complementary nucleotide base pairs. By “hybridize” is meant pair to form a double-stranded molecule between complementary nucleotide bases (e.g., adenine (A) forms a base pair with thymine (T), as does guanine (G) with cytosine (C) in DNA) under suitable conditions of stringency. (See, e.g., Wahl, G. M. and S. L. Berger (1987) Methods Enzymol. 152:399; Kimmel, A. R. (1987) Methods Enzymol. 152:507).

“Cross-Sectional Cell Density” as used herein in connection with a specific cell type is the mean number of that cell type per area of cross section through a tissue in a Representative Microscopy Sample. Cross sections of a given tissue can also be used to determine the number of cells in a given plane. Typically, Cross-sectional Cell Density will be measured by analyzing whole mount preparations of a given tissue and counting the number of a given specific cell type across a given distance in cross sections taken along a portion of the epithelia, as described in a Representative Microscopy Sample.

“Decreasing” or “Decreases” refers to decreasing by at least 5%, for example, 5, 6, 7, 8, 9, 10, 15, 20, 25, 30, 35, 40, 45, 50, 55, 60, 65, 70, 75, 80, 85, 90, 95, 99 or 100%, for example, as compared to the level of reference.

“Decreases” also means decreases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of a reference.

“Dermal Papilla hair follicle culture assay” or “DP hair follicle culture assay” refers to a method of using intact hair follicles to quantify the number of Dermal Papilla (DP) cells or size of the DP area within the follicle when measured at the end of the assay. DP cells are cells that express alkaline phosphatase (AP), and/or Versican, and/or Vimentin, and/or Sox2, and/or CD133. Briefly, microdissection is used to remove intact hair follicles from a specimen. Each hair is trimmed close to the follicle apex and about 3-5 hair follicles are distributed in wells. Culture media with growth factors and/or small molecules are added to the wells, and the follicles are incubated at 37° C. Growth is monitored for one or more desired period of time. EVOS® transmitted light images may be taken at one or more desired period of time. Hair follicles may be processed for alkaline phosphatase staining, 5-ethynyl-2′-deoxyuridine (EdU) staining and/or immunofluorescence to detect markers such as Vimentin, Sox2, Versican, CD133, etc. The total area of a hair follicle staining positive for DP markers may be analyzed and hair shaft growth may be monitored over time. Details of the protocol are provided in the Examples section of the present disclosure.

“Dermal Papilla stem cell” or “DP stem cell” or “DP cell” refers to a cell in the dermal papilla of a hair follicle having the capacity to self-renew.

“Differentiation Period” as used herein is the duration of time in which there is an Effective Sternness Driver Concentration.

“Effective Concentration” may be the “Effective Sternness Driver Concentration” for a Sternness Driver, “Effective Minoxidil Concentration” for Minoxidil, “Effective Shh Concentration” for a Shh pathway activator, or “Effective Wnt Agonist Concentration” for a Wnt Agonist.

“Effective Shh Concentration” is the minimum concentration of a Shh pathway activator that creates a >50% increase in the number of Dermal Papilla (DP) spheroids, size of DP spheroids, or number of DP cells in cell culture when combined with a Sternness Driver, measured at the end of the Stem Cell Proliferation Assay using dermal papilla, compared to the respective increase in the number of Dermal Papilla (DP) spheroids, size of DP spheroids, or number of DP cells in cell culture in absence of a Shh pathway activator with all other components present at the same concentration, measured at the end of the Stem Cell Proliferation Assay using dermal papilla cells. “Effective in vitro Shh Pathway Activation Concentration” refers to the “Effective Shh Concentration” in vitro.

“Effective Wnt Agonist Concentration” is the minimum concentration of a Wnt pathway agonist that creates a >50% increase in the number of Dermal Papilla (DP) spheroids, size of DP spheroids, or number of DP cells in cell culture, measured at the end of the Stem Cell Proliferation Assay using dermal papilla, compared to the respective increase in the number of Dermal Papilla (DP) spheroids, size of DP spheroids, or number of DP cells in cell culture in the absence of the Wnt pathway agonist with all other components present at the same concentration, measured at the end of the Stem Cell Proliferation Assay using dermal papilla. “Effective in vitro Wnt Agonist Concentration” refers to the “Effective Wnt Agonist Concentration” in vitro.

“Effective Minoxidil” is the minimum concentration of minoxidil that simulated hair growth compared hair growth in absence of minoxidil with all other components present at the same concentration. “Effective in vitro minoxidil” refers to the “Effective minoxidil” in vitro.

“Effective Release Rate” (mass/time) as used herein is the Effective Concentration (mass/volume)*30 μL/1 hour.

“Effective Sternness Driver Concentration” is the minimum concentration of a Sternness Driver that induces at least 50% increase in number of cells of a given cell type in a Stem Cell Proliferation Assay compared to the number of cells of a given cell type in a Stem Cell Proliferation Assay performed without the Sternness Driver and with all other components present at the same concentrations. In certain instances, the “Effective Sternness Driver Concentration” may be the “Effective Wnt Agonist Concentration”.

“Eliminate” means to decrease to a level that is undetectable.

“Engraft” or “engraftment” refers to the process of stem or progenitor cell incorporation into a tissue of interest in vivo through contact with existing cells of the tissue. “Epithelial progenitor cell” refers to a multipotent cell which has the potential to become restricted to cell lineages resulting in epithelial cells.

“Epithelial stem cell” refers to a multipotent cell which has the potential to become committed to multiple cell lineages, including cell lineages resulting in epithelial cells.

“FDA Approved Minoxidil Concentration” refers to the concentration for which the FDA has approved (i.e., determined safe and effective) for the use of minoxidil to treat hair loss or high blood pressure. For example, the maximum recommended dosage of minoxidil is 100 mg/day.

“Fragment” refers to a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“GSK3 inhibitor” is a composition that inhibits the activity of GSK3, GSK-3alpha, and/or GSK-3beta.

“GSK3beta,” “GSK33,” and “GSK3B” as used interchangeably herein are acronyms for glycogen synthase kinase 3 beta.

“GSK3beta inhibitor” is a composition that inhibits the activity of GSK3beta.

“Hair follicle stem cell” refers to a multipotent cell in a region of a hair follicle distinct from the dermal papilla that has the capacity to self-renew and to differentiate into multiple cell lineages.

“Hybridize” refers to pairing to form a double-stranded molecule between complementary nucleotide bases (e.g., adenine (A) forms a base pair with thymine (T), as does guanine (G) with cytosine (C) in DNA) under suitable conditions of stringency. (See, e.g., Wahl, G. M. and S. L. Berger (1987) Methods Enzymol. 152:399; Kimmel, A. R. (1987) Methods Enzymol. 152:507).

An “inhibitor” refers to an agent that causes a decrease in the expression or activity of a target gene or protein, respectively. An “antagonist” can be an inhibitor, but is more specifically an agent that binds to a receptor, and which in turn decreases or eliminates binding by other molecules.

As used herein, an “inhibitory nucleic acid” is a double-stranded RNA, RNA interference, miRNA, siRNA, shRNA, or antisense RNA, or a portion thereof, or a mimetic thereof, that when administered to a mammalian cell results in a decrease in the expression of a target gene. Typically, a nucleic acid inhibitor comprises at least a portion of a target nucleic acid molecule, or an ortholog thereof, or comprises at least a portion of the complementary strand of a target nucleic acid molecule. Typically, expression of a target gene is reduced by 10%, 25%, 50%, 75%, or even 90-100%.

“Increases” also means increases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of a as compared to the level of a reference standard.

“Increasing” refers to increasing by at least 5%, for example, 5, 6, 7, 8, 9, 10, 15, 20, 25, 30, 35, 40, 45, 50, 55, 60, 65, 70, 75, 80, 85, 90, 95, 99, 100% or more, for example, as compared to the level of a reference.

“Intradermal administration” refers to administration of a medication, pharmaceutical composition or compound into the dermis, just below the epidermis.

“Isolated” refers to a material that is free to varying degrees from components which normally accompany it as found in its native state. “Isolate” denotes a degree of separation from original source or surroundings.

“Lineage Tracing” as used herein is using a mouse line that enables fate tracing of any cell that expresses a target gene at the time of reporter induction. Examples include Gli1, Krt15, CD34, Lgr5, Lgr6, Lrig1, Sox2, CD133, Vimentin, Versican and/or alkaline phosphatase.

“Mammal” refers to any mammal including but not limited to human, mouse, rat, sheep, monkey, goat, rabbit, hamster, horse, cow or pig.

“Mean Release Time” as used herein is the time in which one-half of an agent is released into phosphate buffered saline from a carrier in a Release Assay.

“Native Morphology” as used herein is means that tissue organization largely reflects the organization in a healthy tissue.

“Non-human mammal”, as used herein, refers to any mammal that is not a human.

As used in relevant context herein, the term “number” of cells can be 0, 1, or more cells.

“Organoid” or “epithelial organoid” refers to a cell cluster or aggregate that resembles an organ, or part of an organ, and possesses cell types relevant to that particular organ.

“Population” of cells refers to any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

“Progenitor cell” as used herein refers to a cell that, like a stem cell, has the tendency to differentiate into a specific type of cell, but is already more specific than a stem cell and is pushed to differentiate into its “target” cell.

“Reference” means a standard or control condition (e.g., untreated with a test agent or combination of test agents).

“Release Assay” as used herein is a test in which the rate of release of an agent from a Biocompatible Matrix through dialysis membrane to a saline environment. An exemplary Release Assay may be performed by placing 30 microliters of a composition in 1 ml Phosphate Buffered Saline inside saline dialysis bag with a suitable cutoff, and placing the dialysis bag within 10 mL of Phosphate Buffered Saline at 37° C. The dialysis membrane size may be chosen based on agent size in order to allow the agent being assessed to exit the membrane. For small molecule release, a 3.5-5 kDa cutoff may be used. The Release Rate for a composition may change over time and may be measured in 1 hour increments.

“Representative Microscopy Sample” as used herein describes a sufficient number of fields of view within a cell culture system, a portion of extracted tissue, or an entire extracted organ that the average feature size or number being measured can reasonably be said to represent the average feature size or number if all relevant fields were measured. A Representative Microscopy sample can include measurements within a field of view, which can be measured as cells per a given distance. A Representative Microscopy sample can be used to assess morphology, such as cell-cell contacts, hair follicle architecture, and cellular components (e,g., bundles, synapses).

“SAG” as used herein for a compound means the compound structure identified as CAS 912545-86-9.

“SAG HCl” as used herein for a compound means the compound structure identified as CAS 912545-86-9 as a hydrochloride salt.

The term “sample” refers to a volume or mass obtained, provided, and/or subjected to analysis. In some embodiments, a sample is or comprises a tissue sample, cell sample, a fluid sample, and the like. In some embodiments, a sample is taken from (or is) a subject (e.g., a human or animal subject). In some embodiments, a tissue sample is or comprises brain, hair (including roots), buccal swabs, blood, saliva, semen, muscle, or from any internal organs, or cancer, precancerous, or tumor cells associated with any one of these. A fluid may be, but is not limited to, urine, blood, ascites, pleural fluid, spinal fluid, and the like. A body tissue can include, but is not limited to, brain, skin, muscle, endometrial, uterine, and cervical tissue or cancer, precancerous, or tumor cells associated with any one of these. In an embodiment, a body tissue is brain tissue or a brain tumor or cancer. Those of ordinary skill in the art will appreciate that, in some embodiments, a “sample” is a “primary sample” in that it is obtained from a source (e.g., a subject); in some embodiments, a “sample” is the result of processing of a primary sample, for example to remove certain potentially contaminating components and/or to isolate or purify certain components of interest.

“Self-renewal” refers to the process by which a stem cell divides to generate one (asymmetric division) or two (symmetric division) daughter cells with development potentials that are indistinguishable from those of the mother cell. Self-renewal involves both proliferation and the maintenance of an undifferentiated state.

“siRNA” refers to a double stranded RNA. Optimally, an siRNA is 18, 19, 20, 21, 22, 23 or 24 nucleotides in length and has a 2 base overhang at its 3′ end. These dsRNAs can be introduced to an individual cell or culture system. Such siRNAs are used to downregulate mRNA levels or promoter activity.

“Sonic Hedgehog (Shh) pathway activator” or “Sonic Hedgehog (Shh) activator” refers to a compound or factor that activates the Sonic Hedgehog signaling pathway. Sonic Hedgehog (Shh) pathway activators may be a SMO agonist, Ptch1 inhibitor, SUFU inhibitor, activator of GLI1 transcription, or other components such that the Sonic Hedgehog pathway or its interactions, e.g. VEGF, FLk1, MEK, ERK, Notch/Hes, NANOG, SOX2, MYC, MYCN, are increased.

“Stem cell” refers to a multipotent cell having the capacity to self-renew and to differentiate into multiple cell lineages. “Stem cells of hair follicles” refer to both stem cells found in the dermal papilla of the hair follicle (“dermal papilla stem cells”, “DP stem cells” or “DP cells”) and stem cells found in another region of the hair follicle (“hair follicle stem cells”).

“Stem Cell Differentiation Assay” as used herein is an assay to determine the differentiation capacity of stem cells.

“Stem Cell Assay” as used herein is an assay in which a cell or a cell population are tested for a series of criteria to determine whether the cell or cell population are stem cells or enriched in stem cells or stem cell markers. In a stem cell assay, the cell/cell population are tested for stem cell characteristics such as expression of Stem Cell Markers, and further optionally are tested for stem cell function, including the capacity of self-renewal and differentiation.

“Stem Cell Proliferator” as used herein is a compound or factor that induces an increase in a population of cells which have the capacity for self-renewal and differentiation.

“Stem Cell Proliferation Assay” as used herein is an assay to determine the capacity for agent(s) to induce the creation of stem cells from a starting cell population. The Stem Cell Proliferation Assay using dermal papilla refers to the increase in the number of Dermal Papilla (DP) spheroids, size of DP spheroids, or number of DP cells at the end of the assay compared to the respective number of Dermal Papilla (DP) spheroids, size of DP spheroids, or number of DP cells at the start of the assay. DP cells refer to cells that express Alkaline Phosphatase (AP), and/or Versican, and/or Vimentin, and/or Sox2, and/or CD133. Briefly, microdissection is used to remove intact hair follicles from a specimen. The cells of the hair follicles are treated with cell dissociation enzymes and strained to obtain a single cell suspension. The single cells are then suspended in media with growth factors, plated at a given cell density in wells and incubated at 37° C. Growth is monitored for one or more desired period of time. EVOS® transmitted light images can be taken. Spheroids can be collected for immunoblotting, qPCR, flow cytometry, and/or applied to glass bottom dishes for alkaline phosphatase staining, 5-ethynyl-2′-deoxyuridine (EdU) staining and/or immunofluorescence to detect markers such as Vimentin, Sox2, Versican, CD133, etc. Details of the protocol are provided in the Examples section of the present disclosure.

“Stem Cell Markers” as used herein can be defined as gene products (e.g. protein, RNA, etc) that specifically expressed in stem cells. One type of stem cell marker is gene products that are directly and specifically support the maintenance of stem cell identity. Examples include Gli1, Krt15, CD34, Lgr5, Lgr6, Lrig1, Sox2, CD133, Vimentin, Versican and/or alkaline phosphatase. Additional stem cell markers can be identified using assays that were described in the literature. To determine whether a gene is required for maintenance of stem cell identity, gain-of-function and loss-of-function studies can be used. In gain-of-function studies, over expression of specific gene product (the stem cell marker) would help maintain the stem cell identity. While in loss-of-function studies, removal of the stem cell marker would cause loss of the stem cell identity or induced the differentiation of stem cells. Another type of stem cell marker is gene that only expressed in stem cells but does not necessary to have specific function to maintain the identity of stem cells. This type of markers can be identified by comparing the gene expression signature of sorted stem cells and non-stem cells by assays such as micro-array and qPCR. This type of stem cell marker can be found in the literature. (e.g. Liu Q. et al., Int J Biochem Cell Biol. 2015 60:99-111. www.ncbi.nlm.nih.gov/pubmed/25582750). Potential stem cell markers include Ccdc121, Gdf10, Opcm1, Phex, etc. The expression of stem cell markers such as CD133 or Sox2 in a given cell or cell population can be measure using assays such as qPCR, immunohistochemistry, western blot, and RNA hybridization. The expression of stem cell markers can also be measured using transgenic cells express reporters which can indicate the expression of the given stem cell markers, e.g. Versican-GFP, CD133-GFP or Sox2-GFP. Flow cytometry analysis can then be used to measure the activity of reporter expression. Fluorescence microscopy can also be used to directly visualize the expression of reporters. The expression of stem cell markers may further be determined using microarray analysis for global gene expression profile analysis. The gene expression profile of a given cell population or purified cell population can be compared with the gene expression profile of the stem cell to determine similarity between the 2 cell populations. Stem cell function can be measured by colony forming assay or sphere forming assay, self-renewal assay and differentiation assay. In colony (or sphere) forming assay, when cultured in appropriate culture media, the stem cell should be able to form colonies, on cell culture surface (e.g. cell culture dish) or embedded in cell culture substrate (e.g. Matrigel) or be able to form spheres when cultured in suspension. In colony/sphere forming assay, single stem cells are seeded at low cell density in appropriate culture media and allowed to proliferate for a given period of time (7-10 days). Colony formed are then counted and scored for stem cell marker expression as an indicator of stemness of the original cell. Optionally, the colonies that formed are then picked and passaged to test its self-renewal and differentiation potential. In self-renewal assay, when cultured in appropriate culture media, the cells should maintain stem cell marker (e.g. CD133) expression over at least one (e.g. 1, 2, 3, 4, 5, 10, 20, etc.) cell divisions. In a Stem Cell Differentiation Assay, when cultured in appropriate differentiation media, the cells should be able to generate hair cell which can be identified by hair cell marker expression measured by qPCR, immunostaining, western blot, RNA hybridization or flow cytometry.

“Stemness Driver” as used herein is a composition that induces proliferation of cells of a given cell type, upregulates gene(s) or biomarker(s) in cells, or maintains gene or biomarker expression in cells, while maintaining the potential for self-renewal and the potential to differentiate into cells of a given cell type, for example, a hair follicle epithelial cell. Generally, stemness drivers upregulate at least one biomarker of post-natal stem cells. Stemness Drivers include but are not limited to Wnt agonists and GSK3 inhibitors.

“Subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

“Synergy” or “synergistic effect” is an effect which is greater than the sum of each of the effects taken separately; a greater than additive effect.

“TGF Beta inhibitor” as used herein is a composition that reduces activity of TGF Beta.

“BMP inhibitor” as used herein is a composition that reduces activity of BMP.

“Notch activator” as used herein is a composition that increases Notch pathway activity.

“mTOR” inhibitor” as used herein is a composition that reduces the mechanistic target of rapamycin (mTOR) activity.

“Tissue” is an ensemble of similar cells from the same origin that together carry out a specific function.

“Treating” as used herein in connection with a cell population means delivering a substance to the population to effect an outcome. In the case of in vitro populations, the substance may be directly (or even indirectly) delivered to the population. In the case of in vivo populations, the substance may be delivered by administration to the host subject.

“Wnt activation” as used herein is an activation of the Wnt signaling pathway.

“Wnt agonist” as used herein is any compound, protein, peptide, or agent that activates the Wnt signaling pathway.

The term “alkyl” as used herein refers to a straight or branched saturated hydrocarbon. For example, an alkyl group can have 1 to 8 carbon atoms (i.e., (C₁-C₈) alkyl) or 1 to 6 carbon atoms (i.e., (C₁-C₆ alkyl) or 1 to 4 carbon atoms.

The term “alkenyl” as used herein refers to a linear or branched hydrocarbon radical which includes one or more double bonds and can include divalent radicals, having from 2 to about 15 carbon atoms. Examples of alkenyl groups include but are not limited to, ethenyl, propenyl, butenyl, and higher homologs and isomers.

The term “alkynyl” as used herein refers to a linear or branched hydrocarbon radical which includes one or more triple bonds and can include divalent radicals, having from 2 to about 15 carbon atoms. Examples of alkynyl groups include but are not limited to, ethynyl, propynyl, butynyl, and higher homologs and isomers.

The term “halo” or “halogen” as used herein refers to fluoro, chloro, bromo and iodo.

The term “aryl” as used herein refers to a single all carbon aromatic ring or a multiple condensed all carbon ring system wherein at least one of the rings is aromatic. For example, an aryl group can have 6 to 20 carbon atoms, 6 to 14 carbon atoms, or 6 to 12 carbon atoms. Aryl includes a phenyl radical. Aryl also includes multiple condensed ring systems (e.g., ring systems comprising 2, 3 or 4 rings) having about 9 to 20 carbon atoms in which at least one ring is aromatic and wherein the other rings may be aromatic or not aromatic (i.e., carbocycle). Such multiple condensed ring systems may be optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring.

The term “heteroaryl” as used herein refers to a single aromatic ring that has at least one atom other than carbon in the ring, wherein the atom is selected from the group consisting of oxygen, nitrogen and sulfur; the term also includes multiple condensed ring systems that have at least one such aromatic ring, which multiple condensed ring systems are further described below. Thus, the term includes single aromatic rings of from about 1 to 6 carbon atoms and about 1-4 heteroatoms selected from the group consisting of oxygen, nitrogen and sulfur in the rings. The sulfur and nitrogen atoms may also be present in an oxidized form provided the ring is aromatic. The term also includes multiple condensed ring systems (e.g., ring systems comprising 2, 3 or 4 rings) wherein a heteroaryl group, as defined above, can be condensed with one or more rings selected from heteroaryls (to form for example a naphthyridinyl such as 1,8-naphthyridinyl), heterocycles, (to form for example a 1,2,3,4-tetrahydronaphthyridinyl such as 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system) has about 1-20 carbon atoms and about 1-6 heteroatoms within the heteroaryl ring. Such multiple condensed ring systems may be optionally substituted with one or more (e.g., 1, 2, 3 or 4) oxo groups on the carbocycle or heterocycle portions of the condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the multiple condensed ring system including a heteroaryl, heterocycle, aryl or carbocycle portion of the multiple condensed ring system and at any suitable atom of the multiple condensed ring system including a carbon atom and heteroatom (e.g., a nitrogen).

The term “cycloalkyl” as used herein refers to a saturated or partially saturated ring structure having about 3 to about 8 ring members that has only carbon atoms as ring atoms and can include divalent radicals. Examples of cycloalkyl groups include but are not limited to, cyclopropyl, cyclobutyl, cyclopentyl, cyclohexyl, cyclohexene, cyclopentenyl, cyclohexenyl.

The terms “heterocyclyl” or “heterocyclic” refer to monocyclic or polycyclic 3 to 24-membered rings containing carbon and heteroatoms selected from oxygen, phosphorous, nitrogen, or sulfur and wherein there are no delocalized 7n electrons (aromaticity) shared among the ring carbon or heteroatoms. Heterocyclyl rings include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl. A heterocyclyl or heterocycloalkyl ring can also be fused or bridged, e.g., can be a bicyclic ring.

The use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, l-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid,/toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. For example, inorganic salts include, but are not limited to, ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Example organic bases used in certain embodiments include isopropylamine, diethylamine, ethanolamine, trimethylamine, dicyclohexylamine, choline and caffeine.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compounds or compositions described herein can be formulated in any manner suitable for a desired delivery route, e.g., transtympanic injection, transtympanic wicks and catheters, and injectable depots. Typically, formulations include all physiologically acceptable compositions including derivatives or prodrugs, solvates, stereoisomers, racemates, or tautomers thereof with any physiologically acceptable carriers, diluents, and/or excipients.

### EXAMPLES

### Example 1: Materials and Methods

Stem Cell Proliferation Assay Using Dermal Papilla

Dermal Papilla (DP) proliferation cell culture assay refers to a cell culture method to quantify the number of Dermal Papilla (DP) spheroids, size of DP spheroids, or number of DP cells in cell culture, which is measured at the end of the assay. DP cells refer to cells that express Alkaline Phosphatase (AP), and/or Versican, and/or Vimentin, and/or Sox2, and/or CD133.

The protocol for the Stem Cell Proliferation Assay using dermal papilla is as follows:

Basal media: DMEM/high-glucose medium and F12, supplemented with N2 1×, B27 1×, Streptomycin sulfate (100 μg/ml), Amphotericin B (2.5 μg/ml), and 1 mM Hepes.

Microdissection is used to remove intact hair follicles from a specimen. Intact follicles are incubated for 40 min at 37° C. in TrypLE™ (Thermo Fisher Scientific) with 1 mg/ml collagenase type 1 (sterilized using 0.2 m syringe filter). The treated follicles are then triturated >30× using a 200 μl pipette tip. The volume is adjusted up to 1 ml with 1×PBS. The treated follicles are then passed through a 40 m cell strainer to obtain a single cell suspension.

Twenty μl are applied to a cell counter to determine the number of cells per 1 ml. The remaining cell suspension is spun down at 1,000×g for 5 min at 4° C.

Standard media is prepared supplemented with growth factors (IGF, EGF, and FGF2 at 50 ng/ml). The cells are suspended in standard media with growth factors at 120,000 cells/ml and 250 μl (30,000 cells) are applied to each well of a 24-well plate (Corning Costar, 3534).

Each treatment condition is prepared at 2× concentration, enough for 250 μl/well for a final dilution of 1× in 500 μl. The final DMSO concentration is at about 0.2% for all.

250 μl 2× treatment media is applied to each well to have a final media volume per well of 500 μl. The final treatment concentration per well is IX. The final cell density is 30,000 cells per well.

The cells are incubated at 37° C. and growth is monitored for 10 days.

EVOS® transmitted light images are taken at 0, 5, 7, and 10 days in culture.

Spheroids are collected at 10 days in culture for immunoblotting, qPCR, or flow cytometry. Alternatively, spheroids may be applied to glass bottom dishes coated with poly-1-lysine for alkaline phosphatase (AP) staining, 5-ethynyl-2′-deoxyuridine (EdU) staining, and/or immunofluorescence for DP markers (e.g. Vimentin, Sox2, Versican, CD133) for microscopy.

Alternatively, cells or colonies may be assessed using microscopy to compare DP properties across conditions.

DP Hair Follicle Culture Assay

Dermal Papilla (DP) hair follicle culture assay refers to a method of using intact hair follicles to quantify the number of Dermal Papilla (DP) cells or size of the DP area within the follicle when measured at the end of the assay. DP cells are cells that express alkaline phosphatase (AP), and/or Versican, and/or Vimentin, and/or Sox2, and/or CD133.

The protocol for the DP hair follicle culture assay is as follows:

Basal media: DMEM/high-glucose medium and F12, supplemented with N2 1×, B27 1×, Streptomycin sulfate (100 μg/ml), Amphotericin B (2.5 μg/ml), and 1 mM Hepes.

Microdissection is used to remove intact hair follicles from a specimen. Each hair follicle is trimmed close to the follicle apex and transferred to a fresh dish with HBSS. 200 μl 1×PBS is applied to wells of a 24-well plate. Using a 200 μl pipette cut wide bore, the hair follicles are carefully distributed in wells with 3-5 follicles per well.

The PBS is carefully removed from the wells, leaving the intact follicles, by pipetting. 0.5 ml media supplemented with growth factors (IGF, EGF, and FGF2 at 50 ng/ml) and small molecules as desired are applied to each well.

The hair follicles are incubated at 37° C. and growth monitored over 7 days.

EVOS® transmitted light images are taken at 0 DIV and 7 DIV.

Hair follicles are processed at 7 DIV for alkaline phosphatase (AP) staining, 5-ethynyl-2′-deoxyuridine (EdU staining), and/or immunofluorescence for DP markers (e.g. Vimentin, Sox2, Versican, CD133) for microscopy.

The total area staining positive for DP markers may be analyzed and hair shaft growth may be monitored over time.

### Example 2: Hair Cell Proliferation in Response to Minoxidil Combination Therapies

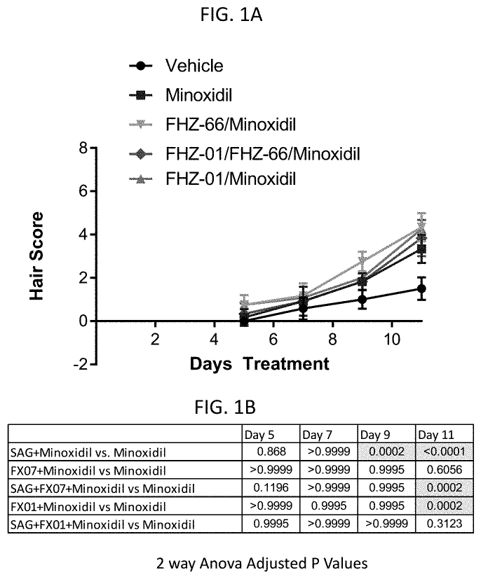

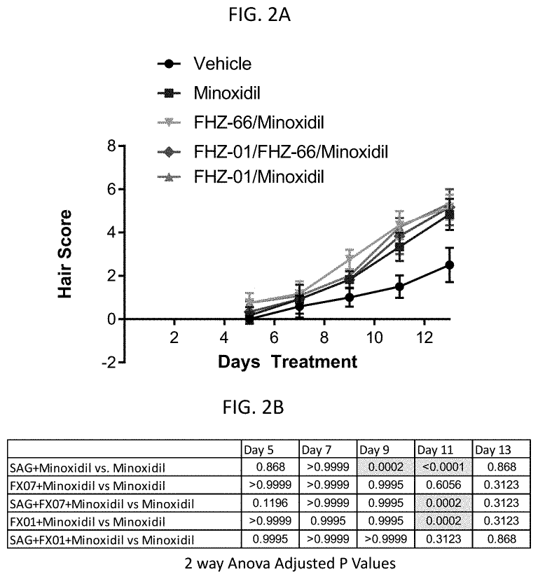

A Stem Cell Proliferation Assay using dermal papilla was carried out as described above in Materials and Methods, and DP colonies were analyzed by light microscopy. DP cells were grown in culture for 13 days in media containing the following agents or combinations of agents: i) vehicle, ii) minoxidil, iii) FHZ-66 (SHH agonist)+minoxidil, iv) FHZ-01 (wnt agonist)+FHZ-66+minoxidil, and v) FHZ-01+minoxidil. Change in hair score for DP cells was assessed at 5, 7, 9, 11, and 13 days post-treatment (FIGS. 1A and 2A). Combination therapies comprising minoxidil showed significantly enhanced hair score values compared to cells treated with minoxidil only or controls cells treated with vehicle. Statistical variance was calculated as 2-way ANOVA P values and are displayed in FIG. 2B.

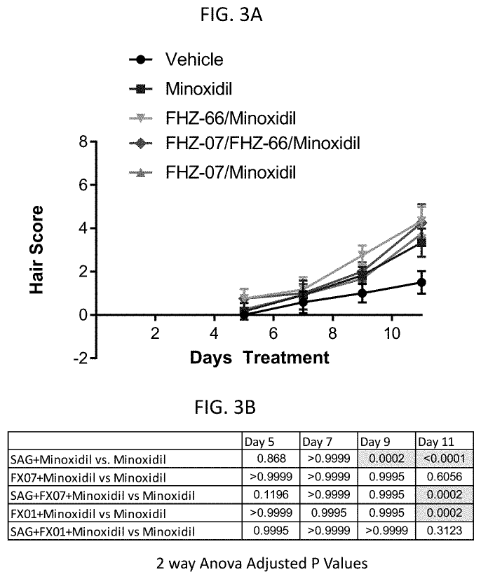

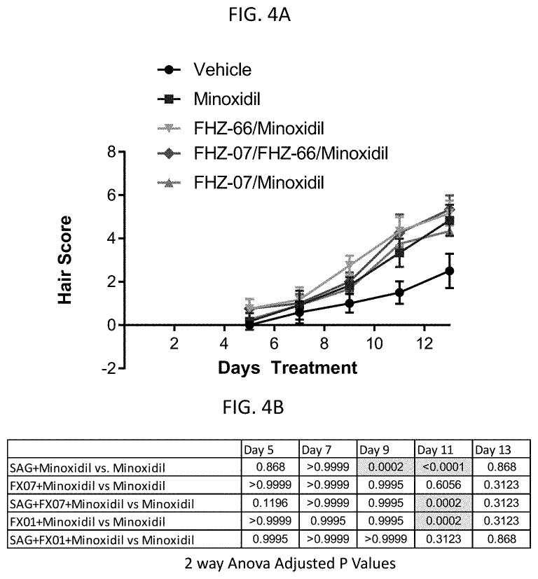

A Stem Cell Proliferation Assay using dermal papilla was carried out as described above in Materials and Methods, and DP colonies were analyzed by light microscopy. DP cells were grown in culture for 13 days in media containing the following agents or combinations of agents: i) vehicle, ii) minoxidil, iii) FHZ-66+minoxidil, iv) FHZ-07 (wnt agonist)+FHZ-66+minoxidil, and v) FHZ-07+minoxidil. Change in hair score for DP cells was assessed at 5, 7, 9, 11, and 13 days post-treatment (FIGS. 3A and 4A). Combination therapies comprising minoxidil showed significantly enhanced hair score values compared to cells treated with minoxidil only or controls cells treated with vehicle. Statistical variance was calculated as 2-way ANOVA P values and are displayed in FIG. 4B.

### REFERENCES ON FORMULATIONS

Ashni Verma, Sukhdev Singh, Rupinder Kaur, Upendra K Jain. Topical Gels as Drug Delivery Systems: A Review. Int. J. Pharm. Sci. Rev. Res., 23(2), 2013, 60, 374-382.
Sonam Vats, Charu Saxena, T S Easwari, V K Shukla. Emulsion Based Gel Technique: Novel Approach for Enhancing Topical Drug Delivery of Hydrophobic Drugs. International Journal for Pharmaceutical Research Scholars (2014), 3(2), 649-660.
Loveleenpreetkaur Prabhjotkaur and M U. Khan. Topical formulations and Hydro-gel: An overview. International Journal of Advances in Pharmacy, Biology and Chemistry (2013), 2(1), 201-206.
Christian Wischke Eckart Rihl Andreas Lendlein. Dermal Drug Delivery by Nanocarriers. Journal of Controlled Release 242 (2016) 1-2.
William Wei Lim Chin, Johannes Parmentier, Michael Widzinski, En Hui Tan, Rajeev Gokhale. A Brief Literature and Patent Review of Nanosuspensions to a Final Drug Product. Journal of Pharmaceutical Sciences, October 2014, Vol. 103(10), pp. 2980-2999.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

From the foregoing description, it will be apparent that variations and modifications may be made to the disclosure described herein to adopt it to various usages and conditions. The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of expanding or generating a population of hair follicle stem cells, comprising contacting the stem cells with minoxidil and one or more Sonic Hedgehog (Shh) pathway activator(s).

2. (canceled)

3. A method of treating a subject who has, or is at risk of developing, alopecia or a disease associated with absence or lack of hair follicle epithelial cells, comprising administering to the subject with minoxidil and one or more Sonic Hedgehog (Shh) pathway activator(s).

4. (canceled)

5. The method of claim 1, further comprising administering one or more Wnt agonists(s).

6. (canceled)

7. The method of claim 1, wherein the stem cells are hair follicle stem cells.

8.-9. (canceled)

10. The method of claim 3, wherein the subject has improved hair growth, improved hair density, and/or improved regenerative cycling of hair follicles compared to a subject not administered the minoxidil and one or more Sonic Hedgehog (Shh) pathway activator(s).

11. The method of claim 1, wherein the minoxidil is at a concentration of about 0.001× to about 10× of an FDA approved minoxidil concentration.

12.-13. (canceled)

14. The method of claim 1, wherein the one or more Shh pathway activator(s) is at a concentration of about 5× to about 1000× of an effective in vitro Shh pathway activation concentration.

15.-16. (canceled)

17. The method of claim 5, wherein the one or more Wnt agonists(s) is at a concentration of about 5× to about 1000× of an effective in vitro Wnt agonist concentration.

18.-19. (canceled)

20. The method of claim 1, wherein the one or more Shh pathway activator(s) is selected from Table 1 or Table 2.

21. The method of claim 1, wherein the one or more Shh pathway activator(s) is selected from purmorphamine, SAG (smoothened agonist), 20-alpha-hydroxy-cholesterol, and SAG HCl.

22.-33. (canceled)

34. The method of claim 5, wherein the one or more Wnt agonists(s) is selected from Table 3.

35. The method of claim 5, wherein the one or more Wnt agonists(s) is a GSK3-alpha inhibitor or a GSK3-beta inhibitor.

36. The method of claim 23, wherein the GSK3-alpha inhibitor is selected from Table 5.

37. The method of claim 23, wherein the GSK3-beta inhibitor is selected from Table 4.

38. The method of claim 5, wherein the one or more Wnt agonists(s) is a compound of Formula I: 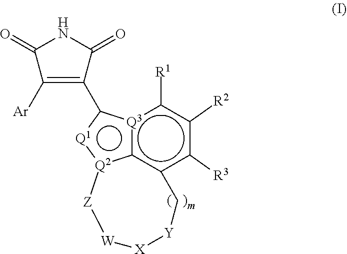
or a pharmaceutically acceptable salt or tautomer thereof, wherein:
Q¹ is CH or N;
Q² is C or N;
Q³ is C or N;
wherein at least one of Q¹, Q², and Q³ is N;
R¹ is selected from the group consisting of hydrogen, halo, C₁-C₄alkyl, C₁-C₄alkenyl, C₁-C₄alkynyl, —CN, —OH, —O—C₁-C₄alkyl, —NH₂, —NHC(O)R^{1a}, and —S(O)₂NH₂; wherein the alkyl is optionally substituted with one to 3 substituents independently selected from the group consisting of halo and —OH; and wherein R^{1a} is C₁-C₄alkyl;
R² is selected from the group consisting of halo, C₁-C₄alkyl, C₁-C₄alkenyl, C₁-C₄alkynyl, —CN, —OH, —O—C₁-C₄alkyl, —NH₂, —NH(C₁-C₄alkyl), —N(C₁-C₄alkyl)₂, —NHC(O)R^{2a}, and —S(O)₂NH₂; wherein the alkyl is optionally substituted with one to 3 substituents independently selected from the group consisting of halo and —OH; and wherein R^{2a} is C₁-C₄alkyl;
R³ is selected from the group consisting of hydrogen, halo, C₁-C₄alkyl, C₁-C₄alkenyl, C₁-C₄alkynyl, —CN, —OH, —O—C₁-C₄alkyl, —NH₂, —NHC(O)R^{3a}, and —S(O)₂NH₂; wherein the alkyl is optionally substituted with one to 3 substituents independently selected from the group consisting of halo and —OH; and wherein R^{3a} is C₁-C₄alkyl;
Ar is selected from the group consisting of 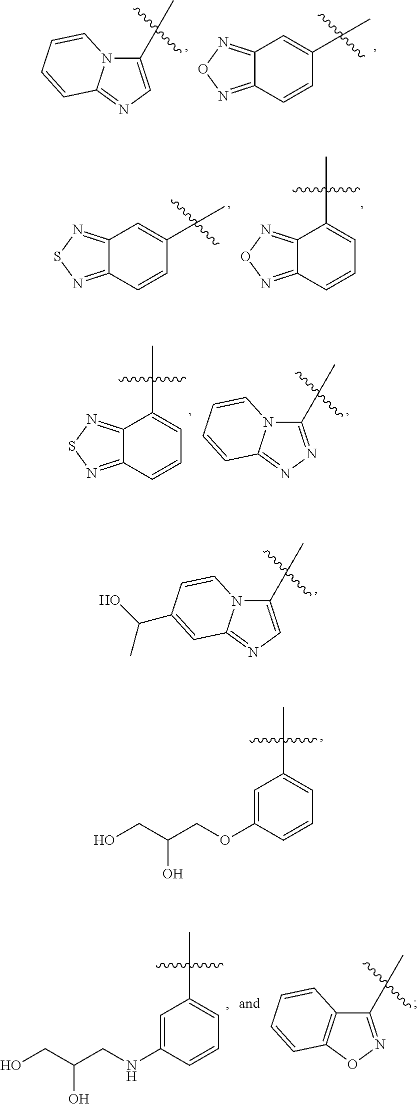
—Z—W—X—Y— is —C(R^{Z})₂—C(R^{W})₂—N(R^{X})—C(R^{Y})₂—, —C(R^{Z})₂—C(R^{W})₂—CH(R^{X})—C(R^{Y})₂—, or —C(R^{W})₂—CH(R^{X})—C(R^{Y})₂—;
each R^{Z} is independently selected from the group consisting of hydrogen, deuterium, halo, and C₁-C₄alkyl, or both R^{Z} groups together form C₃-C₆cycloalkyl or oxo;
each R^{W} is independently selected from the group consisting of hydrogen, deuterium, halo, and C₁-C₄alkyl, or both R^{W} groups together form C₃-C₆cycloalkyl or oxo;
or R^{Z} and R^{W} together with the carbons to which they are attached form a C₃-C₆cycloalkyl;
R^{X} is selected from the group consisting of —COR^{X1}, —SO₂R^{X1}, heteroaryl, and —(C₁-C₄alkylene)-(C₃-C₈cycloalkyl), and wherein the —(C₁-C₄alkylene)-(C₃-C₈cycloalkyl) is optionally substituted with one to four halo on the C₁-C₄alkylene;
wherein R^{X1} is heterocyclic, wherein the heterocyclic is optionally substituted with one to twelve substituents independently selected from the group consisting of deuterium, halo, —[C(R^{X1a})₂]ₚ—CN, —CF₃, C₁-C₄alkyl, —(CH₂)ₚ—OH, —[C(R^{X1a})₂]ₚ—OH, —[C(R^{X1a})₂]ₚ—O—C₁-C₄alkyl, —NHCOC₁-C₄alkyl, —CONHC₁-C₄alkyl, —COH, —CO₂H, —[C(R^{X1a})₂]ₚ—COO—C₁-C₄alkyl, —(CH₂)ₚ—NH₂, —[C(R^{X1a})₂]ₚ—NH₂, —[C(R^{X1a})₂]ₚ—NH—C₁-C₄alkyl, —[C(R^{X1a})₂]ₚ—N—(C₁-C₄alkyl)₂; wherein p is 0, 1, 2, or 3; wherein each R^{X1a} is independently selected from the group consisting of hydrogen, deuterium, halo, and C₁-C₄alkyl, or both R^{X1a} groups together form C₃-C₆cycloalkyl;
each R^{Y} is independently selected from the group consisting of hydrogen, deuterium, halo, and C₁-C₄alkyl, or both R^{R} groups together form C₃-C₆cycloalkyl or oxo; and
m is 0, 1, or 2;
provided that the compound is not

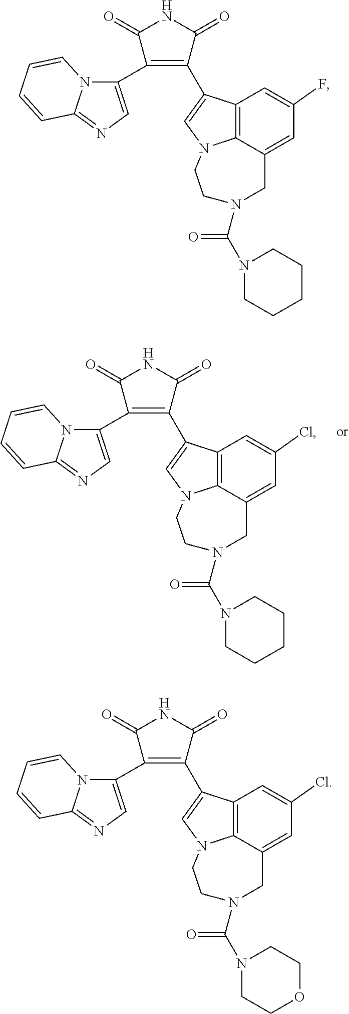 [SMILES: O=C1NC(=O)C(c2cnc3ccccn23)=C1c1cn2c3c(cc(F)cc13)CN(C(=O)N1CCCCC1)CC2 ; O=C1NC(=O)C(c2cnc3ccccn23)=C1c1cn2c3c(cc(Cl)cc13)CN(C(=O)N1CCCCC1)CC2 ; O=C1NC(=O)C(c2cnc3ccccn23)=C1c1cn2c3c(cc(Cl)cc13)CN(C(=O)N1CCOCC1)CC2]

39.-75. (canceled)

76. The method of any one of claim 5, wherein the one or more Wnt agonists(s) is selected from Table 6.

77. The method of claim 5, wherein the one or more Wnt agonists(s) is selected from CHIR99021, LY2090314, AZD1080, GSK3 inhibitor XXII, Compound I-6, Compound I-7, and Compound I-12.

78.-162. (canceled)

163. The method of claim 1, wherein the expression of Gli1, Krt15, CD34, Lgr5, Lgr6, Lrig1, Sox2, CD133, vimentin, versican and/or alkaline phosphatase is increased in hair follicles.

164. A pharmaceutical composition comprising: a pharmaceutically acceptable carrier and (i) minoxidil or a pharmaceutically-acceptable salt thereof, and (ii) a Sonic Hedgehog (Shh) pathway activator or a pharmaceutically-acceptable salt thereof.

165.-323. (canceled)
